(19) United States
(12) Patent Application Publication
(10) Pub. No.: US 2007/0061084 A1
(43) Pub. Date: Mar. 15, 2007

(54) Method, system, and knowledge repository for identifying a secondary metabolite from a microorganism

(75) Inventors: Chris M. Farnet, Montreal (CA); James B. McAlpine, Montreal (CA); Brian O. Bachmann, Nashville, TN (US); Alfredo Staffa, Saint-Laurent (CA); Emmanuel Zazopoulos, Montreal (CA); Zhizi Zhao, Montreal (CA); Sai Man Wong, Saint-Laurent (CA); Nicolas Desjardins, Pointe-Claire (CA)

Correspondence Address: PALMER & DODGE, LLP;KATHLEEN M. WILLIAMS, 111 HUNTINGTON AVENUE, BOSTON, MA, 02199, US

(73) Assignee: Ecopia BioSciences, Inc.

(21) Appl. No.: 11/262,235

(22) Filed: Oct. 28, 2005

Related U.S. Application Data
(63) Continuation-in-part of application No. 10/350,341, filed on Jan. 24, 2003.
(60) Provisional application No. 60/623,642, filed on Oct. 29, 2004.
(60) Provisional application No. 60/350,369, filed on Jan. 24, 2002.
(60) Provisional application No. 60/398,795, filed on Jul. 29, 2002.
(60) Provisional application No. 60/412,580, filed on Sep. 23, 2002.

(51) Int. Cl. C12Q 1/00; C12Q 1/68; G06F 19/00

(52) U.S. Cl. 702/19; 435/4; 435/6; 702/20

(57) ABSTRACT

Described are methods and systems for identifying a secondary metabolite synthesized by a target gene cluster within a microorganism. A putative or confirmed function is attributed to a gene within the gene cluster, and an extract from the microorganism is obtained which is suspected to contain the secondary metabolite synthesized by the gene cluster. The extract is then assessed for chemical, physical or biological properties, and the metabolite is identified and optionally isolated. Further, the invention provides a knowledge repository in which gene cluster information is linked to secondary metabolite production data. The invention further relates to a graphical user interface for accessing the knowledge repository, and a memory for storing data, having a data structure that is stored in the memory.

### RELATED APPLICATIONS

This application claims the benefit of U.S. Provisional Application No. 60/623,642, filed Oct. 29, 2004, and is a continuation-in-part of application U.S. Ser. No. 10/350,341 filed Jan. 24, 2003, which claims the benefit of U.S. Provisional Application No. 60/350,369, filed Jan. 24, 2002, U.S. Provisional Application No. 60/398,795, filed Jul. 29, 2002, and U.S. Provisional Application No. 60/412,580, filed Sep. 23, 2002. The teachings of the above applications are incorporated by reference in their entireties herein.

### FIELD OF THE INVENTION

The present invention relates generally to a bioinformatics method and system for identifying products of secondary metabolism in a microorganism.

### BACKGROUND OF THE INVENTION

Natural product metabolites are widely used as bioactive compounds, dyes, plasticizers, surfactants, scents, flavorings, drugs, herbicides, pesticides and lead compounds for such applications. Improvements in methods of discovery of natural product metabolites would be of benefit to many fields. One field of natural products in which there is an urgent need for improved discovery methods is natural product drug development. While the rate of discovery of new antibiotics has dropped significantly over the past few decades, analysis of antibiotic discovery rates suggests that a large number of antibiotics remain to be discovered from actinomycete natural product metabolites (Watve et al., (2001) Arch. Microbiology 176:386-390).

Recent genome sequencing studies demonstrate that the ability of actinomycetes to produce bioactive secondary metabolites has been vastly underestimated. For example, 25 secondary metabolite gene clusters were identified in the genome of Streptomyces avermitilis by whole genome shotgun sequencing despite the fact that the organism had previously been reported to produce only two natural products (Omura et al. Proc. Natl. Acad. Sci. USA, 98, 12215-12220). Likewise a genome project of Streptomyces coelicolor demonstrated that the S. coelicolor genome contains biosynthetic gene clusters for 12 or more natural products while the organism was previously known to product three or four natural products (Bentley, S. D. et al., Nature, 147, 141-147 (2002)). Conventional methods to obtain the metabolite of a target gene cluster focus on expression of the open readable frames (ORFs) to produce the gene products or proteins and enzymes forming the biosynthetic pathway to the metabolite, and to study the roles of the proteins in the biosynthesis of the final metabolite.

There is a continuing need for improved methods to discover natural product metabolites and genomic analysis of microorganisms provides a basis for the discovery of microbial secondary product metabolites. High-throughput screening methods have been developed for the purpose of small molecule discovery for new drug candidates. The conventional high-throughput screening methods rely on trial-and-error methodologies, and there is a great deal of wasted effort in screening compounds without conducting pre-selection processes. Also, although there is a great deal of genomic information available and there continues to be more sequencing efforts undertaken, there is dearth of information linking genomic information to products of secondary metabolism. Where drug discovery efforts involve genomic analysis, such discovery methods often require time consuming and laborious steps required to identify the structure of the target metabolite. It is desirable to provide a method and system for identifying metabolic products from microorganisms that can be conducted on a high-throughput basis, and allows a high level of predictability based on genomic information.

### SUMMARY OF THE INVENTION

It is an object of the present invention to obviate or mitigate at least one disadvantage of the prior art. It is discovered that a direct link between genomic information and the end metabolite synthesized by a target gene cluster is possible and that this direct link greatly accelerates the process of obtaining the end metabolite by reducing time consuming steps involving transcriptome analysis, i.e. analysis of RNA expression or gene product expression of the open reading frames forming a biosynthetic gene cluster. Advantageously, the methods and compositions of invention provide a direct association between genomic information relating to at least one region of a gene in a gene cluster and the natural product synthesized by all genes in the gene cluster. The direct link may relate to the structure of the natural product or to a characteristic and measurable biological activity of the natural product synthesized by the gene cluster. The direct association may relate to a portion of a single gene product in the gene cluster to the natural product produced by the multiple gene products forming the biosynthetic locus.

In certain embodiments of the invention, one or more of the following are realized. The method and computer readable medium include a predictive aspect derived from previously obtained data. This allows the invention to traverse the “trial-and-error” style repetition normally associated with high throughput applications. Further, the invention advantageously incorporates knowledge of a microorganism's response to varying culture conditions (ingredients, temperature, osmotic pressure, etc), which allows prediction of conditions that may induce expression of a cryptic pathway. Feedback of secondary metabolite information to the knowledge repository gives the system efficiency, and increases the predictive power of the invention. In certain embodiments, linking of genetic capacity of a microorganism to produce a secondary metabolite of a particular chemical family lends efficiency if a compound of a specific chemical family is sought in the discovery process.

In one aspect, the invention provides a method of identifying a natural product synthesized by gene products encoded by a target gene cluster, said method comprising the steps of: (a) providing a microorganism containing the target gene; (b) ascribing a direct association between genomic information relating to at least one region of a gene in the gene cluster and the structure of the natural product synthesized by the gene products encoded by the target gene cluster; (c) culturing said microorganism under conditions conducive to expression of the target gene cluster to yield at least one extract containing the natural product synthesized by the gene products encoded by the target gene cluster; (d) measuring chemical, physical or biological properties of metabolites in the extract obtained at the end of step c); and (e) applying genomics-guided methods to identify from metabolites of step d) the natural product by comparing the chemical, physical or biological properties measured in step d) with the expected chemical, physical or biological properties of the natural product based on the direct association between the genomic information and the natural product, said genomics-guided methods being independent of detection or analysis of the gene products encoded by said target gene cluster. In one embodiment of this aspect, step c) involves growing the microorganism under multiple culture conditions to achieve expression of the target gene cluster and obtaining an extract of the fermentation broth produced under at least some of the culture conditions, and step d) involves measuring chemical, physical or biological properties of the metabolites of at least some of the extracts. In another embodiment of this aspect, step e) further comprises the step of comparing the chemical, physical or biological properties measured in step d) with the chemical, physical or biological properties of known compounds. In another embodiment of this aspect, step a) involves selecting a microorganism by reference to a computer-readable medium with computer-readable code stored therein coding genomic data indicating the presence of a target gene cluster in a microorganism. In another embodiment of this aspect, step c) involves growing the microorganism under multiple culture conditions selected by reference to a computer readable medium with computer readable code coding for secondary metabolite production data providing culture conditions under which the product of at least one secondary metabolic gene cluster is expressed. In another embodiment of this aspect, step e) is under computer control with computer readable medium with computer readable code coding for information pertaining to metabolites synthesized by secondary metabolic gene clusters. In another embodiment of this aspect, step d) involves measuring one or more properties selected from the group consisting of molecular mass, UV spectrum and bioactivity. In another embodiment the gene cluster is endogenous to the microorganism cultured and step d) involves homologous expression of the target gene cluster. In another embodiment, the method includes a step of testing the secondary metabolite produced by the target gene cluster for biological activity, in particular antimicrobial, antifungal or anticancer activity. In another embodiment of this aspect, the method further involves the step of adding to a computer-readable medium as computer-readable code information pertaining to the association between the secondary metabolite and the target cluster; the chemical, physical or biological properties of the secondary metabolite; and the conditions under which the microorganism produces the secondary metabolite.

In a further aspect, the invention provides a method of identifying a natural product of a pre-selected chemical family from a microorganism, said method comprising the steps of: (a) ascribing a direct association between genomic information relating to at least one region of a gene in a target gene cluster and the structure of a natural product of the pre-selected chemical family synthesized by the gene products encoded by the target gene cluster; (b) selecting a microorganism containing the target gene cluster; (c) culturing the microorganism under conditions conducive to expression of the target gene cluster to yield at least one extract containing the natural product synthesized by the target gene cluster; (d) measuring chemical, physical or biological properties of metabolites in the extract obtained at the end of step c); and (e) applying genomic-guided methods to identify from the metabolites of step d) the natural product of the pre-selected chemical family by comparing the chemical, physical or biological properties measured in step d) with the expected chemical, physical or biological properties of the natural product based on the direct association between the genomic information and the natural product, said genomic-guided methods being independent of detection or analysis of gene products encoded by said target gene cluster.

In a further aspect, the invention provides a system for identifying a natural product synthesized by gene products encoded by a target gene cluster, said system comprising: (a) genomic data indicating the presence of target gene cluster within a microorganism, wherein a direct association between genomic information relating to at least one region of a gene cluster and the structure of the natural product synthesized by the gene products encoded by the target gene cluster is ascribed; b) extraction means for obtaining an extract derived from the microorganism, said extract containing metabolites comprising the natural product synthesized by the gene products encoded by the target gene cluster; c) an analyser for measuring chemical, physical or biological properties of metabolites in the extract; and d) a comparator for identifying from the metabolites contained in the extract the natural product synthesized by the gene products encoded by the target gene cluster by comparing the chemical, physical or biological properties measured by the analyser with the expected chemical, physical or biological properties of the natural product based on the direct association between the genomic information and the natural product. In a further embodiment of this aspect, the invention provides a system for identifying a natural product from a pre-selected chemical family, the system comprising: (a) genomic data relating to a target gene cluster encoding gene products responsible for the biosynthesis of said natural product, wherein a direct association between genomic information relating to at least one region of a gene in the gene cluster and the structure of the natural product synthesized by the gene products encoded by the target gene cluster is ascribed; (b) a selector for selecting a microorganism containing the target gene cluster; (c) extraction means for obtaining from the microorganism an extract containing metabolites comprising the natural product synthesized by the gene products encoded by the target gene cluster; (c) an analyser for measuring chemical, physical or biological properties of the metabolites in the extract; and (d) a comparator for identifying from the metabolites analysed by the analyser the natural product from the pre-selected chemical family by comparing the chemical, physical or biological properties of the secondary metabolite with the expected chemical, physical or biological properties of the natural product based on the direct association between the genomic information and the structure of the natural product.

In a further aspect, methods are provided for determining the presence of a secondary metabolite. The methods involve, for example, first predicting a chemical property of a candidate secondary metabolite. The prediction is performed on the basis of a comparison of a target gene cluster of the microorganism with information in a database comprising genomic information from a plurality of microorganisms. The target gene cluster encodes genetic information for the generation of the candidate secondary metabolite. Following the prediction, an extract of a fermentation broth in which the microorganism has been cultured is subjected to detection for the presence of a secondary metabolite that exhibits the chemical property predicted in the first step. Subsequently, the secondary metabolite exhibiting the detected chemical property is isolated from an extract of a fermentation broth in which the microorganism has been cultured. At least one step in the process of isolating is guided by information regarding the isolation of a compound exhibiting the chemical property. In alternative embodiments, the microorganism is cultured under conditions predicted, on the basis, for example, of a database query, to be conducive for the production of a secondary metabolite exhibiting that chemical property.

In another aspect, a method is provided for identifying a microorganism that produces a secondary metabolite belonging to a selected chemical family of compounds. Such method involves: (a) selecting a chemical property that is common to compounds in the selected chemical family; b) identifying in a microorganism known to produce a compound belonging to the selected chemical family, a gene cluster that participates in generating the chemical property of a known secondary metabolite; c) querying a database comprising genomic information with one or more sequences comprised by the first gene cluster in order to identify a candidate microorganism (can be the same or, preferably a different organism) that harbors a gene cluster that is similar to the first identified gene cluster; d) detecting in an extract of a fermentation broth in which the candidate microorganism has been cultured the presence of a compound that exhibits the chemical property. In this manner, the presence of a compound that exhibits the chemical property identifies the candidate microorganism as one that produces a secondary metabolite belonging to the selected chemical family of compounds. In other aspects, the secondary metabolite is isolated and analyzed for structure and useful biological activities.

Also described herein is a computer-readable medium with computer readable code for implementation of the methods and systems of the invention. The computer readable medium is sometimes referred to herein as a “knowledge repository”. Accordingly, in a further aspect, the invention provides a method of building a computer-readable medium with computer-readable code stored therein coding for secondary metabolism data from a microorganism for identifying a natural product synthesized by gene products encoded by a target gene cluster, said method comprising the steps of: (a) assembling on said computer-readable medium, as computer-readable code, genomic data confirming the presence of a target gene cluster within a microorganism, wherein a direct association between genomic information related to at least one region of a gene in the gene cluster and the structure of the natural product synthesized by the gene products encoded by a target gene cluster is ascribed; (b) inputting on said computer-readable medium as computer-readable code extract-characterizing data providing chemical, physical or biological properties of metabolites observed in an extract derived from the microorganism, wherein said metabolites include the natural product synthesized by the gene products encoded by the target gene cluster; and (c) comparing the extract-characterizing data with comparative data representing expected chemical, physical or biological properties of the natural product synthesized by the gene products encoded by the target gene cluster, so as to identify from the metabolites in the extract the natural product based on the direct association between the genomic information and the natural product; and (d) retaining the result of step c) by linking the natural product identified in the comparing step with the genomic data assembled in the assembling step in said computer-readable medium as computer-readable code. In another embodiment, the invention provides a method of building a computer-readable medium with computer-readable code stored therein coding for secondary metabolism data from a microorganism, said medium useful for predicting natural product production from a target gene cluster based on genomic data, said method comprising: (a) assembling on said computer-readable medium as computer-readable code genomic data confirming the presence of a target gene cluster endogenous to a microorganism, wherein a direct association between genomic information relating to at least one region of a gene within the gene cluster and the structure of the natural product synthesized by the gene products encoded by the target gene cluster is ascribed; (b) extracting a sample of fermentation broth from a culture of said microorganism, thereby forming an extract; (c) screening the extract for extract-characterizing data indicative of the presence or absence of the natural product attributable to the target gene cluster based on a pre-selected chemical, physical or biological property; (d) entering on said computer-readable medium as computer-readable code the extract-characterizing data; (e) comparing the extract-characterizing data with comparative data representing expected chemical, physical or biological properties of the natural product synthesized by the gene products encoded by the target gene cluster, so as to identify from the extract the natural product, wherein said expected chemical, physical or biological properties are based on the direct association between the genomic information and the natural product; (f) determining the identity of the natural product; and (g) affirming within the computer-readable medium a correspondence between the genomic data, the pre-selected chemical, physical or biological property, and the identity of the natural product, allowing a cycle of prediction of secondary metabolite production based on the genomic data.

Throughout the specification, the computer-readable medium with computer readable code is sometimes referred to as a “knowledge repository”. In another embodiment of this aspect, the knowledge repository additionally comprising culture conditions data linked to the extract characterizing data, the culture conditions data identifying culture conditions under which a set of extract characterizing data are obtained. In another embodiment of this aspect, the comparative data in the knowledge repository comprises a known compound library holding data characterizing a chemical, physical, or biological property of a plurality of known compounds for comparison with the extract characterizing data. In another embodiment of this aspect, a prediction link is made between a record within the genomic data and a record in the comparative data when a match is established between a secondary metabolite attributable to the target gene cluster within the extract characterizing data and the comparative data. In another embodiment of this aspect, the extract characterizing data of the knowledge repository comprises the biological property of antimicrobial, antifungal or anticancer activity. In another embodiment of this aspect, the knowledge repository of additionally comprising chemical family data linked to the genomic data assigning a chemical family to genomic data indicative of a putative or confirmed function in secondary metabolic pathways leading to synthesis of a member of the chemical family.

In a further aspect, the invention provides a memory for storing secondary metabolism data for access by an application program being executed on a data processing system for identifying a secondary metabolite synthesized by a target gene cluster contained within the genome of a microorganism, said memory comprising: a data structure stored in said memory, the data structure including information resident in a database used by said application program and including: genomic data confirming the presence of a target gene cluster within a microorganism, wherein putative or confirmed function has been attributed to at least one region of a gene in the gene cluster; extract characterizing data providing chemical, physical or biological properties of metabolites contained in an extract derived from the microorganism, wherein said metabolites include a secondary metabolite attributable to the target gene cluster; and comparative data representing expected chemical physical or biological properties of the secondary metabolite synthesized by the target gene cluster, said extract characterizing data being comparable with the comparative data for identifying the metabolites in an extract containing the secondary metabolite synthesized by the target gene cluster based on the putative or confirmed function attributed to said at least one region of a gene in a gene cluster. In a related aspect, the invention provides a computer-readable medium with computer-readable code stored therein coding for secondary metabolism data for access by an application program executed on a data processing system for identifying a natural product synthesized by gene products encoded by a target gene cluster endogenous to a microorganism, said computer-readable medium comprising a data structure as computer-readable code, said data structure including information resident in a database used by said application program and including (i) genomic data relating to the target gene cluster within the microorganism, wherein a direct association between genomic information relating to at least one region of a gene in the gene cluster and the natural product synthesized by the gene products encoded by the gene cluster is ascribed; (ii) extract-characterizing data providing chemical, physical or biological properties of metabolites contained in an extract derived from the microorganism, wherein said metabolites include the natural product; and (iii) comparative data representing expected chemical, physical or biological properties of the natural product based on the direct association between the genomic information the natural product, said extract-characterizing data being comparable with the comparative data for identifying the natural product. In another related aspect, the invention provides a computer-readable medium storing computer-executable instructions for performing the steps of: (a) comparing: (i) comparative data representing expected chemical physical or biological properties of a natural product synthesized by gene products encoded by a target gene cluster endogenous to a microorganism, said expected properties being based on a direct association between genomic information relating to at least one region of a gene within the gene cluster and the natural product; with (ii) extract-characterizing data providing chemical, physical or biological properties of metabolites measured in an extract derived from a culture of a microorganism harbouring the target gene cluster endogenous to the microorganism wherein said metabolites include the natural product synthesized by the gene products encoded by the target gene cluster; and (b) storing, as computer-readable code, results of comparing said comparative data with said extract-characterizing data.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached figures.

FIG. 1a is a schematic illustration of a general method and system for identifying secondary metabolites according to one embodiment of the invention. FIGS. 1b, 1c, 1d, 1e, 1f and 1g illustrate the general method and systems of the FIG. 1a as described in examples 1, 2, 3, 4, 5, and 6 respectively.

FIG. 2 is a schematic illustration of a genomics-guided expression means to obtain, from a microorganism, extracts containing secondary metabolites and a genomics-guided screening technology to measure biological properties of the metabolites according to one embodiment of the invention.

FIG. 3 illustrates a high-throughput CHUMB method to obtain chemical, physical and biological properties of metabolites used in one embodiment of the invention.

FIG. 4 is a schematic illustration of a representative genomics-guided expression and screening technology to identify a metabolite according to one embodiment of the invention.

FIG. 5 is a schematic illustration of a representative genomics-guided extraction technology to isolate a metabolite according to one embodiment of the invention.

FIGS. 6, 7 and 8 are schematic illustration of a representative genomics-guided three-stage extraction/isolation/structure-elucidation protocol according to one embodiment of the invention; wherein Stage I of the protocol is shown in FIG. 6, Stage II of the protocol is shown generally in FIG. 7 (one example of the Stage II protocol of FIG. 7 is also shown in FIG. 6), and Stage II of the protocol is shown in FIG. 8.

FIG. 9 illustrates a schematic representation of a system for identifying a secondary metabolite synthesized by a target gene cluster.

FIG. 10 illustrates a schematic representation of a system for identifying a secondary metabolite from a pre-selected chemical family.

FIG. 11 illustrates a schematic representation of a typical graphical user interface according to the invention.

FIGS. 12a and 12b illustrate the results of a biochemical induction assay to detect enediyne metabolites based on their ability to damage DNA wherein, in FIG. 12a, CALI is calicheamicin, MACR is macromomycin, DYNE is dynemicin, and NEOC is neocarzinostatin, and in FIG. 12b, 007A is the putative enediyne from Amycolatopsis orientalis, 009C is the putative enediyne from Streptomyces ghanaensis, 145B is the putative enediyne from Streptomyces citricolor, and 046E and 171B are putative enediynes from the microorganisms in Ecopia's private culture collection.

FIG. 13 illustrates a graphical depiction of the 024A locus, a putative lipopeptide biosynthetic locus from Streptomyces refuineus, showing at the top of the figure, a scale in base pairs, followed by the coverage of the 024A locus in a single contiguous DNA sequence, the relative position and orientation of the 16 open reading frames (ORFs) forming the locus, indicating in black the unusual C-domain in the NRPS system (ORF 4) of the 024A locus, and finally the structural similarities between the lipopeptide synthesized by 024A (024A compound) and the known lipopeptide A54145 produced by Streptomyces fradiae.

FIGS. 14a and 14b are photographs of plates generated during extraction of an anionic lipopeptide from Streptomyces fradiae, and Streptomyces refuineus NRRL 3143 respectively, both showing an enrichment of activity based on IRA67 anion exchange chromatography consistent with expression of an acidic lipopeptide.

FIG. 15 illustrates analysis of a gene cluster designated 023D by the system of the invention, showing 35 ORFs and corresponding protein families, each protein family indicated by the four-letter designation. FIG. 15 further illustrates an automated domain string capturing the structure of the polyketide backbone in a line notation, and translation of the domain string into a predicted chemical structure.

FIG. 16 illustrates three predicted substructure elements of the metabolite of interest providing a direct association between genomic data and the end metabolite, which substructure elements may be used in genomics-guided methods for isolation of the metabolite.

FIG. 17 illustrates the predicted structure of a natural product metabolite.

FIG. 18 shows a display of chemical, physical and biological properties of metabolites in extracts obtained by growing the organism under multiple media conditions.

FIG. 19 illustrates the final structure of the metabolite of Example 5 as confirmed by multidimensional NMR spectroscopy.

FIG. 20 illustrates a graphical depiction of gene cluster showing ORFs ranging in size from over 100 bp to several kbp. FIG. 20b represents the predicted structure from the PKSs domain arrangements. FIG. 20c represents the determination of individual domains in multi-modular enzymatic systems and evaluation of active site integrity and specificity. FIG. 20d represents the classification of gene clusters based on present enzymatic activities (of gene families) allowing the determination of related gene clusters in the DECIPHER® database.

FIG. 21 illustrates the predicted chemical structure of the polyene polyketide of Example 7. FIG. 21a represents the determination of the enzymes involved in the synthesis of an aminohydroxycyclopentenone unit and its attachment onto the polyketide core structure. FIG. 21b represents the analysis of polyketide synthase system which predicts the synthesis of a defined linear polyketide scaffold loaded with an unusual 4-guanidino-butyrylCoA starter unit. FIG. 21c represents the determination of the enzymes involved in the synthesis of a glucuronic acid moiety and its attachment onto the polyketide core structure and the presence of a specific N-methyltranferase that adds a methyl group onto the secondary amino nitrogen of the guanidino moiety.

FIG. 22 illustrates a computer-generated prediction of the structure of the compound of Example 7 produced by gene cluster 007C. The boxed portions indicate examples of substructure elements that can be used to search against a database of known natural products.

FIG. 23 illustrates computer assisted analysis of fermentation broths. FIG. 23a is a representation of the fractionated methanolic extracts, by HPLC and fraction analysis using polarity determination, mass spectroscopy, UV absorption. FIG. 23b represents bioactivity screening of fractions against Micrococcus luteus (first band), Staphylococcus aureus (second band) and Enterococcus faecalis (third band). FIG. 23c represents the physicochemical properties of the metabolite with (m/z)+ of 838 and UV absorption chromophore, which was expected from the predicted structure.

FIG. 24a illustrates the predicted structure of the polyene polyketide and FIG. 24b represents its confirmation by multidimentional NMR experiments, which confirmed the predicted structure.

FIG. 25 shows the biosynthetic locus producing Compound 1 (0506) in Amycolatopsis orientalis, showing a scale in base pairs units; the position of the two sequence of contiguous nucleic acids of SEQ ID NOs: 1, and 14; the position and orientation of the 12 open reading frames of the biosynthetic locus identified by ORF number; and the coverage of the biosynthetic locus by cosmids 007KA and 007KU respectively having deposit accession nos: IDAC 250505-01 and IDAC 250505-02.

FIGS. 26 to 29 show clustal alignments respectively of condensation, adenylation, thiolation, and epimerization domains of the NRPS of SEQ ID NO: 10 with various domains from known NRPS. In each of the clustal alignments: (i) a line above the alignment is used to mark NRPS conserved motifs; (ii) an asterisk “*” indicates positions which have a single, fully conserved residues; (iii) a colon “:” indicates that one of the following strong groups is fully conserved in a specific position: (S, T or A); (N, E, Q or K); (N, H, Q or K); (N, D, E or Q); (Q, H, R or K); (M, I, L or V); (M, I, L or F); (H or Y); and (F, Y or W); and (iv) a period “.” indicates that one of the following weaker groups is fully conserved: (C, S or A); (A, T or V); (S, A or G); (S, T, N or K); (S, T, P or A); (S, G, N or D); (S, N, D, E, Q or K); (N, D, E, Q, H or K); (N, E, Q, H, R or K); (F, V, L, I or M): and (H, F or Y). The number at the end of each line indicates the position of the last amino acid of the line within the specific domain.

FIG. 26(a and b) show an amino acid alignment comparing the 6 condensation (C) domains present in modules 1 to 6 (CD1-CD6) of the non-ribosomal peptide synthetase (NRPS) system (amino acids 3-421 of SEQ ID NO: 10; amino acids 1043-1471 of SEQ ID NO: 10; amino acids 2466-2906 of SEQ ID NO: 10; amino acids 3922-4362 of SEQ ID NO: 10; amino acids 4924-5363 of SEQ ID NO: 10; and amino acids 5936-6362 of SEQ ID NO: 10) and the condensation domain of gramicidin (SEQ ID NO 28, taken from GrsB, GenBank CAA43838). The boundaries and conserved motifs (C1-C7) of the C domains were chosen as described by Konz et al. (1999), Chemistry and Biology, Vol. 6, R39-R48 and indicated in grey.

FIG. 27(a to d) show an amino acid alignment comparing the 6 adenylation (A) domains present in modules 1 to 6 (AD1-AD6) of the non-ribosomal peptide synthetase (NRPS) system (amino acids 454-946 of SEQ ID NO: 10; amino acids 1495-1981 of SEQ ID NO: 10; amino acids 2965-3836 of SEQ ID NO: 10; amino acids 4387-4840 of SEQ ID NO: 10; amino acids 5388-5837 of SEQ ID NO: 10; and amino acids 6387-6883 of SEQ ID NO: 10) and the adenylation domain of gramicidin (SEQ ID NO: 29, taken from GrsA, GenBank AAA58718). The boundaries and conserved motifs (A1-A10) of A domain were chosen as described by Konz et al. (1999), Chemistry and Biology, Vol. 6, R39-R48 and indicated in grey. Specificity-confering codes (positions 235, 236, 239, 278, 299, 301, 322, 330, of GrsA according to Stachelaus et al. (1999), Chemistry & Biology, vol. 6, 493-505) of the adenylation domains are highlighted in black. The A domain of module 3, which comprises an N-methylation (M) domain, is further compared with the N-Methylation-Adenylation domain (ADME) of Complestatin (SEQ ID NO:30, taken from ComC, GenBank AAK81826). The boundaries and conserved motifs (MI, and motifs II/Y, IV and V) of M domains were chosen as described by Hacker et al. (2000), J. Biol. Chem., vol 275, no 40, 30826-30832.

FIG. 28 shows an amino acid alignment comparing the 6 thiolation (T) domains present in modules 1 to 6 (TH1-TH6) of the non-ribosomal peptide synthetase (NRPS) system (amino acids 950-1016 of SEQ ID NO: 10; amino acids 1984-2041 of SEQ ID NO: 10; amino acids 3841-3908 of SEQ ID NO: 10; amino acids 4844-4911 of SEQ ID NO: 10; amino acids 5838-5904 of SEQ ID NO: 10; and amino acids 6887-6948 of SEQ ID NO: 10). The boundaries and conserved motif (T) of the T domains were chosen as described by Konz et al. (1999), Chemistry and Biology, vol. 6, R39-R48 and indicated in grey.

FIG. 29(a and b) show an amino acid alignment comparing the 2 epimerization (E) domains present in modules 2 and 6 (EP2 and EP6) of the non-ribosomal peptide synthetase (NRPS) system (amino acids 2047-2457 of SEQ ID NO: 10 and amino acids 6950-7400 of SEQ ID NO: 10) and the epimerization domain of Gramicidin (SEQ ID NO: 31, taken from GrsA, GenBank AAA58718). The boundaries and conserved motifs (E1-E7) of the E domains were chosen as described by Konz et al. (1999), Chemistry and Biology, Vol. 6, R39-R48 and are indicated in grey.

FIG. 30 shows a schematic representation of the role of each domain and module of the NRPS system of ORF 5 (SEQ ID NO: 10), in grey, in the biosynthesis of the hexapeptide Compound 1.

FIG. 31 shows a schematic representation of the role of the formyl transferase (FXBA) of ORF 3 (SEQ ID NO: 6) shown in grey, and the H-hydroxylase (OXRK) of ORF 4 (SEQ ID NO: 8) in the biosynthesis of Compound 1. The ornithine residue is N-hydroxylated prior to its incorporation in the peptide as DHOR.

FIG. 32 illustrates the biosynthetic locus producing Compound 3 (05101) in Streptomyces aculeolatus, showing a scale in base pairs units; the position of the sequence of contiguous nucleic acids; the relative position and orientation of the 8 open reading frames of the biosynthetic locus (numbers 1 to 8 respectively corresponding to ORFs 20 to 27) and further identifying the ORFs encoding the 5 polyketide synthase proteins (PKSH) forming a polyketide synthase system; and the coverage of the biosynthetic locus by cosmids having deposit accession NOS: IDAC 051203-01 (051CJ), IDAC 051203-02 (051CG) and IDAC 051203-03 (051CC).

FIG. 33a illustrates biosynthesis of the polyketide core structure of Compound 3 (05101) involving the polyketide synthase system PKS B (modules 1 to 9). Inactive domains present in PKS B modules 7 and 8 are in bold.

FIG. 33b illustrates the formation of the terminal pyran-2-one ring catalyzed by the thioesterase domain present in module 9 of PKS B and the conversion of this structure to furanone through the action of OXRC and HOXC enzymes (respectively designated as ORFs 20, 21 and 27).

FIG. 34a shows a flow chart for steps involved in one embodiment of a method to identify compounds produced by cryptic secondary metabolite gene clusters.

FIG. 34b provides an expanded sub-portion of the flow chart of FIG. 34a, expanding upon steps involved in the comparison of selected cluster sequences versus a database in order to identify related clusters and gene homologs to permit prediction of structural features of the selected gene cluster's product (i.e., between steps *A and *B in the flow chart of FIG. 34a.

FIG. 34c provides an expanded sub-portion of the flow chart of FIG. 34a, noted by *C, expanding upon steps that can be used to isolate, determine the structure and/or test activities of a secondary metabolite natural product of a biosynthetic locus.

FIG. 35 shows a flow chart for steps involved in one embodiment of a method to identify a microorganism that produces a compound in a selected chemical family and thereby to identify a new compound in the selected chemical family. *C in FIG. 35 refers to *C as described by FIG. 34c.

FIG. 36 provides examples of secondary metabolite sub-structures and the enzyme families or domains that are associated with the presence of those sub-structures on the final secondary metabolite products.

### DETAILED DESCRIPTION

The invention relates to an integrated genomics-based discovery platform designed to increase the rate at which products of secondary metabolism are discovered. The approach combines the technologies of traditional metabolite purification and isolation processes with genomic and bioinformatics technologies to identify compounds that are likely to have escaped detection in the past. The invention is genomics-based, and advantageously uses genomic information regarding a target gene cluster involved in a secondary metabolism pathway to predict the chemical, physical and biological properties of the metabolite produced by the target gene cluster, and in some embodiments to further assist in one or more of the following: selection of a target gene cluster or metabolite of interest; selection of a microorganism; and selection of culture conditions under which to grow the microorganism. The invention is computer-assisted and employs bioinformatics techniques. The invention is high-throughput, which allows expedited discovery in a convenient and efficient format. Further, the invention is iterative and the data generated in each iteration is fed back into the knowledge repository to strengthen the predictive and discovery capacity of the method.

A microorganism is provided or selected containing a target gene cluster involved in the synthesis of a secondary metabolite and for which target gene cluster there is genomic information. An extract from the microorganism is obtained which contains the secondary metabolite synthesized by the gene cluster. Chemical, physical or biological properties of metabolites present in the extract are assessed and compared with the chemical, physical or biological properties predicted to be associated with the metabolite based on the genomic information. Genomic-guided expression, screening and isolation is used to identify and isolate the metabolite synthesized by the target gene cluster.

The term “microorganism” refers to any prokaryotic or eukaryotic microorganism known or suspected to contain a gene cluster directed to the synthesis of a secondary metabolite. Bacteria and fungi are preferred microorganisms for use in the invention. Suitable bacterial species include substantially all bacterial species, both animal- and plant-pathogenic and nonpathogenic. Preferred microorganisms include but are not limited to bacteria of the order Actinomycetales, also referred to as actinomycetes. Preferred genera of actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatosporia, Streptomyces, Microbispora, Streptosporangium, Actinomadura. The taxonomy of actinomycetes is complex and reference is made to Goodfellow (1989) Suprageneric classification of actinomycetes, Bergey's Manual of Systematic Bacteriology, Vol. 4, Williams and Wilkins, Baltimore, pp 2322-2339, and to Embley and Stackebrandt, (1994), The molecular phylogeny and systematics of the actinomycetes, Annu. Rev. Microbiol. 48, 257-289, for genera that may also be used with the present invention. In some embodiments, a knowledge repository is consulted to preferentially select a microorganism based on genomic information associated with a class of natural products, the presence of a target gene cluster, or production of a metabolite of interest.

The term “secondary metabolite” may be used interchangeably with the term “metabolite” and refers to a product arising from the biosynthesis involving a gene cluster within a microorganism which is a natural chemical product not normally employed in primary metabolic processes. The metabolite may be a member of a “chemical family” which is a grouping of chemical entities of natural products having a common physical attribute or structural feature. Representative chemical families include, for example, polypeptides (including subgroups thereof such as lipopeptides and glycolipopeptides), terpenes, alkaloids, polysaccharides, enediynes, glycopeptides, orthosomycins, benzodiazepines, aminoglycosides, beta-lactams, amphenicols, lincosamides and polyketides (including subgroups thereof such as macrolides, ansamycins, glycosylated polyketides and aromatic polyketides). One skilled in the art would readily understand that a compound having a polyketide backbone can be said to belong to the chemical family of “polyketides”, or that a compound having a polyene structure can be said to belong to the chemical family of “polyenes” etc. These exemplary chemical families should not be considered as limiting to the invention, as one skilled in the art could easily determine a desirable physical attribute of a chemical family of metabolites other than those exemplified herein.

The term “target gene cluster” refers to a gene, group of genes or a part of a gene involved in the biosynthesis of a secondary metabolite and for which there is genomic information. At a minimum, a target gene cluster encodes a polypeptide domain required for the generation of a chemical, physical or biological property that is characteristic of a given secondary metabolite—i.e., that provides for the discrimination of a given secondary metabolite from among other compounds lacking that chemical, physical or biological property. That domain need not be an entire polypeptide, and can encompass, for example, an enzyme catalytic domain that participates in the formation of a property exhibited by a secondary metabolite. At the other end of the spectrum, a target gene cluster can comprise sequences encoding multiple separate polypeptides that act in a biosynthetic pathway leading to synthesis of a given secondary metabolite. The term “target” is used simply to indicate that this is the particular gene cluster from which a metabolite of interest is expected to arise.

The term “genomic information” refers to the nucleic acid sequence of a target gene cluster or amino acid sequence of the corresponding polypeptide(s), or both, together with functional annotation of the sequence information. Advantageously, the genomic information provides a basis to make a prediction as to the chemical, physical or biological properties of the metabolite produced by a biosynthetic locus including the target gene cluster. In this regard, the genomic information must be sufficient to provide a direct link between the nucleic acid or corresponding polypeptide sequence information relating to at least a portion of a gene in the biosynthetic locus or at least a portion of a gene product encoded by a gene in the biosynthetic locus and the metabolite produced by the biosynthetic locus.

As used herein, the term “genetic information” refers to nucleic acid sequence information, particularly, but not limited to, nucleic acid sequence encoding amino acid sequence.

Many secondary metabolites are synthesized by a large multifunctional protein such as a nonribosomal peptide synthetase (NRPS) gene or a polyketide synthase (PKS) gene, and in such cases a “gene cluster” may be only part of a gene. Polyketides are synthesized by polyketide synthase (PKS) enzymes, which are complexes of multiple large proteins. Type 1 modular PKSs are formed by a set of separate catalytic active sites for each cycle of carbon chain elongation and modification in the polyketide synthesis pathway. Each active site is termed a domain. A set of active sites is termed a module. The typical modular PKS multienzyme system is composed of several large polypeptides, which can be segregated from amino to carboxy termini into a loading module, multiple extender modules, and a releasing module that frequently contains a thioesterase domain. Generally, the loading module is responsible for binding the first building block used to synthesize the polyketide and transferring it to the first extender module. The loading molecule recognizes a particular acyl-CoA and transfers it as a thiol ester to the ACP of the loading module. The AT on each of the extender modules recognizes a particular extender-CoA and transfers it to the ACP of that extender module to form a thioester. Each extender module is responsible for accepting a compound from a prior module, binding a building block, attaching the building block to the compound from the prior module, optionally performing one or more additional functions, and transferring the resulting compound to the next module. Each extender module contains a KS, AT, ACP, and zero, one, two or three domains that modify the beta-carbon of the growing polyketide chain. A typical (non-loading) minimal Type I PKS extender may contain a KS domain, an AT domain, and an ACP domain. Such domains are sufficient to activate an extender unit of 2 or more carbons and attach it to the growing polyketide molecule. The next extender module, in turn, is responsible for attaching the next building block and transferring the growing compound to the next extender module until synthesis is complete. Once the PKS is primed with acyl-ACPs, the acyl group of the loading module is transferred to form a thiol ester (trans-esterification) at the KS of the first extender module; at this stage, extender module one possesses an acyl-KS and a malonyl- (or substituted malonyl-) ACP. The acyl group derived from the loading module is then covalently attached to the alpha-carbon of the malonyl group to form a carbon-carbon bond, driven by concomitant decarboxylation, and generating a new acyl-ACP that has a backbone two carbons longer than the loading building block (elongation or extension).

The polyketide chain, growing by two carbons with each extender module, is sequentially passed as covalently bound thiol esters from extender module to extender module, in an assembly line-like process. The carbon chain produced by this process alone would possess a ketone at every other carbon atom, producing a polyketone, from which the name polyketide arises. Most commonly, however, additional enzymatic activities modify the beta keto group of each two-carbon unit just after it has been added to the growing polyketide chain but before it is transferred to the next module.

In addition to the typical KS, AT, and ACP domains necessary to form the carbon-carbon bond, a module may contain other domains that modify the beta-carbonyl moiety. For example, modules may contain a ketoreductase (KR) domain that reduces the keto group to an alcohol. Modules may also contain a KR domain plus a dehydratase (DH) domain that dehydrates the alcohol to a double bond. Modules may also contain a KR domain, a DH domain, and an enoylreductase (ER) domain that converts the double bond product to a saturated single bond. An extender module can also contain other enzymatic activities, such as, for example, a methylase or dimethylase activity.

After traversing the final extender module, the polyketide encounters a releasing domain that cleaves the polyketide from the PKS and typically cyclizes the polyketide. The polyketide can be further modified by tailoring enzymes; these enzymes add carbohydrate groups or methyl groups, or make other modifications, i.e. oxidation or reduction, on the polyketide core molecule. Domains include ketosynthase (KS), acyl transferase (AT), acyl carrier protein (ACP), dehydratase (DH), ketoreductase (KR), enoylreductase (ER) etc. The order in which individual domains appear in a given polypeptide can be represented as “domain strings” that are characteristic signatures of such multidomain polypeptides such as PKS systems, non-ribosomal peptide synthetases (NRPSs) as well as hybrid PKS/NRPS systems. Given the specificity as to domains and modules in multimodular proteins, a “gene cluster” as used herein may refer to part of gene representing one or more domains or one or more modules of a multimodular system. Similarly “genomic information”, as used herein may refer to genomic information pertaining only to part of gene.

In other embodiments the genomic information relates to a group of genes involved in the biosynthesis of a characteristic moiety of a natural product metabolite. In still other embodiments, the genomic information relates to the full-length biosynthetic locus producing a metabolite, or several partial or full-length loci each producing a metabolite of a single class of natural products. The genomic information may be functional annotation of the gene cluster established by experimental results or a putative function attributed to the gene cluster by computer-assisted sequence comparison with the sequence of other known genes.

Genomic information may be obtained from a knowledge repository of genomic information which may be a computer database wherein the genomic information is electronically recorded and annotated with information available from public sequence databases such as GenBank National Center for Biotechnology Information, NCBI and the Comprehensive Microbial Resource database (The Institute for Genomic Research). Alternatively genetic information may be generated according to any method known in the art such as methods employing nucleic acid probes, transposon-tagging, mutagenesis etc. Genetic information may also be generated by full genome sequencing of a microorganism. Another method that may be used to generate the genomic information is the high-throughput method for discovery of gene clusters described in CA 2,352,451 and U.S. Ser. No. 10/232,370 which advantageously provides a means to identify cryptic gene clusters, i.e. clusters of genes found in the genome of a microorganism and involved in the biosynthesis of a natural product metabolite which the microorganism has not previously been reported to produce. A cryptic gene cluster or biosynthetic locus containing a cryptic gene cluster may be expressed when the microorganism containing the cryptic gene cluster is grown under a particular set of culture conditions which may or may not be established. In some embodiments, the genomic information relates to a metabolite reported to be produced by a microorganism but for which the structure of the metabolite has not been elucidated.

The expression “chemical, physical or biological properties” refers to properties of a metabolite that are predicted based on the genomic data and subsequently measurable on a high throughput basis according to the invention. By “chemical property” is meant any chemical attributes or feature, such as the chemical structure, or the core structure (i.e., that structure shared by members of a class of different compounds defined by or comprising that structure), substructure or moiety of the metabolite of interest, or any chemical substituent, functionality or linkage found in the metabolite of interest. For example, the macrolide lactone ring structure of rosaramicins, the heterocyclic ring structure of benzodiazepines, the chromophore of enediynes, the amino acid residues of a peptide metabolite, the sugar residues in an oligosaccharide chain of a metabolite, the orthoester linkages of orthosomycins, the N-acyl peptide linkage of lipopeptides, the polyketide core structure of piericidins or dorrigocins would all be considered chemical properties of those respective metabolites of interest. The detection of a chemical property, as the term is used herein can be performed by standard methods known to one skilled in the art. Because biological function or activity is ultimately determined by chemical properties, measurement or detection of a chemical property of a secondary metabolite (or of a sub-structure of a secondary metabolite) as the term is used herein can encompass measurement of a biological function or activity characteristic of the presence of that chemical property. To this extent, the terms “chemical property” and “biological property” are coincident.

By “biological property” is meant the bioactivity or biological activity of a metabolite. “Bioactivity” and “biological activity” used herein with reference to a metabolite may be used interchangeably to refer to any observable activity possessed by the metabolite. Such activity may include, but is not limited to, antibacterial (gram-positive and/or gram negative), antifungal, anticancer, apoptotic or antiapoptotic activity or cell damaging activity as well as antiviral, immunosuppressant, hypocholesteremic, antihelmintic (e.g. cestodes, nematodes, schistosomes, trematodes), antiparasitic and insecticidal activities. Testing for such bioactivity or biological activity may be conducted using such tests as are known to those of skill in the art. For example, to test for antibacterial or antifungal activity, the effect of the metabolite on survival of a bacterium or fungus is evaluated. Similarly, anticancer, apoptotic, antiapoptotic, or other observable activities can be evaluated by exposing cells to the metabolite under conditions conducive to a particular activity to be encountered. A biological induction assay (BIA) may be used to detect agents that damage DNA. Cancer cell lines, such as HT-29 for colorectal cancer, SF268 for central nervous system cancers, MDA-MB231 for mammary gland adenocarcinoma and PC-3 for prostate carcinoma may be used in evaluating an anti-cancer efficacy of the secondary metabolite as part of the bioactivity screening. The bioactivity of the secondary metabolite may also be evaluated through the use of any of a variety of enzymatic assays. Such enzyme assays may include, for example, a 5-lipoxygenase assay, an acyl CoA-cholesterol acyltransferase (ACAT) assay, a cyclooxygenase-2 (COX-2) assay, a peripheral benzodiazepine receptor (PBR or PBenzR) binding assay, and a Leukotriene, Cysteinyl (CysLT₁) assay. 5-Lipoxygenase (5-LO) catalyzes the oxidative metabolism of arachidonic acid to 5-hydroxyeicosatetraenoic acid (5-HETE), the initial reaction leading to formation of leukotrienes. Eicosanoids derived from arachidonic acid by the action of lipoxygenases or cyclooxygenases have been found to be involved in acute and chronic inflammatory diseases (i.e. asthma, multiple sclerosis, rheumatoid arthritis, ischemia, edema) as well in neurodegeneration (Alzheimer's disease), aging and various steps of carcinogenesis, including tumor promotion, progression and metastasis. By performing this assay, a person of skill in the art may determine whether a secondary metabolite is able to block the formation of leukotrienes by inhibiting the enzymatic activity of human 5-LO. Acyl CoA-Cholesterol acyltransferase (ACAT) converts cholesterol to cholesteryl esters and is involved in the development of artherioscerosis. Cyclooxygenase-2 (COX-2) enzyme is made only in response to injury or infection. It produces prostaglandins involved in inflammation and the immune response. Elevated levels of COX-2 in the body have been linked to cancer. The peripheral benzodiazepine receptor (PBR or PBenzR) is a well-characterized receptor known to be directly involved in diseases states. PBR is involved in the regulation of immune responses. These diseases states include inflammatory diseases (such as rheumatoid arthritis and lupus), parasitic infections and neurodegenerative diseases (such as Alzheimer's, Huntington's and Multiple Sclerosis). This receptor is known to be involved in anticancer activity of known compounds. Leukotriene, Cysteinyl (CysLT₁) is involved in inflammation and CysLT₁-selective antagonists are used as treatment for bronchial asthma. CysLT₁ and 5-LO were found to be upregulated in colon cancer.

By “physical property” is meant any measurable physical observations of a metabolite, including but not limited to molecular mass and UV spectrum.

The expression of chemical, physical or biological properties may refer to a single property—whether a chemical property, a physical property or a biological property—, or a combination of two or more properties—whether chemical properties, physical properties, biological properties, or a combination of chemical, physical and/or biological properties.

The invention uses genomics-guided expression, screening, isolation and structure elucidation technologies to identify the metabolite of interest from a target gene cluster. The expression “genomics-guided methods” refers to methods for expression, screening, isolating and determining the structure of metabolites, which methods find a basis in genomic information. Such genomics-guided methods are independent of detection or analysis of the gene products encoded by the target gene cluster. By using genomics to guide such decisions as to which microbe to investigate or which culture conditions to utilize in order to achieve synthesis of a metabolite, the random nature of high-throughput screening is traversed. Previous processes using high-throughput screening have not been guided by genetic information, but instead have been guided by such factors as the outcome of biological activity tests (for example, antimicrobial activity). In such cases of high-throughput screening where genomic information is not used, such biological activity tests are conducted on a very large number of products, but few if any will show efficacy. By guiding initial selection of a microbe, or other decisions such as culture conditions or isolation protocols and structure elucidation protocols on the basis of the genomic information that indicates that a microorganism has the ability to produce a secondary metabolite of interest, the number of samples that must be tested in order to obtain positive biological activity outcomes in high-throughput screening tests can be greatly reduced, and the efficiencies of the expression/screening processes are improved. The invention provides methods in which the genomic potential of a microorganism is considered, based on the presence of a target gene cluster within the genome of the microorganism. These methods are thus said to be genomics guided.

The term “extract” refers to a medium or fermentation broth in which a microorganism is cultured, or which is obtained from disrupting or otherwise deriving metabolites from a cell culture following an incubation period. In some embodiments, the extract is obtained by culturing the microorganism under culture conditions based on a link in the knowledge repository that serves to predict the conditions under which the microorganism is likely to express the target gene cluster and synthesize a desired metabolite. In other embodiments the culture conditions are selected with reference to a knowledge repository containing a link between a class of natural products and the culture conditions under which microorganisms have been reported to synthesize a metabolite of that class. Where the genomic information is associated with a cryptic target gene cluster, the microorganism is induced to express the target gene cluster and to synthesize the corresponding metabolite by growing the microorganism under multiple culture conditions. Minor modifications in medium composition and culture conditions can have a major influence of the range of secondary metabolites produced by a microorganism. In some embodiments, the culture conditions are selected to maximize the probability that the natural product metabolite that might be produced by each secondary metabolic pathway present in the genome of a microorganism is expressed. Any conditions related to culture growth may be varied and used in association with the invention, for example pH, temperature, medium composition, humidity, pressure, the addition of pleiotropic factors or signalling molecules, etc. Other environmental conditions commonly known to effect natural product production such as the addition of DNA damaging agents, selective antibiotics and/or exposure to radiation can be used in combination with screening to select for alternate or enhanced natural product production in this invention.

For ease of reference, exemplary culture conditions and aqueous media formulations referred to herein are assigned a two-letter designation used throughout the present description and figures. AA is a medium containing 10 g/l of glucose; 40 g/l of corn dextrin, 15 g/l of sucrose, 10 g/l of casein hydrolysate (N-Z Amine A), 1 g/l of magnesium sulfate (MgSO4.7H₂O), and 2 g/l of calcium carbonate (CaCO₃). AB is a medium containing 24 g/l of glycerol; 25 g/l of mannitol; 25 g/l of soluble starch; 5.84 g/l of glutamine; 1.46 g/l of arginine; 1 g/l of sodium chloride (NaCl); 1 g/l of potassium phosphate, monobasic (KH₂PO₄); 0.5 g/l of magnesium sulfate (MgSO₄.7H₂O); and 2 ml/l of trace element solution and wherein the trace element solution is prepared by dissolving the following in 100 ml deionized, distilled (dd)H₂O: 0.1 g of FeSO₄.7H₂O; 0.01 g of MnSO₄.H₂O; 0.01 g of CuSO₄.5H₂O; 0.01 g of ZnSO₄.7H₂O; and 1 drop of concentrated sulphuric acid (H₂SO₄) is added as a stabilizer. BA is a medium containing 15 g/l of soybean powder; 10 g/l of glucose; 10 g/l of soluble starch; 3 g of sodium chloride (NaCl); 1 g/l of magnesium sulfate (MgSO₄.7H₂O); 1 g/l of potassium phosphate, dibasic (K₂HPO₄); and 1 ml of trace element solution produced by dissolve the following in 100 ml ddH₂O: 0.1 g of FeSO₄.7H₂O; 0.8 g of MnCl₂.4H₂O; 0.7 g of CuSO₄.5H₂O; 0.2 g of ZnSO₄.7H₂O, and 1 drop of concentrated sulphuric acid (H₂SO₄) added as a stabilizer. CA is a medium containing 40 g/l potato dextrin; 15 g/l of cane molasses; 10 g/l of glucose; 10 g/l of casein hydrolysate (N-Z Amine A); 1 g/l of magnesium sulfate (MgSO₄.7H₂O); and 2 g/l of calcium carbonate (CaCO₃). CB is a medium containing 20 g/l of sucrose; 2 g/l of bacto-peptone; 5 g/l of cane molasses; 0.1 g/l of ferrous sulfate heptahydrate (FeSO₄. 7H₂O); 0.2 g/l of magnesium sulfate heptahydrate (MgSO₄. 7H₂O); 0.5 g/l of potassium iodide (KI); 5 g/l of calcium carbonate (CaCO₃). CI is a medium containing 20 g/l of glycerol; 20 g/l of dextrin; 10 g/l of fish meal; 5 g/l of bacto-peptone; 2 g/l of ammonium sulfate (NH₄)₂SO₄; and 2 g/l of calcium carbonate (CaCO₃). DA is a medium containing 20 g/l of potato dextrin; 10 g/l of cane molasses; 10 g/l of glucose; 10 g/l of glycerol; 5 g/l of soluble starch; 5 g/l of soybean flour; 5 g/l of corn steep solids; 3 g/l of calcium carbonate (CaCO₃); 1 g/l of phytic acid; 0.1 g/l of ferrous chloride (FeCl₂.4H₂0); 0.1 g/l of zinc chloride (ZnCl₂); 0.1 g/l of manganese chloride (MnCl₂.4H₂O); 0.5 g/l of magnesium sulfate (MgSO₄.7H₂O). DY is a medium containing 10 g/l of corn starch; 5 g/l of pharmamedia; 1 g/l of CaCO₃; 0.05 g/l of CuSO₄ 5H₂O; 0.0005 g/l of NaI. DZ is a medium containing 15 g/l of soluble starch; 5 g/l of glucose; 10 g/l of cane molasses; 10 g/l of fish meal; and 5 g/l of calcium carbonate (CaCO₃). EA is a medium containing 50 g/l of lactose; 5 g/l of corn steep solids; 5 g/l of glucose; 15 g/l of glycerol; 10 g/l of soybean flour; 5 g/l of bacto-peptone; 3 g/l of calcium carbonate (CaCO₃); 2 g/l of ammonium sulfate (NH₄)2SO₄; 0.1 g/l of ferrous chloride (FeCl₂.4H₂0); 0.1 g/l of zinc chloride (ZnCl₂); 0.1 g/l of manganese chloride (MnCl₂.4H₂O); 0.5 g/l of magnesium sulfate (MgSO₄.7H₂O). ES is a medium containing 40 g/l of glucose; 5 g/l of dried yeast; 1 g/l of K₂HPO₄; 1 g/l of MgSO₄; 1 g/l of NaCl; 2 g/l of (NH₄)2SO₄; 2 g/l of CaCO₃; 0.001 g/l of FeSO₄ 7H₂O; 0.001 g/l of MnCl₂4H₂O; 0.001 g/l of ZnSO₄ 7H₂O; 0.0005 g/l of NaI. ET is a medium containing 60 g/l of molasses; 20 g/l of soluble starch; 20 g/l of fish meal; 0.1 g/l of copper sulfate (CuSO₄.5H₂O); 0.5 mg/l of sodium iodide (NaI); and 2 g/l of calcium carbonate (CaCO₃). FA is a medium containing 40 g/l of potato dextrin; 15 g/l of cane molasses; 10 g/l of glucose; 10 g/l of casein hydrolysate (N-Z Amine A); 3 g/l of sodium phosphate, dibasic, anhydrous (Na₂HPO₄); 1 g/l of magnesium sulfate (MgSO₄.7H₂O); and, after adjusting pH to 7.0, 2 g/l of calcium carbonate (CaCO₃). GA is a medium containing 103 g/l of sucrose; 10 g/l of glucose; 5 g/l of yeast extract; 0.1 g/l of casamino acids; 10.12 g/l of magnesium chloride (MgCl₂.6H₂O); and 0.25 g/l of potassium sulfate (K₂SO₄); and per litre of medium 10 ml of KH₂PO₄ (0.5% solution); 80 ml of CaCl₂.2H₂O (3.68% solution); 15 ml of L-proline (20% solution); 100 ml of TES buffer (5.73% solution, adjusted to pH 7.2); 5 ml of NaOH (1N solution); and 2 ml of trace element solution. HA is a medium containing 340 g/l of sucrose; 10 g/l of glucose; 5 g/l of bacto-peptone; 3 g/l of yeast extract; 3 g/l of malt extract; and 1 g/l of magnesium chloride (MgCl₂.6H₂O). IA is a medium containing: 40 g/l of soybean powder; 30 g/l of soluble starch; 20 g/l of glucose; 3 g/l of ammonium nitrate (NH₄NO₃); and, after adjusting pH to 6.2, 1 g/l of calcium carbonate (CaCO₃). IB is a medium containing 40 g/l of mannitol; 33 g/l of casein hydrolysate (N-Z Amine A); 10 g/l of yeast extract; 9 g/l of potassium phosphate, monobasic (KH₂PO₄); and 5 g/l of ammonium sulfate (NH₄)2SO₄. JA is a medium containing 35 g/l of malt extract; 30 g/l of corn starch; 15 g/l of corn steep liquor; 15 g/l of pharmamedia; and, after adjusting pH to 7.3, 2 g/l of calcium carbonate (CaCO₃). KA is a medium containing 10 g/l of glucose; 10 g/l of corn steep liquor; 10 g/l of soybean powder; 5 g/l of glycerol; 5 g/l of dry yeast; 5 g/l of sodium chloride (NaCl); and, after adjusting pH to 5.7, 2 g/l of calcium carbonate (CaCO₃). KC is a medium containing 40 g/l of tomato puree; 2 g/l of glucose; 15 g/l of oatmeal; 50 mcg/l of CoCl2.2H2O. KD is a medium containing 15 g/l of dextrin; 20 g/l of soluble starch; 10 g/l of soybean meal; 3 g/l of meat extract; 3 g/l of polypeptone; 3 g/l of yeast extract; 3 g/l of calcium carbonate; and 1 g/l of sodium chloride. KE is a medium containing 30 g/l of glycerol; 15 g/l of distiller's solubles; 10 g/l of pharmamedia; 10 g/l of fish meal; and 6 g/l of calcium carbonate (CaCO₃). KF is a medium containing 1 g/l of glucose; 24 g/l of soluble starch; 3 g/l of bacto peptone; 3 g/l of meat extract; 5 g/l of yeast extract; and 4 g/l of calcium carbonate. KG is a medium containing 10 g/l of bacto-peptone; 10 g/l of glucose; 20 g/l of cane molasses; 1 g/l of calcium carbonate; and 0.1 g/l of ferric ammonium citrate. LA is a medium containing 25 g/l of soluble starch; 15 g/l of soybean powder; 5 g/l of dry yeast; and 2 g/l of calcium carbonate (CaCO₃). MA is a medium containing 25 g/l of soluble starch; 15 g/l of soybean powder; 2 g/l of dry yeast; 5 g/l of sodium chloride (NaCl); 4 g/l of calcium carbonate (CaCO₃); and 2 g/l of ammonium sulfate (NH₄)2SO₄. MC is a medium containing 10 g/l of glucose; 10 g/l of starch; 15 g/l of soybean meal; 1 g/l of KH₂PO₄; 3 g/l of NaCl; 1 g/l of MgSO₄7H₂O; 0.007 g/l of CuSO₄5H₂O; 0.001 g/l of FeSO₄ 7H₂O; 0.008 g/l of MnCl₂ 4H₂O; 0.002 g/l of ZnSO₄ 5H₂O; MU is a medium containing 25 g/l of mannitol; 10 g/l of soybean powder; 10 g/l of beef extract; 5 g/l of bacto-peptone; 5 g/l of glucose; 2 g/l of sodium chloride (NaCl); 3 g/l of calcium carbonate (CaCO₃). NA is a medium containing 20 g/l of glycerol; 10 g/l of cane molasses; 5 g/l of caseamino acids; 1 g/l of bacto-peptone; 4 g/l of calcium carbonate (CaCO₃). NE is a medium containing 30 g/l of glucose; 5 g/l of bacto-peptone; 5 g/l of beef extract; 5 g/l of sodium chloride (NaCl); 2 g/l of calcium carbonate (CaCO₃). NF is a medium containing 20 g/l of soluble starch; 20 g/l of soybean meal; 5 g/l of NaCl; 5 g/l of yeast extract; 2 g/l of CaCO₃; 0.005 g/l of MnSO₄;0.005 g of CuSO₄; 0.005 g/l of ZnSO₄. NG is a medium containing 40 g/l glucose; 15 g/l of caseamino acids; 5 g/l of NaCl; 2 g/l of CaCO₃; 1 g/l of K₂HPO₄; 12.5 g/l of MgSO₄. OA is a medium containing 10 g/l of glucose; 5 g/l of glycerol; 3 g/l of corn steep liquor; 3 g/l of beef extract; 3 g/l of malt extract; 3 g/l of yeast extract; 2 g/l of calcium carbonate (CaCO₃); 0.1 g/l of thiamine. PA is a medium containing 10 g/l of soluble starch; 10 g/l of glycerol; 5 g/l of glucose; 5 g/l of beef extract; 3 g/l of bacto-peptone; 2 g/l of yeast extract; 1 g/l of casamino acids; 2 g/l of calcium carbonate (CaCO₃); 0.01 g/l of thiamine. PB is a medium containing 25 g/l of soybean meal; 7.5 g/l of soluble starch; 22.5 g/l of glucose; 3.5 g/l of dry yeast; 0.5 g of zinc sulfate (ZnSO₄.7H₂O); 6 g/l of calcium carbonate (CaCO₃). QB is a medium containing 10 g/l of soluble starch; 12 g/l of glucose; 10 g/l of Pharmamedia™; 5 g/l of corn steep liquor; 4 ml/l of proflo oil. RA is a medium containing: 20 g/l of soluble starch; 5 g/l of pharmamedia; 2.5 g/l of yeast extract; 1 g/l of sodium chloride (NaCl); 0.75 g/l of potassium phosphate, dibasic (K₂HPO₄); 1 g/l of magnesium sulfate (MgSO₄.7H₂O); 3 g of calcium carbonate (CaCO₃). RB is a medium containing 60 g/l of corn starch; 15 g/l of linseed meal; 10 g/l of glucose; 5 g/l of yeast extract; 1 g/l of ferrous sulfate (FeSO₄.7H₂O); 1 g/l of ammonium sulfate (NH₄)2SO₄; 1 g/l of ammonium phosphate (NH4H2PO4); 10 g/l of calcium carbonate (CaCO₃). RC is a medium containing 10 g/l of corn dextrin; 10 g/l of bacto-tryptone; 10 g/l of molasses; 2 g/l of sodium chloride (NaCl); 5 g/l of calcium carbonate (CaCO₃). RM is a medium containing 100 g/l of sucrose; 0.25 g/l of K₂SO₄; 10.128 g/l of MgCl₂.6H₂O; 21 g/l of MOPS; 10 g/l of glucose; 0.1 g/l of casamino acids; 5 g/l of yeast extract; 2 ml/l of trace elements. KH is a medium containing: 10 g/l of glucose; 20 g/l of potato dextrin; 5 g/l of yeast extract; 5 g/l of NZ Amine A; and 1 g/l of Mississippi lime (substitute CaCO₃). SF is a medium containing 25 g/l of glucose; 18.75 g/l of soybean powder; 3.75 g/l of cane molasses; 1.25 g/l of casein hydrolysate (N-Z Amine A); 8 g/l of sodium acetate; and 3 g/l of calcium carbonate (CaCO₃). SM is a medium containing 5 g/l of glucose; 5 g/l of starch; 7.5 g/l of soybean powder; 0.5 g/l of K₂HPO₄; 1.5 g/l of NaCl; 0.5 g/l of MgSO₄; 0.500 ml/l of 1000× metal salts; and 500 ml/l of H₂O. SP is a medium containing 20 g/l of glucose; 5 g/l of bacto-peptone; 5 g/l of beef extract; 5 g/l of sodium chloride (NaCl); 3 g/l of yeast extract; and 3 g/l of calcium carbonate (CaCO₃). QB is a medium containing: 5 g/l of starch; 6 g/l of glucose; 2.5 g/l of corn steep liquor; 5 g/l of pharmamedia; 2 ml/l of proflo oil. TA is a medium containing 103 g of sucrose; 5 g of yeast extract; 0.1 g of caseamino acids; 10.12 g of magnesium chloride (MgCl₂.6H₂O); 0.25 g of potassium sulfate (K₂SO₄); and after autoclaving, 10 ml of KH₂PO₄ (0.5% solution); 80 ml of CaCl₂.2H₂O (3.68% solution); 15 ml of L-proline (20% solution); 100 ml of TES buffer (5.73% solution, adjusted to pH 7.2); 5 ml of NaOH (1N solution); and 2 ml of trace element solution. VA is a medium containing 50 g/l of glucose; 30 g/l of soybean flour; 5 g/l of sodium chloride (NaCl); 3 g/l of ammonium sulfate (NH4)2SO₄; and 6 g/l of calcium carbonate (CaCO₃). VB is a medium containing 20 g/l of sucrose; 20 g/l of cane molasses; 10 g/l of glucose; 5 g/l of soytone-peptone; and 2.5 g/l of calcium carbonate (CaCO₃). WA is a medium containing 0.8 g/l of yeast extract; 0.5 g/l of casamino acids; 0.4 g/l of glucose; 2 g/l of potassium phosphate, dibasic (K₂HPO₄). XA is a medium containing 10 g/l of yeast extract; 10 g/l of casein hydrolysate (N-Z Amine A); 5 g/l of beef extract; 3 g/l of magnesium sulfate (MgSO₄.7H₂O); and 1 g/l of potassium phosphate, dibasic (K₂HPO₄). YA is a medium containing 10 g/l of bacto-peptone; 8 g/l of beef extract; 3 g/l of yeast extract; 5 g/l of glucose; 5 g/l of lactose; 2.5 g/l of potassium phosphate, dibasic (K₂HPO₄); 2.5 g/l of potassium phosphate, monobasic (KH₂PO₄); 0.2 g/l of magnesium sulfate (MgSO₄.7H₂O); and 0.05 g/l of manganese sulfate (MnSO₄.H₂O). ZA is a medium containing 10 g/l of sucrose; 8 g/l of casein hydrolysate (N-Z Amine A); 4 g/l of yeast extract; 3 g/l of potassium phosphate, dibasic (K₂HPO₄); and 0.3 g/l of magnesium sulfate (MgSO₄.7H₂O).

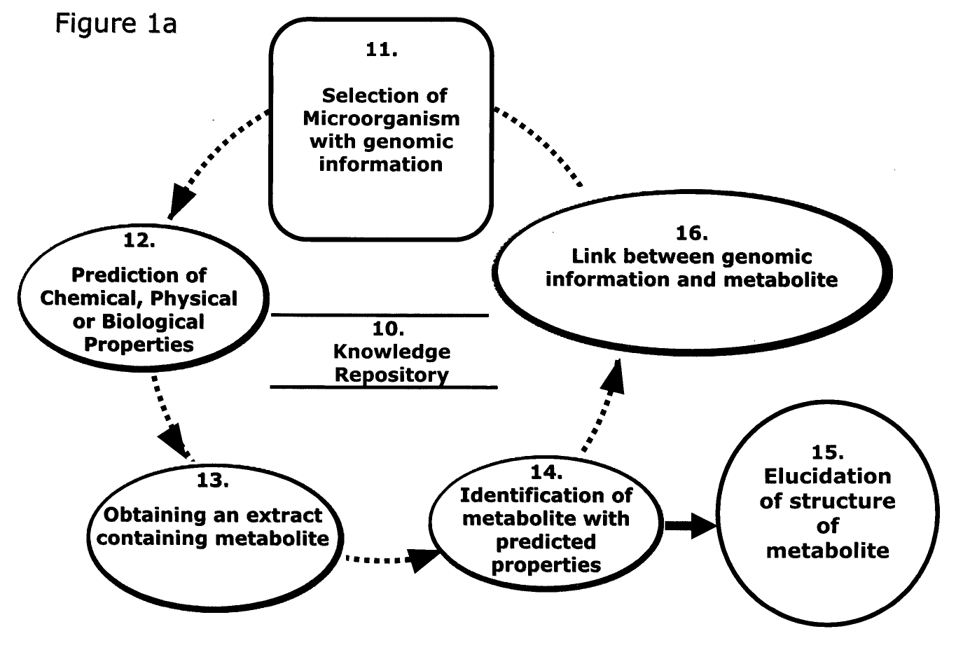

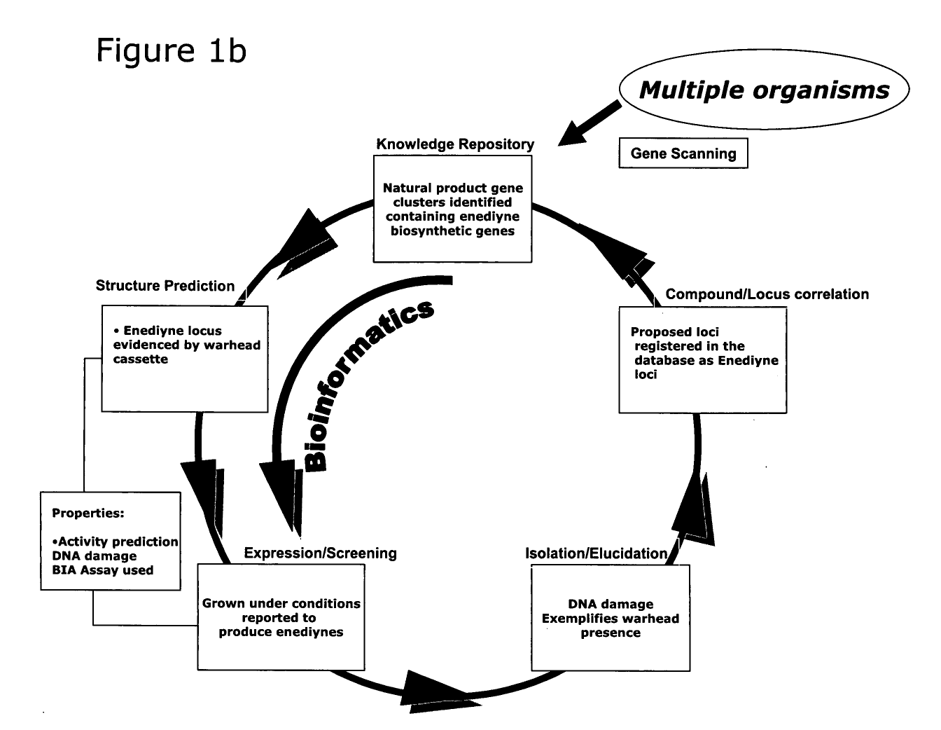

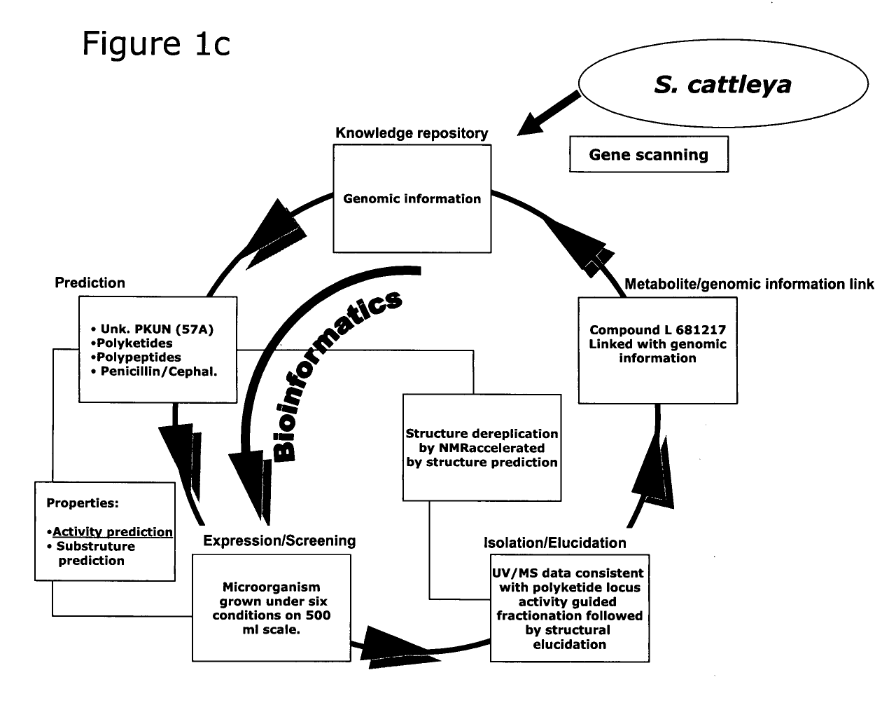

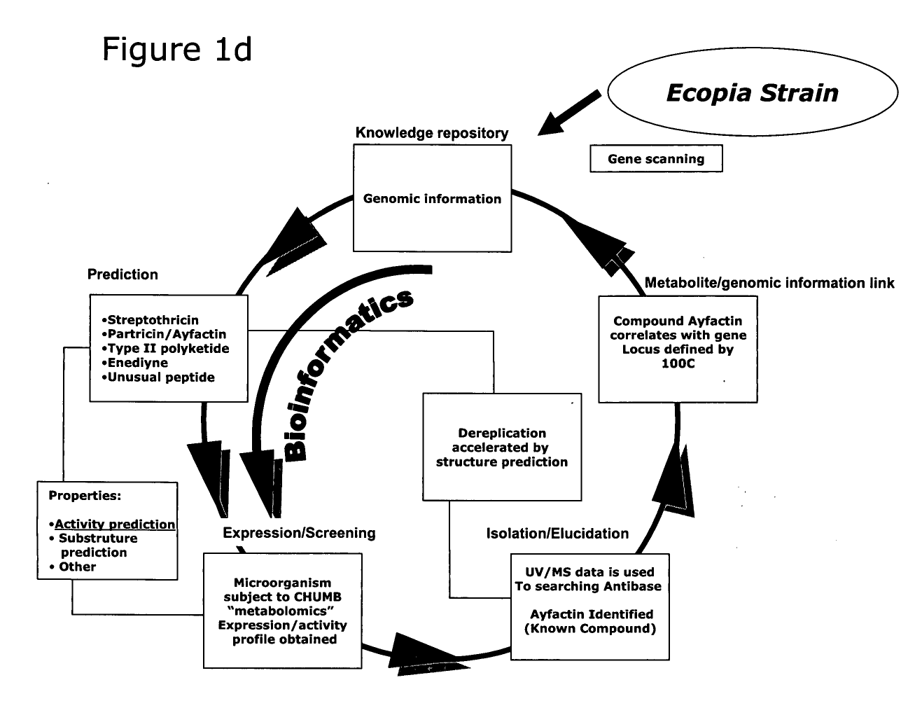

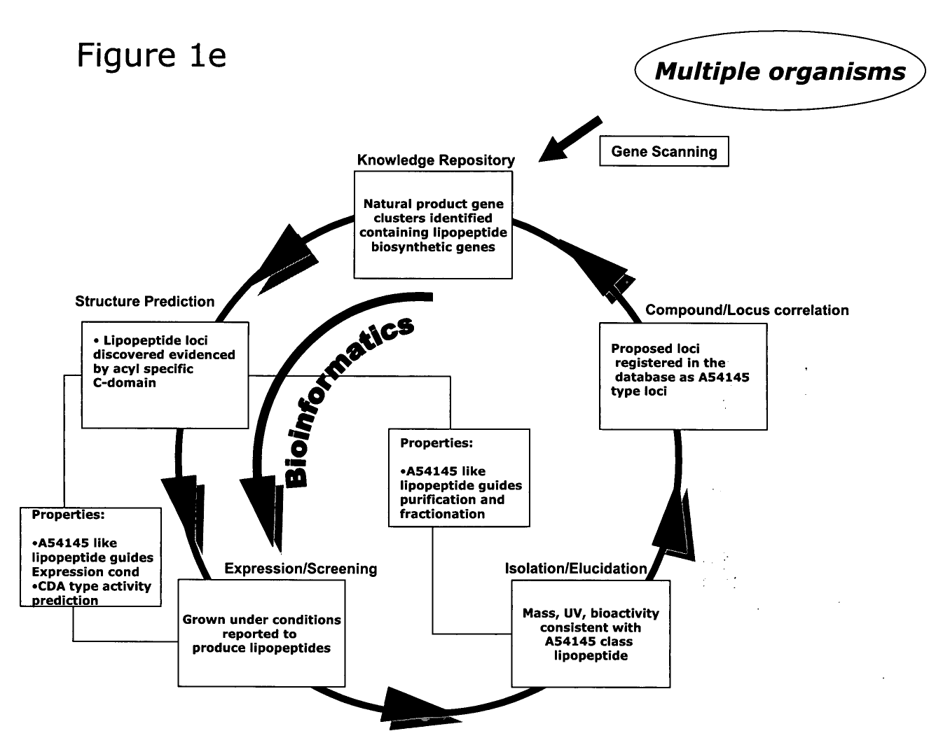

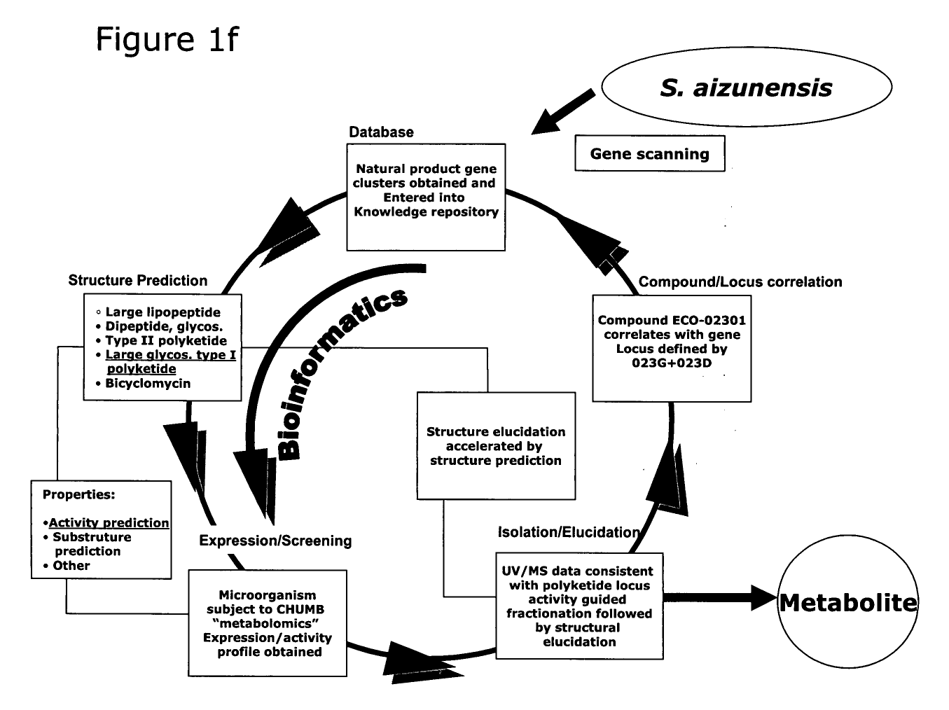

As illustrated in FIG. 1a, a microorganism (11) is selected. The microorganism contains a target gene cluster for which there is genomic information. The genomic information is used as a basis to make predictions (12) regarding chemical, physical or biological properties of the metabolite of interest. The predicted chemical, physical or biological properties direct the subsequent steps. The microorganism is induced to produce the metabolite synthesized by the target gene cluster and an extract with the metabolite of interest is obtained (13). Chemical, physical or biological properties of the metabolites in the extract are measured. The metabolite of interest is identified from the extract (14) by comparing the measured chemical, physical or biological properties with the predicted chemical, physical or biological properties of the metabolite of interest. A link (16) may be made in the knowledge repository between the metabolite and the target gene cluster. In some embodiments, the complete structure is elucidated (15) using genomic-guided methods. FIGS. 1b, 1c, 1d, 1e, 1f and 1g are embodiments of the method of FIG. 1a as described in each of examples 2, 3, 4, 5 and 6 respectively. FIG. 1b illustrates an embodiment where multiple metabolites of a pre-selected chemical family are identified. FIGS. 1c, 1d and 1f illustrate embodiments where the optional computer-assisted dereplication aspect of the invention is used. FIGS. 1c, 1d and 1f further illustrate embodiments where the optional structure elucidation step of the metabolite-of interest is performed. FIG. 1e illustrates an embodiment where the gene cluster is composed merely of part of a single gene. FIG. 1c illustrates an embodiment where a microorganism is randomly-selected and its genome is analyzed for the presence of cryptic gene clusters.

The invention is iterative and information generated during each iteration of the invention as well as links or associations between data elements established during each iteration of the invention may be fed back and stored into a knowledge repository to strengthen the predictive capacity of the invention. By way of example, in one embodiment, a link is made between the target gene cluster and the metabolite produced. In another embodiment a link is made between the metabolite produced and the microorganism selected. In a further embodiment a link is made between the genomic information and a chemical family. In a further embodiment a link is made between the culture conditions under which a microorganism is induced to synthesize a metabolite and the metabolite. In a further embodiment a link can be made between chemical, physical and biological properties and a metabolite of interest. It is to be understood that the invention does not require any particular link to be created and stored in the knowledge repository in order that the method or system of the invention achieve its objective of identifying a secondary metabolites. However, various embodiments may include a step wherein any one or more of the above links are created, fed-back and stored in the knowledge repository.

The invention contemplates use of conventional expression, screening, isolation and structure elucidation technologies and one skilled in the art could readily select appropriate technologies for use with the invention having regard to any one or more of the following factors: the target gene cluster, the metabolite of interest, the chemical class of interest, the microorganism selected, the predicted chemical, physical and biological properties etc. Preferred expression, screening, isolation and structure elucidation technologies are high-throughput or genomics-guided or both high-throughput and genomics-guided. By way of example, an appropriate screening technology would allow for the use of a battery of assays. In one embodiment an antibiotic screening assay for use with the invention incorporates a multi-well plate format (for example, a 96-well plate) to increase throughput. In another embodiment, the screening technology selected allows for the simultaneous screening of thousands of fermentation broths for antimicrobial activities.

In some embodiments, genomics-guided biological screening steps may be used to identify the best candidates for a more time-consuming chemistry isolation process. For example, if the genomics information indicates that the microorganism contains a gene clusters producing a compound of a class known to have activity against certain set of indicator organisms (Gram-positive, Gram-negative or activity against a particular organism), then the bioassay results may be used to select appropriate broths or extracts for chemical analysis. Alternatively, if the genomics information indicates that a microorganism may produce a previously-identified compound with known activity against certain indicator organisms, then it may be desirable to disfavor extracts that display activity against those indicator organisms when selecting extracts for chemical analysis.

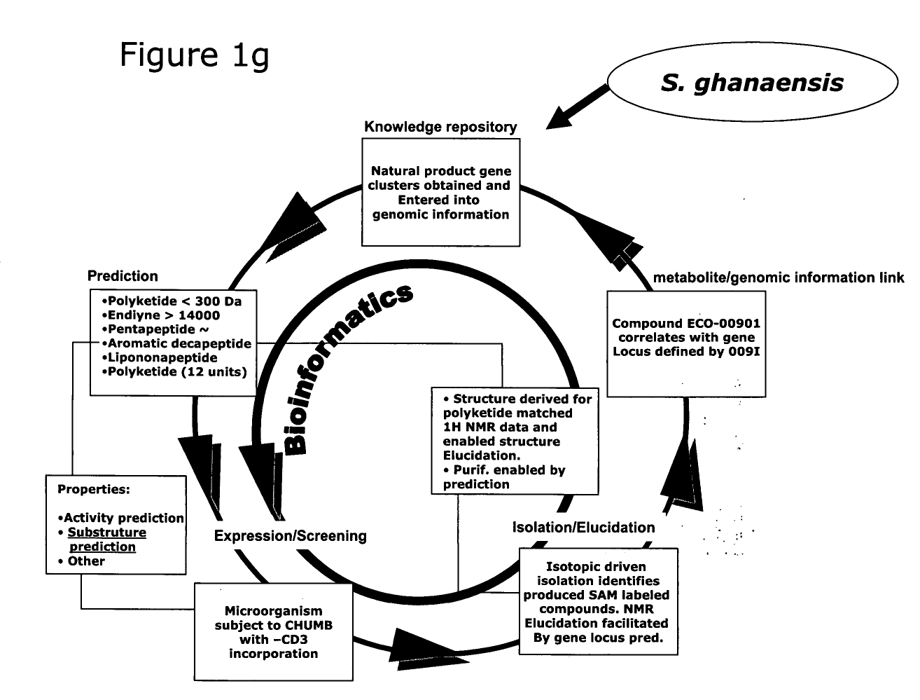

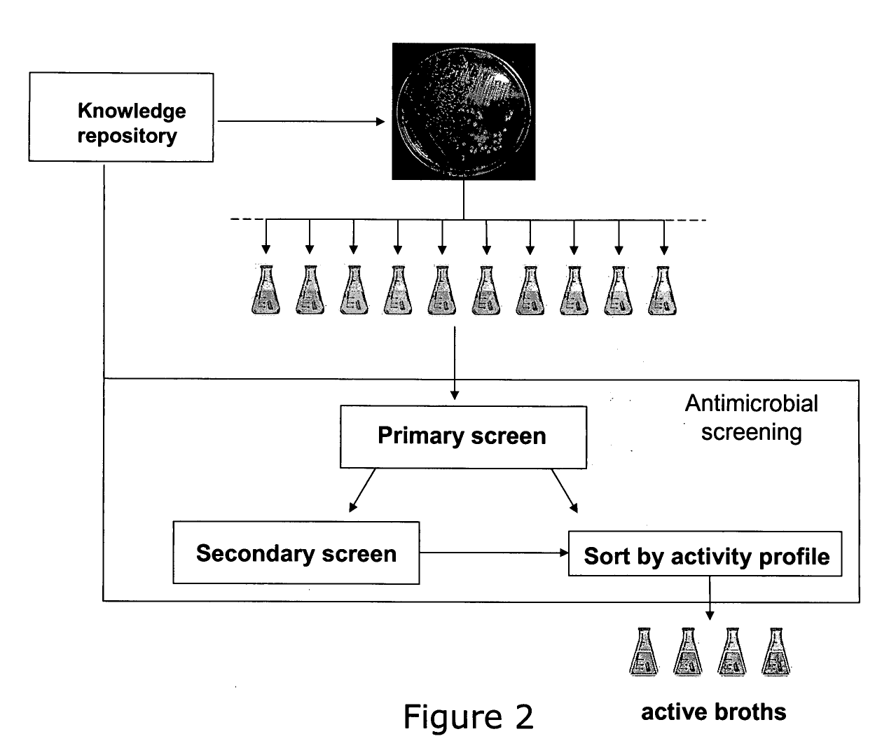

FIG. 2 illustrates one appropriate expression and screening technology for measuring biological properties of metabolites. In FIG. 2, extracts are screened against a panel of indicator microorganisms to identify metabolites with a particular biological activity. Extracts are tested for antibiotic activity against a panel of indicator strains, which may include bacterial (gram-positive and gram-negative) and fungal pathogens. Active extracts are sorted according to activity profile and representative extracts are selected for chemical analysis. In some embodiments, biological screening steps may be used to identify the best candidates for a more time-consuming chemistry isolation process.

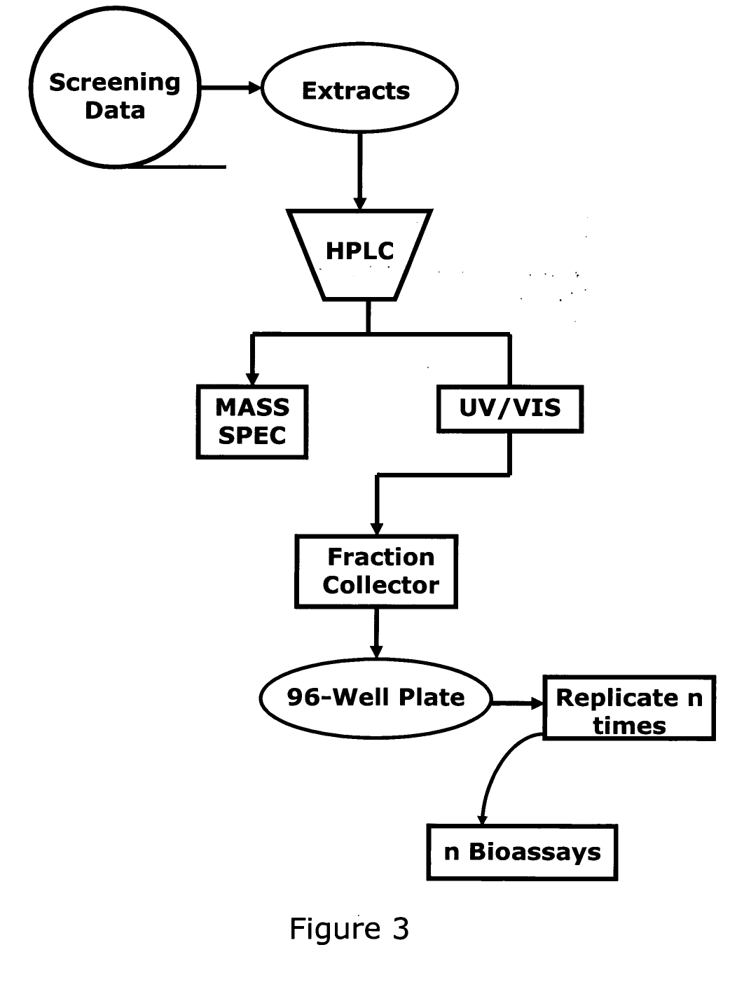

A convenient high-throughput protocol to assess chemical, physical and biological properties appropriate for use with the invention is referred to in the description and figures as CHUMB. As illustrated in FIG. 3, the CHUMB method fractionates extracts and generates data for each fraction in a given extract, including a UV trace by chromatographic mobility, a mass trace by chromatographic mobility providing the molecular weight of compounds in the fraction, and a bioactivity assessment of the compounds in the fraction, in a form which may readily be fed back to and stored in the knowledge repository. Using the CHUMB method, an extract is run through a chromatography column and is fractionated according to the mechanism of the chromatography media selected. For instance, a C-18 (octadecyl silane-functionalized silica gel) column run with an organic solvent gradient tends to separate compounds on the basis of their hydrophobicity. The output flow from the column is split with about 10% of flow provided for mass spectrometer analysis and about 90% flowing through a UV detector and then directed to a 96-well plate, fractionated by hydrophobicity. Bioactivity of the samples in the 96-well plate is assessed using one or more indicator strains or biological/biochemical assays to identify the bioactive fractions.

The metabolites produced by the target gene clusters are isolated from the samples of crude extract obtained from fermentation of a pure culture of the selected microorganism. Each sample would be expected to contain secondary metabolites exhibiting bioactivity against indicator strains, primary metabolites not generally exhibiting bioactivity against indicator strains, enzymes and fragments of enzymes involved in the biosynthesis of primary or secondary metabolic compounds, as well as biomass from media and whole cells. The crude extract is fractionated using known methods that are guided by the comparison of the measured chemical, physical and biological properties of the metabolites in each sample with the predicted chemical, physical and biological properties of the metabolite based on the genomic information to obtain purified samples containing single natural product metabolites. For example, the mass, UV and bioactivity of metabolites in each fraction may be compared with a database of known natural products in a dereplication step. A knowledge repository or database may be used in the dereplication step by comparing chemical, physical or biological data measured with the predicted chemical physical and biological properties based on genomic information from the microorganism used. Finally, the structure of the metabolite is solved, using well-known analytical methods, and the structure information fed back to and stored in the knowledge repository.

Genomics-based expression protocols employ conventional microbial growth fermentation methods, but give consideration to genomic information so as to make a rational selection regarding the culture conditions that will likely induce a microorganism to express a target gene cluster. One standard fermentation method that may be used is as follows. An agar plate of an appropriate medium is streaked with a glycerol stock of the desired organism and incubated at 30° C. for 2-7 days until colonies appear. The colonies are examined for contamination by microscopic analysis. Several loops of mycelia and/or spores are transferred to a sterile centrifuge tube along with a sterile medium (e.g. TSB medium), and crushed with a sterile centrifuge tube cell crusher. The crushed cell suspension is transferred to a sterile flask with appropriate seed culture medium (e.g. TSB), and 3 glass beads. The seed culture is shaken at about 250 rpm at 30° C. for 2-3 days until substantial cell density is present. Culture is again examined for contamination by microscopic analysis. For fermentation, about 25 to 500 mL of fermentation medium is prepared and sterilized in a large Erlenmeyer flask (125 ml to 4L). Two to ten ml of seed culture is added to an appropriate volume of culture medium in the fermentation flask and incubated at 30° C. for 2-7 days with shaking at 250 rpm. The culture is examined for contamination by microscopic analysis.

Samples of the fermentation broth from the culture conditions used are collected and chemical, physical or biological properties of the metabolites in the samples are measured. The chemical, physical or biological properties may be assayed by using many conventional methods including, but not limited to, spectroscopic, chromatographic, or biological methods or assays. Spectroscopic characterization methods include mass spectrometry, UV spectroscopy, NMR spectroscopy, IR spectroscopy, and X-ray diffraction analysis. Chromatographic methods characterize compounds on the basis of their mobility, or the lack thereof, in chromatographic systems such as such size exclusion chromatography, adsorption chromatography, partition chromatography, hydrophobic interaction chromatography, ion-exchange chromatography, and affinity chromatography. Biological assays include, but are not limited to cell-based methods such as antibacterial, antifungal, antiviral, antiprotozoal or eukaryotic cell differentiation, metabolism or cytotoxicity assays; multicellular organism-based assays such as insecticidal or antihelminthic (e.g. cestodes, nematodes, schistosomes, trematodes etc.) assays; or in vivo/in vitro biological assays, such as enzyme inhibition, DNA damage detection, immunological assays, ligand binding or other biochemical assays. Isotopic precursor and precursor analog incorporation methods provide a ready access to precursor and product functionality. It is generally known that supplementing fermentation growth media with isotopically labeled precursors or precursor analogs results in the partial (0.05-60% or more) incorporation of such isotopically- or chemically-labeled precursors into secondary metabolites which are biosynthesized via said precursors. Such incorporation can be investigated by a variety of analytical methods including, but not limited to, radiometry (e,g, ¹⁴C, ³H, ³²P, ³⁵S incorporation for isotopically-radiolabeled precursors), mass spectrometry (for stable and unstable isotopically labeled precursors and precursor analogs), or NMR (for spin-active nuclides). Precursors may include, but are not limited to primary metabolites, secondary metabolic intermediates, and precursor analogs. Genomic information regarding a target gene cluster and the metabolite of interest in a given organism allows for labeled precursors to be rationally selected, supplemented into the growth media, and the cryptic products of fermentation to be detected and resolved on the basis of the properties of the isotope-enriched products.

The metabolites synthesized by the target gene cluster are isolated from fermentation broths by a series of isolation and extraction steps designed to compare the measured chemical, physical or biological properties of the metabolites in the samples and the predicted chemical, physical or biological properties based on the genomic information.

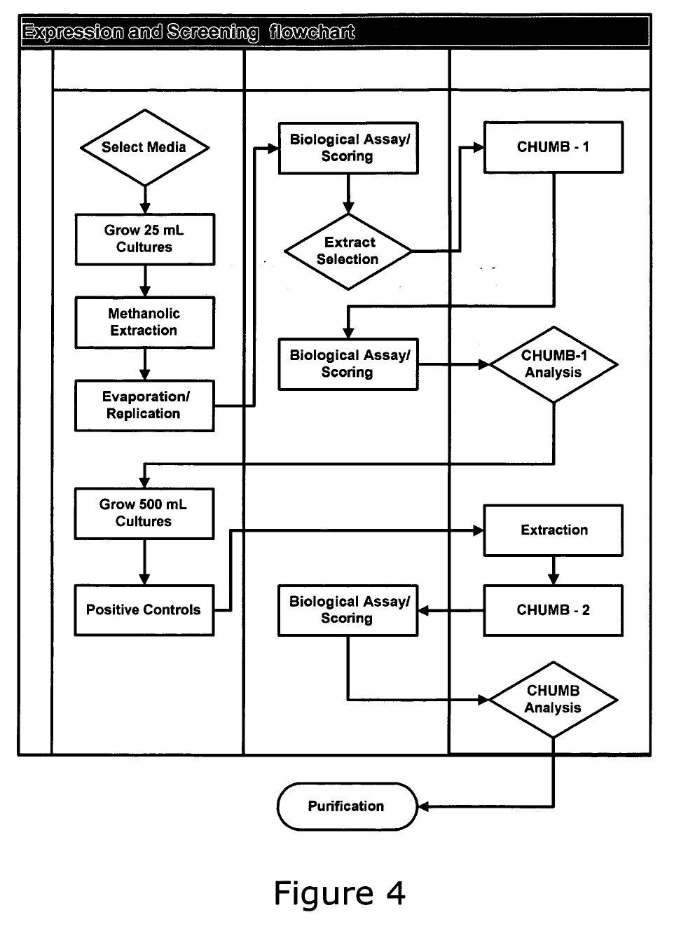

A representative genomics-guided expression and screening scheme for metabolite identification according to one embodiment of the invention is illustrated in FIG. 4. A candidate pure culture microorganism is grown under a wide variety of conditions to maximize the probability that all of its pathways will be expressed. Culture broths are tested for antibiotic activity against a panel of indicator strains for activity against various non-pathogenic microbial strains as well as pathogens, e.g. methicillin-resistant Staphyloccus aureus (MRSA) (ATCC-700699), Staphylococcus aureus (NRLLB-313), vancomycin-resistant Enterococcus faecalis (VRE) (ATCC-29212 and -51299), Micrococcus luteus (NRLLB-1018), Escherichia coli (ATCC-25922), Klebsiella pneumoniae (ATCC-10031) and Pseudomonas aeruginosa (ATCC-27853), and strains of fungal pathogens such as Candida albicans (ATCC-10231) and Candida glabrata (ATCC-90030), including those resistant to azole or polyene drugs Candida albicans (ATCC-204276). If the crude extract contains one or more bioactive compounds, the extract proceeds to a first CHUMB assessment. Mass spectra, UV spectra, and retention time are collected along with the screening activity data points for each test strain and the activity profiles are stored in the knowledge repository. This knowledge repository allows correlations to be made between pathway class, optimal expression conditions, and antimicrobial spectrum and physical properties. The global analysis of CHUMB assays for a number of growth conditions is referred to as CHUMB-1 analysis. Analysis of CHUMB-1 UV/mass spectral data allows, in some cases, dereplication, and in other cases partial structure elucidation or functional group identification. Based on correlations within the knowledge repository, conditions are selected for scale up fermentation required for structural elucidation. An extraction procedure is used to capture all metabolites from the large-scale fermentations. For example one general procedure described below localizes a given metabolite in one or more of five fractions based on cellular location and polarity. These extracts are also subject to the CHUMB process and then analysed to verify the presence of the metabolites targeted in the CHUMB-1 analysis. Analysis of the general extraction fractions of a given large scale fermentation is referred to as CHUMB-2 analysis.

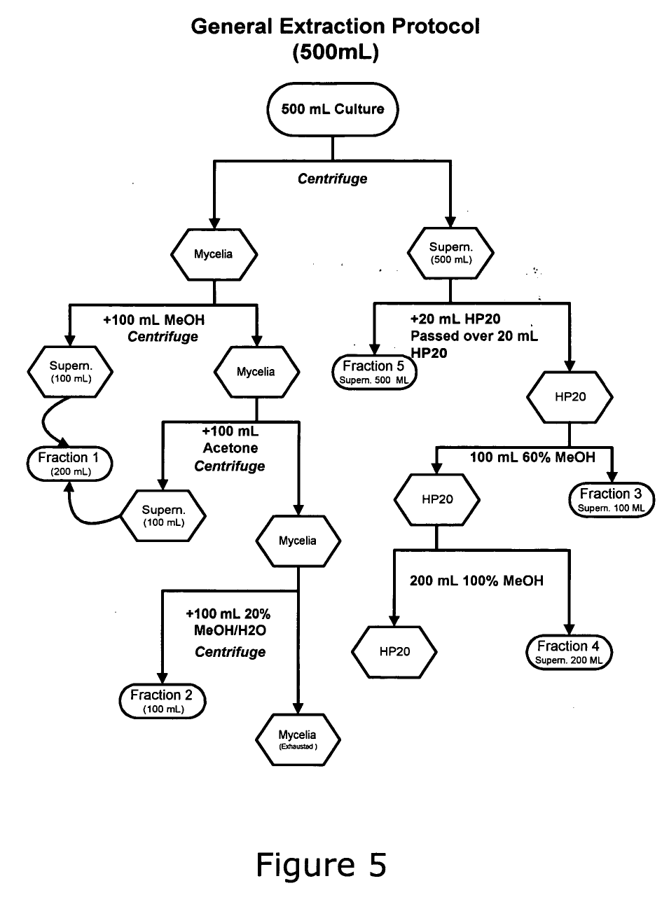

One general extraction procedure, illustrated in FIG. 5 is described as follows. Centrifuge the fermentation broth (500 ml) and decant to separate the supernatant from the mycelia. To the supernatant is added 30 ml of HP-20™ re-sin. This slurry is stirred for 20 minutes after which it is filtered through a short column of HP-20™ resin (30 ml). The column is then washed with 100 ml of wa-ter. The wash is combined with the initial eluate and labeled as extract no. 5. The column is then eluted with 100 ml of 60% MeOH/water and the eluate labeled as extract no. 3. The column is then eluted with 100 ml of 100% MeOH and then with 100 ml of acetonitrile. Combine these as extract no. 4. To the mycelia is added 100 ml of 100% MeOH, stirred for 10 minutes, centrifuged for 15 minutes, and the supernatant is decanted. To the mycelia is added 100 ml of acetone. The mixture is stirred for 10 minutes, centrifuged for 15 minutes and the supernatant decanted, adding it to the previous methanolic supernatant. This mixture is labelled as extract no. 1. To the mycelia is added 100 ml of 20% MeOH/Water. This mixture is stirred for 10 minutes, centrifuged for 15 minutes and decanted. Label this supernatant liquid as extract no. 2. Discard spent mycelia.

To summarize, metabolic components for a given organism grown under multiple conditions can be identified by CHUMB-1 analysis and “dereplicated” (distinguished from known compounds) by comparison to a knowledge repository of known compounds, or identified as potentially new compounds. After targets are selected, representing potentially new compounds, scale-up fermentations are performed to produce and isolate sufficient quantities of the compounds for structural elucidation by spectral analysis or other means. The efficiency of the discovery process increases with each chemical structure that is assigned to a biosynthetic pathway in the knowledge repository.

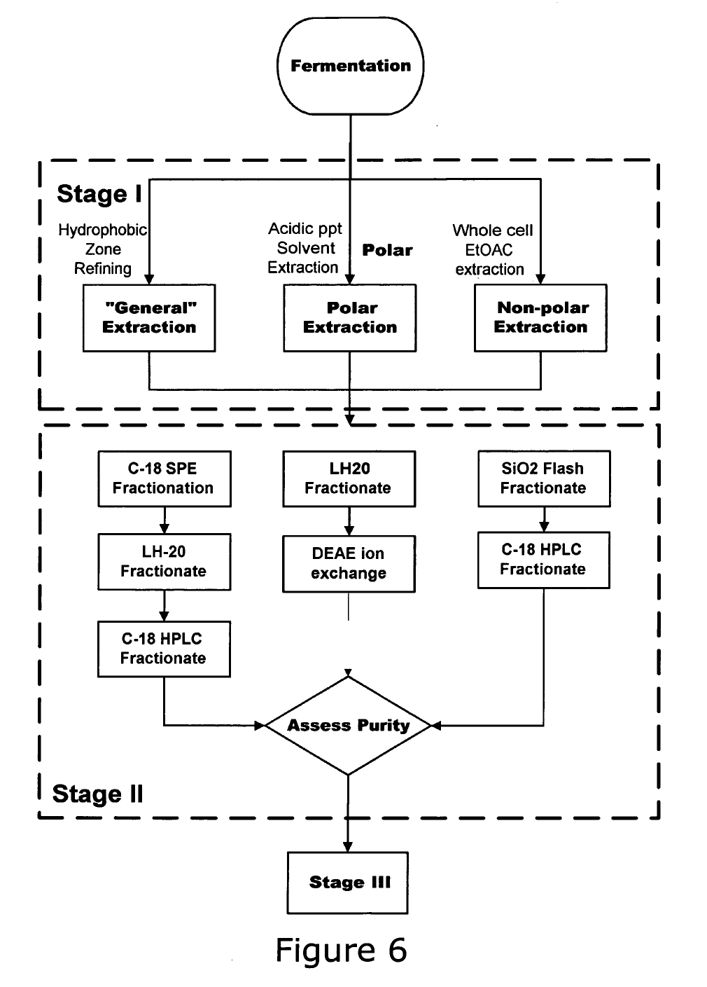

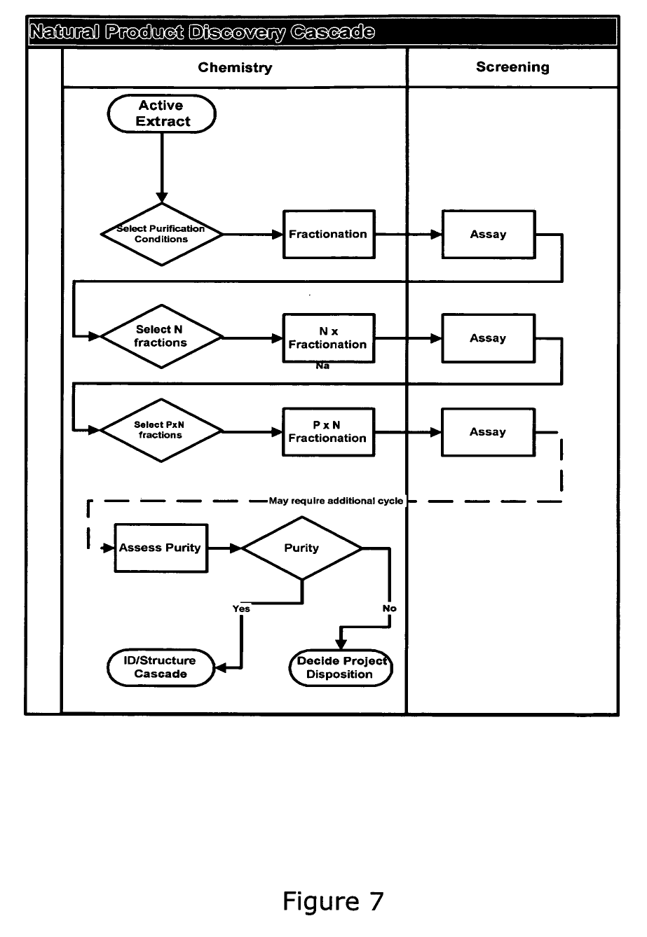

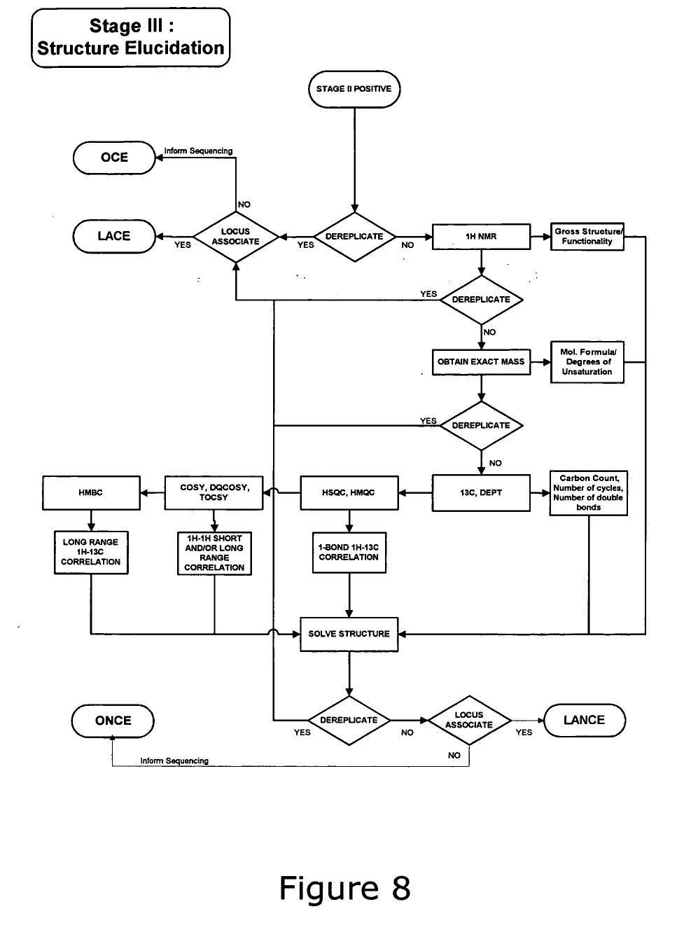

FIGS. 6, 7 and 8 provide an overview of a three-phase genomics-guided extraction/isolation/structure-elucidation protocol that may be used to discover natural product metabolites according to one embodiment of the invention. FIGS. 6, 7 and 8 illustrate a scheme wherein an extract is taken through a three-stage purification process that is designed to rapidly assess if the active component(s) are known compounds or are likely to be new. Genomic information from a knowledge repository facilitates compound identification at each stage by defining the range of chemical compounds that can be expected. Stage I and Stage II (FIGS. 6 and 7) are multi-step purification protocols, and the procedure used depends on whether the target compound is polar or non-polar, for example as may be determined by pre-screening CHUMB and genomics information. Stage II of the protocol is illustrated generally in FIG. 7. Stage III (FIG. 8) provides a structure elucidation cascade. Stage I (FIG. 6) is intended to extract and enrich bioactive components from a fermentation broth. At the end of Stage I there may still be thousands of compounds in the remaining slurry. In one embodiment, Stage I begins with about 500 ml to 2 L of crude fermentation broth which, at the end of Stage I extraction and enrichment, is reduced to about 2 ml for use in Stage II (FIG. 7) and Stage III (FIG. 8). The actual steps and order of steps in the extraction process of Stage I may be varied depending on the nature of the target compound. The invention may incorporate standard procedures for isolation of hydrophobic compounds using non-polar solvents such as ethyl acetate or acetone. Other protocols may be adapted or developed to allow for isolation of hydrophilic compounds. Examples of non-polar compounds include polyketides and polysaccharides; examples of polar compounds include peptide-based small molecules such as daptomycin, β-lactams, ramoplanin and vancomycin. In one embodiment, polar compounds are extracted from a fermentation broth by acidic solvent extraction, i.e. if the pH of the slurry is lowered to about pH 3, some polar compounds become soluble in organic solvents. Crude broths are extracted and fractionated using a variety of chromatographic procedures and the initial chemical properties of the active component(s) are determined. Chromatography results may be fed-back to and stored in the knowledge repository and linked to the locus information for the microorganism thereby providing an early opportunity to determine if the active component is a known compound.

One embodiment of the general protocol of FIG. 7 is shown as Stage II in FIG. 6, wherein active components in the remaining slurry produced in Stage I (FIG. 6) may be isolated and identified. The chromatography systems used and order of steps in the purification process may be varied depending on the nature of the target compound. Chromatographic devices are well known in the art, and may include for example, those such as a HPLC 2690 system by Waters Corporation (Milford, Mass.), and devices described in U.S. Pat. No. 5,670,054. A polar protocol that can be used in the invention involves LH20 fractionation (fractionation by size and polarity), followed by DEAE anionic exchange that fractionates positively charged compounds, and CHUMB. A non-polar protocol that can be used with the invention involves standard silica dioxide fractionation, followed by CHUMB. After purity assessment, the compound continues to stage III, structural elucidation.

FIG. 8 schematically illustrates a Stages III structure elucidation component of a three stage extraction/isolation/structure-elucidation protocol according to one embodiment illustrated in FIGS. 6, 7 and 8. Compounds that are not dereplicatively identified in Stage II (FIG. 6), and thus have the potential or being new chemical entities (NCEs), may be analyzed by UV/visible, infrared, tandem mass spectral and ¹H-NMR, ¹³C-NMR and multidimensional NMR methods to provide definitive structural information. These may include DEPT, HSQC, HMQC, COSY, DQCOSY, TOCSY, and HMBC NMR pulse sequences, which acronyms stand for distortionless enhancement of polarization transfer, heteronuclear single quantum coherence, heteronuclear multiple quantum coherence, correlation spectroscopy, double quantum-filtered correlation spectroscopy, total correlation spectroscopy, and heteronuclear multiple bond coherence respectively. Numerous NMR devices are known in the art, an example of which includes the UNITY INOVA 500™ device available from Varian (Palo Alto, Calif.). FIG. 8 provides one scheme for structure elucidation. In the embodiment illustrated in FIG. 8, the NMR procedures require an aliquot of the isolate obtained from Stage II (FIG. 6). In the case of peptides, amino acid analysis (PICOTAG or MS/MS analysis) requires just picomole amounts of material. Adequate quantities can be obtained from CHUMB plates to obtain amino acid residue identification. Referring to FIG. 8, the schematic starts with a stage II purified compound having no match among known chemical entities. Further characterization of compounds are conducted and dereplication is again employed to ensure that subsequent steps proceed only when there is no indication that the secondary metabolite of interest corresponds to a known entity. The designation LANCE refers to a locus-associated new chemical entities which means an NCE that is linked to a gene cluster for which there is genomic information; the designation ONCE refers to an orphan new chemical entities which means an NCE that is not yet linked to a gene cluster for which there is genomic information; the designation OCE refers to an orphan chemical entity which means a metabolite that is dereplicated at any point in the structure elucidation cascade, i.e. found to be identical to a previously described compound, and that is not linked to a gene cluster for which there is genomic information; the designation LACE refers to a locus associated chemical entity which means a metabolite that is dereplicated and that is linked to a gene cluster for which there is genomic information.

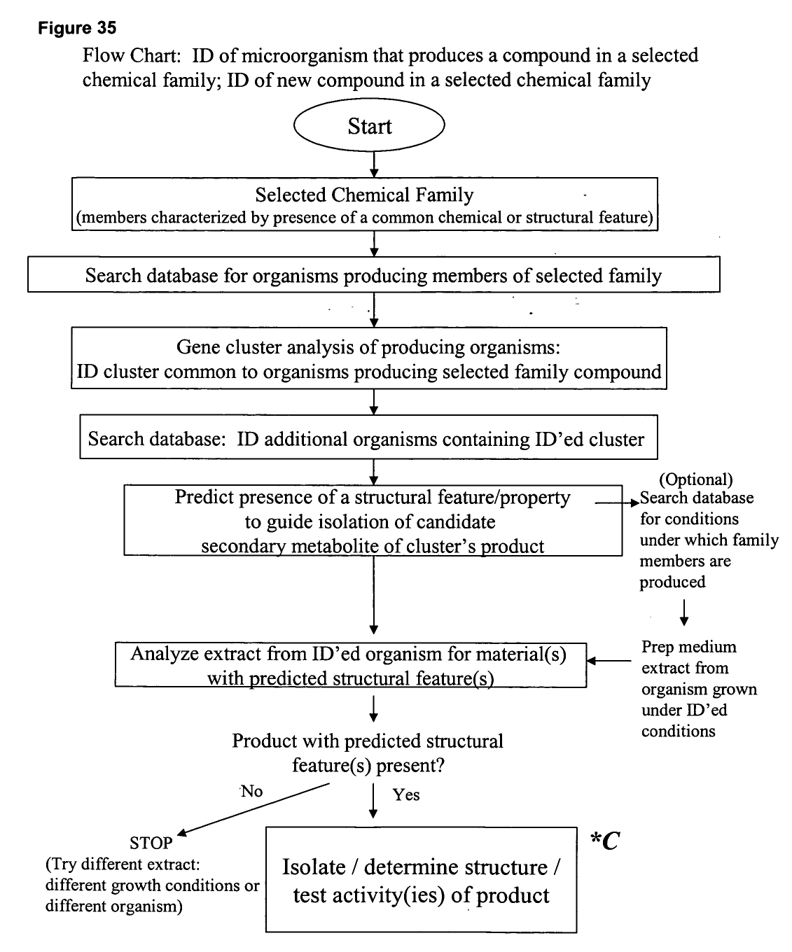

Also provided herein are methods for the identification of a microorganism that produces a secondary metabolite natural compound in a selected chemical family of interest. The approach can identify novel compounds in the selected chemical family through a genomic-guided process. The process is diagrammed in FIG. 35. The use of this approach to identify a microorganism that produces a lipopeptide natural product is also described, for example, in Example 4.

In this approach, a target class of natural compound, i.e., a class or family of compounds that share or are characterized by a common chemical property or structural factor is selected. A non-limiting example would be the lipopeptide family of secondary metabolite compounds, each member of which is characterized by the presence of a lipid moiety.

As a first step, a database is searched to identify organisms known to produce one or more members of the target family of secondary metabolite natural products. This process involves, for example, use of a web/LASSY GUI for keyword searching of an annotated database, such as the DECIPHER® database. Alternatively, for example, the process can involve a search of a literature database to identify organisms producing a member of the target family.

Next, biosynthetic gene cluster data from organisms that produce one or more members of the target family is compared, again using, e.g., a web/LASSY GUI, to identify one or more gene clusters common to organisms that produce the selected family compound. This step can identify a hallmark gene cluster for loci producing secondary metabolite natural products within the target class. The gene cluster identified in this way can then serve as a handle for identifying other microorganisms that produce secondary metabolites in the chosen target class.

Once such a hallmark gene cluster is identified, an annotated database comprising microbial secondary metabolite genomic information is queried to identify one or more additional organisms that contain the hallmark gene cluster. In this step, for example, a user will observe top-relatives hits (displayed, for example, in a LASSY interface) from querying of a database such as the DECIPHER® database or its equivalent. Alternatively, software such as BLAST, FASTA or other software capable of pairwise comparison or homolog detection can be used for querying any database comprising microbial genomic sequence information to identify organisms with genomic information that is significantly similar (as evidenced in, for example, E-values as described herein) to the identified hallmark gene cluster.

An organism comprising a hallmark gene cluster is identified as potentially capable of producing a candidate secondary metabolite in the chosen target class. Where the organism is not previously known to produce such a secondary metabolite, the organism is highlighted as potentially producing a novel secondary metabolite in the selected target class. From this point, an analysis of the secondary metabolite gene cluster in the identified organism permits the prediction of the presence of a structural or chemical property useful to guide the isolation of the candidate secondary metabolite. The analysis is performed using software tools such as HMMER, etc. and information in an annotated database comprising microbial genomic information correlated with secondary metabolite natural product production as described elsewhere herein.

Once the presence of a chemical or structural feature or property useful to guide the isolation of the candidate secondary metabolite is predicted, one can then analyze extracts prepared from the identified organism or its growth media for the presence of materials with the predicted feature(s) or property(ies). Where such a material is determined to be present, that material can then be isolated. The isolation can be further guided, for example, by information gleaned by querying an annotated database comprising information regarding the isolation of compounds in the selected chemical family, or by searching, for example, the published literature to identify likely isolation parameters for related compounds.

Prior to the preparation of extracts, one may optionally consult an annotated database to identify growth conditions under which members of the selected chemical family tend to be produced, in order to increase the chances that the candidate secondary metabolite will be produced. This optional step can alternatively involve consulting the published literature regarding microorganisms known to produce secondary metabolite natural products in the selected chemical family.

BLAST: While other alignment software can also be used to determine similarity between sequences, BLAST is applicable in several of the methods described herein. The use of BLAST is well known to those of skill in the art. However, for completeness, parameters for using BLAST within the methods described herein are discussed below. The BLAST algorithm, e.g., version 2.2.11 (available for use on the NCBI website or, alternatively, available for download from that site) is described in detail at the world wide web site (“www”) of the National Center for Biotechnology Information (“.ncbi”) of the National Institutes of Health (“nih”) of the U.S. government (“.gov”), in the “/Blast!” directory, in the “blast_help.html” file. The search parameters are defined as follows, and are advantageously set to the defined default parameters.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, 20 Proc. Natl. Acad. Sci. USA 87(6):2264-8 (see the “blast_help.html” file, as described above) with a few enhancements. The BLAST programs were tailored for sequence similarity searching, for example to identify homologues to a query sequence. For a discussion of basic issues in similarity searching of sequence databases, see Altschul et al. (1994), Nature Genetics 6(2), 119-129.

The BLAST programs available at the National Center for Biotechnology Information web site perform at least the following tasks: “blastp” compares an amino acid query sequence against a protein sequence database; “blastn” compares a nucleotide query sequence against a nucleotide sequence database; “blastx” compares the six-frame conceptual translation products of a nucleotide query sequence (both strands) against a protein sequence database; “tblastn” compares a protein query sequence against a nucleotide sequence database dynamically translated in all six reading frames (both strands). “tblastx” compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual available from the BLAST website as noted above).

DESCRIPTIONS Restricts the number of short descriptions of matching sequences reported to the number specified; default limit is 100 descriptions. (See parameter V in the manual). See also EXPECT and CUTOFF.

ALIGNMENTS Restricts database sequences to the number specified for which high scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992, Proc. Natl. 30 Acad. Sci. USA 89(22):10915-9). The valid alternative choices include: PAM40, PAM120, PAM:250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States, 1993, Computers and Chemistry 17:191-201, or, for BLASTN, by the DUST program of Tatusov and Lipman (see the world wide web site of the NCBI). Filtering can eliminate statistically significant but biologically uninteresting reports from the blast output (e.g., hits against common acidic-, basic- or proline-rich regions), leaving the more biologically interesting regions of the query sequence available for specific matching against database sequences. Low complexity sequence found by a filter program is substituted using the letter “N” in nucleotide sequence (e.g., “N” repeated 13 times) and the letter “X” in protein sequences (e.g., “X” repeated 9 times).

Filtering is only applied to the query sequence (or its translation products), not to database sequences. Default filtering is DUST for BLASTN, SEG for other programs. It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi causes NCBI gi identifiers to be shown in the output, in addition to the accession and/or locus name.

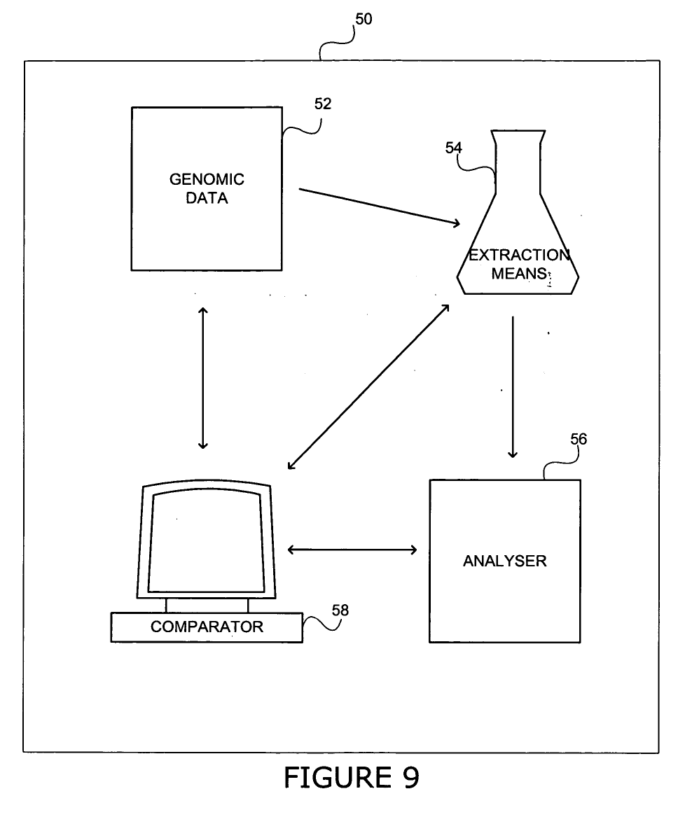

System: The invention provides a system for identifying a secondary metabolite synthesized by a target gene cluster contained within the genome of a microorganism, which system may be computerized or contain a computerized component. FIG. 9 illustrates a system (50) for identifying a secondary metabolite synthesized by a target gene cluster includes genomic data (52), an extraction means (54), an analyser (56) and a comparator (58), each of which is described in more detail below. The genomic data is also referred to as genomic information in the present specification.

An extraction means is used in the system, which is capable of obtaining an extract from the microorganism which contains the metabolite of interest produced by the target gene cluster. Such an extraction means may be a culture system which may incubate the cells under a selected group of conditions, and which thus derives extract from the cells after suitable incubation either by obtaining products exuded by cells in culture, or by disrupting cells at the end of an incubation period. Such methods would be known to or practicable by one skilled in the art.

The system further contains an analyser used to measure chemical, physical or biological properties of metabolites within the extract. As discussed herein, UV spectrum, HPLC, activity assays, chromatography, and other means of detecting chemical, physical or biological properties of metabolites may be used in the analyser component of the system.

The comparator of the system is used to identify, from these measured properties obtained by the analyser, the presence of the metabolite of interest. The comparator may be a computer system adapted to accept inquiries from a user, or may be programmed in such a way as to effect inquiries in a pre-determined manner. The comparator may function not only to effect comparison, but may optionally have interaction with any or all other components of the system, for example by housing data derived from the individual components of the system.

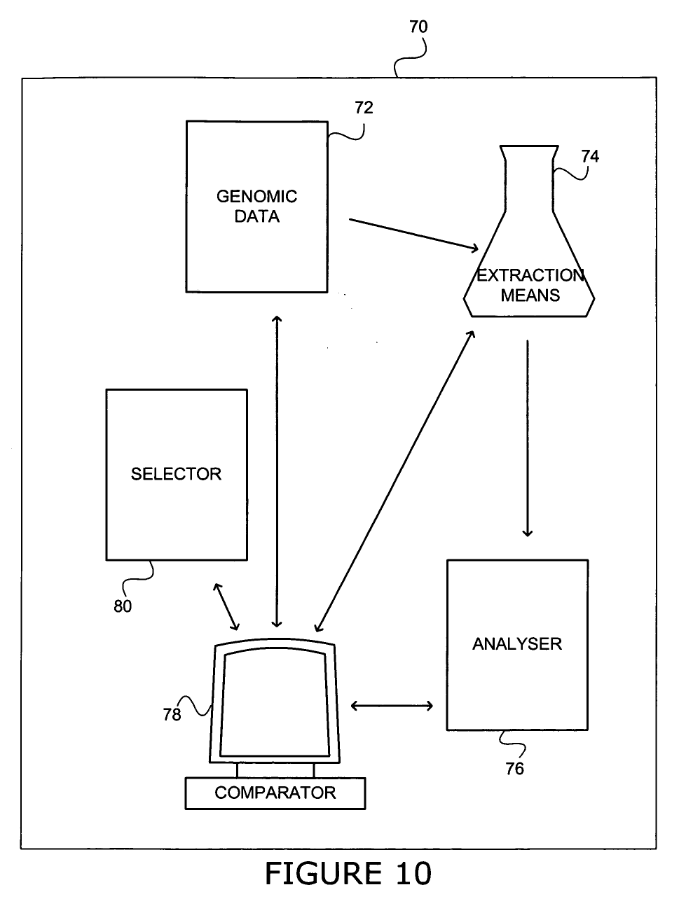

Similarly, the invention provides a system for identifying a secondary metabolite from a pre-selected chemical family. FIG. 10 provides a schematic representation of such a system. The system (70) includes the components discussed above, namely: genomic data (52), an extraction means (54), an analyser (56) and a comparator (58), but also includes a selector (80) for selecting a microorganism containing a target gene cluster. The selector may be, for example, a selectable item accessed from a graphical user interface. In this way, the system according to the invention allows selection of an appropriate microorganism capable of producing a particular desired metabolite from a class (or family) of metabolites on the basis of available genomic data. The comparator may function not only to effect comparison, but may optionally have interaction with any or all other components of the system, for example by housing data derived from the individual components of the system.

Knowledge Repository: According to the invention, a knowledge repository or computer-readable medium is provided, which houses secondary metabolism data from a microorganism. The repository can be used to identify a secondary metabolite synthesized by a target gene cluster contained within the genome of a microorganism. The repository comprises genomic data confirming the presence of a target gene cluster within a microorganism and genomic information pertaining to the gene cluster. Further, the repository houses extract characterizing data providing chemical, physical or biological properties of metabolites contained in an extract derived from the microorganism. These metabolites include a secondary metabolite attributable to a target gene cluster. Additionally, the repository includes comparative data, representing predicted chemical, physical or biological properties of the secondary metabolite synthesized by the target gene cluster. Within the knowledge repository, the extract-characterizing data is comparable with the comparative data for identifying a secondary metabolite the metabolites in an extract.

A knowledge repository may be, for example, a location at which data is stored or a grouping of data within one or more databases. According to the invention, the knowledge repository allows related information to be stored, added, correlated, compared and retrieved as required. The knowledge repository may be under computer control, and may store a variety of types of information such as chemical, physical and biological properties of a metabolite (for example, structure, molecular mass, UV spectrum or bioactivity), genetic information relating to a microorganism, or culture conditions under which a microorganism produces a metabolite. The knowledge repository may include previously established data obtained through accessing public or private databases, as well as newly generated data obtained according to the invention.

The knowledge repository may provide a “prediction link” between individual records within the repository. For example, genomic data and comparative data (representing expected chemical, physical or biological properties of a metabolite) may be correlated via a prediction link if it is established through actual observation that a metabolite of a target gene cluster possesses the expected properties. Such prediction links formed within the knowledge repository strengthen the predictive value of the knowledge repository when a new microorganism possessing a target gene cluster or a portion thereof is identified. In this way, the knowledge repository advantageously benefits from previously established data and new data added thereto, to predict the potential of a new microorganism (one for which secondary metabolism data has yet to be fully elucidated) to provide a member of a given class or family of compounds.

In related aspects, the invention provides a knowledge repository in which gene cluster information is linked to secondary metabolite production data. The invention further relates to a graphical user interface for accessing the knowledge repository. Further, according to embodiments of the invention, a memory for storing data may be considered a component of the knowledge repository, the memory having a data structure stored therein. The memory may include links between certain types of data. For example, in some embodiments the data representing a chemical structure of a metabolite is linked to a gene cluster or a genetic locus within the genomic data housed in the knowledge repository, thereby increasing the predictive power of the invention and allowing known compounds or compound classes (within a chemical family) to be identified earlier in the purification process. As used herein, a “link” may also refer to a point-and-click mechanism implemented on a user device connectable to the system. In this context, the link allows a viewer to link (or jump) from one display or interface where information is referred to (a “link source”) to other screen displays where more information exists (a “link destination”). The term “link” encompasses both a display element that indicates that the information is available and a program that finds the information (e.g. from within the knowledge repository) and displays the information on the GUI.

The invention further provides a memory for storing secondary metabolism data for access by an application program being executed on a data processing system for identifying a secondary metabolite synthesized by a target gene cluster contained within the genome of a microorganism. The memory comprises a data structure stored therein, the data structure including information resident in a database that is used by the application program. This database includes (i) genomic data confirming the presence of a target gene cluster within a microorganism, wherein a putative or confirmed function has been attributed to at least one region of a gene in the gene cluster; (ii) extract-characterizing data providing chemical, physical or biological properties of metabolites contained in an extract derived from the microorganism, wherein said metabolites include a secondary metabolite attributable to the target gene cluster; and (iii) comparative data representing expected chemical, physical or biological properties of the secondary metabolite synthesized by the target gene cluster. The extract-characterizing data is comparable with the comparative data for identifying from the metabolites in an extract the secondary metabolite synthesized by the target gene cluster, based on the putative or confirmed function attributed to the at least one region of a gene in a gene cluster.

The invention also relates to a method of building a knowledge repository housing secondary metabolism data from a microorganism. This method comprises the following steps. Genomic data is assembled, confirming the presence of a target gene cluster within a microorganism, wherein a putative or confirmed function has been attributed to at least one region of a gene in the gene cluster. Extract-characterizing data is input, so as to provide chemical, physical or biological properties of metabolites observed in an extract derived from the microorganism, wherein the metabolites include a secondary metabolite attributable to the target gene cluster.

Further, the extract-characterizing data are compared with comparative data representing expected chemical, physical or biological properties of the secondary metabolite synthesized by the target gene cluster. This step allows identification, from the metabolites in an extract, of the secondary metabolite synthesized by the target gene cluster based on the putative or confirmed function attributed to the at least one region of a gene in a gene cluster. Finally, the result of the extract-characterizing step is retained by linking a secondary metabolite identified in the comparing step with the genomic data assembled in the assembling step.

The step of inputting extract-characterizing data may optionally comprise inputting culture conditions under which an extract is derived, and the step of retaining the result may additionally comprise linking culture conditions to both the secondary metabolite identified in the comparing step and the genomic data assembled in the assembling step. The step of inputting extract-characterizing data may comprise inputting a biological property, such as antibacterial, antifungal or anticancer activity.

Similarly, another method of building a knowledge repository housing secondary metabolism data from a microorganism for predicting secondary metabolite production from a target gene cluster based on genomic data is provided according to the invention. This method comprises assembling genomic data confirming the presence of a target gene cluster within a microorganism, wherein a putative or confirmed function has been attributed to at least one region of a gene within the gene cluster. The following steps are also included: extracting a medium containing said microorganism, thereby forming an extract; screening the extract for extract-characterizing data indicative of the presence or absence of a secondary metabolite attributable to the target gene cluster based on a pre-selected chemical, physical or biological property; entering the extract-characterizing data into the knowledge repository; comparing the extract characterizing data with comparative data representing expected chemical, physical or biological properties of a secondary metabolite synthesized by the target gene cluster, so as to identify from the extract a secondary metabolite synthesized by the target gene cluster based on the putative or confirmed function; determining the identity of a secondary metabolite extracted; and affirming within the knowledge repository a correspondence between genomic data, the pre-selected chemical, physical or biological property, and the identity of the secondary metabolite, allowing a cycle of prediction of secondary metabolite production based on genomic data.

Feed Back into Knowledge Repository: The invention contemplates that chemical, physical or biological properties are measured in regard to metabolites produced by microorganisms. Screening activity data-points are collected for each microorganism that enters an expression/screening process. In some embodiments, the activity profiles are stored in a knowledge repository. For example, the results of any bioassay used to determine biological activity are fed-back to and stored in a computer and presented graphically or as a colored bar graph, indicating which of the fractions are bioactive. The activity profiles allow correlations to be made between pathways, chemical class or chemical family, optimal expression conditions and antimicrobial (or other bioactivity) spectrum. Similarly, data regarding physical properties of a metabolite (such as UV spectrum and mass obtained during CHUMB steps) is fed-back and stored in a knowledge repository. This increases the predictive value of the database, as more data is added and more correlations are found, to assist in forming prediction links.

Graphical User Interface: According to the invention, a graphical user interface (GUI) may be provided for subscribing to a knowledge repository. By “subscribing” to the repository, it is meant accessing, adding or modifying data within, producing reports from, or searching within the knowledge repository. The repository houses secondary metabolite data from at least one microorganism for identifying a secondary metabolite synthesized by a target gene cluster. Optionally, data from more than one organism may be housed in the repository, and there is no upper limit on the number of observations or organisms for which data may be housed in the repository. Indeed data derived from thousands of microorganisms may be housed in the repository.

The graphical user interface comprises a genomic access element for accessing from within the knowledge repository genomic data. This genomic data confirms the presence of a target gene cluster within a microorganism, wherein a putative or confirmed function has been attributed to at least one region of a gene in a gene cluster. The genomic access element may be positioned on a computer screen, and may access the genomic data within the repository when a command is received from a user at the interface, for example using a selectable pull-down menu, by entering a microorganism name, or by clicking on (selecting) an icon or other representation of a genomic region of interest.

The graphical user interface also comprises an extract-characterizing access element for accessing from within the knowledge repository chemical, physical or biological properties of metabolites contained in an extract derived from the microorganism. The extract-characterizing access element may be positioned on a computer screen, allowing access to the knowledge repository through a selectable pull-down menu, by entering terms indicative of extract-characterizing properties, or by clicking on (selecting) an icon representing certain extract-characterizing data such as media type, culture conditions, or biological activity. This element may be configured so as to provide searchable access to media composition and growth conditions under which a microorganism extract was obtained. This is a particularly helpful query if a user is attempting to determine conditions under which a certain cryptic pathway is “turned on”, if a metabolite not normally generally produced by a particular organism is shown to be present in a particular extract. Those conditions so located could be used in an effort to turn on similar metabolic pathways in other microorganisms shown to have similar target clusters within their genomic data.

Further, the graphical user interface includes a comparative access element for effecting a comparison of a selected chemical, physical or biological property which may be desired with chemical, physical or biological properties measured or detected within an extract. This comparison is made to allow for identification of a metabolite synthesized by the target gene cluster within a microorganism. Thus, the graphical user interface of the invention allows searchable or query-based access to the knowledge repository of the invention.

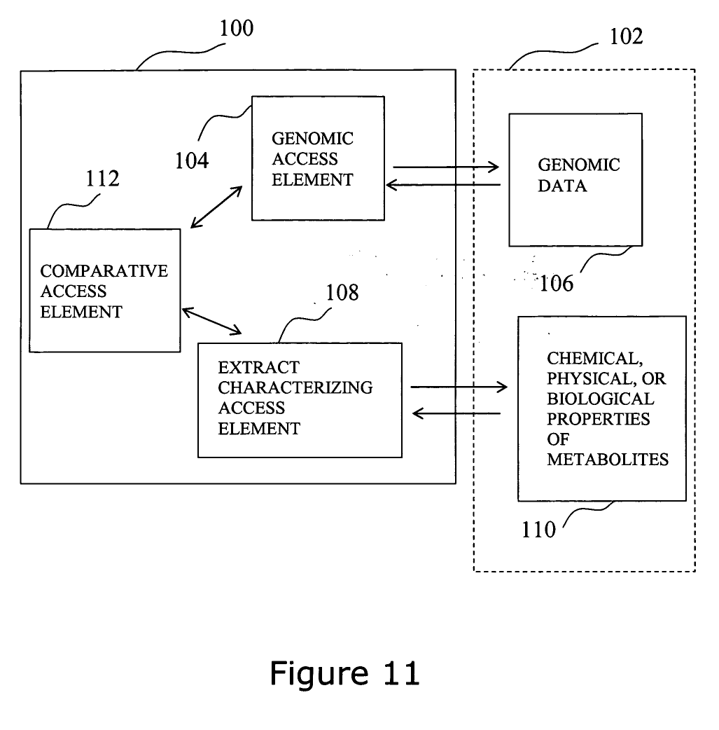

FIG. 11 provides a schematic representation of a typical graphical user interface according to the invention. The graphical user interface (100) is used to subscribe to a knowledge repository (102). The interface comprises a genomic access element (104) for accessing genomic data (106) within the knowledge repository. An extract-characterizing access element (108) is provided for accessing the chemical, physical, or biological properties of metabolites (110) from within the knowledge repository. A comparative access element (112) is also provided which allows a comparison to be effected between an expected or desired property, based on genomic data, with actual properties of metabolites in order to identify a metabolite synthesized by a target gene cluster within a microorganism.

Many variations in the appearance of a graphical user interface (GUI) can be conceived for organizing and displaying data according to the invention, and these would fall within the scope of the graphical user interface of the invention.

The status of different stages or procedures according to certain embodiments of the invention may be displayed on computer medium in the form of reports illustrated on a computer screen. Such reports may also be produced in printed form. The stages of analysis for each extract may be provided within such a report, and success qualifiers for each stage can be provided.

As an example of such a status report, information relating to the chemistry aspects of a project run using the method or system of the invention can be produced in a “Chemistry Project Report”. The Chemistry Project Report may include such parameters as microbial identification data, extract and medium identification data, the scientist responsible for a particular entry in the report, the date on which an entry was made in the report, or the phase status of a particular extract. The phase status may be, for example, a report of whether a stage of a discovery platform has been completed. Evaluation and monitoring of the phase status may be done in any number of ways, such as by assigning a success qualifier to each discrete state of the natural product discovery cascade. A success qualifier may be, for example, a visual differentiator, such as different colors or patterns displayed on the report to indicate success according to a legend. For example, in a Chemistry Project Report, Stage I processes may involve extraction, initial fractionation, and bioassay of a given microorganism in a media formulation; Stage II processes may involve identifying the active component of the extract and determining its molecular weight via HPLC/MS; and Stage III processes may involve isolation of significant quantities of an active component and its structural elucidation. Each of these stages can be evaluated and the status provided in the report.

If visual differentiators are used, the color of each qualifier can be defined in a legend. As an example of color-based visual differentiators: a green success qualifier can be used to indicate that a project was attempted and the result was positive; a red success qualifier may be used to indicate a project was attempted and negative results were obtained; a yellow success qualifier may be used to indicate that a project was completed; a purple success qualifier can be used to indicate that a project was discontinued; and a blue success qualifier may be used to indicate that a project is ongoing. By using visual differentiators, the Chemistry Project Report produced at the Graphic User Interface provides immediate visual assistance to a user, to a greater extent than is available from simply displaying data values, for example.

The reports available may display any number of columns and/or rows of information, as required, and a comments column may also be used to relate observations on the secondary metabolites and/or activity levels detected in a particular extract.

Other types of reports can be provided, including screening tables representing results for a large scale primary screen of extracts from an organism. Screening results from those organisms within a culture collection may be provided in a report format. In one column of such a report the media growth conditions used can be provided, and various test organisms used to asses biological activity (for example antibacterial or antifungal activity) may be listed in a row so as to provide a biological activity array in table format. Biological activity can be rated according to potency, and groups of organisms with unique activities may be ascertained in this manner and submitted for primary CHUMB analysis.

Once CHUMB analysis is completed, the data may be input into the system so as to build the knowledge repository. This data may be accessed through the graphical user interface. The data may be displayed via a “CHUMB” graph of the CHUMB parameters (C18, HPLC, UV, mass and bioactivity). In a typical CHUMB graph, each point in a chromatogram can be assessed in terms of UV spectrum, mass spectrum, and bioactivity. For example, hundreds of separate CHUMB fractions may be used to construct the graph. This adds a chromatographic dimension to traditional screening data and provides indication of groups of compounds with a broad range of polarities that are active against the various test organisms under various conditions. Investigation of the spectra of the bioactive points is used for identification of known compounds (dereplication) and assignment of possible new chemical entities.

According to the invention, the graphical user interface may be used to illustrate the results of a screening matrix representing extracts derived from any particular organism grown under a variety of conditions. Growth conditions may be displayed on the interface or may be accessed through a hierarchy, the top level of which is displayed on the screening matrix. The matrix may be sortable by clicking on a row header. For example, it is possible for a user to sort by “state”, which displays the activity profile of a given medium across a panel of indicators. This would help group media by similar activity profiles.

The graphical user interface may access sources other than the knowledge repository. For example, the interface may allow the user to access a publicly available or private databases through an internet connection, or based on electronic information stored on a CD. Such databases of known natural products which can be searched by physical properties of a compound include the Dictionary of Natural Products and Antibase. Any appropriate database or website could be accessed by the graphical user interface according to the invention.

The graphical user interface may be used to “dereplicate” a data point for example, if a predicted mass derived from a database of known compounds indicates the presence of a particular metabolite. If the organism of interest was previously shown to make the known compound, the compound can be dereplicated from the information contained in the knowledge repository at this point. For those compounds which are not dereplicated during the CHUMB process, (i.e. have no match in the knowledge repository), such compound can be considered as potential new chemical entities.

The graphical user interface may allow query on the basis of the presence of a particular biosynthetic locus. An identified locus within the knowledge repository may be represented by an icon or other representation that may be selected (clicked on) to allow a user to access information as to what type of metabolites are encoded by this locus.

The graphical user interface may also allow a particular genomic sequence to be “BLASTed™” against the genomic information in the database report, which is to say, the sequence (amino acid or nucleic acid) is aligned and compared with other sequences within the knowledge repository for matches as determined using bioinformatics analysis. The sensitivity of such a query (the percentage of identity required to qualify a sequence as a match) may be set by the user.

Sub-Structure Prediction:

Sub-structure prediction on the basis of amino acid or nucleotide sequence in an identified locus is known in the art and described herein. The subject is reviewed, for example, in Martin & Liras, 1989, Ann. Rev. Microbiol. 43: 173-206 and in Floss, 2001, J. Indust. Microbiol. Biotechnol. 27: 183-194. Polyketide synthesis is reviewed in, for example, Staunton & Weissman, 2001, Nat. Prod. Rep. 18: 380-416, as well as in Moore & Hertweck, 2002, Nat. Prod. Rev. 19: 70-99. The prediction of NRPS-derived structures from the sequences of their encoding loci is described in, for example, Conti et al., 1997, EMBO J. 16: 4174-4183, Stachelhaus et al., 1999, Chem. & Biol. 6: 493-505, and Challis et al., 2000, Chem. & Biol. 7: 211-224. Each of these references is incorporated herein by reference in its entirety.

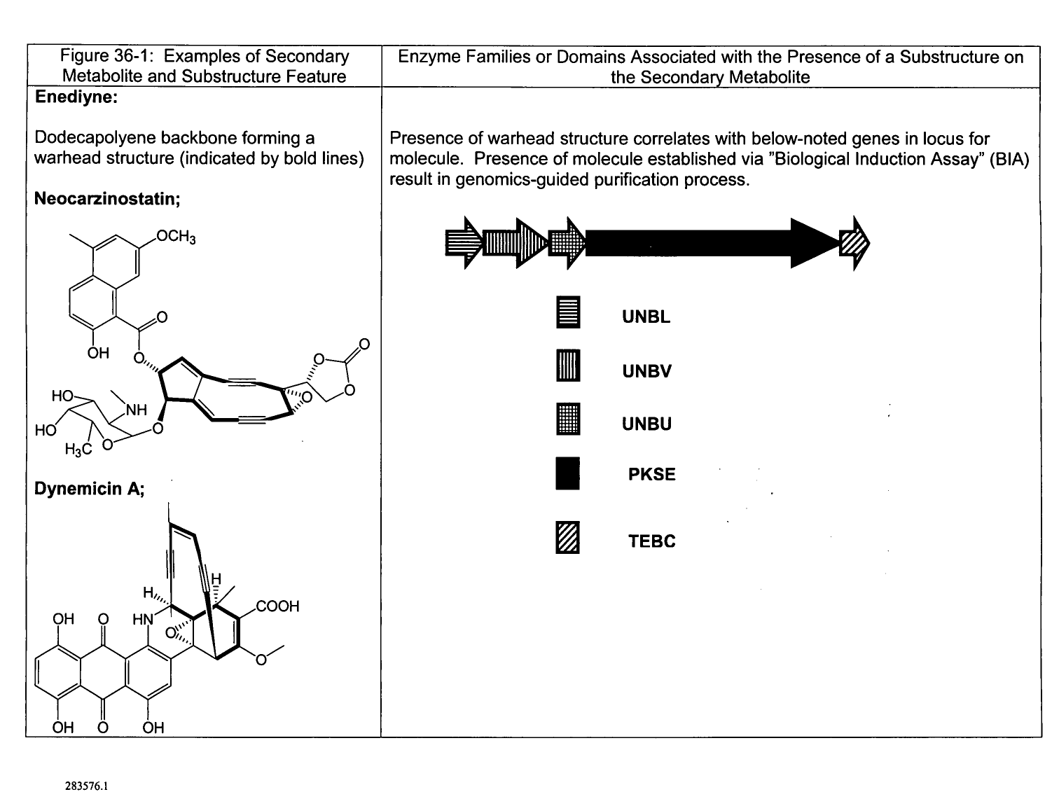

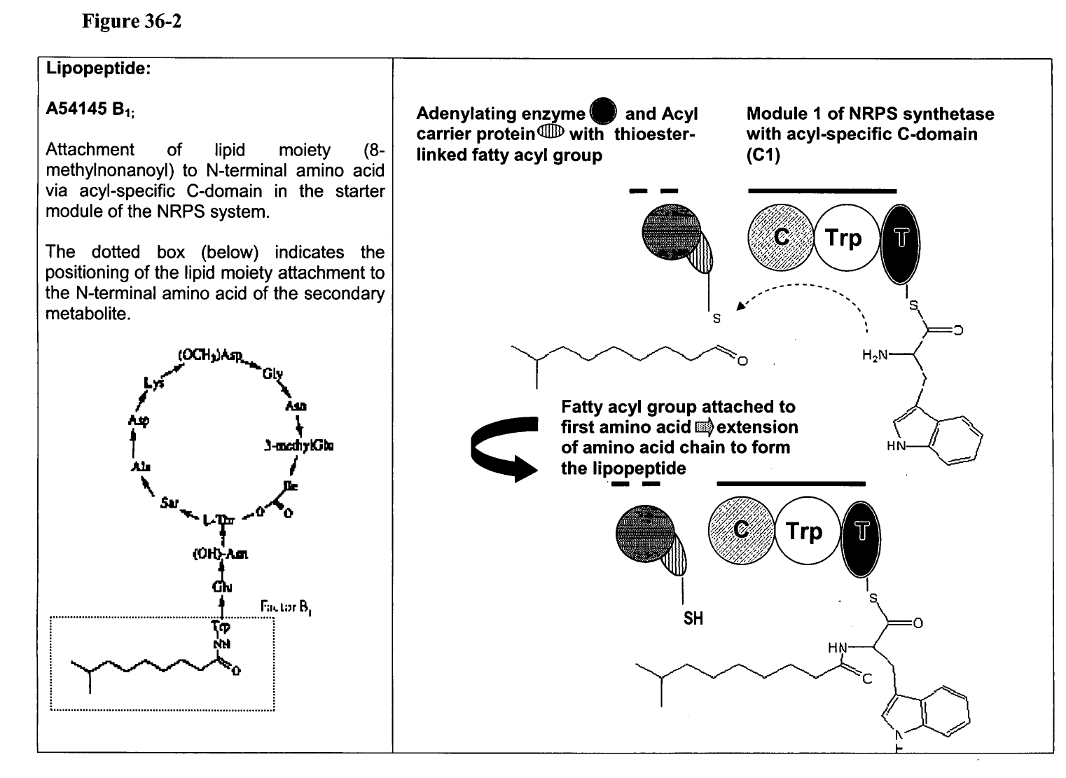

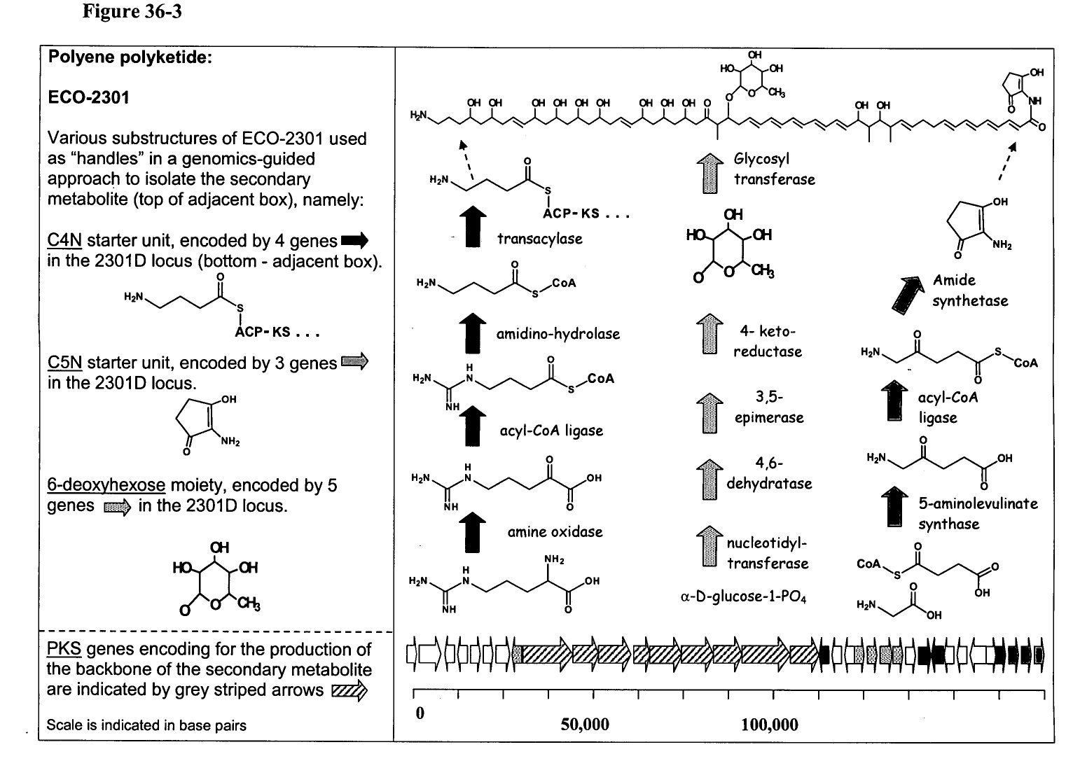

The Examples herein below provide description of the methods described herein as applied to PKS and NRPS biosynthetic loci as well as to loci in which one or more various functionalities are not PKS or NRPS derived. FIG. 36 also summarizes various examples of the genes, enzyme families or domains associated with the biosynthesis and/or presence of sub-structures in various secondary metabolite compounds, and that were utilized in the genomics-guided purification process as further described in the Examples.

### EXAMPLES

### Example 1

### Discovering and Expressing Cryptic Enediyne Natural Product Biosynthetic Pathways

Genomic information related to a conserved group of genes involved in the synthesis of the highly reactive chromophore, ring structure or “warhead” that characterizes all enediynes was generated as described in U.S. Ser. No. 10/152,886 and U.S. Ser. No. 60/398,795. The conserved genes are generally arranged in an operon structure with unidirectional transcription and frequent overlap of translational start and stop codons, suggesting that their gene products are coordinately expressed and functionally related. These genes are from five distinct protein families based on sequence homology and, in some cases, domain organization. The families are referred to as PKSE, TEBC, UNBL, UNBV and UNBU the sequence information for which is provided in U.S. Ser. No. 10/152,886. The PKSE family consists of unimodular iterative polyketide synthases (PKSs) composed of several domains in an unusual order described in more detail below. A putative function was attributed to PKSE, TEBC, UNBL, UNBV and UNBU by comparing their protein sequences to those present in the GenBank™ nonredundant database. PKSE is distantly related to other types of PKSs. The TEBC proteins were found to be similar to the 4-hydroxybenzoyl-CoA thioesterase (1BVQ) of Pseudomonas sp. strain CBS-3 in regions of the protein that have been shown to play an important role in catalysis (Benning, M. M. et al., J. Biol. Chem. 273, 33572-33579 (1998)) and thus may be involved in polyketide chain release and/or cyclization. The UNBL, UNBV and UNBU proteins show no significant homology to proteins in the public databases and therefore represent novel protein families that appear to be specific to enediyne biosynthetic loci. PSORT analysis (Nakai, K. & Horton, Trends Biochem. Sci. 24, 34-36 (1999)) of the UNBV proteins predicts that they are secreted proteins having N-terminal signal sequences, while the UNBU proteins are predicted to be integral membrane proteins with seven or eight putative membrane-spanning alpha helices.

Nucleic acid sequences were provided in U.S. Ser. No. 10/152,886, teaching the presence of “enediyne-specific nucleic acid codes” and “enediyne-specific polypeptide codes”. Furthermore, U.S. Ser. No. 10/152,886 describes a computer system, which is wholly encompassed by the present invention, and which comprises a sequence comparison software for comparing the nucleic acid codes of a query sequence stored on a computer readable medium to a subject sequence which is also stored on a computer readable medium; or for comparing the polypeptide code of a query sequence stored on a computer readable medium to a subject sequence which is also stored on computer readable medium. As provided in U.S. Ser. No. 10/152,886, the sequence comparison software will typically employ one or more specialized comparator algorithms. Protein and/or nucleic acid sequence similarities may be evaluated using any of the variety of sequence comparator algorithms and programs known in the art. Such algorithms and programs include, but are no way limited to, TBLASTN, BLASTN, BLASTP, FASTA, TFASTA, CLUSTAL, HMMER, MAST, or other suitable algorithm known to those skilled in the art (Pearson and Lipman, 1988, Proc. Natl. Acad. Sci USA 85(8): 2444-2448; Altschul et al, 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J. Mol. Biol. 215(3):403-410; Altschul et al., 1993, Nature Genetics 3:266-272; Eddy S. R., Bioinformatics 14:755-763, 1998; Bailey T L et al., 1997, J. Steroid Biochem. Mol. Biol., vol. 62(1): 29-44).

As provided in U.S. Ser. No. 10/152,886, the sequence comparison software will typically employ one or more specialized analyzer algorithms. Any appropriate analyzer algorithm can be used to evaluate similarities, determined by the comparator algorithm, between a query sequence and a subject sequence (referred to herein as a query/subject pair). Based on context specific rules, the annotation of a subject sequence may be assigned to the query sequence. A skilled artisan can readily determine the selection of an appropriate analyzer algorithm and appropriate context specific rules.

As more particularly described in U.S. Ser. No. 10/152,886, the sequence comparison software determines if a gene or set of genes represented by their nucleotide sequence, polypeptide sequence or other representation (the query sequence) is significantly similar to the enediyne-specific nucleic acid codes, a subset thereof, enediyne-specific polypeptide codes, a subset thereof, of the invention (the subject sequence). The software may be implemented in the C or C++ programming language, Java, Perl or other suitable programming language known to a person skilled in the art. As provided in U.S. Ser. No. 10/152,886 the sequence comparison software invokes a “comparator algorithm” that executes the pairwise comparisons between a query sequence and subject sequence for “comparison subprocess”. As noted in U.S. Ser. No. 10/152,886, the comparator algorithm is described to include any algorithm that acts on a query/subject pair, including but not limited to homology algorithms such as BLAST, Smith Waterman, Fasta, or statistical representation/probabilistic algorithms such as Markov models exemplified by HMMER, or other suitable algorithm known to one skilled in the art. Suitable algorithms would generally require a query/subject pair as input and return a score (an indication of likeness between the query and subject), usually through the use of appropriate statistical methods such as Karlin Altschul statistics used in BLAST, Forward or Viterbi algorithms used in Markov models, or other suitable statistics known to those skilled in the art. Furthermore, as described in U.S. Ser. No. 10/152,886, the sequence comparison software also comprises a means of analysis of the results of the pairwise comparisons performed by the comparator algorithm. For an “analysis subprocess”, as described in U.S. Ser. No. 10/152,886, is a process by which the analyzer algorithm is invoked by the software, and the “analyzer algorithm” refers to a process by which annotation of a subject is assigned to the query based on query/subject similarity as determined by the comparator algorithm according to context-specific rules coded into the program or dynamically loaded at runtime. Context-specific rules are what the program uses to determine if the annotation of the subject can be assigned to the query given the context of the comparison. These rules allow the software to qualify the overall meaning of the results of the comparator algorithm. In the various embodiments described in U.S. Ser. No. 10/152,886, the context-specific rules may state that for a set of query sequences to be considered representative of an enediyne locus the comparator algorithm must determine that the set of query sequences contain at least one query sequence that shows a statistical similarity to reference sequences corresponding to a nucleic acid sequence coding for the various polypeptide sequences, or fragments thereof, as provided in U.S. Ser. No. 10/152,886. Likewise, as provided in U.S. Ser. No. 10/152,886, context-specific rules may state that for a query sequence to be considered an enediyne polyketide synthase, the comparator algorithm must determine that the query sequence shows a statistical similarity to subject sequences corresponding to a nucleic acid sequence coding for various polypeptide sequences, or fragments thereof, as provided in U.S. Ser. No. 10/152,886. As provided in U.S. Ser. No. 10/152,886, the analysis subprocess may be employed in conjunction with any other context specific rules and may be adapted to suit different embodiments. The principal function of the analyzer algorithm is to assign meaning or a diagnosis to a query or set of queries based on context specific rules that are application specific and may be changed without altering the overall role of the analyzer algorithm.

As noted in U.S. Ser. No. 10/152,886, the sequence comparison software further comprises a means of returning of the results of the comparisons by the comparator algorithm and analyzed by the analyzer algorithm to the user or process that requested the comparison or comparisons. As mentioned in U.S. Ser. No. 10/152,886, returned results may be written to a file, displayed in some user interface such as a console, custom graphical interface, web interface, or other suitable implementation specific interface, or uploaded to some database such as a relational database, or other suitable implementation specific database. Exit of the sequence comparison program occurs upon return of the results to the user or process that requested the comparison or comparisons.

As described in U.S. Ser. No. 10/152,886, the comparator algorithm may be represented in pseudocode as follows:

| | | |
|---|---|---|
| INPUT: | Q[m]: | query, m is the length |
| | S[n]: | subject, n is the length |
| | x: | x is the size of a segment |

START:
for each i in [1,n] do
for each j in [1,m] do

if ( j + x − 1 ) <= m and ( i + x −1 ) <= n then

```
if Q(j, j+x−1) = S(i, i+x−1) then
    k=1;
    while Q(j, j+x−1+k ) = S(i, i+x−1+ k) do
        k++;
    Store highest local homology
```

Compute overall homology score
Return local and overall homology scores
END.

Furthermore, as described U.S. Ser. No. 10/152,886, the comparator algorithm may be written for use on nucleotide sequences, in which case the scoring scheme would be implemented so as to calculate scores and apply penalties based on the chemical nature of nucleotides. The comparator algorithm may also provide for the presence of gaps in the scoring method for nucleotide or polypeptide sequences. As more particularly described in U.S. Ser. No. 10/152,886, BLAST is one implementation of the comparator algorithm, while HMMER is another implementation of the comparator algorithm based on Markov model analysis. In a HMMER implementation a query sequence would be compared to a mathematical model representative of a subject sequence or sequences rather than using sequence homology.

As further described in U.S. Ser. No. 10/152,886, the analyzer algorithm receives as its input an array of pairs that had been matched by the comparator algorithm The array consists of at least a query identifier, a subject identifier and the associated value of the measure of their similarity. In the context of U.S. Ser. No. 10/152,386, to determine if a group of query sequences includes sequences diagnostic of an enediyne biosynthetic gene cluster, a reference or diagnostic array is generated by accessing a data source and retrieving enediyne specific information relating to enediyne-specific nucleic acid codes and enediyne-specific polypeptide codes. Diagnostic array consists at least of subject identifiers and their associated annotation. Annotation may include reference to the five protein families diagnostic of enediyne biosynthetic genes clusters, i.e. PKSE, TEBC, UNBL, UNBV and UNBU. Annotation may also include information regarding exclusive presence in loci of a specific structural class or may include previously computed matches to other databases, for example databases of motifs. Once the algorithm has successfully generated or received the two necessary arrays, and holds in memory any context specific rules, each matched pair as determined by the comparator algorithm can be evaluated. The algorithm will perform an evaluation of each matched pair and based on the context specific rules confirm or fail to confirm the match as valid. In cases of successful confirmation of the match the annotation of the subject is assigned to the query. Results of each comparison are stored. As noted in U.S. Ser. No. 10/152,886, completion of the analysis of all matched pairs occurs when the end of the query/subject array is reached. Once all query/subject pairs have been evaluated against enediyne-specific nucleic acid codes and enediyne-specific polypeptide codes, a final determination can be made if the query set of ORFs represents an enediyne locus. The algorithm then returns the overall diagnosis and an array of characterized query/subject pairs along with supporting evidence to the calling program or process and then terminates.

As described in U.S. Ser. No. 10/152,886, the analyzer algorithm may be configured to dynamically load different diagnostic arrays and context specific rules. It may be used for example in the comparison of query/subject pairs with diagnostic subjects for other biosynthetic pathways, such as chromoprotein enediyne-specific nucleic acid codes or non-chromoprotein enediyne-specific polypeptide codes, or other sets of annotated subjects.

The DECIPHER® database (Ecopia BioSciences Inc., St.-Laurent, QC, CANADA) was consulted to identify microorganisms containing the enediyne warhead cassette cluster but not previously reported to produce enediyne compounds. Such cryptic enediyne gene clusters were identified in Amycolatopsis orientalis ATCC 43491 (a known vancomycin producer), Streptomyces ghanaensis NRRL B-12104 (a known moenomycin procucer), Kitasatosporia sp. CECT 4991 (a known taxane producer), Micromonospora megalomicea subsp. nigra NRRL 3275 (a known megalomicin producer), Streptomyces cavourensis subsp. washingtonensis NRRL B-8030 (a known chromomycin producer), Saccharothrix aerocolonigenes ATCC 39243 (a known rebeccamycin producer), Streptomyces kaniharaensis ATCC 21070 (a known coformycin producer), Streptomyces citricolor IFO 13005 (a known aristeromycin and neplanocin A producer). The cryptic enediyne biosynthetic loci were identified by the presence of the conserved enediyne warhead cassette genes as well as other flanking genes frequently found in biosynthetic loci encoding other natural product classes.

As PKSE, TEBC, UNBL, UNBV and UNBU are the only genes common to all enediyne loci and the single structural feature found in all known enediynes is the warhead (Nicolaou, K. C. et al., Proc. Natl. Acad. Sci. USA, 90, 5881-5888 (1993)), a genomics-based correlation between PKSE, TEBC, UNBL, UNBV and UNBU genes as a functional unit responsible for the biogenesis of the warhead was established. The PKSEs are likely to generate the carbon skeleton of the warhead by catalysing iterative cycles of acyl-coenzyme A (acyl-CoA) condensation, ketoreduction and dehydration, using an acyl carrier protein (ACP) domain as a covalent attachment site for the growing carbon chain. The PKSEs contain enzymatic domains characteristic of known PKSs, including ketoacyl synthase (KS), acyltransferase (AT), ketoreductase (KR) and dehydratase (DH) domains, as well as ACP domains. Additional analysis of the PKSE sequences further revealed a domain in the C-terminal region of the protein that is similar to 4′-phosphopantetheinyl transferases (PPTases) (Walsh, C. T., et al., Curr. Opin. Chem. Biol. 1, 309-315 (1997)) and is likely to be involved in posttranslational autoactivation of the PKSE. While the functions of the TEBC, UNBL, UNBV and UNBU proteins remain unknown, the strict association of these proteins with the warhead PKS and their presence in all enediyne biosynthetic loci strongly suggests that they play essential roles in the formation, stabilization or transport of the enediyne warhead.

The shared warhead structure provides all enediyne with the ability to damage DNA. The mechanism of action of enediynes involves binding of the enediyne compound to DNA and the warhead chromophore undergoing the thermodynamically favorable Bergman cyclization resulting in strand cleavage of genomic DNA. The biochemical induction assay (BIA) is a modified prophage induction assay that detects agents that damage DNA (Elespuru, R. K. & Yarmolinsky, M. B., Environmental Mutagenesis. 1, 65-78 (1979)). It is predicted that strains harbouring the warhead genes, when cultured in particular fermentation conditions to induce expression of the gene cluster associated with the enediyne genes will produce an enediyne natural product which in turn can be detected using the BIA.

The microorganisms containing the cryptic enediyne biosynthetic loci were grown under multiple culture conditions to obtain extracts containing the enediyne metabolites. The strains found to contain a putative enediyne biosynthetic locus were cultured in a variety of fermentation media. Organisms were initially grown in 25 ml of TSB seed medium (Kieser, T. et al., Practical Streptomyces Genetics, The John Innes Foundation, Norwich, United Kingdom, (2000)) for 60 h at 28° C. and then diluted 30-fold in 25 ml production media. Production cultures (25 ml) were incubated for 7 days at 28° C. under constant agitation. Two milliliters of culture were removed and clarified by centrifugation to provide supernatant samples. The rest of the culture (supernatant and mycelia) was extracted with an equal volume of methanol under agitation for 30 min. Extracts were clarified by centrifugation and diluted accordingly in their respective media supplemented with 50% methanol. The BIA was performed as described in Elespuru, R. K. & Yarmolinsky, M. B., Environmental Mutagenesis. 1, 65-78 (1979). Briefly, 10 μl of supernatant or extract and two-fold serial dilutions thereof were applied to agar plates seeded with Escherichia coli BR513 and incubated for 3 hours at 37° C. Soft agar containing 0.7 mg/ml of X-Gal was added onto the plate and colour development was observed within 30 min.

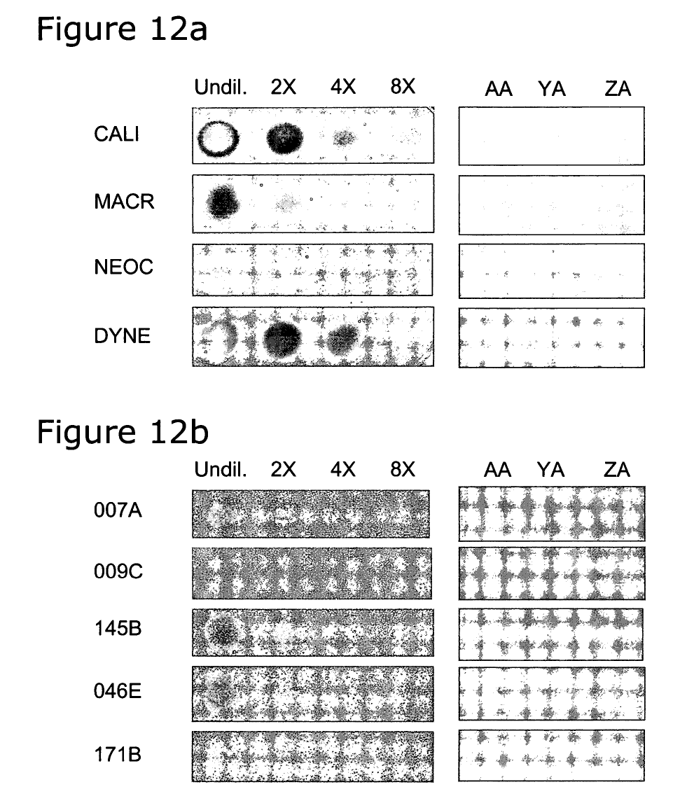

All production media used in this study were assayed alone. Growth of the strains in most media failed to result in detectable BIA activity. However, all strains produced BIA activity when grown in specialized media selected for their ability to support enediyne production (FIG. 12). For calicheamicin, macromomycin and dynemicin, the production media that triggered expression of the enediyne biosynthetic locus were CB, ES and DY. The production media that triggered expression of the neocarzinostatin enediyne biosynthetic locus was NG. The production media supporting expression of the cryptic enediyne biosynthetic locus in Amycolatopsis orientalis was CB. The production media that supported expression of the cryptic enediyne biosynthetic locus in Streptomyces ghanaensis was KE. The production media that supported expression of the cryptic enediyne biosynthetic locus in Saccharothrix aerocolonigenes was ET. The production media that supported expression of the cryptic enediyne biosynthetic locus in Streptomyces kaniharaensis was ET. The production media that supported expression of the cryptic enediyne biosynthetic locus in Ecopia strain 171 was DY. The production media that supported expression of the cryptic enediyne biosynthetic locus in Streptomyces citricolor was MC. The production media that supported expression of the cryptic enediyne biosynthetic locus in Ecopia strain 046 was MC. The production media that supported expression of the cryptic enediyne biosynthetic locus in Streptomyces cavourensis subsp. washingtonensis was SP. Examples of media not supporting enediyne production include CECT media 32 and 131 (Colección Españiola de Cultivos Tipo, Valencia, Spain) herein referred to as media YA and ZA, respectively.

The data generated, including (i) the presence of the PKSE, TEBC, UNBL, UNBU and UNBV genes in each of the microorganisms, notably those not previously reported to produce an enediyne metabolite; (ii) the putative function attributed to the PKSE, TEBC, UNBL, UNBU and UNBV proteins in the enediyne loci; (iii) the multiple culture conditions under which the strains were grown; and (iv) the results of the biochemical induction assay and other bioassays were added to the DECIPHER® database. These data facilitates subsequent comparisons and dereplication of enediyne activities.

### Example 2

### Isolation and Structure Elucidation of a Metabolite from a Cryptic Biosynthetic Locus

The systems, methods and knowledge repository of the invention can be used to isolate and elucidate the structure of a metabolite synthesized by a cryptic biosynthetic locus, the product of which is unknown. A sample of the organism Streptomyces cattleya (NRRL 8057) was obtained from the Agricultural Research Service Culture Collection, Peoria, Ill. 61604). A literature search (PubMed) revealed Streptomyces cattleya (NRRL 8057) had not been reported to produce any natural products other than thienamycin and other beta-lactam class compounds (U.S. Pat. No. 3,950,357).

Streptomyces cattleya was subject to the genome scanning method described in U.S. Ser. No. 10/232,370 which resulted in the discovery in the Streptomyces cattleya genome of at least 12 putative natural product biosynthetic loci. These were further characterized by sequence analysis and determined to be distinct biosynthetic loci. Sequence analysis was performed using a 3700 ABI capillary electrophoresis DNA sequencer (Applied Biosystems) and open reading frames were identified from the sequence information. The DNA sequences of the ORFs were translated into amino acid sequences and compared to the National Center for Biotechnology Information (NCBI) nonredundant protein database using the BLASTP™ algorithm with the default parameters (Altschul et al., supra). Sequence similarity with known proteins of defined function resulted in a putative function being attributed to a number of genes in each of the 12 biosynthetic loci. As described in U.S. Ser. No. 10/232,370, sequence similarity can be assessed by percent identity or by E value. The E value relates the expected number of chance alignments with an alignment score at least equal to the observed alignment score. An E value of 0.00 indicates a perfect homolog. The E values are calculated as described in Altschul et al. J. Mol. Biol., October 5; 215(3) 403-10, the teachings of which are incorporated herein by reference. The E value assists in the determination of whether two sequences display sufficient similarity to justify an inference of homology. An E value of 10⁻¹⁰ or less will generally be indicative of two proteins that are significantly related to one another, an E value of 10⁻¹⁵ being especially significant. However the length and accuracy of the sequenced being compared with the database will strongly influence the value of E considered significant. The use of a filter to mask stretches of low complexity or highly biased amino acid sequences can be used to increase the specificity of homology comparisons. As further described in U.S. Ser. No. 10/232,370 sequence alignments displaying an E value of at least 10⁻⁵ were considered as significantly homologous and retained for further evaluation.

Sequence alignments or comparisons for the determination of sequence similarity can be performed using standard software. The most commonly used software packages include BLAST and FASTA.

Of the 12 biosynthetic loci discovered six of them included putative polyketide synthases (PKS) of different varieties based on domain organization.

Streptomyces cattleya was grown in six media formulations, namely BA, DA, EA, KA, NA, OA, for a period of 7 days. Non-polar extraction procedures were employed to capture polyketide based natural products from the culture broths. An equal volume of ethyl acetate was added to the whole broth, which was subsequently agitated on an orbital shaker for 30 minutes. The organic layer was separated, dried over magnesium sulfate, and evaporated to yield a crude extract. The extracts were analyzed by thin-layer chromatography and overlay bioassay using several indicator strains (B. subtillis, S. aureus, E. coli, C. albicans, M. luteus, K pneumonia, P. aeruginosa). Multiple zones of antimicrobial activity were observed in the overlay assays in the extracts derived from the various media. These antimicrobial/antifungal activities are commonly associated with secondary metabolites in Steptomyces and provide convenient assays which can be used to follow progress in purification (bioassay-guided fractionation). Extracts from media DA exhibited substantial Micrococcus luteus activity, and was selected for purification by flash chromatography (SiO₂ plug, 5% MeOH/CH₂Cl₂—100% MeOH) followed by Sephadex™ LH-20 chromatography (100% MeOH) resulting in a compound that was pure by TLC analysis. ¹H NMR analysis verified that the compound was substantially pure and suggested a polyketide class molecule with multiple double bonds, as evidenced by peaks at 5.5-6.5 ppm (consistent with alkenic double bonds), peaks at 3.5-4.5 (consistent with hydroxyl attached C—H bonds), and 0.5-3 (consistent with alkyl groups).

Genomics information from a knowledge repository assisted in the structure elucidation process. The DECIPHER® database was consulted to associate the measured chemical, physical and biological properties of the polyketide metabolite with one of the “cryptic” biosynthetic loci (the target locus) from Streptomyces cattleya. PKS domain identification was performed on the target locus. Genomics analysis allowed deduction of a biosynthetic scheme for production of the polyketide metabolite by the target locus, using bioinformatic analysis of the polyketide chain and comparative analysis with the structure of other PKS enzymes in the DECIPHER® database. In particular, the analysis suggested domain strings from which various structural elements were derived. A portion of the genomic deductions and the corresponding structural deductions are represented below:

[KS-IX-KR-MT-ACP] [KS-IX-KR-ACP] [KS-IX-ACP]

[C-A(Gly_-ACP] [KS] [IX-DH-KR-ACP] [KS-IX-DH-KR-MT-ACP] [KS-IX-ACP] [KS-IX-KR-ACP]

[KS-IX-KR-ACP] [KS] [DH-ACP-KR] [KS-IX-DH-KR-ACP] [KS-IX-DH-KR-ACP] 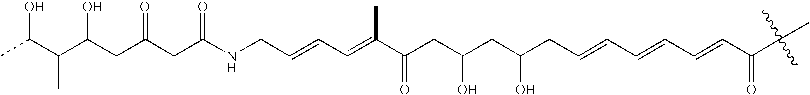
where abbreviations describe processive enzymatic activities or other functions corresponding to ketoacyl synthase (KS), acyltransferase interaction domain (IX), ketoreductase (KR), dehydratase (DH), and enoyl reductase (ER), acyl carrier protein (ACP), methyltransferase (MT), and thioesterase (TE) activity involved in polyketide synthesis, as well as condensation (C) and adenylation (A) activities.

These structural elements were used as possible starting points for structure elucidation studies with multidimensional NMR experiments such as DQCOSY, TOCSY, HSQC, and HMBC. The structural elements deduced from the genomic information matched the experimental NMR data and facilitated the solving of partial structures. The partial structures thus obtained were used to query a database of known natural products and the known compound L-681,217 was identified. The reported spectroscopic data for compound L-681,217 was an exact match to the spectroscopic data collected for the compound isolated from Streptomyces cattleya. The structure of compound L-681,217 is shown below:

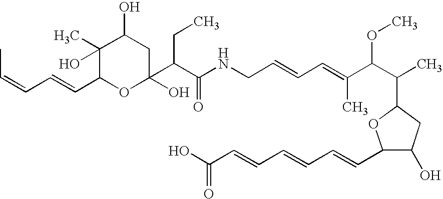

The structure of compound L-681,217 was associated with the biosynthetic locus from Streptomyces cattleya and a link between the structure data and genomics data was made in the DECIPHER® database. This association was, in turn, used to link or associate a separate locus in another organism with a structurally similar compound that is known to be produced by that organism (Streptomyces filippiniensis, heneicomycin). In particular, a comparison of the structures of L-681,217 and heneicomycin led to the prediction that a domain string would be found in the heneicomycin-producer Streptomyces filippiniensis. In support of this prediction, a target locus encoding such a domain string was identified in the genomic data from Streptomyces filippiniensis, as shown below: 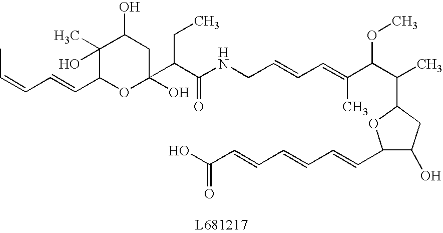
Domains of L681217 locus
[TP]
[ACP] [KS-IX-ACP] [KS]
[DH-ACP-KR][KS-IX-KR-MT-ACP] [KS-IX-KR-ACP] [KS-IX-ACP]
[C-A(Gly_)-ACP] [KS]
[IX-DH-KR-ACP] [KS-IX-DH-KR-MT-ACP] [KS-IX-ACP] [KS-IX-KR-ACP] [KS-IX-KR-ACP][KS]
[DH-ACP-KR] [KS-IX-DH-KR-ACP]
[KS-IX-DH-KR-ACP] [ks-at]
[AT] [AT] [NPDC-XX] 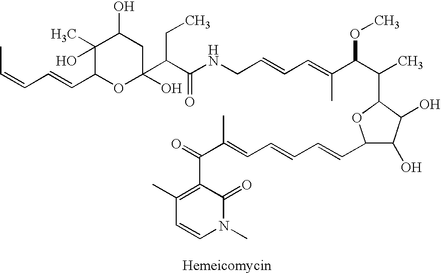
Partial domain string
. . . [ACP] [KS-IX-KR-ACP] [KS]
[DH-ACP-KR] [KS-IX-KR-MT-ACP] [KS-IX-KR-ACP] [KS-IX-ACP]
[C-A(Gly_)-ACP] [KS]
[DH-KR-ACP] [KS-IX-DH-KR-MT] [KS-IX-ACP] [KS-IX-KR-ACP] [KS-IX-ACP] [KS]

### Example 3

### Identifying a Secondary Metabolite of a Pre-Selected Chemical Family

The methods, systems and knowledge repositories of the invention can be used to identify a secondary metabolite of a pre-selected chemical family. In this example we describe the identification of the antifungal polyketide Ayfactin™, a member of the pre-selected chemical family of “polyenes”.

A knowledge repository was consulted to determine chemical family data for a polyene polyketide. A target gene cluster encoding a putative polyene metabolite was identified based on bioinformatic analysis of genomic information present in the DECIPHER® database (Ecopia Biosciences Inc., St.-Laurent, Canada). The target gene cluster encodes polyketide synthases as well as other proteins similar to those encoded by previously sequenced antifungal polyene biosynthetic loci such as those for partricin, candicidin and nystatin. In particular, the domain structure of the sequenced polyketide synthases includes a partial domain string deduced to be . . . DH-KR-ACP] [KS-AT-DH-KR-ACP] [KS-AT-DH-KR-ACP] [KS-AT-DH-KR-ACP] [KS-AT-DH-KR-ACP] [KS-AT-DH-KR-ACP][KS-AT-DH-KR-ACP] . . . corresponding to the synthesis of a polyketide chain with seven or more conjugated double bonds, a structural feature consistent with polyenes such as candicidin. All the AT domains in the domain string were predicted to be specific for malonyl-CoA extender units. The gene cluster also includes genes that are most closely related to genes found in the Streptomyces griseus IMRU 3570 biosynthetic gene cluster encoding candicidin, a polyene compound. These genes include a para-aminobenzoic acid synthase that displays 77% identity and 82% similarity to a synthase in the candicidin cluster (GenBank accession CAC22117); a thioesterase that displays 69% identity and 81% similarity to a thioesterase in the candicidin cluster (GenBank accession CAC22116); and an aminotransferase that displays 79% identity and 89% similarity to an aminotransferase in the candicidin cluster (GenBank accession CAC22113).

The microorganism containing the target gene cluster identified from the DECIPHER® database (designated herein as organism 100) was one from the Ecopia culture collection. Organism 100 had been analyzed using the genome scanning method referred to in Example 1 which resulted in the discovery of several natural product biosynthetic loci, seven of which were further characterized by high-throughput sequencing. The results of the genome scanning and the high throughput sequencing had been entered into the DECIPHER® database. Thus, organism 100 was predicted to contain a biosynthetic locus (designated herein as locus 100C) coding for the production of a putative antifungal polyene containing seven or more conjugated double bonds.

An extract containing the putative polyene was obtained from organism 100 using a metabolomic approach to identify conditions under which the product of locus 100C was expressed. This approach obtains analytical measurement of all low molecular weight metabolites in a given organism at a specific time when grown under specific culture conditions. Organism 100 was grown in 48 different media, namely AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA. Metabolites were extracted from whole cell cultures by adding of an equal volume of methanol. After removal of solid debris, the extract was concentrated and injected into an HPLC/MS system in which the metabolites were analyzed to obtain UV and mass data and purified fractions are collected in 96-well plates and assayed for multiple activities including antibiotic activity against gram-positive and gram-negative bacteria, and fungi. Analysis of the chromatographic and bioactivity profiles indicated the presence of a potent antifungal activity in a number of extracts. For example, media RM produced substantial quantities of a chromatographically distinct compound that discplayed antifungal activity against Candida indicators.

Finally, the extracts generated by growth of organism 100 under each of the 48 media were analyzed for metabolites having physical, chemical and biological characteristics of polyenes. This analysis identified a compound of mass 1113 Da having an extended UV chromophore consistent with a heptaene (i.e. having 7 conjugated double bonds) and antifungal activity. Searching a database of greater than 25000 known microbial natural products with this mass, UV, and bioactivity data provided conclusive evidence that the polyene is the known antifungal agent ayfactin, the structure of which is shown below:

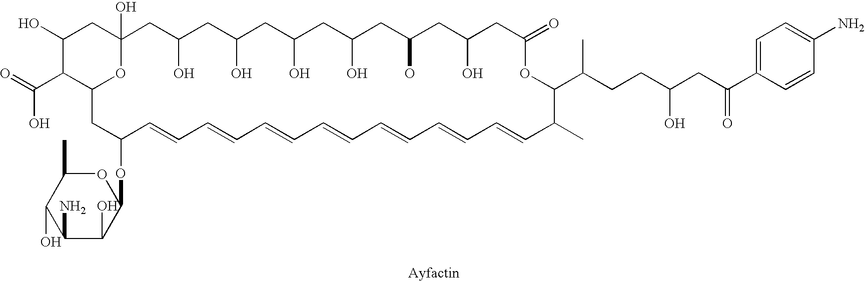

The measured chemical, physical and biological properties of the product of locus 100° C. were found to be consistent with the reported chemical, physical and biological properties for ayfactin, and are in precise agreement with the bioinformatic predictions made in regard to an antifungal polyene. The DECIPHER® database was updated to establish a link that associates locus 100° C. in organism 100 with the chemical structure of ayfactin.

### Example 4

### Detection of a lipopeptide metabolite from Streptomyces refuineus subsp. thermotolerans NRRL 3143

Lipopeptides are natural products that exhibit potent, broad-spectrum antibiotic activity with a high potential for biotechnological and pharmaceutical applications as antimicrobial, antifungal, or antiviral agents. A single microorganism may produce a mixture of related lipopeptides that differ in the lipid moiety that is attached to the peptide core via a free amine, usually the N-terminal amine of the peptide core. The lipid moiety can have a major influence on the biological properties of lipopeptide natural products.

Lipopeptides produced by bacteria are synthesized nonribosomally on large multifunctional proteins termed nonribosomal peptide synthetases (NRPSs) (Doekel and Marahiel, 2001, Metabolic Engineering, Vol. 3, pp. 64-77). NRPSs are modular proteins that consist of one or more polyfunctional polypeptides each of which is made up of modules. The amino-terminal to carboxy-terminal order and specificities of the individual modules correspond to the sequential order and identity of the amino acid residues of the peptide product. Each NRPS module recognizes a specific amino acid substrate and catalyzes the stepwise condensation to form a growing peptide chain. The identity of the amino acid recognized by a particular unit can be determined by comparison with other units of known specificity (Challis and Ravel, 2000, FEMS Microbiology Letters, Vol. 187, pp. 111-114). In many peptide synthetases, there is a strict correlation between the order of repeated units in a peptide synthetase and the order in which the respective amino acids appear in the peptide product, making it possible to correlate peptides of known structure with putative genes encoding their synthesis, as demonstrated by the identification of the mycobactin biosynthetic gene cluster from the genome of Mycobacterium tuberculosis (Quadri et al., 1998, Chem. Biol. Vol. 5, pp. 631-645).

The modules of a peptide synthetase are composed of smaller units or “domains” that each carry out a specific role in the recognition, activation, modification and joining of amino acid precursors to form the peptide product. One type of domain, the adenylation (A) domain, is responsible for selectively recognizing and activating the amino acid that is to be incorporated by a particular unit of the peptide synthetase. The activated amino acid is covalently attached to the peptide synthetase through another type of domain, the thiolation (T) domain, that is generally located adjacent to the A domain. Amino acids joined to successive units of the peptide synthetase are subsequently covalently linked together by the formation of amide bonds catalyzed by another type of domain, the condensation (C) domain. NRPS modules can also occasionally contain additional functional domains that carry out auxiliary reactions, the most common being epimerization of an amino acid substrate from the L- to the D-form. This reaction is catalyzed by a domain referred to as an epimerization (E) domain that is generally located adjacent to the T domain of a given NRPS module. Thus, a typical NRPS module has the following domain organization: C-A-T-(E).

Lipopeptides differ from regular peptides in that they contain a lipid moiety usually attached at the N-terminal amine of the peptide core structure. In contrast to regular peptides, in lipopeptide-encoding NRPS clusters the adenylation domain responsible for the activation and tethering of the first amino acid residue of the peptide core is preceded by an unusual condensation domain (C-domain). The genomic information pertaining to the unusual C-domain was generated as described in co-pending applications U.S. Ser. No. 10/329,027 filed Dec. 24, 2002 entitled Compositions, methods and systems for discovery of lipopeptides and U.S. Ser. No. 10/329,079 entitled Genes and proteins involved in the biosynthesis of lipopeptides, the contents of which are incorporated herein by reference. The unusual C-domain is referred to as an “acyl-specific C-domain” in co-pending applications U.S. Ser. No. 10/329,027 and 10/329,079. As further described in reference to a first and a second consensus sequences provided in U.S. Ser. No. 10/329,027, the acyl-specific C-domain is defined structurally as a polypeptide sequence that produces an alignment with at least 45% identity to one of the first two consensus sequences using the BLASTP 2.0.10 algorithm (with the filter option -F set to false, the gap opening penalty -G set to 11, the gap extension penalty -E set to 1, and all remaining options set to default values. Furthermore, as provided in U.S. Ser. No. 10/329,027, the consensus sequences were generated as follows. First, the listed sequences were aligned with the ClustalX 1.81 program using default settings. Then a profile hidden Markov model (HMM) was made from the alignment file with the hmmbuild program of the HMMER 2.2 package (Sean Eddy, Washington University; world-wide-web hmmer.wustl.edu/) and was calibrated with the hmmcalibrate program of the HMMER package, both using default settings. Briefly, a profile hidden Markov model is a statistical description of a sequence family's consensus. HMMER is a freely distributable implementation of profile HMM software for protein sequence analysis and is available from the above web site. Finally, the consensus sequences were generated from the HMM with the hmmemit program of the HMMER package using the -c option so as to predict a single majority rule consensus sequence from the HMM's probability distribution. The presence of an acyl-specific C-domain in an NRPS system along with the specific location of this domain in the starter module of the NRPS system indicate that the product encoded by the NRPS system is likely to be a lipopeptide.

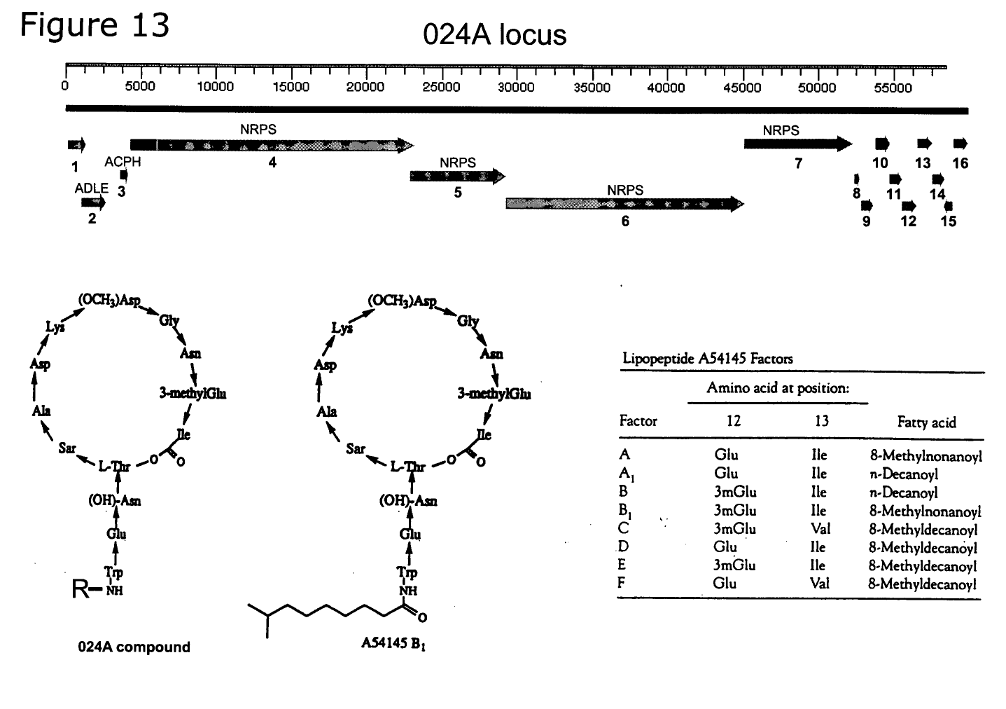

To search for microorganisms that may produce lipopeptides, the DECIPHER® database was consulted to identify microorganisms which contain in their genome an acyl-specific C-domain. One of the microorganisms selected from the DECIPHER® database that clearly contained an acyl-specific C-domain was Streptomyces refuineus NRRL 3143. Further analysis, described in detail in co-pending applications U.S. Ser. No. 10/329,027 and U.S. Ser. No. 10/329,079, established that this unusual condensation domain was contained in a large NRPS system in Streptomyces refuineus, herein referred to as locus 024A. The precise location of the acyl-specific C-domain was determined to be in the starter loading domain of the NRPS system, indicating that 024A was encoding an N-acylated lipopeptide product (FIG. 13).

Analysis of genomic information contained in the DECIPHER® database allowed the prediction that the NRPS system containing the unusual C-domain in the Streptomyces refuineus 024A locus would direct the synthesis of a polypeptide scaffold identical to that of the known lipopeptide A54145 produced by Streptomyces fradiae (FIG. 13). The genetic locus responsible for biosynthesis of the lipopeptide A54145 is present in the DECIPHER® database. The overall genetic similarity observed between the 024A and A54145 biosynthetic loci also indicated that both loci would be expressed under similar growth conditions in the two Streptomyces species (U.S. Ser. No. 10/329,079 and Zazopoulos et al., 2003, Nature Biotechnol., Vol 21) Based on the prediction of structural similarity between the two compounds, it was also expected that the 024A-encoded lipopeptide would have chemical, physical and biological properties similar to those of A54145.

A patent database was then consulted to identify culture conditions under which lipopeptide A54145 in Streptomyces fradiae is expressed (U.S. Pat. No. 4,977,083). Streptomyces fradiae and Streptomyces refuineus were grown under identical culture conditions to assess induction of locus 024A and determine the nature of the specified product.

Both microorganisms were grown at 30° C. for 48 hour in a rotary shaker in 25 mL of a seed medium consisting of glucose (10 g/L), potato starch (30 g/L), soy flour (20 g/L), Pharmamedia (20 g/L), and CaCO₃ (2 g/L) in tap water. Five mL of this seed culture was used to inoculate 500 mL of production media in a 4L baffled flask. Production media consisted of glucose (25 g/L), soy grits (18.75 g/L), Blackstrap molasses (3.75 g/L), casein (1.25 g/L), sodium acetate (8 g/L), and CaCO₃ (3.13 g/L) in tap water, and proceeded for 7 days at 30° C. on a rotary shaker. The production culture was centrifuged and filtered to remove mycelia and solid matter. The pH was adjusted to 6.4 and 46 mL of Diaion HP20 was added and stirred for 30 minutes. HP20 resin was collected by Buchner filtration and washed successively with 140 mL water and 90 mL 15% CH₃CN/H₂0, and the wash was discarded. HP20 resin was then eluted with 140 mL 50% CH₃CN/H₂0 (fraction HP20 E2). This pool was passed over a 5 mL Amberlite IRA67 column (acetate cycle) and the flow through (fraction IRA FT) was reserved for bioassay. The column was washed with 25 mL 50% CH₃CN/H₂0 and eluted with 25 mL 50% CH₃CN/H₂0 containing 0.1 N HOAc (fraction RA E1), and then eluted with 25 mL 50% CH₃CN/H₂0 containing 1.0 N HOAc (fraction IRA E2). Biological activity was followed during purification by bioassay with Micrococcus luteus in Nutrient Agar containing 5 mM CaCl₂.

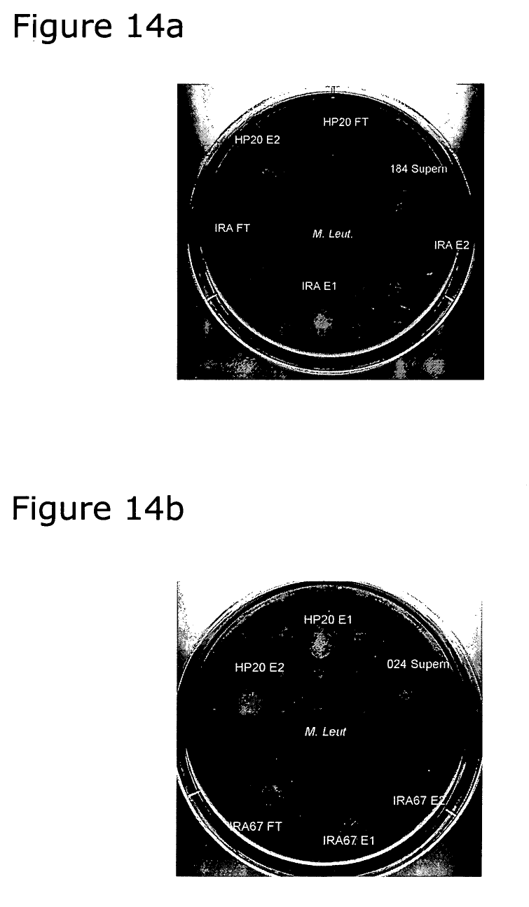

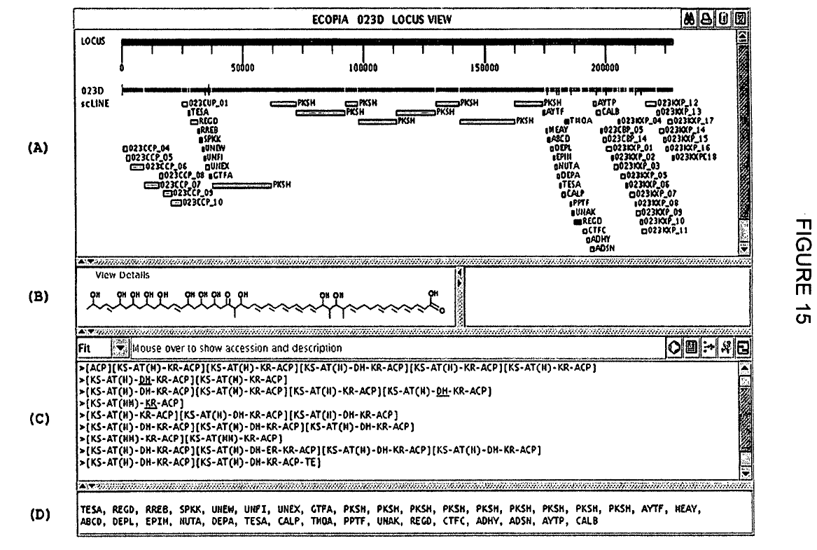

FIG. 14a is a photograph of a plate generated during extraction of an anionic lipopeptide from Streptomyces fradiae, showing an enrichment of activity based on IRA67 anion exchange chromatography consistent with expression of an acidic lipopeptide. This activity is concentrated during the extraction procedure as indicated by the increased diameter of lysis rings. A54145 was detected via HPLC/MS in fraction IRA E2 as evidenced by mass ion ES²⁺=830.5 consistent with the structures of A54145C,D (U.S. Pat. No. 4,994,270). FIG. 14b is a photograph of a plate generated during a similar extraction scheme performed on extracts from Streptomyces refuineus NRRL 3143, showing a similar enrichment of activity based on IRA67 anion exchange chromatography consistent with expression of an acidic lipopeptide.

This activity is concentrated during the extraction procedure as indicated by the increased diameter of lysis rings. A mass ion of ES²⁺=830.5, identical to that of A54145, was present in fraction IRA E2 confirming that an N-acylated acidic lipopeptide, identical to A54145C and D, is produced by 024A in Streptomyces refuineus subsp. thermotolerans as predicted from the genomic data contained in the DECIPHER® database.

### Example 5

### Identifying a Novel Polyketide from Cryptic Biosynthetic Loci Via Metabolomic Analysis

Streptomyces aizunensis was subject to the genome scanning method described in U.S. Ser. No. 10/152,886 noted Example 1, which resulted in the discovery in the Streptomyces aizunensis genome of many putative natural product biosynthetic loci. One of the gene clusters was predicted to encode a compound similar to the known antibiotic streptothricin based on computer-based comparisons to other clusters in the database. This information proved was useful in the genomics-guided methods for obtaining the metabolite associated with the target gene cluster, as a streptothricin-like compound was detected during subsequent fermentation experiments. Knowing in advance that this compound would be produced, genomics-guided methods were devised to avoid the streptothricin-like compound while purifying other compounds from the fermentation broths. Of the five biosynthetic loci referred to below, three contained NRPS genes and were predicted to encode for the production of peptides (locus designations 023B, 023C, and 023F), and one was predicted to code for the production of a large polyketide (locus designation 023D). Based upon the genomic information approximate chemical structures were predicted for compounds encoded by loci 023B, 023C, 023F and 023D.

| | Mass | | Poss. | | | |
|---|---|---|---|---|---|---|
| Locus | Range | UV | Activity | Class | AA Composition | Notes |
| 023B | >300 | none | — | Glycosylated | Ile/Leu dimer | |
| | | pred. | | dipeptide | | |
| 023C | >2000 | 250, 280 | antibacterial | glycosylated | XNXGNXFGXXXXNNNDDXNAGXAADX | multiple |
| | | | | lipopetide | | glycosyl |
| | | | | | | transferases |
| 023D | >1199 | >300 | antifungal | polyketide | n/a | 26 modules, |
| | | | | | | multiple |
| | | | | | | double bonds, |
| | | | | | | glycosyl |
| | | | | | | transferase |
| | | | | | | and deoxy |
| | | | | | | sugar genes. |
| 023F | >1000 | 280 | | decapeptide | XXVXXXXXXN | |
| SRCB | >300 | none | Broad | streptothiricin | | |
| | | pred. | spectrum | | | |

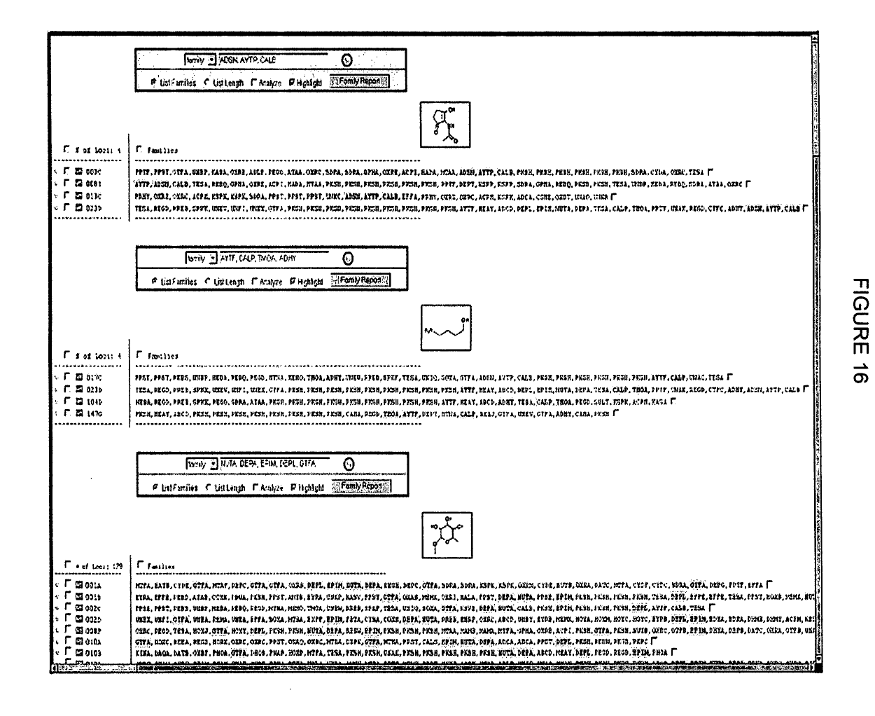

Automated analysis of the S. aizunensis gene cluster designated 023D, is shown in FIG. 15. In this cluster, the computer assigned 35 ORFs to protein families based on homology comparisons to proteins in the DECIPHER® database (Ecopia BioSciences Inc., St.-Laurent, Quebec, Canada). The disposition of these ORFs in the cluster is shown in window A of FIG. 15, where each ORF carries a four-letter code indicating the protein family to which it was assigned. Nine of the ORFs in the 023D cluster were designated as polyketide synthases (PKSs). PKSs and other multimodular protein families (such as non-ribosomal peptide synthetases) were further processed by an automated software application that parses the proteins into individual enzymatic domains. Each domain sequence was then compared to a series of protein models of active domains to identify domains that are likely to be nonfunctional. Additional computer scripts were invoked when particular domains are encountered. For example, the substrate specificity of each acyltransferase (AT) domain was readily assigned by a phylogenetic comparison to AT domains of known specificity, while a similar analysis of thioesterase (TE) domains very effectively distinguished domains that generate linear polyketide products from those that catalyze the formation of cyclic products. In the result, an automated “domain string” was generated as shown in FIG. 15, window C. The domain string captures the structure of the polyketide backbone in a line notation and was then translated into the chemical structure prediction shown in FIG. 15, window B. Automated analysis of the 023D PKS system predicted a long, linear polyketide chain bearing polyene chromophores. While many cyclic (macrolide) polyene natural products are known, linear polyenes remain relatively rare. Chemical substructure searches using the predicted polyketide backbone identified no similar structures in natural products databases, providing the first indication that the 023D gene cluster encoded a NCE.

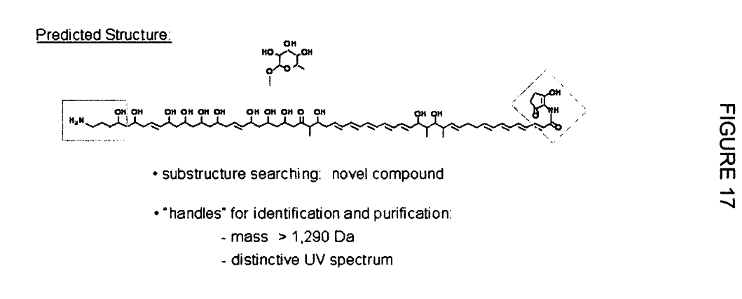

The “family string” generated by the analysis cascade (shown in window D of FIG. 15) provided a representation of the cluster that was used in an automated of search of the DECIPHER® database to identify gene clusters with similar protein families. The structures of the compounds linked to these clusters were compared to identify common structural elements. For example, three gene clusters in the DECIPHER® database contained the ADSN, AYTP, CALB families found in the 023D cluster. Structure analysis of the corresponding compounds identified a single common structural element, a 2-amino-3-hydroxycyclopentenone (C5N) group in amide linkage to a polyketide carboxylate (FIG. 16, upper panel). Inspection of the computer-predicted function of each family suggested a plausible pathway for the biosynthesis of C5N group from glycine and 5-aminolevulinic acid. Thus, the presence of these three genes in a cluster provides a marker for the presence of this functional group in a natural product. Similarly, computer analysis correlated four families in the 023D cluster with the presence of a four-carbon, amine-containing (C4N) polyketide starter unit (FIG. 16, middle panel) and five families with a 6-deoxyhexose sugar moiety (FIG. 16, lower panel). In both cases the predicted functions of the families suggested likely biosynthetic pathways and strongly supported the gene-structure correlations.

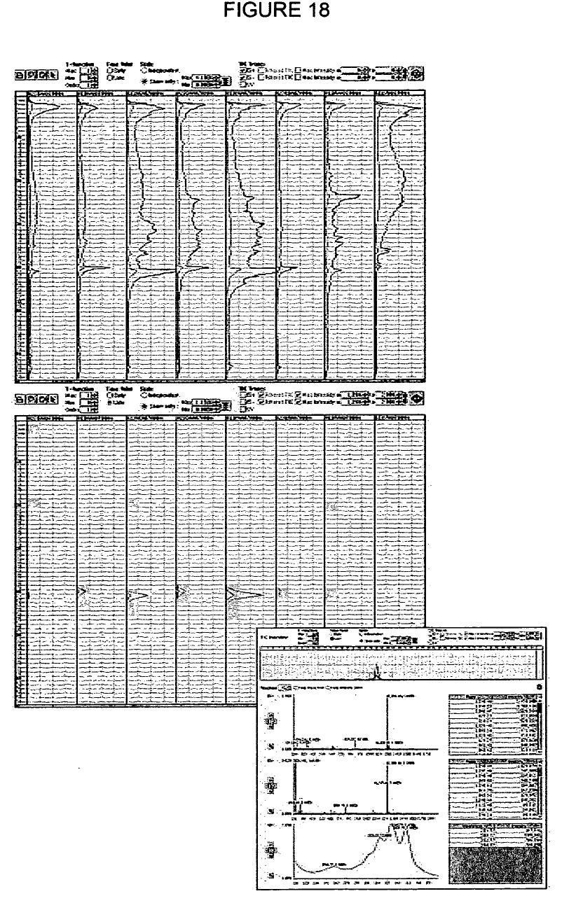

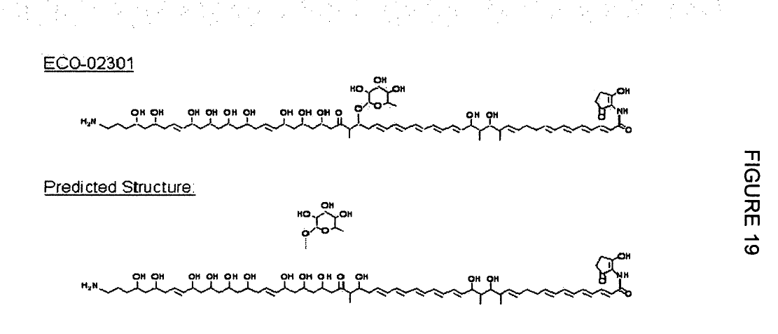

The results of the automated analysis cascade provided a very precise prediction of the structure of the compound encoded by the 023D gene cluster, as shown in FIG. 17. Tools for substructure searching are also integrated into the discovery platform and allow a scientist to quickly assess if a predicted structure has already been reported, providing an early opportunity for in silico dereplication. For example, substructure searches of the Antibase™ database (Wiley Publishers, 2003) of over 30,000 microbial natural products revealed only 61 products that contain the C5N group. Thus, even a small amount of structure information can greatly limit the number of structures that need to be considered as candidate products. More importantly, the addition of a second structure element to the query returned no hits from the database, further indicating novelty of the compound encoded by the 023D gene cluster (FIG. 17).

The compound encoded by the 023D gene cluster was targeted for purification. The structure prediction immediately identified physicochemical properties or “handles” that could be used to guide the purification of the compound. For example, the compound was predicted to have a molecular mass in excess of 1,290 Daltons (Da) and a distinctive UV spectrum imparted by the pentaene chromophore.

To ensure that the gene cluster was expressed, S. aizunensis was grown in 48 different media, namely AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA, which are representative of media reported to support the production of a wide range of natural products. S. aizunensis was grown in 25 ml shake flask cultures. Methanol extracts of each culture were subjected to HPLC-UV-MS analysis. Metabolites in each extract were monitored using a specially designed system that makes it possible to analyze HPLC fractions simultaneously across all the different media conditions (FIG. 18, top panel). An overview of the MS traces showed that the profile of metabolites varied considerably from medium to medium. The interface shown in FIG. 18A is fully interactive, so that the underlying chemical data can be rapidly searched using queries that incorporate multiple physicochemical parameters. For example, a mass filtering function allows the user to search all fractions for masses within a particular range. A search for metabolites having a mass greater that 1290 Da identified a single peak that appeared in some fermentation media but not others (FIG. 18, middle panel). Clicking on any fraction pops up a new window that displays the full spectral data set for that fraction. When this was done for one of the peak fractions from the mass filtered search, the data revealed molecular ions consistent with a compound of mass 1297 Da and a UV absorption spectrum characteristic of a pentaene (FIG. 18, bottom panel), fully consistent with the structure predictions generated by computer analysis. Thus, the chemical data indicated that the 1297 Da metabolite corresponded to the compound encoded by the 023D gene cluster. In addition to the chemical data, aliquots of each HPLC fraction were tested for antimicrobial activity against a panel of bacterial and fungal pathogens. As shown in FIG. 18 screening data for Candida albicans indicates the fractions containing the 1297 Da compound exhibited a potent antifingal activity in the bioassay screens.

The final structure, as confirmed by multidimensional NMR spectrometry, agreed entirely with the structure prediction generated by gene sequence analysis (FIG. 19).

### Example 6

### Identifying a Novel Polyketide from a Cryptic Biosynthetic Locus Via Isotope Incorporation Experiments

Streptomyces ghanaensis (NRRL B-12104) was subject to the genome scanning method described in U.S. Ser. No. 10/152,886 noted in Example 1, which resulted in the discovery in the Streptomyces ghanaensis genome of many putative natural product biosynthetic loci, seven of which were further characterized by sequence analysis and determined to be distinct biosynthetic loci. Of the seven biosynthetic loci analyzed, four contained NRPS genes and were predicted to encode the production of peptides (locus designations 009D, 009E, 009F, 009H), and two were predicted to encode for the production of a large polyketide (locus designation 009B and 009I). Based upon the genomic information, approximate chemical structures were predicted for the compounds encoded by loci of Streptomyces ghanaensis:

| | Mass | | Poss. | | AA | |
|---|---|---|---|---|---|---|
| Locus | Range | UV | Activity | Class | Composition | Notes |
| 009B | — | — | — | unusual | n/a | cryptic, v. small |
| | | | | polyketide | | unusual |
| 009C | >14,000 | >270 | Broad | chromoprotein | large peptide | Enediyne, non- |
| | | | spectrum | enediyne | chromoprotein | covalently binds to |
| | | | | | (ribosomal- | a chromoprotein |
| | | | | | encoded) | |
| 009D | >500 | | — | peptide | XXTXX | pentapeptide |
| 009E | >1000 | >250 | — | peptide | TFXTXXXTTX | decapeptide with |
| | | | | | | possible aromatic |
| | | | | | | moiety |
| 009F | — | — | — | peptide/ | X | cryptic, v. small |
| | | | | ketide | | |
| 009H | >1000 | 250 | — | (lipo)peptide | VFNTV*XXXX | nonapeptide, |
| | | | | | | possibly w/ N- |
| | | | | | | terminal lipid, * N- |
| | | | | | | methyl valine |
| 009I | >500 | 250 | antifungal | polyketide | n/a | 12-ketide, |
| | | | | | | hygrolidin like, |
| | | | | | | methylated, 3 |
| | | | | | | conjugated double |
| | | | | | | bonds |

For instance, 009H and 009I contain gene sequences similar to genes coding for the production methylation enzymes, or methyltransferases. In the case of the hypothetical metabolites coded for by loci 009H and 009I, the sequence similarity suggested that the biosynthetic precursor for the methyl groups was S-adenosyl methionine, which is biosynthesized via methionine in primary metabolism. Partial deduction of the structures of the compounds produced by 009H and 009I suggested that they were a polypeptide and a polyketide, respectively. The proposed domain organization of the polyketide synthase of 009I was predicted and a structure derived from this data:
[KS-AT(MM)-ACP] [KS-AT(MM)-KR-ACP] [KS-AT(M)-KR-ACP] [KS-AT(MM)-ACP] [KS-AT(MM)-KR-ACP] [KS-AT(M(OCH3)M)-KR-ACP] [KS-AT(M)-DH-KR-ACP] [KS-AT(MM)-DH-KR-ACP] [KS-AT(MM)-DH-ER-KR-ACP] [KS-AT(MM)-KR-ACP] [KS-AT(MM)-DH-KR-ACP] [KS-AT(MM)-DH-KR-ACP-TE]

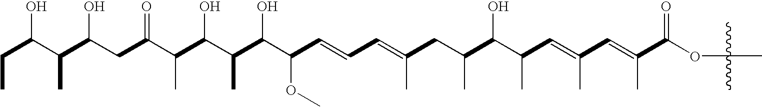

where abbreviations describe processive enzymatic activities corresponding to ketoacyl synthase (KS), acyltransferase (AT), ketoreductase (KR), dehydratase (DH), and enoyl reductase (ER) activity, as well as acyl carrier protein (ACP) and thioesterase (TE) activity. The methoxymalonyl (M(OCH₃)M) specificity of the sixth AT domain was discovered by domain comparison to a database of AT domains in the DECIPHER® database and supported by the presence of genes encoding enzymes known to produce methoxymalonyl-ACP, the precursor for this functionality in the metabolite encoded by locus 009I.

Thus, supplementation of multiple production media of Streptomyces ghanaensis with labeled methionine, specifically trideuteromethionine (methyl-D₃) was predicted to facilitate scanning the metabolome for the presence of metabolites incorporating N, O or S methyl groups. Such metabolites incorporating the methyl-D₃ from the labelled methionine were predicted to show mass spectral patterns consisting of a molecular ion plus a related molecular ion of different intensity but three daltons larger than the parent.

A metabolomics approach was subsequently used to identify conditions under which to express secondary metabolites, analyze them, and correlate them to the aforementioned biosynthetic loci based on isotopic incorporation patterns. This approach obtains analytical measurement of all low molecular weight metabolites (0-5000 Da) in a given organism at a specific time under specific culture conditions. Streptomyces ghanaensis was grown in 48 different media (AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA), many of which are representative of media reported to support the production of a wide range of natural products. Each medium was supplemented with trideuteromethionine (methyl-D₃, 1-5 mM). Metabolites were extracted from whole cell cultures by adding of an equal volume of methanol. After removal of solid debris, the extracts were concentrated and analyzed by the CHUMB method. Analysis of the chromatographic and bioactivity profiles indicated the presence, in a number of extracts, especially those derived from growth in medium RM, of chromatographically distinct peaks which demonstrated isotopic incorporation of trideutreromethyl as evidenced by the presence of a parent molecular ion corresponding to a mass of 574 Da plus a related ion three daltons larger than the parent ion at a ratio of parent:“+3 ion” of approximately 10:1 to 2:1.

Medium RM was selected for scale-up of fermentation to 500 mL and harvested after 10 days of growth. After completion of the general extraction procedure (supra), fractions 1 and 2 were found to contain the target ion. One of the methylated targets was isolated by C-18 solid phase extraction followed by C-18 HPLC. NMR data was collected for this compound including proton, carbon, COSY, HSQC, and HMBC spectra. The spectrocopic data was first used to edit the polyketide backbone derived from the locus prediction, which accelerated the elucidation of the structure. The only discrepancy between the genomic data and the NMR data was the apparent dehydration of the second hydroxyl in the predicted structure to yield the acrylate functionality. HMBC data confirmed the regiochemistry of lactone bond formation that describes the structure. Upon a search in the Dictionary of Natural Products, the isolated compound was revealed to be the known compound oxohygrolidin (shown below), which was not previously known to be produced by this organism.

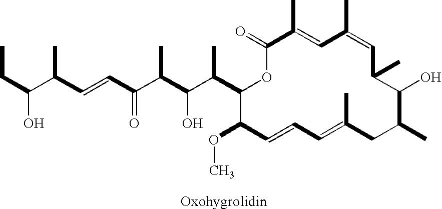

### Example 7

### Identification of a New Bioactive Polyene Polyketide by Association to a Target Gene Cluster

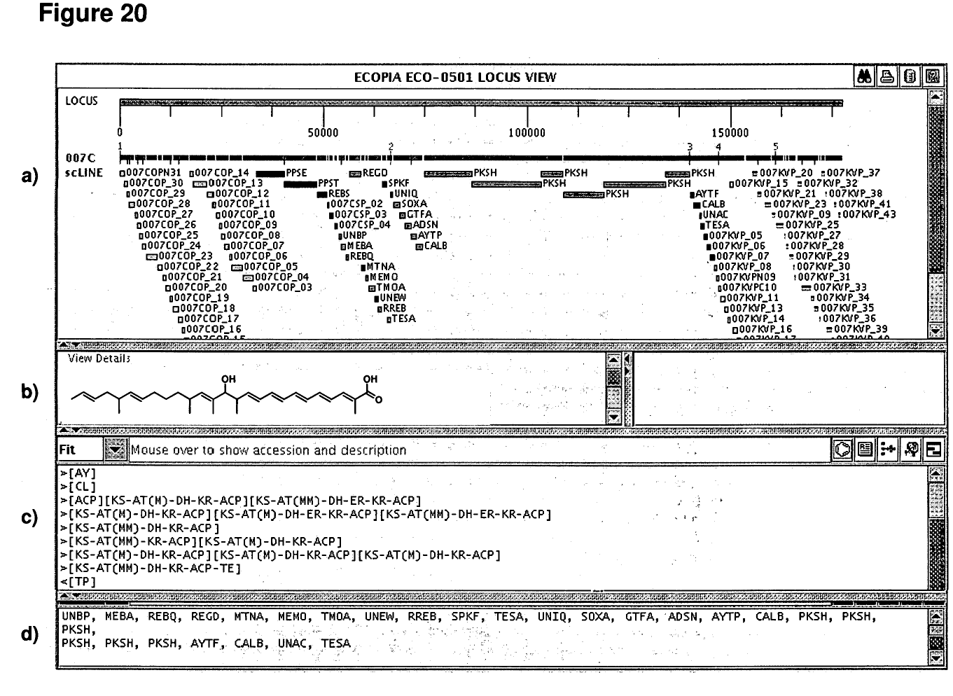

A novel bioactive polyene polyetide produced by the actinomycete Amycolatopsis orientalis (ATCC 43491) was discovered. The polyene polyketide identification and purification were guided by genomics information. Analysis of the A. orientalis gene cluster designated 007C, is shown in FIG. 20. In this cluster, 30 ORFs were assigned to protein families based on homology comparisons to proteins in the DECIPHER® database. In FIG. 20a, each ORF carries a four-letter code indicating the protein family to which it was assigned. Six of the ORFs in the 007C cluster were designated as PKSH: polyketide synthases (PKSs). Each PKS domains: ketoacyl synthase (KS), acyltransferase (AT), ketoreductase (KR), dehydratase (DH), and enoyl reductase (ER) activity, as well as acyl carrier protein (ACP) and thioesterase (TE) activity were also identified. The substrate specificity of each acyltransferase (AT) domain is readily assigned by a phylogenetic comparison to AT domains of known specificity, while a similar analysis of thioesterase (TE) domains distinguishes domains that generate linear polyketide products from those that catalyze the formation of cyclic products. The “domain string” obtained (FIG. 20c) captured the structure of the polyketide backbone that was translated into the chemical structure prediction presented in FIG. 20b.

The analysis of the 007C PKS system predicted a long, linear polyketide chain bearing polyene chromophores. No similar structures were identified in the natural products databases, providing the first indication that the 007C gene cluster encoded a new chemical entity.

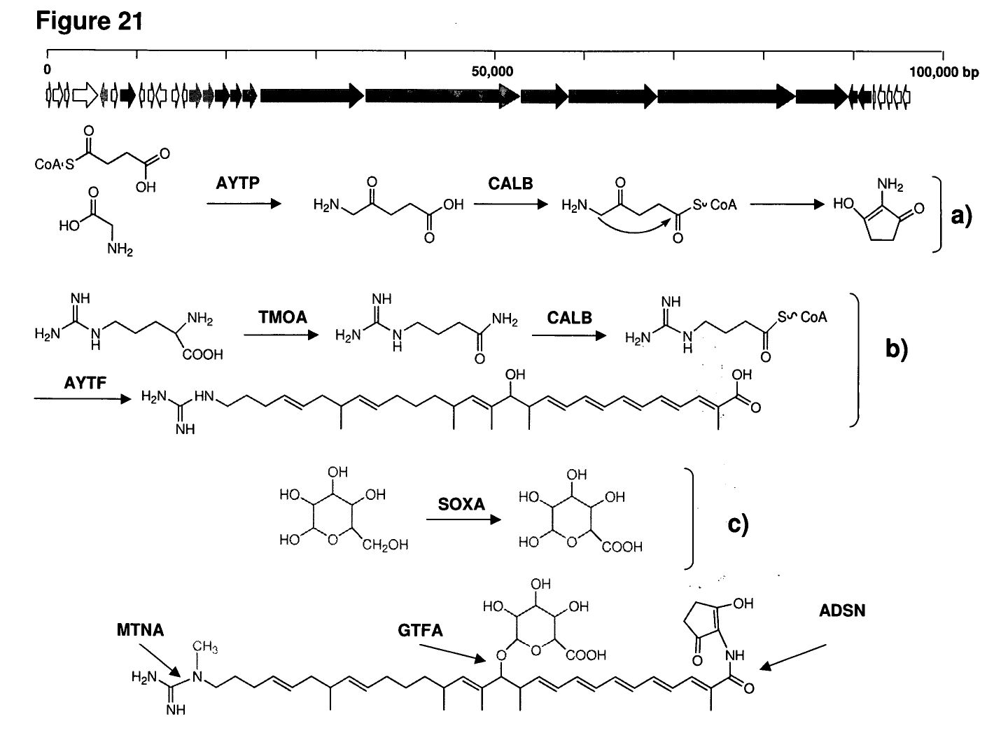

The “family string” (FIG. 20d) provides a representation of the cluster, which was compared to gene clusters with similar families. These clusters are then compared to identify common structural elements. For example, three gene clusters in the DECIPHER® database contain the ADSN, AYTP, CALB families found in the 007C gene cluster. Structure analysis of the corresponding compounds identified a single common structural element, a 2-amino-3-hydroxycyclopentenone (C₅N) group in amide linkage to a polyketide carboxylate. The biosynthesis of C₅N group from glycine and 5-aminolevulinic acid (FIG. 21a). Thus, the presence of these three genes in a cluster provided a marker for the presence of this functional group in a natural product. Similarly, computer analysis correlated three families in the 007C cluster with one indicating the presence of a guanidino-substituted four-carbon polyketide starter unit (C₅N₃) (FIG. 21b) and one family leading to a glucuronic acid moiety (FIG. 21c).

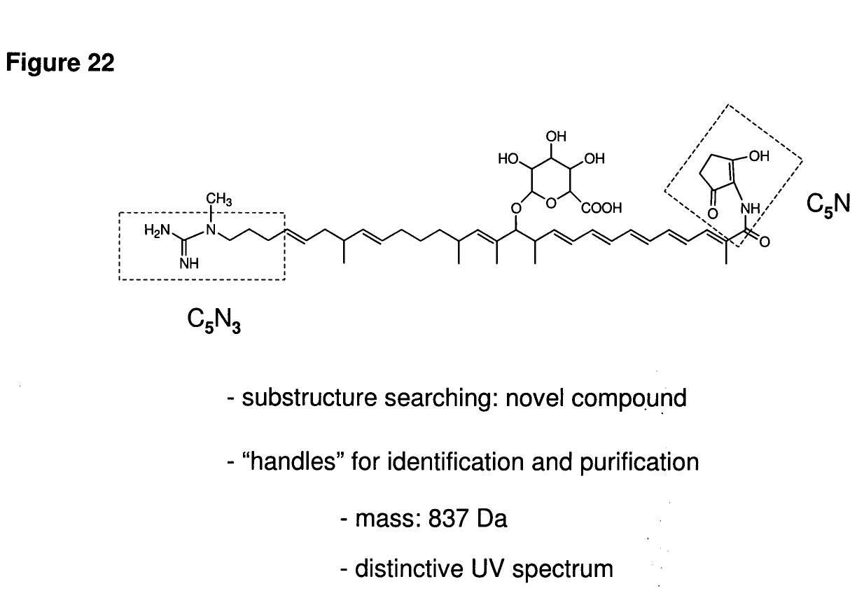

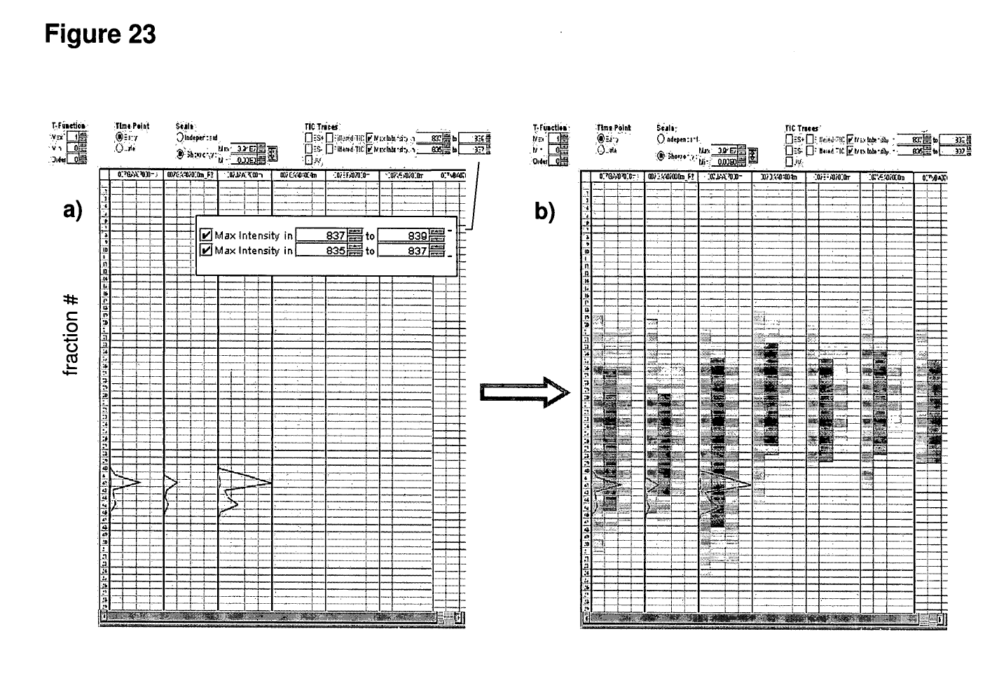

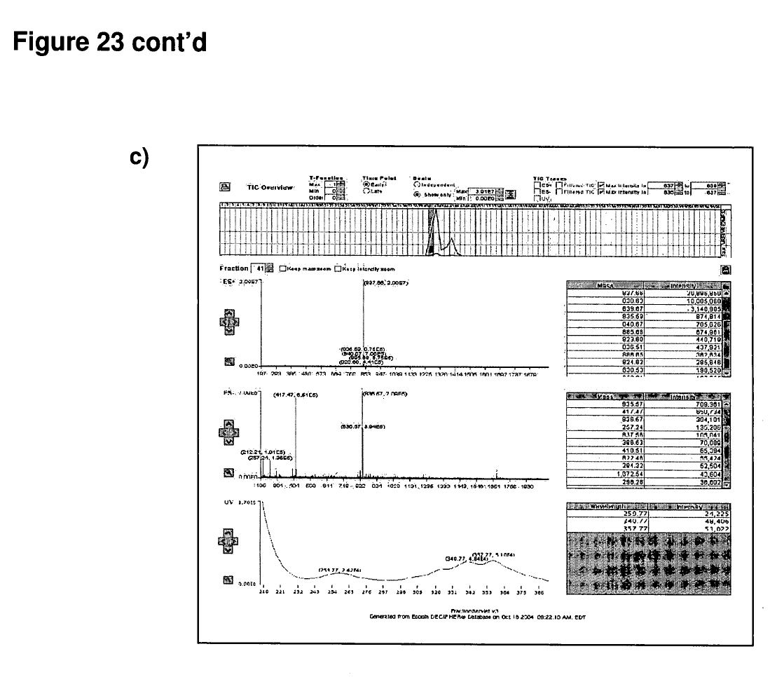

The complete structure predicted by association to the genomics is shown in FIG. 22. The compound encoded by the 007C gene cluster was targeted for purification. The structure prediction immediately identified physicochemical properties or “handles” that could be used to guide the purification of the compound. For example, the compound was predicted to have a molecular mass of 837 Daltons (Da) and a distinctive UV spectrum imparted by the pentaene chromophore. To ensure that the gene cluster was expressed, A. orientalis was grown in more than 50 different fermentation media in 25 ml shake flask cultures. Methanol extracts of each culture were subjected to HPLC-UV-MS analysis, and metabolites were monitored simultaneously across all the different media conditions (FIG. 23a). Multiple physicochemical parameters were searched to identify the broth containing the desired molecule. For example, a mass filtering function allowed search for metabolites having a mass of 837 Da that was identified as a main peak that appeared in some fermentations (FIG. 23). The data revealed molecular ions consistent with a compound of mass 837 Da and a UV absorption spectrum characteristic of a pentaene (FIG. 23c), fully consistent with the structure predictions generated by computer analysis. In addition to the chemical data, 7 aliquots were screened for antibacterial activity (FIG. 23b) against 20 Micrococcus luteus (first band), Staphylococcus aureus (second band) and Enterococcus faecalis (third band) and the fractions containing the 837 Da compound exhibited potent antibacterial activity.

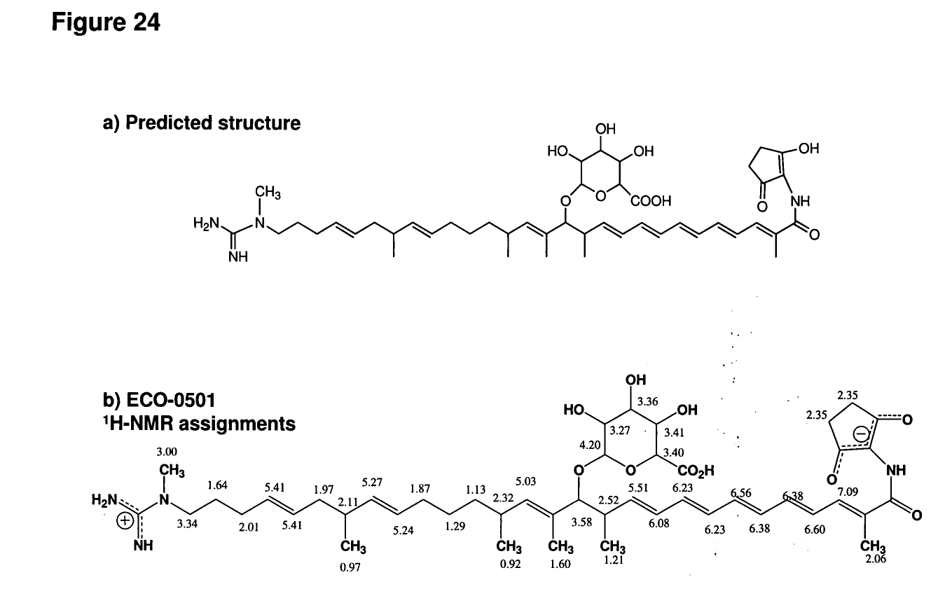

Having the mass, UV and bioactivity data in hand, the subsequent purification of the 837 Da compound was straightforward. The elucidation was greatly facilitated by the structure prediction. The final structure, as confirmed by multidimensional NMR spectrometry, agreed entirely with the structure prediction generated by gene sequence analysis (FIG. 24).

### Example 8

### Identification of a Novel Bioactive Hexapeptide from Amycolatopsis orientalis ATCC 43491

Amycolatopsis orientalis ATCC™ 43491 was obtained from the American Type Culture Collection (P.O. Box 1549, Manassas, Va. 20108, USA). The biosynthetic locus for the production of a hexapeptide designated Compound 1 (0506) was identified in the genome of Amycolatopsis orientalis ATCC™ 43491 using the genome scanning method described in United States Patent Application U.S. Ser. No. 10/232,370, Canadian Patent Application CA 2,352,451 and Zazopoulos et. al., Nature Biotechnol., 21, 187-190 (2003).

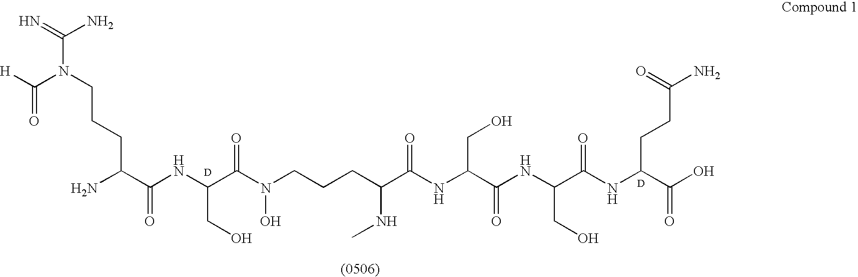

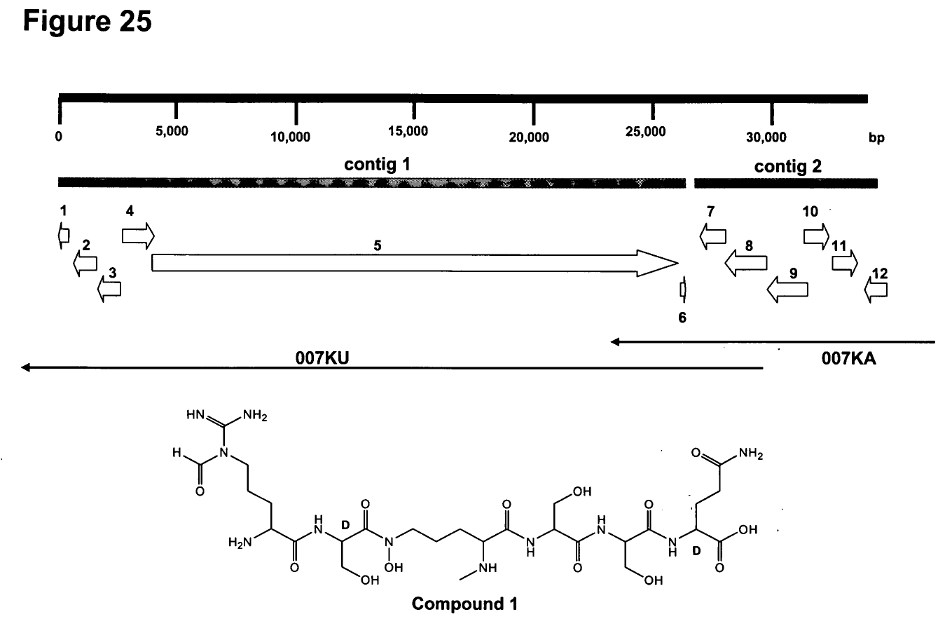

The biosynthetic locus spans approximately 34,180 base pairs of DNA and encodes 12 proteins. More than 10 kilobases of DNA sequence were analyzed on each side of the locus and these regions were deemed to contain primary genes or genes unrelated to the synthesis of Compound 1 (0506). As illustrated in FIG. 25, the locus is contained within two sequences of contiguous base pairs, namely Contig 1 having the 26,490 contiguous base pairs of SEQ ID NO: 1 and comprising ORFs 1 to 6 (SEQ ID NOS: 3, 5, 7, 9, 11 and 13), and Contig 2 having the 7,697 contiguous base pairs of SEQ ID NO: 14 and comprising ORFs 7 to 12 (SEQ ID NOS: 16, 18, 20, 22, 24 and 26). The order, relative position and orientation of the 12 open reading frames representing the proteins of the biosynthetic locus are illustrated schematically in FIG. 25. The top line in FIG. 25 provides a scale in base pairs. The black bars depict the two DNA contigs (SEQ ID NOS: 1 and 14) that cover the locus. The empty arrows represent the 12 open reading frames of this biosynthetic locus. The black arrows represent the two deposited cosmid clones covering the locus.

The biosynthetic locus will further be understood with reference to the sequence listing which provides contiguous nucleotide sequences and deduced amino acid sequences of the locus from Amycolatopsis orientalis ATCC™ 43491. The contiguous nucleotide sequences are arranged such that, as found within the biosynthetic locus, Contig 1 (SEQ ID NO: 1) is adjacent to the 5′ end of Contig 2 (SEQ ID NO: 14). The ORFs illustrated in FIG. 25 and provided in the sequence listing represent open reading frames deduced from the nucleotide sequences of Contigs 1 and 2 (SEQ ID NOS: 1 and 14). Referring to the Sequence Listing, ORF 1 (SEQ ID NO: 3) is the polynucleotide drawn from residues 513 to 76 (antisense strand) of SEQ ID NO: 1, and SEQ ID NO: 2 represents that polypeptide deduced from SEQ ID NO: 3. ORF 2 (SEQ ID NO: 5) is the polynucleotide drawn from residues 1490 to 513 (antisense strand) of SEQ ID NO: 1, and SEQ ID NO: 4 represents the polypeptide deduced from SEQ ID NO: 5. ORF 3 (SEQ ID NO: 7) is the polynucleotide drawn from residues 2526 to 1579 (antisense strand) of SEQ ID NO: 1, and SEQ ID NO: 6 represents the polypeptide deduced from SEQ ID NO: 7. ORF 4 (SEQ ID NO: 9) is the polynucleotide drawn from residues 2664 to 4007 (sense strand) of SEQ ID NO: 1, and SEQ ID NO: 8 represents the polypeptide deduced from SEQ ID NO: 9. ORF 5 (SEQ ID NO: 11) is the polynucleotide drawn from residues 4053 to 26264 (sense strand) of SEQ ID NO: 1, and SEQ ID NO: 10 represents the polypeptide deduced from SEQ ID NO: 11. ORF 6 (SEQ ID NO: 13) is the polynucleotide drawn from residues 26261 to 26470 (sense strand) of SEQ ID NO: 1, and SEQ ID NO: 12 represents the polypeptide deduced from SEQ ID NO: 13. ORF 7 (SEQ ID NO: 16) is the polynucleotide drawn from residues 1114 to 26 (antisense strand) of SEQ ID NO: 14, and SEQ ID NO: 15 represents the polypeptide deduced from SEQ ID NO: 16. ORF 8 (SEQ ID NO: 18) is the polynucleotide drawn from residues 2976 to 1225 (antisense strand) of SEQ ID NO: 14, and SEQ ID NO: 17 represents the polypeptide deduced from SEQ ID NO: 18. ORF 9 (SEQ ID NO: 20) is the polynucleotide drawn from residues 4658 to 2973 (antisense strand) of SEQ ID NO: 14, and SEQ ID NO: 19 represents the polypeptide deduced from SEQ ID NO: 20. ORF 10 (SEQ ID NO: 22) is the polynucleotide drawn from residues 4761 to 5804 (sense strand) of SEQ ID NO: 14, and SEQ ID NO: 21 represents the polypeptide deduced from SEQ ID NO: 22. ORF 11 (SEQ ID NO: 24) is the polynucleotide drawn from residues 5828 to 6865 (sense strand) of SEQ ID NO: 14, and SEQ ID NO: 23 represents the polypeptide deduced from SEQ ID NO: 24. ORF 12 (SEQ ID NO: 26) is the polynucleotide drawn from residues 7697 to 6846 (antisense strand) of SEQ ID NO: 14, and SEQ ID NO: 25 represents the polypeptide deduced from SEQ ID NO: 26.

Two open reading frames provided in the Sequence Listing, namely ORF 9 (SEQ ID NO: 19) and ORF 10 (SEQ ID NO: 21), initiate with non-standard initiation codons (e.g. GTG—Valine, or CTG—Leucine) rather than standard initiation codon ATG methionine. All ORFs are listed with the appropriate M, V or L amino acids at the amino-terminal position to indicate the specificity of the first codon of the ORF. It is expected, however, that in all cases the biosynthesized protein will contain a methionine residue, and more specifically a formylmethionine residue, at the amino terminal position, in keeping with the widely accepted principle that protein synthesis in bacteria initiates with methionine (formylmethionine) even when the encoding gene specifies a non-standard initiation codon (e.g. Stryer BioChemistry 3^{rd} edition, 1998, W.H. Freeman and Co., New York, pp. 752-754).

Two deposits of E. coli DH10B vectors (007KA and 007KU), each harbouring a cosmid clone of a partial biosynthetic locus for Compound 1 (0506) from Amycolatopsis orientalis (ATCC™ 43491) and together spanning the full biosynthetic locus for production of Compound 1 (0506) have been both deposited with the International Depositary Authority of Canada, Bureau of Microbiology, Health Canada, 1015 Arlington Street, Winnipeg, Manitoba, Canada R3E 3R2 on May 25, 2005 and were assigned respectively deposit accession numbers IDAC 250505-01 and IDAC 250505-02 respectively. The cosmid of deposit IDAC 250505-02 (007KU) covers residue 1 of Contig 1 (SEQ ID NO: 1) to residue 3015 of Contig 2 (SEQ ID NO: 14). The cosmid of deposit IDAC 250505-01 (007KA) covers residue 23620 of Contig 1 (SEQ ID NO: 1) to residue 7697 of Contig 2 (SEQ ID NO: 14). The sequence of the polynucleotides comprised in the deposited strains, as well as the amino acid sequence of any polypeptide encoded thereby are controlling in the event of any conflict with any description of sequences herein.

The deposit of the deposited strains has been made under the terms of the Budapest Treaty on the International Recognition of the Deposit of Micro-organisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement, such as that required under 35 U.S.C. § 112. A license may be required to make, use or sell the deposited strains, and compounds derived therefrom, and no such license is hereby granted.

In order to identify the function of the proteins coded by the genes forming the biosynthetic locus for the production of Compound 1 (0506) the gene products of ORFs 1 to 12, namely SEQ ID NOS: 2, 4, 6, 8, 10, 12, 15, 17, 19, 21, 23, and 25, were compared, using the BLASTP version 2.2.6 algorithm with the default parameters, with the exception of the low complexity filter which was deactivated, to sequences in the National Center for Biotechnology Information (NCBI) nonredundant protein database and the DECIPHER® database of microbial genes, pathways and natural products (Ecopia BioSciences Inc. St.-Laurent, QC, Canada).

The accession numbers of the top GenBank™ hits of this BLAST analysis are presented along with the corresponding E values in the table noted immediately below. The E value relates the expected number of chance alignments with an alignment score at least equal to the observed alignment score. An E value of 0.00 indicates a perfect homolog. The E values are calculated as described in Altschul et al. J. Mol. Biol., 215, 403-410 (1990). The E value assists in the determination of whether two sequences display sufficient similarity to justify an inference of homology.

Sequence comparison and ORF correlation

| | SEQ | | | GenBank | | % identity | proposed function of GenBank |
|---|---|---|---|---|---|---|---|
| ORF | ID | #aa | Family | homology | probability | (% similarity) | match |
| 1 | 2 | 145 | | BAC69289.1 | 3E−27 | 42% (65%) | Hypothetical protein |
| | | | | 145aa | | | (S. avermitilis) |
| | | | | ZP_00187552.2 | 1E−26 | 45% (61%) | Putative flavin-nucleotide - |
| | | | | 141aa | | | binding protein |
| | | | | CAB88940.1 | 2E−26 | 44% (64%) | Hypothetical protein (S. coelicolor) |
| | | | | 142aa | | | |
| 2 | 4 | 325 | | NP_103590.1 | 2E−57 | 44% (54%) | RNA polymerase sigma subunit |
| | | | | 350aa | | | |
| | | | | ZP_00402093.1 | 1E−50 | 44% (55%) | Sigma-70 region 2 |
| | | | | 313aa | | | |
| | | | | CAC33060.1 | 2E−50 | 40% (55%) | Putative ECF sigma factor |
| | | | | 334aa | | | |
| 3 | 6 | 315 | FXBA | CAB53329.1 | 1E−137 | 73% (86%) | Putativeformyltransferase |
| | | | | 315aa | | | |
| | | | | AAC43261.1 | 3E−79 | 59% (75%) | Ferric exochelin biosynthesis |
| | | | | 360aa | | | protein |
| | | | | ZP_00298869.1 | 8e−41 | 35% (52%) | Methionyl-tRNA |
| | | | | 311aa | | | formyltransferase |
| 4 | 8 | 447 | OXRK | CAB53328.1 | 1E−162 | 66% (78%) | Putative peptide monooxygenase |
| | | | | 451aa | | | |
| | | | | ZP_00294186.1 | 1E−132 | 58% (70%) | Lysine/ornithine N- |
| | | | | 438aa | | | monooxygenase |
| | | | | ZP_00212672.1 | 2E−72 | 39% (52%) | Lysine/ornithine N- |
| | | | | 458aa | | | monooxygenase |
| 5 | 10 | 7403 | NRPS | NP_960354.1 | 0.0 | 39% (52%) | Hypothetical protein MAP1420 |
| | | | | 6384aa | | | |
| | | | | YP_121249.1 | 0.0 | 42% (56%) | Putative non-ribosomal peptide |
| | | | | 14474aa | | | synthetase |
| | | | | CAA72312.1 | 0.0 | 44% (56%) | Pristinamycin synthetase3 and 4 |
| | | | | 4848aa | | | |
| 6 | 12 | 69 | UNKC | AAK81828.1 | 2E−25 | 75% (84%) | Hypothetical protein |
| | | | | 73aa | | | |
| | | | | CAB38589.1 | 1E−24 | 72% (81%) | Putative small conserved |
| | | | | 71aa | | | hypothetical protein |
| | | | | AAX31560.1 | 9E−24 | 67% (83%) | Putative esterase |
| | | | | 1 | | | |
| | | | | 75aa | | | |
| 7 | 15 | 362 | FETB | ZP_00357504.1 | 3E−68 | 42% (60%) | ABC-type Fe3+-hydroxamate |
| | | | | 362aa | | | transport system |
| | | | | ZP_00200024.1 | 2E−63 | 44% (62%) | ABC-type Fe3+-hydroxamate |
| | | | | 347aa | | | transport system |
| | | | | ZP_00187388.1 | 9E−55 | 39% (55%) | ABC-type Fe3+-hydroxamate |
| | | | | 344aa | | | transport system |
| 8 | 17 | 583 | ABCA | AAC82548.1 | 1E−133 | 47% (62%) | Unknown |
| | | | | 589aa | | | |
| | | | | ACC32046.1 | 1E−133 | 47% (62%) | ExiT |
| | | | | 1122aa | | | |
| | | | | CAB53323.1 | 1E−120 | 45% (60%) | Putative ABC-transporter |
| | | | | 601aa | | | |
| 9 | 19 | 561 | ABCA | CAB53321.1 | 1E−95 | 41% (58%) | Putative ABC-transporter |
| | | | | 540aa | | | |
| | | | | AAC82547.1 | 1E−90 | 42% (58%) | Unknown |
| | | | | 574aa | | | |
| | | | | YP_054817.1 | 6E−73 | 36% (51%) | ABC transporter |
| | | | | 588aa | | | |
| 10 | 21 | 347 | EXPF | ZP_00209225.1 | 1E−105 | 59% (73%) | ABC-type Fe3+-hydroxamate |
| | | | | 362aa | | | transport system |
| | | | | CAB52851.1 | 1E−97 | 52% (69%) | Putative iron-siderophore uptake |
| | | | | 348aa | | | system |
| | | | | BAC74202.1 | 1E−96 | 51% (69%) | Putative ferrichrome ABC |
| | | | | 347aa | | | transporter system |
| 11 | 23 | 345 | EXPF | BAC74203.1 | 7E−89 | 52% (68%) | Putative ferrichrome ABC |
| | | | | 352aa | | | transporter system |
| | | | | CAB52850.1 | 7E−88 | 52% (68%) | Putative iron-siderophore uptake |
| | | | | 375aa | | | system |
| | | | | ZP_00292632.1 | 7E−86 | 54% (67%) | ABC-type enterobactin transport |
| | | | | 346aa | | | system |
| 12 | 25 | 283 | | BAC71135.1 | 2E−65 | 52% (67%) | Putative integral membrane |
| | | | | 274aa | | | protein |
| | | | | CAA20164.1 | 7E−59 | 48% (62%) | Putative integral membrane |
| | | | | 299aa | | | protein |
| | | | | NP_940092.1 | 1E−35 | 38% (55%) | Putative membrane protein |
| | | | | 268aa | | | |

The ORFs encoding proteins involved in the biosynthesis of Compound 1 (0506) were assigned a function and grouped together in families based on sequence similarity to known proteins. To correlate structure and function, the protein families were given a four-letter designation as follows: ABCA designates an ABC membrane protein transporter; EXPF designates a membrane protein ferric transporter; FETB designates an iron-transporting lipoprotein; FXBA designates a formyl transferase; NRPS designates a nonribosomal peptide synthetase; OXRK designates an N-hydroxylase; UNKC designates a protein of unassigned function.

Biosynthesis of Compound 1 (0506) was predicted to result from the action of a multimodular nonribosomal peptide synthetase system (NRPS) corresponding to ORF 5 (SEQ ID NO: 10). Each NRPS module contains at least three domains: a condensation domain (C), an adenylation domain (A) and a thiolation domain (T). NRPS domains, as per domains for the polyketide synthase systems described in the present application, were determined using software that utilizes HMMER, that being hidden Markov model analysis software developed by and freely available through Dr. Sean Eddy et al. (Washington University at St. Louis). A person skilled in the art would appreciate that other software programs for hidden Markov model analysis, such as the SAM (Sequence Alignment and Modeling System) program available from the University of California at Santa Cruz, could be implemented to perform the domain analysis. Domains conferring additional enzymatic activities such as epimerization domain (E), and N-methylation domain (M) can also be found in the NRPS modules. These additional domains result in various modifications of the growing peptide chain. N-Methylation domains may act as separate domains or be included within an adenylation domain. Each module is responsible for one round of coupling resulting in the addition of one amino acid unit. As a result, there is a direct correlation between the number of modules and the length of the peptide chain as well as between the domain composition of the modules and the type of amino acid incorporated. The genetic organization of most NRPS enzymes is colinear with the order of biochemical reactions giving rise to the peptide chain. This feature allows prediction of peptide core structure based on the architecture of the NRPS modules found in a given biosynthetic pathway (Marahiel et al. (1997) Chem. Rev., 97, 2651-2673), and the specificity-conferring codes of adenylation domains (Challis et al. (2000), Chem. Biol., vol 7, no 3, 211-224, and in Stachelhaus et al. (1999), Chem. Biol. 6, 493-505).

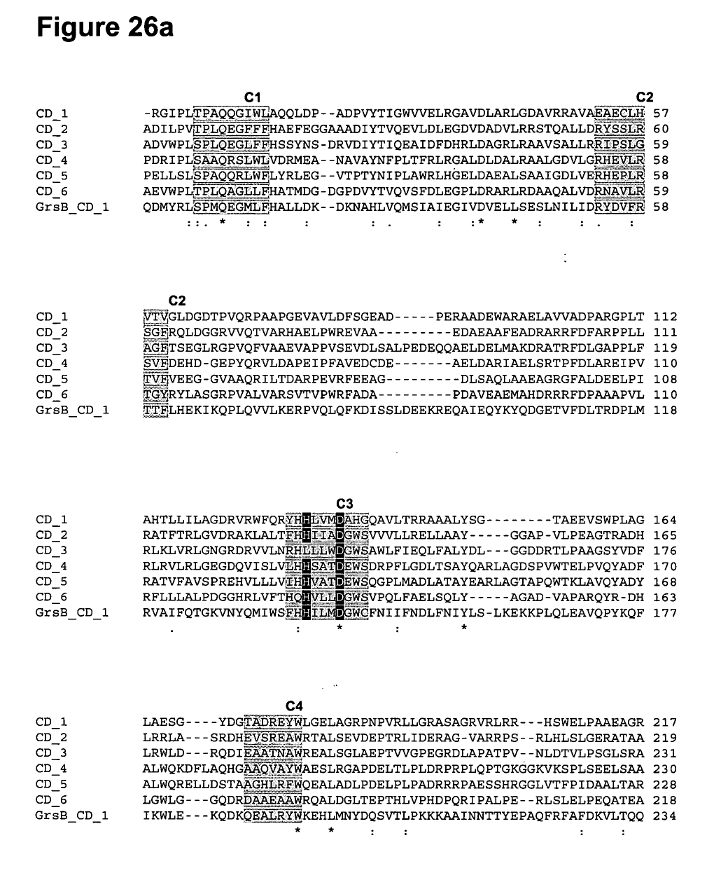

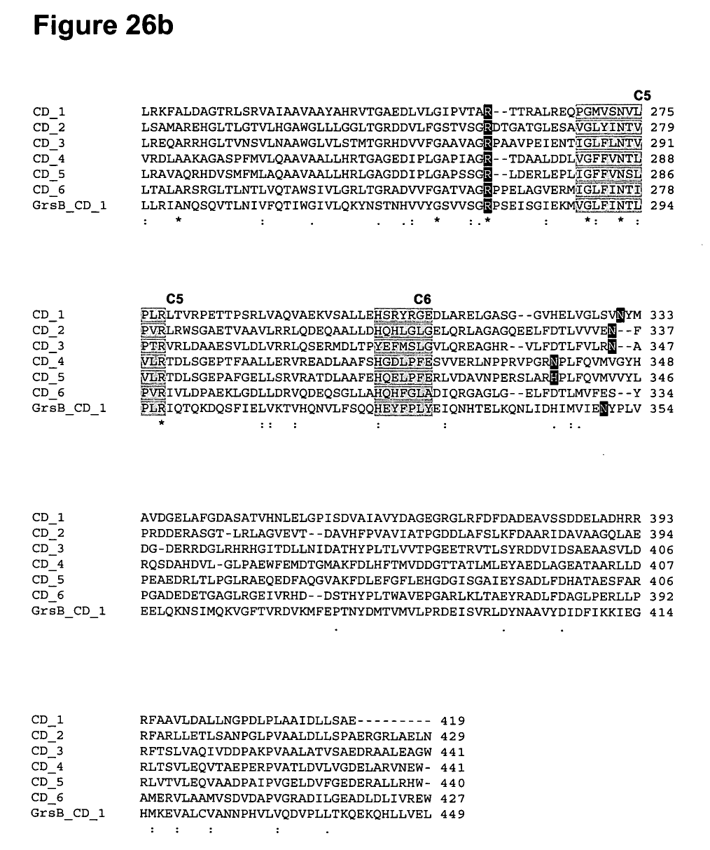

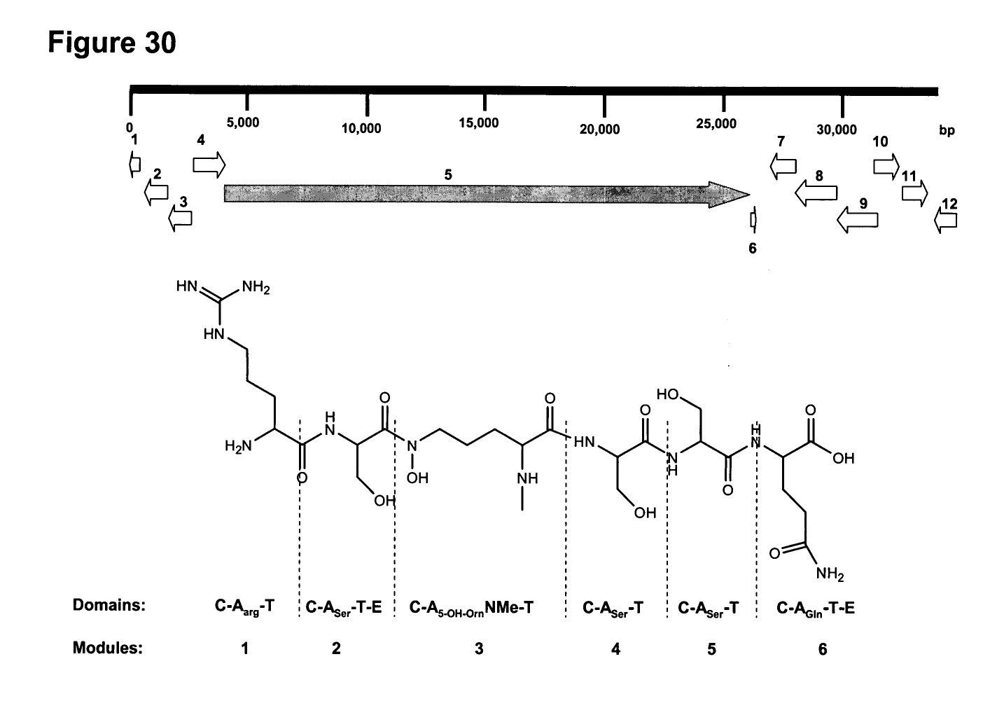

The NRPS system in the biosynthetic locus for the production of Compound 1 (0506) was determined to be composed of ORF 5 (SEQ ID NO: 10) and comprise a total of 6 modules as described in the table presented immediately below. Clustal™ alignment analysis of the NRPS domains revealed that all domains were complete and contained known motifs and conserved amino acid residues required for activity (FIGS. 26 to 29). The role of each domain of SEQ ID NO: 10 in the biosynthesis of Compound 1 (0506) is also shown schematically in FIG. 30.

Compound 1 (0506) locus NRPS domain coordinates

| | SEQ ID NO | | | | |
|---|---|---|---|---|---|
| ORF | Amino acid/ | | | | Module |
| no. | Nucleic acid | Amino Acids | Nucleic Acids | Function | no. |
| 5 | 10/11 | 3-421 | 7-1263 | C | 1 |
| | | 454-946 | 1360-2838 | A | |
| | | 950-1016 | 2848-3048 | T | |
| | | 1043-1471 | 3127-4413 | C | 2 |
| | | 1495-1981 | 4483-5943 | A | |
| | | 1984-2041 | 5950-6123 | T | |
| | | 2047-2457 | 6139-7371 | E | |
| | | 2466-2906 | 7396-8718 | C | 3 |
| | | 2965-3836 | 8893-11508 | A | |
| | | | | (N—Me) | |
| | | 3841-3908 | 11521-11724 | T | |
| | | 3922-4362 | 11764-13086 | C | 4 |
| | | 4387-4840 | 13159-14520 | A | |
| | | 4844-4911 | 14530-14733 | T | |
| | | 4924-5363 | 14770-16089 | C | 5 |
| | | 5388-5837 | 16162-17511 | A | |
| | | 5838-5904 | 17512-17712 | T | |
| | | 5936-6362 | 17806-19086 | C | 6 |
| | | 6387-6883 | 19159-20649 | A | |
| | | 6887-6948 | 20659-20844 | T | |
| | | 6950-7400 | 20848-22200 | E | |

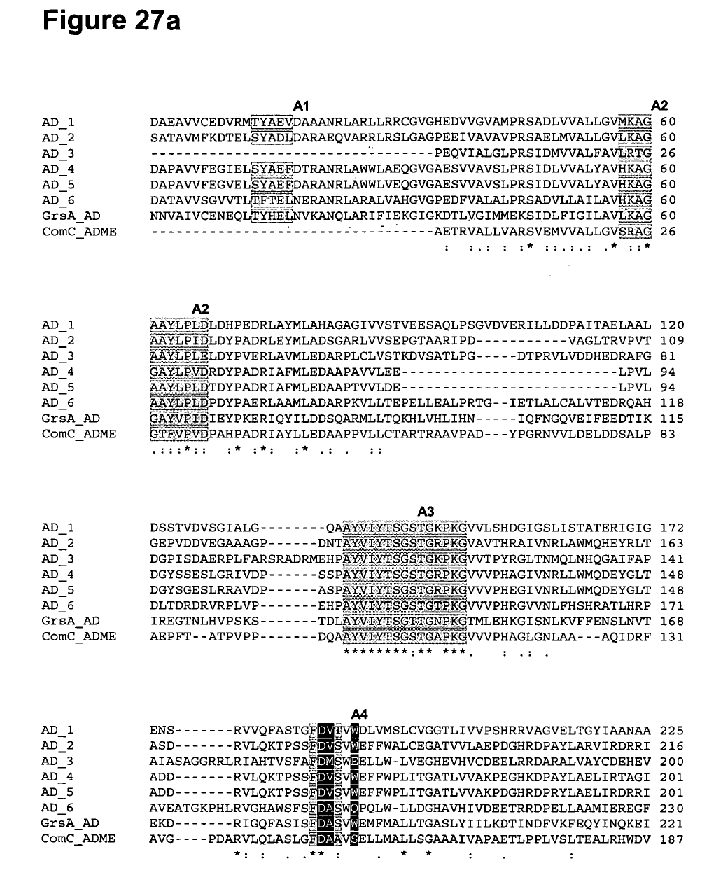

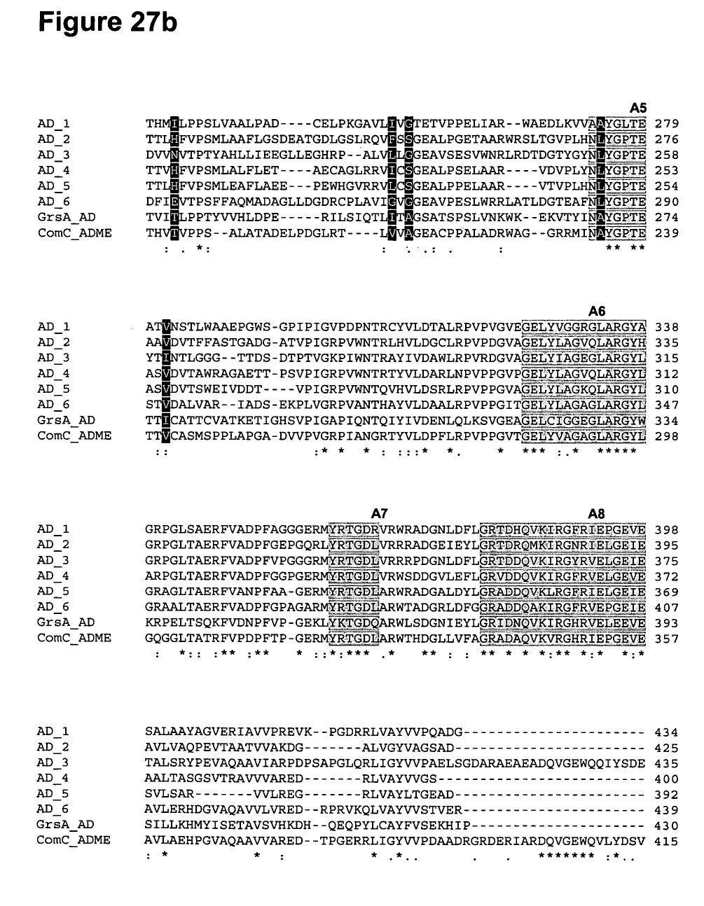

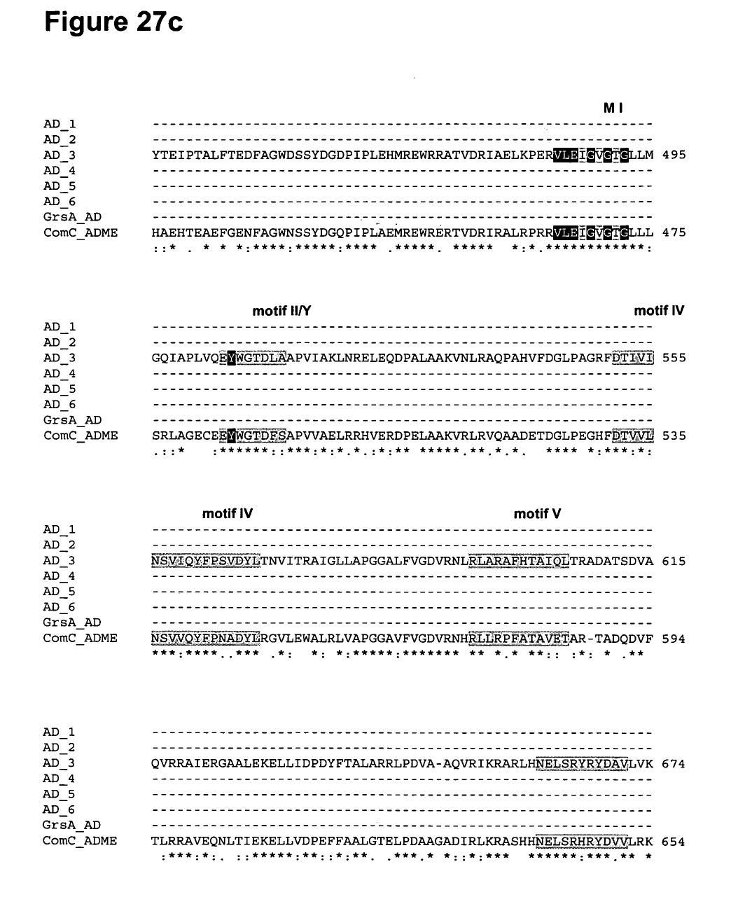

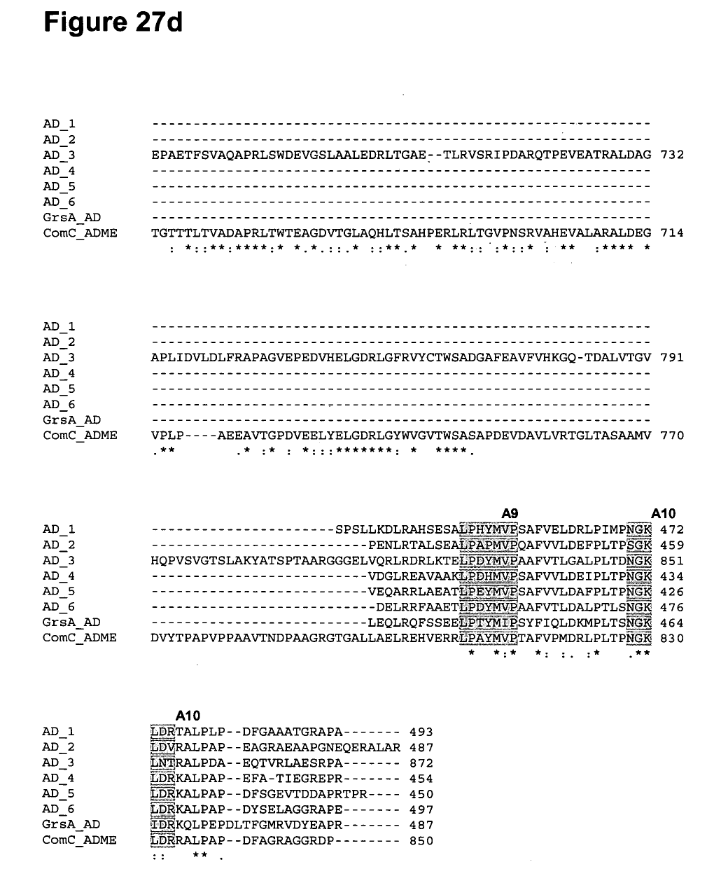

Multiple amino acid alignment of C domains present in the NRPS system together with the first condensation domain of GrsB encoded by the gramicidin S biosynthetic locus (Kratzshmar et al. (1989), J. Bacteriol., 171, 5422-5429) described in FIG. 26(a-b), shows an overall similarity of domains and conservation of amino acid motifs (C1 to C6) important for activity indicating that all C domains are functional. Similarly, multiple amino acid alignment of A domains together with adenylation domains present in GrsA and ComC, in the gramicidin S (Kratzshmar et al., ibid.) and complestatin (Chiu et al. (2001), Proc. Natl. Acad. Sci. USA, 98, 8548-8553) biosynthetic pathways respectively, (described in FIG. 27(a-d)), shows an overall similarity of domains and conservation of amino acid motifs (A1 to A10) important for activity indicating that all A domains are functional. Alignments of FIG. 27(a-d) also shows N-methylation activity contained within the adenylation domain of module 3, and its alignment with the complestatin ComC adenylation domain (Chiu et al., supra) that contains a N-methyltransferase domain imbeded between motifs A8 and A9. Comparison of the two N-methyltransferase domains present in adenylation domain of module 3 and in ComC shows conservation of motifs MI, II/Y, IV and V, important for cofactor binding and activity (Hacker et al. (2000), J. Biol. Chem., 275, 30826-30832).

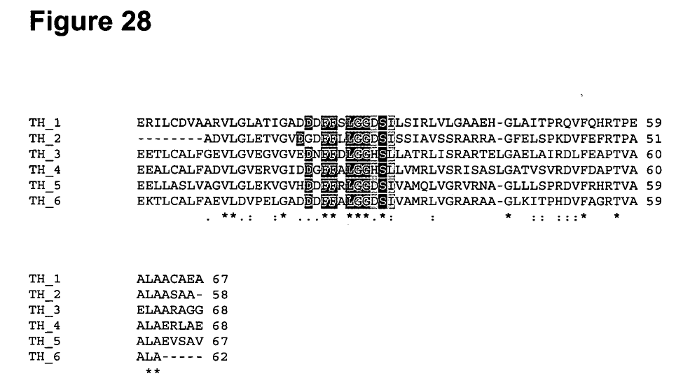

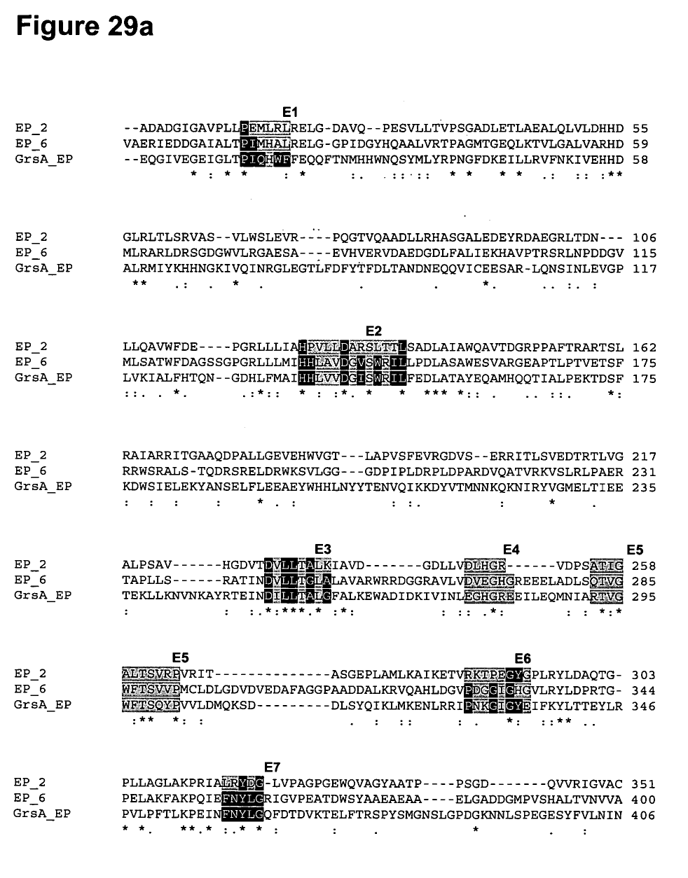

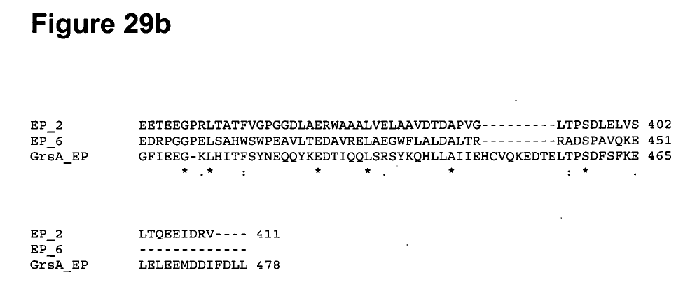

Multiple amino acid alignment of T domains of modules 1 to 6 (described in FIG. 28) shows an overall similarity of domains and conservation of the serine amino acid residue essential for tethering the amino acids that will be incorporated in the peptide onto the NRPS multifunctional enzyme, indicating that all T domains are functional. Multiple amino acid alignment of E domains of modules 2 and 6 together with the epimerization domain present in GrsA, present in the Gramicidin S synthetase (described in FIG. 29(a-b)), shows an overall similarity of domains and conservation of amino acid motifs (E1 to E7) important for activity indicating that all E domains are functional (Stachelhaus and Walsh (2000), Biochemistry, 39, 5775-5787).

The incorporation and condensation of the various amino acid residues present in Compound 1 (0506) were predicted to proceed as follows and as described in FIG. 30.

The first module is composed of three domains, C, A and T. This is the loading module of the NRPS system and is likely that the condensation domain CD_{—}1 is skipped during the synthesis of the polypeptide chain. Analysis of the specificity-conferring code of the AD_{—}1 domain confirms the incorporation of an arginine residue. The second module contains C, A, T and E domains. Analysis of the specificity-conferring code of the AD_{—}2 domain confirms the incorporation of a serine residue, which is subsequently epimerized to D-serine by the EP_{—}2 domain. The third module comprises C, ADME (adenylation/methylation) and T domains. Analysis of the specificity-conferring code of the ADME_{—}3 domain confirms the incorporation of a δ-aminohydroxyornithine (DHOR). Additionally, the methylation domain present within the adenylation domain ADME_{—}3 catalyzes methylation of the δ-amino group of the δ-aminohydroxyornithine amino acid residue. The fourth module comprises C, A and T domains. Analysis of the specificity-conferring code of the AD_{—}4 domain confirms the incorporation of a serine. The fifth module comprises C, A and T domains. Analysis of the specificity-conferring code of the AD_{—}5 domain confirms the incorporation of another serine residue. The sixth module contains C, A and E domains. Analysis of the specificity-conferring code of the AD_{—}6 domain confirms the incorporation of a glutamine, which is subsequently epimerized to D-glutamine by the EP_{—}6 domain.

Specificity-conferring codes for the determination of the selectivity of adenylation domains are shown in table noted immediately below and were based on state of the art methods, such as taught in Challis et al. (2000), Chem. Biol., vol 7, no 3, 211-224, and in Stachelhaus et al. (1999), Chem. Biol. 6, 493-505. Also shown in the table is a comparison between the codes of the A domains included in SEQ ID NO: 10 and codes of other known sequences (with GenBank accession and module number in brackets). Analysis of the adenylation domains found in the NRPS allowed the incorporated amino acid in each unit to be identified. Specificity-conferring codes are highlighted in black in FIG. 27(a-d).

SPECIFICITY-CONFERRING CODES OF ADENYLATION DOMAINS

| | POSITION BASED ON GRSA | | | | | | | | AMINO ACID |
|---|---|---|---|---|---|---|---|---|---|
| MODULE No. | 235 | 236 | 239 | 278 | 299 | 301 | 322 | 330 | Coupled |
| AD1 OF SEQ ID NO: | D | V | W | I | I | G | A | V | ARG |
| 10 | | | | | | | | | |
| MCYC (BAA83994, | D | V | W | T | I | G | A | V | |
| M1) | | | | | | | | | |
| AD2 OF SEQ ID NO: | D | V | W | H | F | S | L | V | SER |
| 10 | | | | | | | | | |
| AD4 OF SEQ ID NO: | D | V | W | H | I | S | L | V | |
| 10 | | | | | | | | | |
| AD5 OF SEQ ID NO: | D | V | W | H | L | S | L | V | |
| 10 | | | | | | | | | |
| PEP1 (CAB38518, M1) | D | V | W | H | F | S | L | V | |
| ENTF (AAC73687, | D | V | W | H | F | S | L | V | |
| M1) | | | | | | | | | |
| PEP2 (AAX31558, | D | V | W | H | I | S | L | I | |
| M6) | | | | | | | | | |
| SYPC (AAO72425, | D | V | W | H | L | S | L | I | |
| M6) | | | | | | | | | |
| AD3 OF SEQ ID NO: | D | M | E | N | L | G | L | I | DHOR |
| 10 | | | | | | | | | |
| PEP1 (CAB53322, M3) | D | M | E | N | L | G | L | I | |
| ENTF (AAC73687, | D | M | E | N | L | G | L | I | |
| M1) | | | | | | | | | |
| AD6 OF SEQ ID NO: | D | A | Q | E | G | G | L | V | GLN |
| 10 | | | | | | | | | |
| LCHAA (CAA06323, | D | A | Q | D | L | G | V | V | |
| M1) | | | | | | | | | |
| ITUB (BAB69699, | D | A | Q | D | L | G | V | V | |
| M3) | | | | | | | | | |

Module 3 contains an adenylation-N-methyltransferase domain responsible for activation and tethering of □-aminohydroxyornithine that is subsequently N-methylated to give the N_{δ}-hydroxy-linked N_{α}-methylornithine (DHOR) found at amino acid position 3 in the final hexapeptide Compound 1 (0506). Epimerization activities in modules 2 and 6 are responsible for the presence of D-serine and D-glutamine in positions 2 and 6 respectively.

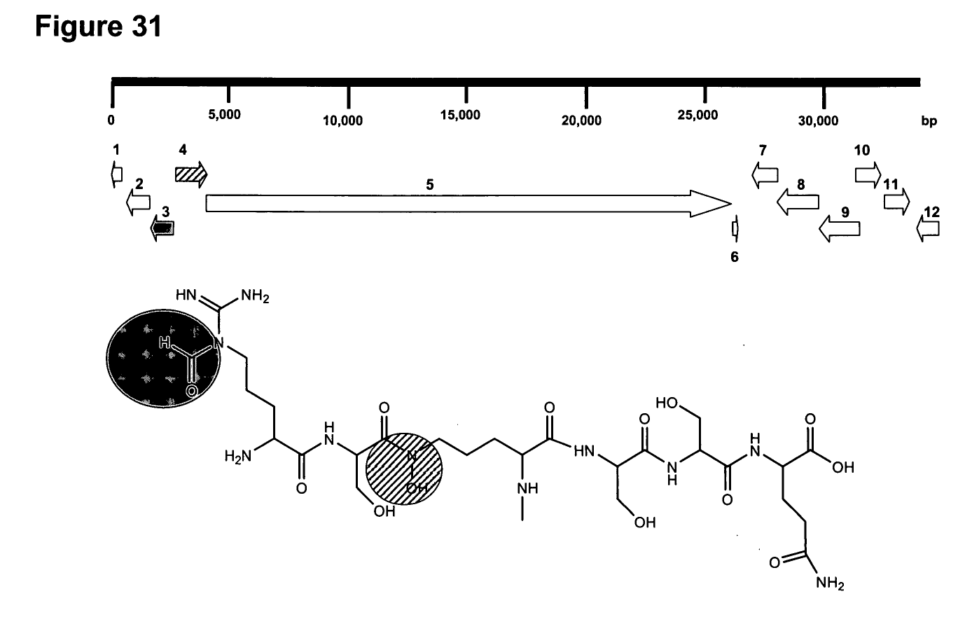

The role of three other enzymes is also depicted in FIG. 31. The mature hexapeptide Compound 1 (0506) was predicted to also contain a formyl group on the terminal nitrogen of the arginine of position 1, this formylation being catalyzed by the formyl transferase (FXBA) encoded by ORF 3. Similarly, ornithine would be delta-oxidized by the N-hydroxylase (OXRK) encoded by ORF 4, prior to its predicted incorporation as the third amino acid of the hexapeptide.

The order of modules as well as the predicted amino acid substrate specificities of the peptide synthetase repeating units were found to be in precise agreement with the structure of the hexapeptide later elucidated as detailed below, providing conclusive evidence that the herein described genetic locus is responsible for the biosynthesis of the isolated hexapeptide Compound 1 (0506).

Other ORFs present in the biosynthetic locus include ORF 1 (SEQ ID NO: 2) and ORF6 (SEQ ID NO: 12) of unknown function; ORF 2 (SEQ ID NO: 4) that is expected to be a transcriptional regulator and to regulate biosynthesis of Compound 1; ORF 7 (SEQ ID NO: 15), ORF 8 (SEQ ID NO: 17), ORF 9 (SEQ ID NO: 19), ORF 10 (SEQ ID NO: 21), ORF 11 (SEQ ID NO: 23) and ORF 12 (SEQ ID NO: 25) that are expected to be involved in transmembrane transport.

Strain [S01G01]007 (IDAC 210605-01) was used to produce Compound 1 (0506). The strain is a natural mutant of Amycolatopsis orientalis ATCC 43491 that was selected for resistance to streptomycin (300 μg/ml) and gentamicin (150 μg/ml). [S01G01]007 was cultivated under aerobic conditions in an aqueous nutrient medium containing assimilable sources of carbon, assimilable sources of nitrogen, inorganic salts and vitamins. Thus, for instance, preferred carbon sources were, soluble starch, glucose, glycerol and the like. Preferred nitrogen sources were Pharmamedia™, yeast extract, malt extract, and the like. Certain media were preferred for production of Compound 1 (0506). This strain is preferably grown at temperatures of about 28° C. to 30° C.

Strain [S01G01]007 was maintained and sporulated on agar plates of ISP2 medium (Difco). The inoculum for the production phase was prepared by adding two loopfulls of the spores obtained from the surface of the ISP2 agar plate to a 125-ml flask containing 25 ml of ITSB medium composed of 30 g trypticase soy broth (Bacto), 3 g yeast extract, 2 g MgSO₄, 5 g glucose, and 4 g maltose, and made up to one litre with distilled water. The flasks were shaken (250 rpm) for about 70 hours at 28° C. and then 10 ml of the pre-culture was used to inoculate each 2-L flasks containing 500 ml of sterile production medium (RA) consisting of 20 g soluble starch, 5 g Pharmamedia™, 2.5 g yeast extract, 1 g sodium chloride, 0.75 g K₂HPO₄, 1 g MgSO₄.7H₂O, 3 g CaCO₃. The medium was adjusted at pH 7.5 before sterilization. The fermentation batches were incubated aerobically under stirring (250 rpm) at 28° C. for a period of 96 hours.

In addition to the above media, Compound 1 (0506) was produced in media OA, NA and KA. Compound 1 (0506) was also produced using Amycolatopsis orientalis ATCC 43491 strain.

The mycelia and broth of the culture media (4×500 mL) were separated by centrifugation (3000 rpm, 15 min). The mycelia cake was extracted consecutively with 400 mL methanol and 400 mL acetone to produce an organic extract of the cells. The residual mycelia cake was re-extracted with 400 mL 20% aqueous methanol. The organic content of the broth was adsorbed (slurry-mode) on 240 mL Dianion HP-20 resin, which was subsequently washed with 400 mL water and eluted with a step gradient of 400 mL 60:40 methanol/water, 400 mL methanol, and 400 mL acetonitrile. The two latter fractions were combined and evaporated to produce an enriched organic extract of the broth for further purification.

The organic extract of the broth was suspended in 100 mL water and extracted with 2×100 mL n-butanol. The residual water phase was evaporated, redissolved in methanol, and coated onto 30 mL Diaion™ HP-20 resin by rotatory evaporation. The coated sample was applied on a 25×100 mm column of Diaion™ HP-20 and eluted with a 100 mL water/methanol step gradient (100:0, 80:20, 50:50, 0:100). The 50:50 eluate was evaporated, redissolved in 2 mL of 95:5 water/acetonitrile, and filtered through a 0.45 μm Acrodisc GHP 13 mm syringe filter.

Compound 1 (0506) was isolated on a Waters MS-based auto-purification system equipped with a 19×250 mm YMC ODS-AQ HPLC column, employing a gradient of 10 mM ammonium acetate/acetonitrile at a flow of 20 mL/min (95:5 for 1 min, 95:5-83.2:16.8 zero to 9 min.). Multiple partial samples of 200-500 μL were injected and the mass-directed fractions were combined, evaporated, and lyophilized to produce 63.4 mg of Compound 1 (0506).

The structure of Compound 1 (0506) was confirmed from spectroscopic data, including mass, UV, and NMR spectroscopy, as described in further detail below.

The calculated molecular weight (735.74) and formula (C₂₇H₄₉N₁₁O₁₃) of Compound 1 (0506) was confirmed by mass spectral analysis: negative ionization gave an [M-H]⁻ molecular ion of 734 and positive ionization gave an [M+H]⁺ molecular ion of 736. Strong fragments were also seen at m/z 536, 305, 218 and 131 respectively corresponding to fragments a), b), c) and d) as shown below.

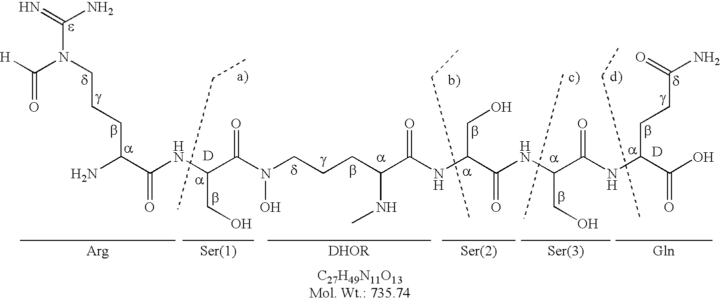

NMR data were collected from samples dissolved in D₂O (deuterated water), including proton, and multidimensional pulse sequences including CIGAR experiments. Assignment of proton and carbon NMR signals are shown in the table noted immediately below.

¹H and ¹³C NMR (δ_{H}, ppm) Data of Compound 1 (0506) in D₂O

| Amino | Position | ¹H | ¹³C | Group |
|---|---|---|---|---|
| Arg | C(O) | — | 174.0 | C |
| | α | 4.24 | 53.6 | CH |
| | β | 1.59, 1.69 | 28.2 | CH₂ |
| | γ | 1.65 | 22.8 | CH₂ |
| | δ | 3.52, 3.57 | 50.2 | CH₂ |
| | ε | — | 166.8 | C |
| | Nδ-CHO | 7.84, 8.18^{a} | 159.8 | CHO |
| D-Ser1 | C(O) | — | 170.5 | C |
| | α | 4.96 | 52.7 | CH |
| | β | 3.74 | 60.6 | CH₂ |
| DHOR | C(O) | — | 168.9 | C |
| | Nα-Me | 2.59 | 31.7 | CH₃ |
| | α | 3.85 | 61.1 | CH |
| | β | 1.75, 7.81 | 27.1 | CH₂ |
| | γ | 1.61 | 21.3 | CH₂ |
| | δ | 3.39, 3.53 | 47.8 | CH₂ |
| Ser2 | C(O) | — | 171.4 | C |
| | α | 4.50 | 55.9 | CH |
| | β | 3.79 | 61.2 | CH₂ |
| Ser3 | C(O) | — | 171.4 | C |
| | α | 4.35 | 55.9 | CH |
| | β | 3.75 | 61.3 | CH₂ |
| D-Gln | C(O)OH | — | 166.8 | C |
| | α | 4.40 | 50.6 | CH |
| | β | 1.66, 1.93 | 22.9 | CH₂ |
| | γ | 1.89 | 20.2 | CH₂ |
| | δ | — | 177.5 | C(O)NH₂ |

^{a}Rotamers

Characteristic chemical shifts for the amino acid residues were also assigned based on multidimensional pulse sequences CIGAR experiments. Correlations were found between a number of carbon atoms, as shown by the rows on the structure below, and a number of cross peaks in the 2D spectra of Compound 1 (0506) were key in the structural determination. For example, the formyl group is placed on the δ-Nitrogen of Arginine by a cross peak between the signals of the protons attached to Cδ and the quaternary carbon of the formyl group. The methyl group was similarly assigned to the α-Nitrogen by a cross peak between the protons of the methyl group and the α-Carbon atom. The sequence of amino acids was also determined by the presence of cross peaks between the amide carbon of each amino acid and the alpha proton of the next amino acid, or in the case of the carbonyl of the first serine to the protons of the methylene of the adjacent ornithine.

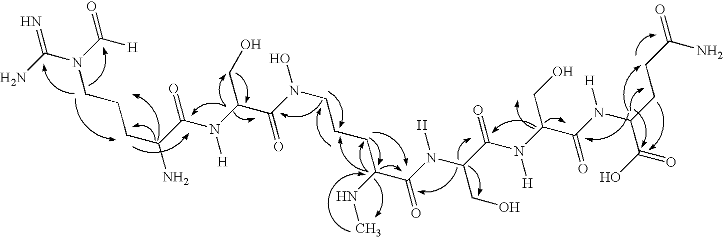

Antifungal bioactivity of Compound 1 (0506), in comparison to Amphotericin B, against Saccharomyces cerevisiae FHCRC 50514 was determined using a disk diffusion assay. Such an assay is commonly used to reveal activity of antifungal drugs against fungi (Wong G K, Griffith S, Kojima I and Demain A L, J. Antibiotics, 51(5): 487-491, 1998).

Indicator strain was diluted in saline solution (0.85% NaCl) and the innoculum density was adjusted to an O.D.₆₀₀ₙₘ of about 0.1±0.05. Inoculum (100 μL) was added to 100 mL of RPMI agar (kept at 55° C.) to give a final suspension of about 1×10⁵ CFU/mL, and poured into 150 mm Petri dishes. Plates were used immediately upon setting of the agar.

Compound 1 (0506) was diluted in DMSO to a concentration of 3.2 mg/mL. A 20 μL sample (64 μg) was deposited on the agar surface of prepared plates. Amphotericin solution in DMSO (5 mg/mL; 10 μL; 50 μg) was also deposited, in a separate location, on the agar surface. A negative control (10 μL of stock solution) was also deposited in a separate location on the surface of the agar. The plates were incubated at 37° C. for 24 hours.

Results showed a zone of inhibition (measured as the diameter from the location where the compound was deposited on the agar) for Compound 1 (0506) against S. cerevisiae FHCRC 50514 of 36.4 mm (at 64 μg), compared to 23.9 mm (at 50 μg) for Amphotericin B.

Compound 1 (0506) was also tested for its activity against enzymes and/or receptors known to be involved in several diseases.

The Angiotensin Converting Enzyme (ACE) has been found to be involved in the regulation of blood pressure, body fluid homeostasis and cell growth. Disease states, which involve ACE, include pathogenesis of arterial hypertension, congestive heart failure, left ventricular remodeling after myocardial infarction and other cardiovascular diseases. Compound 1 (0506) was assessed for its ability to block the activity of this enzyme.

MEK1 (for Map/Erk kinase-1) lies upstream of MAP kinase and stimulates the enzymatic activity of MAP kinase. MEKs have unusually restricted substrate specificity in that they phosphorylate and regulate only a very small number, in most cases one or two, downstream MAPKs. Enhanced MEK1 activity has been detected in a significant number of primary human tumor cells: inhibitors of this enzyme are thus being developed as therapeutic agents for the treatment of cancer. MEK1 activity also has been shown to be involved with ischemia.

Adenosine exerts its effects through four subtypes of a 7-transmembrane domain G-protein-coupled receptor: A₁, A_{2A}, A_{2B} and A₃. The adenosine A₃ receptor subtype is found mostly in brain, lung, liver, heart, kidney and testis. The adenosine A₃ receptor was shown to be implicated in cell cycle progression and cell growth, modulation of apoptosis, mast cell degranulation, ischemic preconditioning in the heart, neuroprotection and pro- and anti-inflammatory modulation. Both A₃ receptor-agonists and -antagonists are being pursued, for example, for the treatment of cancer, heart conditions, pain, asthma, inflammation and other immune implications.

The procedures used in the present invention in assessing the enzyme inhibition or receptor-binding of Compound 1 (0506) were based on known assays utilized in the art field: ACE (from rabbit; Ref: Bunning et al. (1983), Biochemistry, vol. 22, 103-110), MEK1 (rabbit; Ref: Reiners et al. (1998), Mol. Pharmacol., vol. 53, 438-445), and Adenosine A₃ (human; Ref: Olah et al. (1994), Mol. Pharmacol., vol. 45, 978-982, and Salvatore et al (1993), Proc. Natl. Acad. Sci. USA, vol. 90, no 21, 10365-10369). The procedures used were based on the respective references mentioned above, and the conditions are summarized as follows:

ACE: Source: rabbit lung; substrate: 500 μM (N-3-[2-furyl] acryloyl)-Phe-Gly-Gly (FAPGG); vehicle: 1% DMSO; Pre-incubation time and temperature: 15 min at 25° C.; Incubation time and temperature: 30 min at 25° C.; Incubation buffer: 50 mM HEPES, 300 nM NaCl, at pH 7.5; Quantitation method: Spectrometric quantitation of FAPGG; Significance criteria: ≧50% of max stimulation or inhibition. Reference compound for ACE inhibition was Captopril, which had an IC₅₀ of 7.4 nM (historical 10 nM).

MEK1: Source: rabbit recombinant E. coli; substrate: 10 μg/mL Myelin Basic Protein (MBP); vehicle: 1% DMSO; Pre-incubation time and temperature: 15 min at 25° C.; Incubation time and temperature: 60 min at 25° C.; Incubation buffer: 50 mM HEPES, 20 mM MgCl₂, 0.2 mM Na₃VO₄, 1 mM DTT, at pH 7.4; Quantitation method: Elisa quantitation of MBP-P; Significance criteria: ≧50% of maximum stimulation or inhibition. Staurosporine was the reference compound for MEK1 inhibition and had a 2.1 nM (IC₅₀) activity (historical 3.8 nM).

Adenosine A₃: Source: human recombinant CHO-K1 cells; Ligand: 0.5 nM [H₃] Prazosin; vehicle: 1% DMSO; Incubation time and temperature: 30 min at 25° C.; Incubation buffer: 50 mM Tris-HCl, 0.1% ascorbic acid, 10 μM pargyline; Non-specific ligand: 0.1 μM Prazosin; K_{D}: 0.29 nM (historical value); Bₘₐₓ: 0.095 pmole/mg protein (historical value); Specific binding: 90% (historical value); Quantitation method: Radioligand Binding; Significance criteria: ≧50% of max stimulation or inhibition. Reference compound used for Adenosine A₃ binding assay is IB-MECA, with an IC₅₀ of 8.2 nM (historical 5.2 nM), and an historical Kᵢ of 4.7 nM and n_{H} value of 0.7.

IC₅₀ values of reference standards were determined by a non-linear, least square regression analysis using Data Anaysis Toolbox™ (MDL Information Systems, San Leandro, Calif., USA).

Compound 1 was tested at a constant concentration (in 1% DMSO) of 10 μM, and the results obtained for inhibition/binding assays are summarized in the table presented immediately below. An inhibition rate 50% and above was considered significant. A negative value indicated stimulation of binding or enzyme activity.

Biochemical results of Compound 1 (0506)

| Enzyme/Receptor | Species | Concentration | % inhibition |
|---|---|---|---|
| Angiotensin Converting Enzyme | Rabbit | 10 μM | 50 |
| Protein Serine/Threonine | Rabbit | 10 μM | 63 |
| Kinase MEK1 | | | |
| Adenosine A3 Receptor | Human | 10 μM | −101 |

### Example 9

### Identification of a Novel Polyketide Furanone from Streptomyces aculeolatus NRRL 18422

Streptomyces aculeolatus NRRL 18422 was obtained from the Agricultural Research Service Culture Collection (1815 N. University Street, Peoria, Ill. 61604, USA). Using the genome scanning method described in U.S. patent application Ser. No. U.S. Ser. No. 10/232,370, Canadian Patent Application CA 2,352,451 and Zazopoulos et. al., Nature Biotechnol., 21, 187-190 (2003), was identified in the genome of Streptomyces aculeolatus NRRL 18422 and predicted to be the biosynthetic locus for the production of a polyketide designated as Compound 2 (05101).

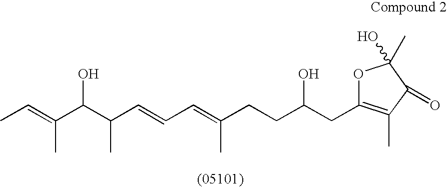

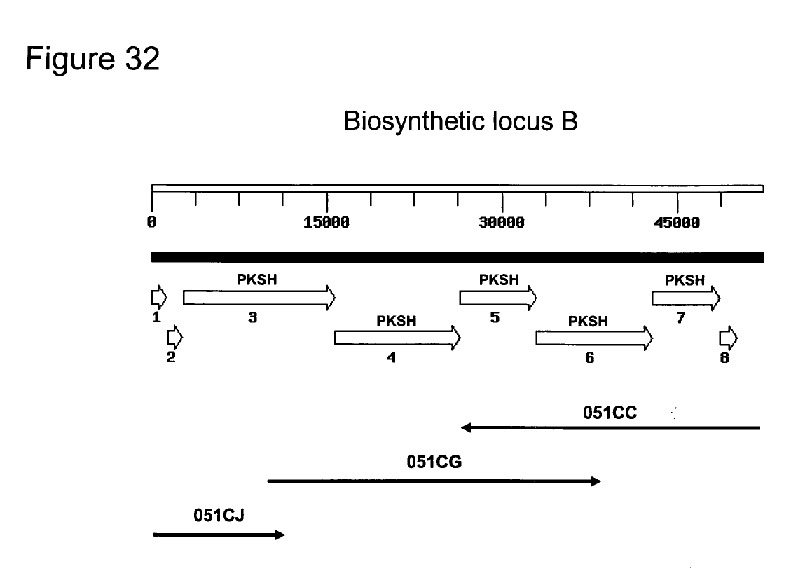

The order, relative position and orientation of the 8 open reading frames representing the predicted proteins of the biosynthetic locus are illustrated schematically in FIG. 32. The top line in FIG. 32 provides a scale in base pairs. The black bar depicts the DNA contig that covering the locus. The empty arrows represent the 8 open reading frames of this biosynthetic locus. The black arrows represent the three deposited cosmid clones covering the locus. The biosynthetic locus B spans approximately 50,000 base pairs of DNA. More than 10 kilobases of DNA sequence were analyzed on each side of the locus and these regions were deemed to contain primary genes or genes unrelated to the synthesis of Compound 2 (05101).

To identify the function of the proteins encoded by the genes forming the biosynthetic locus B for the production of Compound 2 (05101), the encoded proteins were compared, using the BLASTP version 2.2.10 algorithm with the default parameters, to sequences in the National Center for Biotechnology Information (NCBI) nonredundant protein database and the DECIPHER® database of microbial genes, pathways and natural products (Ecopia BioSciences Inc. St.-Laurent, QC, Canada). Five of the ORFs (ORFs 22 to 26) in the locus B cluster were designated as belonging to the type I polyketide synthase family. These type I polyketide synthases of locus B were further processed by an automated software application that parses the proteins into individual enzymatic modules and domains. As described above in Example 5, each domain sequence was compared to a series of protein models of active domains to identify domains that were likely to be nonfunctional. Additional computer scripts were invoked when particular domains are encountered.

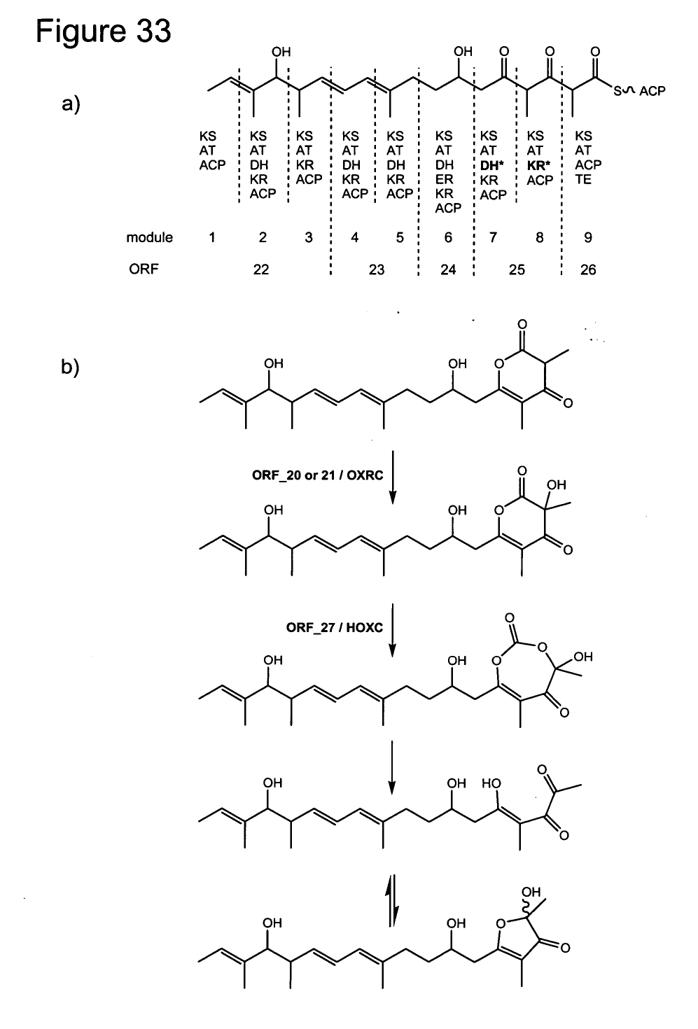

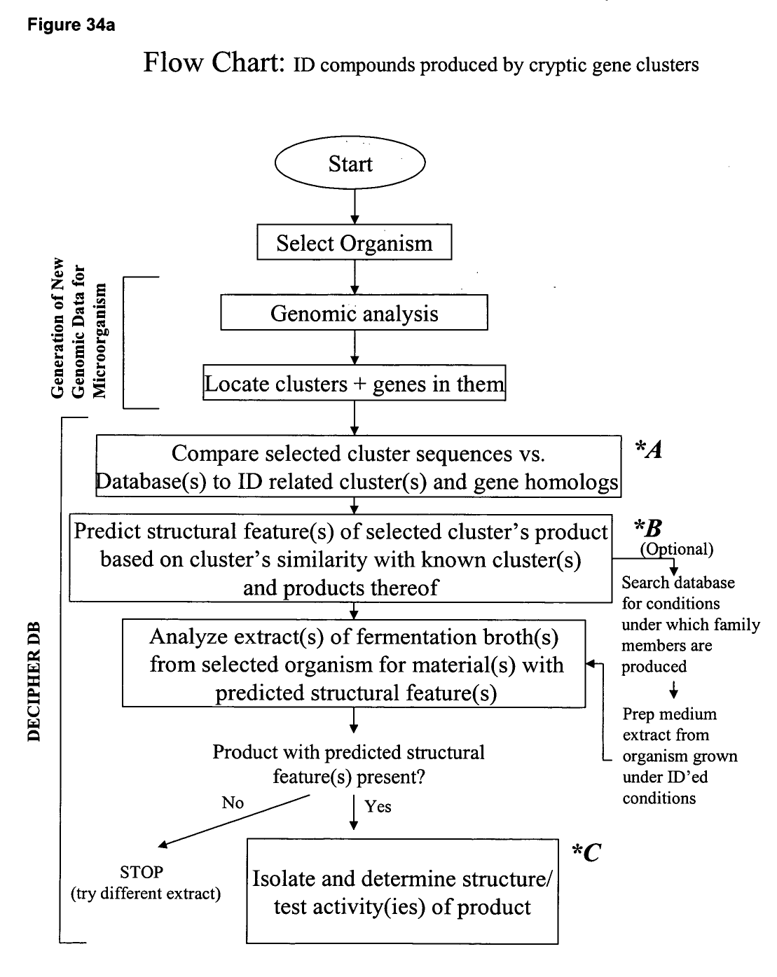

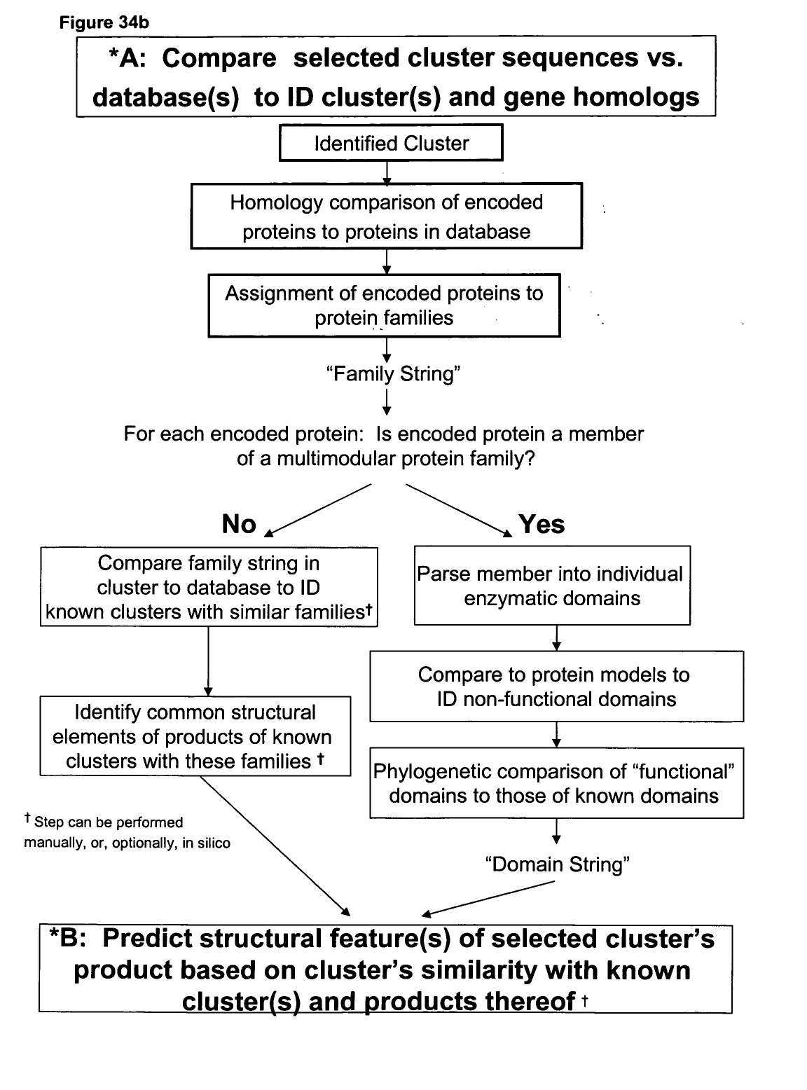

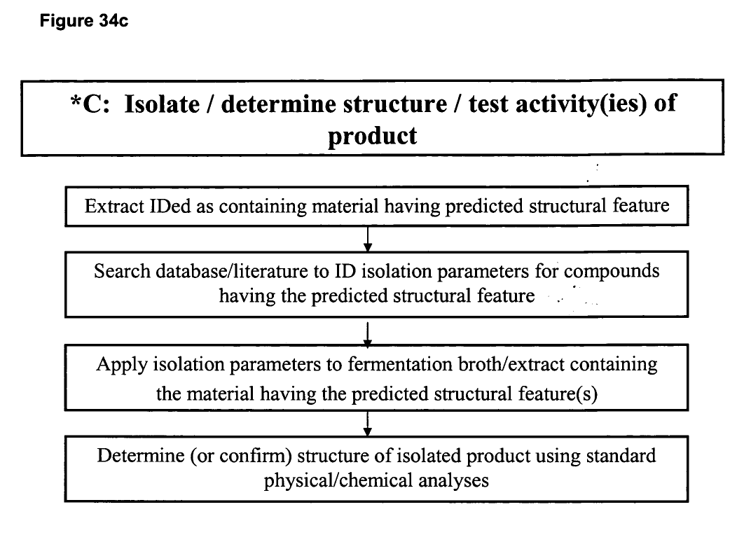

Clustal™ alignment analysis of the PKS domains revealed that all domains were complete and contained known motifs and conserved amino acid residues required for activity, except for the dehydratase (DH) domain of module 7 and the ketoreductase (KR) domain of module 8, which was consistent with the subsequent structure confirmation of Compound 2 (05101). The predicted role of each domain of the PKS system in the biosynthesis of Compound 2 (05101) is also shown schematically in FIG. 33.

Phylogenetic analysis of the acyltransferase (AT) domains in the PKS B system was conducted to assess the nature of the 3-keto acyl units that are incorporated in the growing polyketide chain. The AT domains of the PKS B system were compared to two AT domains, AAF71775mod 1 and AAF71776mod 3 (National Center for Biotechnology Information (NCBI) nonredundant protein database), derived from the nystatin PKS system (Brautaset et al. (1997), Chemistry and Biology, vol 7, 395-403) and responsible for the incorporation of methylmalonyl-CoA and malonyl-CoA respectively. Results from the phylogenetic analysis indicated that, in the PKS B system for production of Compound 3 (05101), modules 2, 3, 5, 8 and 9 were predicted to incorporate methylmalonate units in the polyketide backbone of Compound 2 (05101), whereas all remaining AT domains, namely modules 1, 4, 6 and 7 incorporate malonate extender β-keto acyl units in the polyketide backbone of Compound 2(05101).

Type I PKS domains and the reactions they carry out are well known to those skilled in the art and well documented in the literature (see, for example, Hopwood, 1997, Chem. Rev., vol. 97, 2465-2497). Those skilled in the art will readily appreciate that it is possible to determine the polyketide core structure produced by PKS systems through domain analysis. While not intending to be limited to any particular mode of action or biosynthetic scheme, FIG. 33 describes production of Compound 2 (05101) using the genes and proteins detailed above. FIG. 33a schematically describes a series of reactions catalyzed by the PKS system based on the correlation between the deduced domain architecture and the polyketide core of Compound 2 (05101). Referring to FIG. 33a, the acyltransferase of the loading module (module 1) loads a malonyl-CoA extender unit onto the ACP domain. This extender unit is subsequently decarboxylated through the action of the KS domain contained in module 1. The polyketide chain continues to grow by the sequential condensation of malonyl-CoA and methylmalonyl-CoA extender units that are further reduced by specific domains to various degrees. The dehydratase domain found in module 7, as well as the ketoreductase domain in module 8, are inactive and consequently do not catalyze their respective enzymatic functions (FIG. 33a). The mature polyketide chain is cyclized between the thioester carbonyl group and the internal carbonyl incorporated by module 7 through the action of the thioesterase domain found in module 9 and subsequently released from the polyketide synthase (FIG. 33b). The pyran-2-one moiety formed by the thioesterase is further modified by hydroxylation catalyzed by cytochrome P-450 enzymes (OXRCs) encoded by two of the ORFs (ORFs 20 or 21) of locus B, and by oxygenation through the action of an oxygenase enzyme (HOXC) encoded by one of the ORFs (ORF 27) of locus B, and which would catalyze a Bayer-Villiger monooxygenation reaction using NADPH as a cofactor (FIG. 33b). The resulting highly oxidized ring would be extremely unstable and subject of hydrolytic ring cleavage with spontaneous loss of one carbon unit, presumably as carbon dioxide. Various tautomeric forms could be generated, two of which are depicted in FIG. 33b, although the compound would exist principally as a furan-3-one form shown in FIG. 33b.

Three deposits of E. coli DH10B vectors, each harbouring a cosmid clone of a partial biosynthetic locus from Streptomyces aculeolatus NRRL 18422 and together spanning the full biosynthetic locus B for producing Compound 3 (05101) have been deposited with the International Depositary Authority of Canada, Bureau of Microbiology, Health Canada, 1015 Arlington Street, Winnipeg, Manitoba, Canada R3E 3R2 on Dec. 5, 2003 and were assigned deposit accession numbers IDAC 051203-01 (051CJ), IDAC 051203-02 (051 CG) and IDAC 051203-03 (051 CC) respectively, as shown in FIG. 32.

The deposit of the deposited strains has been made under the terms of the Budapest Treaty on the International Recognition of the Deposit of Micro-organisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement, such as that required under 35 U.S.C. §112. A license may be required to make, use or sell the deposited strains, and compounds derived therefrom, and no such license is hereby granted.

For production of Compound 2 (05101), a vial containing frozen mycelium or spores of Streptomyces aculeolatus NRRL 18422 was taken out of freezer and kept on dry ice. Under aseptic conditions, a loopful of the frozen stock was taken and streaked on the surface of NS agar plate and incubated at 28° C. for 10⁻¹⁵ days until vegetative mycelium appeared. Longer incubation is required for sporulation.

Streptomyces aculeolatus NRRL 18422 was cultivated under aerobic conditions in an aqueous nutrient medium containing assimilable sources of carbon, assimilable sources of nitrogen and inorganic salts. Thus, for instance, preferred nitrogen sources are Pharmamedia, fishmeal, dry yeast and the like. Certain media are preferred for production of Compound 2 (05101). This strain is preferably grown at temperatures between 28° C. and 30° C.

To prepare a vegetative culture, the surface growth from the agar plate was homogenized and transferred to a 125 mL flask containing 25 mL of sterile medium ITSB composed of 30 g trypticase soy broth (Bacto), 3 g yeast extract, 2 g MgSO₄, 5 g glucose, 4 g maltose to which one litter distilled water was added. This vegetative culture was incubated at 28° C. for about 70 hours on a shaker set at 250 rpm.

From the vegetative culture, 10 mL was used to inoculate 2 L baffled flasks each containing 500 mL of sterile production medium JA consisting of 35 g malt extract, 30 g corn starch, 15 g corn steep liquor, 15 g Pharmamedia and 2 g calcium carbonate (CaCO₃). The medium was adjusted at pH 7.3 before sterilization. The fermentation batches were incubated aerobically under stirring (250 rpm) at 28° C. for a 4 day period.

Alternatively, from the vegetative culture, 10 mL was used to inoculate 2 L baffled flasks each containing 500 mL of sterile production medium ET consisting of 60 g molasses, 20 g soluble starch, 20 g fish meal, 0.1 g copper sulfate pentahydrate (CuSO₄.5H₂O), 0.5 mg sodium iodide (NaI) and 2 g calcium carbonate (CaCO₃). The fermentation batches were incubated aerobically under stirring (250 rpm) at 28° C. for a 7 day period.

Other media that could be used for production of Compound 2 (05101) are KA, EA, RA, CA and CB.

For isolation and purification of Compound 2 (05101), the mycelia and broth of the culture media (5 to 8 L) was separated by centrifugation (3000 rpm, 15 min). The mycelia cake was extracted consecutively with 200 mL of methanol and 200 mL of acetone to produce an organic extract of the cells. The organic content of the broth was adsorbed (slurry-mode) on 120 mL Diaion HP-20 resin, which was subsequently washed with 200 mL water and eluted with a step gradient of 200 mL 60:40 methanol/water, 200 mL methanol, and 200 mL acetonitrile. The two latter fractions were combined with the organic cell extract and evaporated to produce the total crude extract. The solid crude extract was extracted with 2×500 mL ethyl acetate, which was evaporated and resuspended in 50 mL methanol/water 90:10. This methanolic phase was defatted by extraction with 50 mL n-heptane and evaporated to produce an HPLC-compatible sample.

The sample was dissolved in a minimal amount of dimethyl sulfoxide and filtered through a 0.45 μm 13 mm Acrodisc GHP syringe filter. Multiple injections of no more than 500 μL sample on a Waters RCM Nova-Pak HR C18 6 μm 60 Å25×200 mm column (water/acetonitrile 70:30-43:57 zero to 12 min+43:57-0:100 12 to 16 min at 20 mL/min) afforded Compound 2 (05101) as a semi-pure solid (2-3 mg/L broth).

A final step of normal phase HPLC was needed to purify Compound 2 (05101). A Waters Nova-Pak Silica 6 μm 19×300 mm column eluted with chloroform/methanol 98:2 under isocratic conditions at 20 mL/min yielded pure Compound 2 (05101) (0.9 mg/L broth).

The polyketide core structure described in FIG. 33b, based on the architecture of the PKS B system of the biosynthetic locus for the production of Compound 2 (05101), was entirely consistent with the polyketide portion of the chemical structure, as described below, as determined by MS sprectra data, ¹H- and ¹³C NMR spectra data, thereby demonstrating that locus B is responsible for the biosynthesis of Compound 2 (05101).

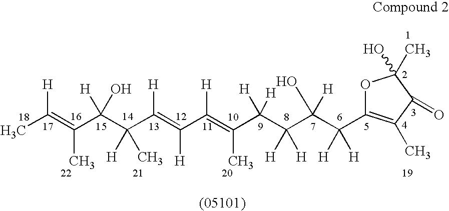

The structure of Compound 2 (05101) was determined from spectroscopic data including NMR spectroscopy. The molecular weight of Compound 2 (05101) was determined to be 378.24 by electrospray mass spectrometry shown in the table immediately below and gave molecular formulae of C₂₂H₃₄O₅.

Mass Spectrometry data for Compound 2 (05101)

| Compound | Ionization | Mass (m/z) | Fragment |
|---|---|---|---|
| | | 401.20 | (M + Na) |
| 3 | ES⁺ | 361.25 | (M + H − H₂O) |
| | | 343.23 | (M + H − 2H₂O) |
| 3 | ES⁻ | 377.17 | (M − H) |

The ¹H NMR spectrum for Compound 2 and multidimensional pulse sequences experiments, gCOSY, NOESY, gHSQC, gHMBC and CIGAR was measured at about 500 MHz on a sample dissolved in MeOH-d₄. The ¹³C NMR spectrum of Compound 2 was measured at 125 MHz on a sample dissolved in MeOH-d₄. Proton and carbon signals respectively shown in the two tables presented immediately below were assigned from ¹H and ¹³C NMR data and correlations observed in gCOSY, NOESY, gHSQC, gHMBC and CIGAR spectral analysis.

¹H NMR Data (δ, ppm) for Compound 2 (05101) in MeOH-D₄

| Assignment | Chemical shift | Group |
|---|---|---|
| 1 | 1.44 | CH₃ |
| 6 | 2.68, 2.73 | CH₂ |
| 7 | 3.96 | CH |
| 8 | 1.64, 1.70 | CH₂ |
| 9 | 2.16, 2.24 | CH₂ |
| 11 | 5.87 | CH |
| 12 | 6.31 | CH |
| 13 | 5.55 | CH |
| 14 | 2.35 | CH |
| 16 | 3.64 | CH |
| 17 | 5.44 | CH |
| 18 | 1.63 | CH₃ |
| 19 | 1.68 | CH₃ |
| 20 | 1.76 | CH₃ |
| 21 | 0.86 | CH₃ |
| 22 | 1.60 | CH₃ |

¹³C NMR Data (δ, ppm) for Compounds 1 (05101) in
MeOH-D₄

| Assignment | 3 | Group |
|---|---|---|
| 1 | 21.0 | CH₃ |
| 2 | 103.0 | C |
| 3 | 204.1 | C |
| 4 | 109.2 | C |
| 5 | 185.8 | C |
| 6 | 37.1 | CH₂ |
| 7 | 68.4 | CH |
| 8 | 35.5 | CH₂ |
| 9 | 35.6 | CH₂ |
| 10 | 135.5 | C |
| 11 | 125.7 | CH |
| 12 | 126.8 | CH |
| 13 | 135.3 | CH |
| 14 | 40.7 | CH |
| 15 | 82.3 | CH |
| 16 | 136.7 | C |
| 17 | 121.7 | CH |
| 18 | 11.9 | CH₃ |
| 19 | 4.6 | CH₃ |
| 20 | 15.4 | CH₃ |
| 21 | 16.9 | CH₃ |
| 22 | 9.8 | CH₃ |

Compound 2 (05101) was shown principally as mixture of tautomeric forms, the two anomeric hemiactetals were mostly observed. This mixture is about 1:1 in MeOH-d₄. In benzene-d₆, one anomeric form is slightly predominant. For the purpose of clarity, only signals from one tautomeric form were described in the two above-noted tables.

Antifungal activity of Compound 2 (05101) against Saccharomyces cerevisiaeΔpdr1/pdr3/erg6 (FHCRC 50514) was determined according to NCCLS protocols (NCCLS. Reference Methods for Broth Dilution Antifungal Susceptibility Testing of yeasts; Approved Standard. NCCLS document M27A (ISBN 1-56238-328-0), NCCLS, Wayne, Pa., USA, available at the worldwide web address of “nccls.org”). Test article was prepared the day of the experiment as a 100× stock solution in DMSO, with concentrations ranging from 6.4 mg/mL to 0.0125 mg/mL (two-fold dilution series over 10 points). An aliquot of the (100×) solution was then diluted 50-fold in the test medium (RPMI 1640 supplemented with adenine sulfate at 24 μg/mL) to give a set of 10 (2×) solutions. 50 μL aliquots of each (2×) solution were distributed (in duplicate) into wells of a 96-well plate (two rows of wells 1 to 10). The final wells (column 12) were filled with 100 μL of medium for sterility control. 50 μL aliquots of medium were added to wells of column 11 for growth control.

Positive control (prepared the day of the experiment) used was Fluconazole™. A stock solution (100×) were prepared in methanol, with concentrations ranging from 3.2 mg/mL to 0.0065 mg/mL (a two-fold dilution over 10 points). An aliquot of each solution was further diluted 50-fold in test medium to give a set of 10 (2×) solutions. 50 μL aliquots of the (2×) solutions, and sterility and growth controls were also added as done for the test article.

S. cerevisiae strain was diluted in saline solution (0.85% NaCl) and innoculum density adjusted to an O.D._{600 nm} of about 0.1±0.05. Indicator strain was then diluted ( 1/1000) in test medium (RPMI 1640 supplemented with adenine sulfate at 24 μg/mL). Aliquots of the strain in test medium (50 μL) were added to each well (except row 12, sterility control wells) to bring the final dilution of the test articles and positive control to 1×.

Assay plates were incubated at 35° C. for 48 hours. The minimum inhibitory concentration (MIC) was determined as the lowest concentration of test or control sample that results in total absence of growth and was determined as mean value of duplicate experiments. Compound 2 exhibited an MIC of 64 μg/mL against S. cerevisiae Δpdr1/pdr3/erg6 (FHCRC 50514). Fluconazole™ as positive control exhibited an MIC of 0.25-0.5 μg/mL.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A method of determining the presence of a secondary metabolite synthesized by a microorganism, the method comprising:
a) predicting a chemical property of a candidate secondary metabolite, wherein said predicting is performed on the basis of a comparison of a target gene cluster of said microorganism with information in a database comprising genomic information from a plurality of microorganisms, said target gene cluster encoding genetic information for the generation of said candidate secondary metabolite;
b) detecting in an extract of a fermentation broth in which said microorganism has been cultured the presence of a secondary metabolite that exhibits said chemical property predicted in step (a); and
c) isolating said secondary metabolite exhibiting said property from an extract of a fermentation broth in which said microorganism has been cultured, wherein at least one step in the process of said isolating is guided by information regarding the isolation of a compound exhibiting said property,
whereby the presence of a secondary metabolite produced by said microorganism is determined.

2. The method of claim 1 wherein said chemical property of said secondary metabolite correlates with a biological activity of said secondary metabolite, such that the presence of said biological activity indicates the presence of said chemical property.

3. The method of claim 2 wherein said detecting step (b) comprises measurement of a biological activity of said secondary metabolite.

4. The method of claim 1, further comprising the step, prior to step (b), of preparing an extract of a fermentation broth in which said microorganism has been cultured.

5. The method of claim 3, wherein a plurality of extracts are prepared from a plurality of fermentation broths in which said microorganism has been cultured under a plurality of growth conditions.

6. The method of claim 1, further comprising the step, prior to step (b), of predicting a culture condition under which a secondary metabolite exhibiting said property will be produced by said microorganism, wherein said predicting is performed on the basis of a query of information in a database comprising information regarding secondary metabolite production.

7. The method of claim 5 further comprising the step of culturing said microorganism under the culture condition predicted in claim 5.

8. The method of claim 1 wherein said extract is prepared from a fermentation broth in which said microorganism was cultured, wherein said microorganism was cultured under a condition predicted to induce the production of a secondary metabolite exhibiting said property.

9. The method of claim 1 wherein said information regarding the isolation of a compound is obtained by querying a database.

10. The method of claim 1 wherein detecting step (b) comprises fractionating said extract.

11. The method of claim 1 wherein detecting step (b) comprises measuring one or more properties selected from the group consisting of molecular mass, UV spectrum and a biological activity.

12. The method of claim 1 wherein isolating step (c) comprises the step of fractionating said extract.

13. The method of claim 1 wherein detecting step (b) comprises assaying said extract, or a fraction thereof, for a biological activity.

14. The method of claim 3 wherein said biological activity is selected from the group consisting of anti-bacterial, anti-fungal or anti-cancer activity.

15. The method of claim 1 wherein isolating step (c) comprises measuring one or more properties selected from the group consisting of molecular mass, UV spectrum and a biological activity.

16. The method of claim 1 wherein isolating step (c) comprises the step of measuring a biological activity in said extract or a fraction thereof.

17. The method of claim 1, further comprising the step of determining a sub-structure of the secondary metabolite isolated in step (c).

18. The method of claim 17, wherein at least one step in the process of determining the structure of the secondary metabolite isolated in step (c) is guided by information obtained from a database regarding the structural determination of a compound exhibiting said property.

19. The method of claim 1 wherein two or more chemical properties are predicted in step (a), and wherein said secondary metabolite isolated in step (c) exhibits said two or more chemical properties predicted in step (a).

20. The method of claim 19 wherein the structure of said candidate secondary metabolite is predicted in step (a).

21. The method of claim 1 wherein said target gene cluster comprises a sequence encoding a polypeptide or polypeptide domain that has sequence similarity, as evidenced by an E value of 10⁻⁵ or lower to a polyketide synthase enzyme or domain thereof.

22. The method of claim 1 wherein said target gene cluster comprises a sequence encoding a polypeptide or polypeptide domain that has sequence similarity, as evidenced by an E value of 10⁻⁵ or lower to a non-ribosomal peptide synthetase enzyme or a domain thereof.

23. The method of claim 1 wherein said database comprises genomic information linked to or annotated with additional information selected from one or more of: a chemical property, a physical property or a biological activity of a secondary metabolite; and a chemical, physical or biological property of a metabolite biosynthesized by a gene product encoded by a secondary metabolite gene cluster.

24. The method of claim 23 wherein said property of the metabolite biosynthesized by a gene product encoded by a secondary metabolite gene cluster correlates with the presence of a polypeptide domain encoded by the secondary metabolite gene cluster.

25. The method of claim 1 wherein information pertaining to one or more of:
a) the correlation between the secondary metabolite isolated in step (c) and the target gene cluster;
b) chemical, physical or biological properties of said secondary metabolite isolated in step (c); and
c) conditions under which the microorganism synthesizes said secondary metabolite isolated in step (c);
is added to said database.

26. A method of determining the presence of a secondary metabolite synthesized by a microorganism, the method comprising:
a) predicting a chemical property of a said candidate secondary metabolite, wherein said predicting is performed on the basis of a comparison of a target gene cluster of said microorganism with information in a database comprising genomic information from a plurality of microorganisms, said target gene cluster encoding genetic information for the generation of said candidate secondary metabolite
b) culturing said microorganism in a fermentation broth under conditions predicted to induce the production of a secondary metabolite having said property by said microorganism;
c) detecting in an extract of said fermentation broth the presence of a secondary metabolite that exhibits said chemical property predicted in step (a); and
d) isolating said secondary metabolite exhibiting said property from an extract of a fermentation broth in which said microorganism has been cultured, wherein at least one step in the process of said isolating is guided by information regarding the isolation of a compound exhibiting said property,
whereby the presence of a secondary metabolite produced by said microorganism is determined.

27. The method of claim 26 wherein said chemical property of said secondary metabolite correlates with a biological activity of said secondary metabolite, such that the presence of said biological activity indicates the presence of said chemical property.

28. The method of claim 27 wherein said detecting step (c) comprises measurement of a biological activity of said extract.

29. The method of claim 28 wherein said biological activity is selected from the group consisting of anti-bacterial, anti-fungal or anti-cancer activity.

30. The method of claim 26 wherein said conditions predicted to induce the production of a secondary metabolite are selected on the basis of a query of a database comprising information regarding secondary metabolite production by a microorganism.

31. The method of claim 26 wherein step (b) comprises culturing said microorganism under a plurality of different conditions.

32. The method of claim 26, further comprising the step, prior to step (c), of preparing an extract of a fermentation broth in which said microorganism has been cultured.

33. The method of claim 26 wherein detecting step (c) comprises fractionating said extract.

34. The method of claim 26 wherein detecting step (c) comprises measuring one or more properties selected from the group consisting of molecular mass, UV spectrum and a biological activity.

35. The method of claim 26 wherein said information regarding the isolation of a compound exhibiting said property is obtained by querying a database.

36. The method of claim 26 wherein isolating step (d) comprises the step of fractionating said extract.

37. The method of claim 26 wherein isolating step (d) comprises measuring one or more properties selected from the group consisting of molecular mass, UV spectrum and a biological activity.

38. The method of claim 26 wherein isolating step (d) comprises the step of measuring a biological activity in said extract or a fraction thereof.

39. The method of claim 26, further comprising the step of determining a sub-structure of the secondary metabolite isolated in step (d).

40. The method of claim 26 wherein two or more properties are predicted in step (a), and wherein said secondary metabolite isolated in step (d) exhibits said two or more properties predicted in step (a).

41. The method of claim 26 wherein the structure of said candidate secondary metabolite is predicted in step (a).

42. The method of claim 26 wherein said target gene cluster comprises a sequence encoding a polypeptide or polypeptide domain that has sequence similarity, as evidenced by an E value of 10⁻⁵ or lower to a polyketide synthase enzyme or enzyme domain.

43. The method of claim 26 wherein said target gene cluster comprises a sequence encoding a polypeptide or polypeptide domain that has sequence similarity, as evidenced by an E value of 10⁻⁵ or lower to a non-ribosomal peptide synthetase enzyme or a domain thereof.

44. The method of claim 26 wherein said database comprises genomic information linked to or annotated with additional information selected from one or more of: a chemical property, a physical property or a biological activity of a secondary metabolite; and a chemical, physical or biological property of a metabolite biosynthesized by a gene product encoded by a secondary metabolite gene cluster.

45. The method of claim 26 wherein information pertaining to one or more of:
a) the correlation between the secondary metabolite isolated in step (d) and the target gene cluster;
b) chemical, physical or biological properties of said secondary metabolite isolated in step (d); and
c) conditions under which the microorganism synthesizes said secondary metabolite isolated in step (d);
is added to said database.

46. A method of identifying a microorganism that produces a secondary metabolite belonging to a selected chemical family of compounds, the method comprising:
a) selecting a chemical property that is common to compounds in said selected chemical family;
b) identifying in a microorganism known to produce a compound belonging to said selected chemical family, a first gene cluster that participates in generating said chemical property of a secondary metabolite;
c) querying a database comprising genomic information with one or more sequences comprised by said first gene cluster to identify a candidate microorganism comprising a gene cluster comprising a sequence encoding a polypeptide or protein having a sequence similarity, as evidenced by an E value of 10⁻⁵ or lower, to a polypeptide or protein encoded by said first gene cluster;
d) detecting in an extract of a fermentation broth in which said candidate microorganism has been cultured the presence of a compound that exhibits said chemical property, whereby the presence of a compound that exhibits said chemical property identifies said candidate microorganism as one that produces a secondary metabolite belonging to said selected chemical family of compounds.

47. The method of claim 46 wherein said candidate microorganism and said microorganism known to produce a compound belonging to said selected chemical family are the same organism.

48. The method of claim 46 further comprising the step of isolating said secondary metabolite belonging to said selected chemical family from a fermentation broth in which said candidate microorganism has been cultured.

49. The method of claim 46, further comprising the step, prior to detecting step (d), of predicting a culture condition under which said candidate microorganism will produce a secondary metabolite in said selected chemical family, wherein said predicting is performed on the basis of a query of information in a database comprising information regarding secondary metabolite production.

50. The method of claim 49 further comprising the step of culturing said candidate microorganism under the culture condition predicted in claim 49.

51. The method of claim 50, further comprising the step of preparing an extract of fermentation broth in which said candidate microorganism was cultured under said predicted culture condition.

52. The method of claim 46 wherein said extract is prepared from a fermentation broth in which said microorganism was cultured, wherein said microorganism was cultured under a condition predicted to induce the production of a secondary metabolite exhibiting said property.

53. The method of claim 46, wherein a plurality of extracts are analyzed, said plurality of extracts prepared from a plurality of fermentation broths in which said microorganism has been cultured under a plurality of growth conditions.

54. The method of claim 46 wherein detecting step (d) comprises fractionating said extract.

55. The method of claim 46 wherein said chemical property of said secondary metabolite correlates with a biological activity of said secondary metabolite, such that the presence of said biological activity indicates the presence of said chemical property.

56. The method of claim 55 wherein said detecting step (d) comprises measurement of a biological activity of said extract or a fraction thereof.

57. The method of claim 56 wherein said biological activity is selected from the group consisting of anti-bacterial, anti-fungal and anti-cancer activity.

58. The method of claim 46 wherein detecting step (d) comprises measuring one or more properties selected from the group consisting of molecular mass, UV spectrum and a biological activity.

59. The method of claim 46 further comprising the step of isolating said secondary metabolite from fermentation broth of in which said candidate microorganism has been cultured.

60. The method of claim 59 wherein at least one step in the process of said isolating is guided by information obtained from a database regarding the isolation of a compound exhibiting said property.

61. The method of claim 59 wherein said candidate microorganism was cultured in said fermentation broth under conditions predicted to induce the production of a secondary metabolite in said selected chemical family, wherein the prediction is made on the basis of a query of information in a database comprising information regarding secondary metabolite production.

62. The method of claim 59 wherein said step of isolating comprises the step of fractionating said extract.

63. The method of claim 59 wherein said step of isolating comprises measuring one or more properties selected from the group consisting of molecular mass, UV spectrum and a biological activity.

64. The method of claim 59 wherein said step of isolating comprises the step of measuring a biological activity in said extract or a fraction thereof.

65. The method of claim 59, further comprising the step of determining the structure of the secondary metabolite belonging to said selected chemical family of compounds determined to be produced by said candidate microorganism.

66. The method of claim 65, wherein at least one step in the process of determining the structure of said secondary metabolite is guided by information obtained from a database regarding the structural determination of a compound exhibiting said property.

67. The method of claim 46 wherein two or more properties common to compounds in said selected chemical family are selected in step (a), and wherein said secondary metabolite determined in step (d) exhibits said two or more properties.

68. The method of claim 46 wherein said database comprises genomic information linked to or annotated with additional information selected from one or more of: a chemical property, a physical property or a biological activity of a secondary metabolite; and a chemical property, physical property or biological activity of a metabolite biosynthesized by a gene product encoded by a secondary metabolite gene cluster.

69. The method of claim 46 wherein information pertaining to one or more of:
a) the correlation between the secondary metabolite determined in step (d) and the candidate microorganism's secondary metabolite gene cluster;
b) chemical, physical or biological properties of said secondary metabolite determined in step (d); and
c) conditions under which the candidate microorganism produces said secondary metabolite determined in step (d);
is added to said database.

70. A method of identifying a secondary metabolite belonging to a selected chemical family of compounds, the method comprising:
a) selecting a chemical property that is common to compounds in said selected chemical family;
b) identifying in a microorganism known to produce a compound belonging to said selected chemical family, a first gene cluster that participates in generating said chemical property of a secondary metabolite;
c) querying a database comprising genomic information with one or more sequences comprised by said first gene cluster to identify a candidate microorganism comprising a gene cluster comprising a sequence encoding a polypeptide or protein that has a sequence similarity, as evidenced by an E value of 10⁻⁵ or lower, to a polypeptide or protein encoded by said first gene cluster;
d) detecting in an extract of a fermentation broth in which said candidate microorganism has been cultured the presence of a compound that exhibits said chemical property, whereby the presence of a compound that exhibits said chemical property identifies said candidate microorganism as one that produces a secondary metabolite belonging to said selected chemical family of compounds.
e) isolating said secondary metabolite from a fermentation broth in which said candidate microorganism has been cultured; and
f) determining a structure of said secondary metabolite isolated in step (e), wherein a secondary metabolite belonging to said selected chemical family is identified.

71. The method of claim 70 wherein said candidate microorganism and said microorganism known to produce a compound belonging to said selected chemical family are the same organism.

72. The method of claim 70 wherein at least one step in the process of said isolating is guided by information obtained from a database regarding the isolation of a compound exhibiting said property.

73. The method of claim 70, further comprising the step, prior to detecting step (d), of predicting a culture condition under which said candidate microorganism will produce a secondary metabolite in said selected chemical family, wherein said predicting is performed on the basis of a query of information in a database comprising information regarding secondary metabolite production.

74. The method of claim 73 further comprising the step of culturing said candidate microorganism under the culture condition predicted in claim 73.

75. The method of claim 73, further comprising the step of preparing an extract of a fermentation broth in which said candidate microorganism was cultured under said predicted culture condition.

76. The method of claim 74 wherein said extract is prepared from a fermentation broth in which said microorganism was cultured, wherein said microorganism was cultured under a condition predicted to induce the production of a secondary metabolite exhibiting said property.

77. The method of claim 74, comprising performing said detecting step (d) on a plurality of extracts, said plurality of extracts prepared from a plurality of fermentation broths in which said microorganism has been cultured under a plurality of growth conditions.

78. The method of claim 70 wherein said structure of said secondary metabolite correlates with a biological activity of said secondary metabolite, such that the presence of said biological activity indicates the presence of said structural feature.

79. The method of claim 78 wherein said detecting step comprises measurement of said biological activity.

80. The method of claim 70 wherein detecting step (d) comprises fractionating said extract.

81. The method of claim 70 wherein detecting step (d) comprises measuring one or more properties selected from the group consisting of molecular mass, UV spectrum and a biological activity.

82. The method of claim 70 wherein said step (e) of isolating comprises the step of fractionating said extract.

83. The method of claim 70 wherein said step (e) of isolating comprises the step of measuring a biological activity in said extract or a fraction thereof.

84. The method of claim 70, wherein at least one step in the process of determining the structure of said secondary metabolite is guided by information obtained from a database regarding the structural determination of a compound exhibiting said property.

85. The method of claim 70 wherein two or more properties common to compounds in said selected chemical family are selected in step (a), and wherein said secondary metabolite detected in step (d) comprises said two or more properties.

86. The method of claim 70 wherein said database comprises genomic information linked to or annotated with additional information selected from one or more of: a chemical property, a physical property or a biological activity of a secondary metabolite; and a chemical property, physical property or biological activity of a metabolite biosynthesized by a gene product encoded by a secondary metabolite gene cluster.

87. The method of claim 70 wherein information pertaining to one or more of:
a) the correlation between the secondary metabolite determined in step (d) and the candidate microorganism's secondary metabolite gene cluster;
b) chemical, physical or biological properties of said secondary metabolite determined in step (d); and
c) conditions under which the candidate microorganism produces said secondary metabolite determined in step (d);
is added to said database.

88. A computerized system for guiding a determination of a secondary metabolite produced by a target microorganism, the system comprising:
a) genomic information regarding a candidate secondary metabolite gene cluster comprised by said target microorganism;
b) a database comprising genomic information from one or more microorganisms, wherein said genomic information comprises sequence information encoding for biosynthesis of a known secondary metabolite gene cluster, linked to at least:
i) information regarding a chemical property of a known secondary metabolite produced by said known secondary metabolite gene cluster; and
ii) information regarding a condition under which said known secondary metabolite is produced by said one or more microorganisms;
c) a means for assisting the prediction of a chemical property of a candidate secondary metabolite produced by said candidate secondary metabolite gene cluster, said means for assisting comprising at least:
i) a means for comparing sequence information regarding said candidate secondary metabolite gene cluster with sequence information regarding a known secondary metabolite gene cluster in said database; wherein said comparing permits establishing of a correlation between an activity encoded by said known secondary metabolite gene cluster and a predicted chemical property of said candidate secondary metabolite; and
d) a means for assisting the prediction of a condition under which production of said candidate secondary metabolite is induced in a said target microorganism, wherein said means comprises at least a means for identifying in said database a condition under which a known secondary metabolite having said chemical property is produced by said one or more microorganisms.

89. The system of claim 88 wherein said database comprises domain string data and/or family string data for one or more polypeptides encoded by said known secondary metabolite gene cluster.

90. The system of claim 88 wherein said means for assisting the prediction of a chemical property comprises a means for identifying polypeptide domains comprised by a polypeptide encoded by a secondary metabolite gene cluster, and a means for comparing a domain string thus identified in a polypeptide sequence encoded by a known secondary metabolite gene cluster with a set of domains comprised in a polypeptide sequence encoded by said candidate gene cluster.

91. The system of claim 88 wherein said means for comparing sequence information comprises performing a BLAST analysis.

92. The system of claim 88 wherein said means for assisting the prediction of a chemical property further comprises a means for comparing a family string for one or more polypeptides encoded by said known secondary metabolite gene cluster to a family string comprised by said candidate secondary metabolite gene cluster.

93. The system of claim 88 wherein said database comprises information previously determined using said system.

94. The system of claim 88 wherein said chemical property correlates with a biological activity, such that measuring said biological activity indicates the presence of said structural feature.

95. The system of claim 94 wherein said biological activity is selected from the group consisting of anti-bacterial, anti-fungal, and anti-neoplastic activities.

96. The system of claim 88 wherein said chemical property is selected from the group consisting of a chemical structure, a core or backbone structure, a substructure and a chemical moiety.

97. A computerized system for guiding an identification of a microorganism that produces a secondary metabolite belonging to a selected chemical family of compounds, the system comprising:
a) a database comprising genomic information from one or more microorganisms, wherein said genomic information comprises sequence information regarding a known secondary metabolite gene cluster and links said sequence information with the production of a secondary metabolite comprising a chemical property common to secondary metabolites belonging to said selected chemical family of compounds, and wherein said genomic information is linked to at least:
i) information regarding the identity of a microorganism that produces a secondary metabolite comprising said property; and
ii) information regarding a condition under which said known secondary metabolite is produced by said one or more microorganisms;
b) a means for comparing sequence information in said database regarding said known secondary metabolite gene cluster to identify in said database a candidate secondary metabolite gene cluster predicted to encode a secondary metabolite comprising said property, wherein said comparing permits the prediction of a microorganism that produces a candidate secondary metabolite comprising said property;
c) a means for assisting the prediction of a condition under which production of said candidate secondary metabolite is induced in a said target microorganism, wherein said means for assisting the prediction of a condition comprises at least a means for identifying in said database a condition under which a known secondary metabolite having said property is produced by said one or more microorganisms; and
wherein said computerized system can guide the identification of a microorganism that produces a secondary metabolite belonging to said selected chemical family of compounds.

98. A computerized system for guiding an identification of a secondary metabolite belonging to a selected chemical family of compounds, the system comprising:
a) a database comprising genomic information from one or more microorganisms, wherein said genomic information comprises sequence information regarding known or predicted secondary metabolite gene clusters, wherein information regarding a said known secondary metabolite gene cluster is linked to information regarding a known secondary metabolite produced by one or more polypeptides or proteins encoded by said known secondary metabolite gene cluster, said genomic information additionally linked to at least:
i) information regarding chemical properties of said known secondary metabolite,
ii) information regarding the identity of a microorganism that produces said known secondary metabolite; and
iii) information regarding a condition under which said known secondary metabolite is produced by said one or more microorganisms;
b) a means for comparing chemical properties of secondary metabolites represented in said database to identify a microorganism that produces a known secondary metabolite that comprises a chemical property common to secondary metabolites in said selected chemical family of compounds, wherein the identification of said microorganism identifies a gene cluster that directs the synthesis of a known secondary metabolite comprising said property common to secondary metabolites in said selected chemical family;
c) a means for comparing a gene cluster that directs the synthesis of a known secondary metabolite comprising said common property to other gene clusters represented in said database to identify a candidate microorganism comprising a gene cluster predicted to produce a candidate secondary metabolite belonging to said selected chemical family; and
d) a means for assisting the prediction of a condition under which production of said candidate secondary metabolite is induced in said candidate microorganism, wherein said means for assisting the prediction of a condition comprises at least a means for identifying in said database a condition under which a known secondary metabolite having said property is produced by said one or more microorganisms represented in said database; and
wherein said system guides the identification of a secondary metabolite belonging to said selected chemical family of compounds.


SEQUENCE LISTING (31 sequences)

SEQ ID NO: 1 - DNA, 26490 bp, Amycolatopsis orientalis
cggagctcgggccagcaggggtacagaggacacggctggctctaaccggcctgaccacttacgaggtcgaagcggtcagccgccgaccttcgcgatcgccacgaaatgctccggccgcacccgcacgatcaccttgcccggcgaagtcgcgtacgccagccagccgtcgatggtctcctgcccagcctcgccgaggtaccggcgggcgatccgttcggagccgagacgcacgccgtccggatccgaaacgatctccgcgacaccctcgacgctgacgaacgagtacggcgggagcggatcgtcggcgaccaccgagacccgcggattctccttgagcgccttgcctttgacggtgtccgcaccgaggttgaccaggatgtcgtccccgtcgagagcgaaccagaccggcaccgcgtgcggcctgccgtccgggcggaccgtcgcgaagacggcggtgcgggtgccctcggtgaggaaggcgcggcgctggtcgtcggtcatcttctcgggcattactttccctccaggacggcgcccaggccgaaggccgggaacaggtggctgtcgaacgtggtgaactccgcggccttgccgccgacgacgcggacgaggtcgagcttgaaggcgcggaagacggtgtcgccgggacgccgcaggtacgaggcgaaggcgggcatccggttggcgtcgaccgcgacggcacgccattcgcccatctcttcggcgatcctgccgtgccgggcgaggtgttcgaggccgatgtagcagtggggctccggcggcatggtcacgcggacgtcgtcgcggaccaggctgctcaatgtgccgaagtcgccgttgtcgtgggcttcgacgagccgccggaccagtgcgcgttcctcttcggagggagcctggaccgaccattcgccgcgctgctgcgggaggcgttcgcgcagggtcgcgcggccccgctgcagggcgctgttcgcggcggggaccgagagatcgagcagttcggcggtttcggcggcctgccagccgagcacgtcgcgcaggatcagcaccgcgcgttgccggggcggcagcatctgcaagaccgcgaggaaggcgagttcgatggtctccctggcgacggcgagcgcgtgcggttcggcctcgggcggcgcgatctcgtccagcaggcggtccggatacggctggatccaagggacttcggcgaacgagcggaccgacggcacccgccggtccttcttgcggatcgcgtcgaggcaggcgttggtggcgatccggtacagccacgcgcggaagttgccgccgtcgaaggtgtccagcccgcgccacgcgttgagcaacgcctcctggacgacgtcttccgcctcgtcgaacgagccgagcatccggtagcagtggacgtggagttcccggcggtgccgcccggtgagcgcggcgaaggcggtttcgtctcccgccttcgccgcggcgatcgtgtcggtcatggcgcctcccatcgagtgttcttacccgatgtaacggcgcccttcgccggaactcatcggtcccgcgatgactttctcgcgggaccgatggaaccgcaggtcaggggcgggtcgtgaggtatccgcccatggtccggaagtagtcggtggcggccagttccgtgccgtcctcggtccgcacccgctcgatgacgagcccgtgcgaacggccgcggcgcgactcggggccggcgacgatcacgacgccttcaccctctcggatgaagatccggccgggggtgccgccgtactggcccaccgacaccgaggagcggatcacgcgcagctgcttgccgcggtggtaggtgaaggcgttggggtacgggtcggactgggcgcgcaccagccgctcgatctcgtccgcgggccacgaccagtcgatgaggctgtcttcgagggcgcgcttgtggaagaaactggccttcgacctgtcctgtttgaccggggtgaagtcccccttcgcgatcagctcgatggcttcggtggtgatcggcgcgatgaggtcgaccgtccggtggaacagatcggtcgtcgtgtccttcggtccgacggggacggagcgctgcagcacgatgtcgcccgcgtcgaggtcggcgtccatcatgtgggcggtgaggccgacttccttctcgccgttgatgagcgcccagatcagcggcgagaagccggcgtaggcgggcagcagcgagtcgtggacgttcagcgtgccgtaccgcggcatctcgaagatctcgggcggcagccaggtgcgccagttgttggcgacgatcaggtcgagatcggcttccttcagctccgccaggatttccgcgtcgtcgggacgctgccgcagcagcgcgcggatcccgttctcctcggcgaggtccgcgacggagtcggaccagatcttctcgtaggcgtggtcgctcttcgggtgggtgacgacgagggcgacatcgtggcccgcgtcgatcaacgcccgcagggtgcggtgtccccaggtctggtatccgaacatggcgacccgcatgggaccggcctcctcctggtcgagggatgaacatcaccactgaaagtactagttgagcctcacctaacttaggttagggttccctggtctttgttgcgaagtaccgagatgcggctgagccgcgcaaggggttcaagatggcacgagcagtggtcggtgaacgggttccggtctacgacgtggtcggggtcgggttcggtccctcgaacctcgcgctcgccatcgccgtcaccgagcacaacgccgcgcccggggtcgagaccgtcacggcccacttcctcgagcgtcaggctcgtttcggctggcaccgcgggatgttgatcgacaacgcgaccatgcaggtctccttcctcaaggacctggcgacgatgcggaacccgaccagttcgttcagcttcctgtcgtatttgcacagcaaggaccgattggtcgatttcatcaaccacaagaacctgttcccgttgcggatcgagttccacgactacttcgaatgggcggcagaaaaggtcgacgacctggtttcctatggcacggaagtgctttccgtcacgccggtgttcgacggcgacgagatcgtgttcttcgacgtgcacgcccgcaccgacggcgagctggtgaacctgcgcgcccgcaacctggtgatgggcaccggtctgcgcccgaatctccccgaaggtgtgacgccgggcacacgcatctggcacaacagcgaactcctgcaccggctcgagggcatggccgccgaggaaccgcgccggttcgtcgtcgtcggcgccggccagagcgcggcggaggtcagcgcgctgctgcacgaccgcttcccccgcgccgaggtgtgcgcggtgttcgcgcggtacggctacagcccggccgacgacagcgcgttcgccaaccggatcttcgaccccgaggcggtcggccggttctacgaggcgccggaagcggtcaaggaccgcctcatgcgctaccacggcgcgaccaactactcggccgtggacatcgagctcatcgacgagctgtaccggcgcgtctaccgcgagaaggtgcaggggatcgagcggctgaagctgatcaacgtctcccggcccaccgaggtcaccgacaccggttccgaagtgcgggtcaccgtcgaggcgctggagaccggggcgcggaccaggctcgacgccgatttcgtcgtgtacgccaccgggtacagcccggcggacccgacgccgttgctcggcgagctcgcctccacctgcgcgcgcgacgacgagggcaggctccgcgtcgagcgcgactaccggatcgtcaccgagccgccgctcgacggcgggatctacctgcagggcggcaccgagcacacgcacggcatcacgtcttcgctgctgtccaacaccgcggtccgggtcggcgagatcctccagtccattgtggacaggcgtgcggccggcgcgtcccggcccgcgtacgcggtgagcgggtcccggcccgcctgaggccaggacgatcccaagcgtttccgaacggtggaggcaaacccgatgaaccgcggcattcccctgaccccggcacagcaggggatctggctcgcgcagcagctcgacccggccgatccggtctacacgatcggctgggtcgtggagcttcgcggcgccgtcgacctcgcccggctgggtgacgccgtccggcgggcggtcgcggaggcggaatgcctgcacgtgacggtcgggctggacggtgacacgccggtccagcgtccggccgctcccggtgaggtcgccgtgctcgacttcagtggtgaggcggatccggagcgcgccgccgacgagtgggcgcgggccgaactcgccgtcgtggccgaccccgccagggggccgctcaccgcgcacacgctgctgatcctggccggggaccgggtccgctggttccagcgttaccaccatctcgtgatggacgcgcacgggcaggccgtcctgacccgccgcgcggcggcgctctattcgggtaccgccgaggaggtctcttggccgctggcggggttggccgagtccggctacgacgggacggccgaccgggagtactggctgggtgaactcgcgggcaggccgaacccggtgcggctgctcggccgcgcgtcggcgggccgggtccggctccggcggcattcgtgggaactgcccgcggccgaggcaggccgcctgcggaagttcgcgctggacgcggggacgaggttgtcccgggtcgccatcgccgccgtggcggcgtacgcccaccgcgtgacaggcgccgaagacctcgtcctaggcatccccgttaccgcgcgcaccacccgcgccctgcgtgaacagccgggcatggtctccaacgtcctgccgctgcggctgaccgtgcggccggagacgacgccgtcgcgactcgtcgcccaggtcgcggagaaggtgtccgcgctgctggaacacagccggtaccgcggggaggacctggcccgcgaactcggcgcgagcggcggggtgcacgaactcgtcgggctgagcgtgaactacatggccgtggacggcgaactcgcgttcggtgacgcgagcgcgacggtgcacaacctcgaactcgggccgatcagcgacgtcgccatcgccgtgtacgacgccggcgagggccggggtctccgcttcgacttcgacgccgacgaggccgtctcgtccgacgacgaactcgccgaccatcgccgccggttcgccgccgtactggacgccctgctgaatggcccggacctgccgctggccgccatcgacctgctctcggcggaggagcggtcccgggtcttggagctcgggaccgcggtcgcggagtcgcccgaggtgtcctggcccgaggcgttcgcccgggtggtggcgcggaagccggacgccgaggccgtcgtctgcgaggacgtccggatgacctacgccgaggtcgacgccgccgccaaccggctcgcgcggctgctgcgccggtgcggggtcggtcacgaggacgtcgtcggcgtggccatgccgcgttcggccgatctcgtggtcgccctgctcggcgtgatgaaggccggggccgcgtatctcccgctcgacctggaccaccccgaggaccgcctcgcgtacatgctcgcgcacgcgggcgcggggatcgtggtgtccactgtggaggaatcggcccagctcccgtccggggtggacgtcgagcggatcctgctcgacgacccggcgatcaccgccgagcttgcggcgctcgattcgtccactgtggacgtgagtgggatcgcgctgggccaggccgcgtacgtcatctacacctctggctcgacggggaagccgaagggcgtggtcctgtcccacgacgggatcggcagcctgatcagcacggcgacagagcggatcggcatcggcgaaaacagccgggtcgtgcagttcgcctccaccgggttcgacgtgacggtctgggatctggtcatgtcgctgtgcgtcggcgggacgctgatcgtcgtgccgtcgcaccgccgcgtcgcgggtgtggagctgaccgggtacatcgccgccaacgcggcgacgcatatgatcctgccgccgtcgctggtcgcggccctcccggcggactgcgagctgccgaagggcgcggtgctgatcgtcggcaccgagaccgtcccgccggaactgatcgcgcgctgggccgaagacctgaaggtcgtcgccgcgtacgggctgaccgaggcgacggtgaactcgacgctgtgggcggccgagcccggctggagcgggccgatcccgatcggtgtcccggatccgaacacccgctgctatgtcctcgacaccgccctgcgaccggttccggtcggtgtcgaaggggagctgtacgtcggcggacgcggtctcgcccggggctacgcgggacgtccggggctgtcggcggagcgtttcgtcgccgacccgttcgccggtggcggcgagaggatgtaccgcaccggcgaccgcgtccgctggcgcgcggacgggaacctcgacttcctcggccgcaccgatcaccaggtcaagatccgcggattccggatcgaacccggtgaagtcgaatcggcactcgccgcgtacgccggggtcgagcggatcgccgtcgtgccccgcgaggtcaaaccgggggaccggcgcctggtggcgtacgtcgtgccgcaggcggacggatcgccgtcactgctgaaagacctgcgcgcccactcggaatcggcgttgccgcactacatggtgccgtcggccttcgtggaactcgaccggctgccgatcatgccgaacggcaaactcgaccggaccgcgttgccgctgccggacttcggcgccgccgcgaccggccgcgcgccggccaccgaacgggaacggatcctctgcgacgtcgccgcacgagtcctcggcctggccacgatcggcgcggacgatgacttcttctcgctcggcggcgacagcatcctgtccatccggctggtgctgggcgcggcggaacacggtctcgcgatcaccccacgccaggttttccagcacaggactcccgaagcgctggccgcctgcgctgaagcggtgcgcgccgagtccactgtggacgaccgaccgctactggagctgaccgacgccgaacgtgcgtcgctcgccgagtacgccgatatcctgcctgtgacaccgttgcaggaagggttcttcttccacgcggaattcgaaggcggtgcggccgccgacatctacacggtccaggaggtgctggacctcgaaggcgatgtcgacgccgacgtgttgcgccggtcgacgcaggcgctgctggaccggtattcgtcgctgcggtccggtttccgccagctcgacggcggccgggtcgtccagacggtggcacggcacgccgaactgccttggcgcgaggtggccgccgaagacgccgaggcggccttcgaggccgatcgtgcgcgccggttcgacttcgcccggcctccgctgctgcgggcgaccttcacccggctcggcgtggatcgcgcgaagctggcgctcacgttccatcacatcatcgccgacggctggtcggtcgtggtgctgctgcgcgaactgctcgcggcgtacgggggagcacccgtactgccggaagcgggcacccgcgccgatcacctgcggcggctcgcgtcacgtgatcacgaggtctcacgcgaggcttggcggacggcgttgtccgaagtggacgaacccacccggctgatcgacgaacgagccggggtggcgagacggccgtcgcggctgcacctgagcctcggcgagcgggcgacggcggcgttgtccgcgatggcccgcgagcacgggctcaccctcggtactgtcctgcacggtgcctggggactcctgctcggcggcctcaccggacgggacgacgtcctgttcgggagcacggtctcgggccgtgacaccggcgccaccggtctcgaatcggcggtcggcctctacatcaacacggtgccggtgcggctgcgctggtcgggggccgagaccgtcgccgccgtgctgcgacggctgcaggacgagcaggcggccctgctggaccaccagcatctcggcctcggcgaactgcagcgcctcgcgggcgcggggcaggaagaactgttcgacacccttgtcgtcgtcgagaacttcccgcgcgacgacgagcgcgcctccggaaccctccgcctcgccggcgtcgaagtcaccgacgccgtgcacttcccggtcgcggtgatcgcgacaccgggggacgatctggcgttcagcctcaagttcgacgcggcgaggatcgacgcggtcgcggccgggcagctcgcggaacggttcgcgcggctgctggagacgctgagcgcgaaccccggtctgccggtcgccgcgctcgacctcctgtcgcccgccgaacgcggccgcctcgccgaactcaacgcgacctcgcaccccgtgccggagcgtacgctggccgaggccttcgccgagcaggtcgccaagaccccgtccgcgacggcggtgatgttcaaggacaccgaactgtcctatgcggacctcgacgcccgcgcggagcaggtggcacggaggctgcgttcgctgggagccggtcccgaagagatcgtggccgtcgccgtgccgaggtcggccgagctgatggtcgcattgctgggtgtcctcaaggccggggccgcctacctgccgatcgacctcgactatccggccgaccggctcgaatacatgctggccgattccggtgcgcgtctcgtggtctccgaaccggggacggccgcgcggatcccggatgtcgccgggctgactcgggtccccgtgaccggcgagcccgtcgacgatgtcgagggtgcggcagccggaccggacaacaccgcgtacgtcatctacacgtcgggttcgaccgggcggccgaagggcgtggccgtcacgcatcgcgcgatcgtcaaccggctcgcgtggatgcagcacgagtaccggctcacggcctcggaccgcgtgctgcagaagacgccgtcgagtttcgacgtgtccgtgtgggagttcttctgggcgctgtgcgaaggcgccacggtggtgctggccgaaccggacggccaccgcgatcccgcctatctagcgcgagtgatccgggaccggcggatcaccaccctgcacttcgtcccgtcgatgctcgcggccttcctcggcagcgacgaggccaccggcgatctcggatcgctgcgtcaggtgttctccagcggtgaggcactgcccggcgagaccgccgcacggtggcgctcgctcaccggtgtcccgctgcacaacctctacggccccaccgaggcggccgtggacgtgaccttcttcgccagcaccggtgccgacggcgccaccgtccccatcggacgtccggtgtggaacacgcggctgcacgtcctcgacggctgccttcgcccggtgcccgacggcgtggcgggcgagctgtacctcgccggtgtccagctggcccgcggctatcacggacggccggggctgaccgccgaacgtttcgtcgccgacccgttcggcgaaccggggcagcggttgtaccggaccggcgacctggtgcgccgccgcgccgacggcgagatcgagtacctcggccgcaccgatcggcagatgaagatccgcggcaaccggatcgagctcggcgagatcgaagcggttctcgtcgcacagccggaggtcacggccgcgacggtcgtggcgaaggacggcgcgctcgtcggctacgtcgccgggagcgcggatccggagaatctgcgaaccgccctcagtgaggcactgcccgcaccgatggtcccgcaggcgttcgtcgtcctcgacgaattccccttgacccccagcggaaagctcgacgtccgggccctgcccgcgcccgaggccggccgggccgaagccgcacccgggaacgagcaggagcgcgcgctcgcgcggatcttcgccgacgtcctcggcctggagaccgtcggcgtcgacggggacttcttcctgctgggcggcgacagcatctcgtcgatcgccgtgtcgagccgtgcccggcgggccggattcgagctgagccccaaggacgtgttcgaattccggaccccggcggcgctcgccgcgtccgccgcgccggcggagccgatggccgacgctgacggcatcggtgccgtccccctgctgccggaaatgctccggctgcgtgagctgggcgacgcggtccagccggagtccgtactgctgaccgtgccctccggcgccgacctcgagacgctcgccgaggccctgcagctcgtgctcgaccaccacgacggcctgcggctcacgctgtcgcgggtcgcctcggtgctctggtcgctcgaagtccgtccacaagggaccgtccaggccgcggacctgctccgtcacgcatccggagccctcgaagacgagtaccgggacgccgagggcaggctgaccgacaacctcctgcaggccgtctggttcgacgaaccgggcaggctgctgctgatcgcgcatccggtcctgctggacgcgcggagcctgacgacgctgtccgccgatctcgcgatcgcctggcaggccgtgaccgacggccgcccgccggcgttcacgcgagcgcggacgtcgctgcgggccatcgcccgccggatcaccggcgcggcacaggatcccgcgctgctcggcgaggtcgagcactgggtcgggacgctggcacccgtttccttcgaggtccgcggcgatgtcagcgaacgccggatcacgctgtccgtcgaggacacccggacgctcgtcggcgcgctgccgtccgccgtgcacggcgacgtgaccgacgtcctcctgaccgcgttgaagatcgccgtcgacggtgacctgctcgtcgatctgcacggccgggtcgacccgtcggccacgatcggcgcgctgacctcggtccgcccggtgcggatcaccgcgtccggcgagccgctcgcgatgctcaaggccatcaaggaaaccgtccggaagacccccgagggctacggcccgctgcggtacctcgacgcccagaccggaccgctgctcgccgggctagcgaagccgcggatcgccctgcgatacgacggcctcgtcccggccggaccgggggagtggcaggtcgccggatacgcggccacgccgccgtccggcgaccaggtcgtgcgtatcggcgtcgcctgcgaggagaccgaggaaggcccgaggctgaccgcgacgttcgtgggtccgggcggagacctggccgaacgctgggccgccgccctggtcgaactcgcggccgtggacaccgacgccccggtggggctgacgccgtccgacctcgaactggtgtcgctgacgcaggaggagatcgaccgcgtcgagcggtccagcccggtgccggtcgccgacgtctggccgttgtcgccgctgcaggaaggcctcttcttccactccagctacaactccgatcgggtggacatctacacgatccaggaggcgatcgacttcgatcaccgcctggacgccggcaggctccgggcggcggtgtccgcgctgctgcggcggatcccgagcctcggcgccggtttcaccagcgaagggctgcgcggtccggtccagttcgtggcggccgaggtcgcgccgccggtgtccgaagtggacctttcggcgctgcccgaagacgagcaacaggcggagctcgacgagctgatggccaaggatcgcgcgacgcggttcgacctcggtgccccgccgctgttccgcctgaaactggtgcggctcgggaacggccgcgaccgggtggtgctgaaccggcacctgctgctctgggacggctggtcggcctggctgttcatcgagcaactgttcgcactgtacgacctgggcggcgacgaccggacacttcccgcggccgggtcctatgtggacttcctgcgctggctggaccggcaggacatcgaggccgcgacgaacgcgtggcgcgaggcgctgtccggcctcgcggagccgaccgtcgtcgggcccgaaggccgcgacctggctccggcgaccccggtcaacctcgacaccgtgctgccctccgggctcagccgggccttgcgcgagcaggcccgcaggcacggcctcacggtgaactccgtgctcaacgccgcctggggcctggtcctgtcgaccatgaccgggcgccacgacgtcgtgttcggtgccgcggtcgcgggcaggcccgccgccgtgcccgagatcgagaacaccatcggcctgttcctcaacaccgtgcccacccgggtacggctcgacgcggccgaatccgtgctggacctggtgcggcgcctgcagtccgagcggatggacctgacgccgtacgagttcatgagcctcggggtgctgcaacgcgaggccgggcaccgtgtcctcttcgacacgctgttcgtgctgcgcaacgccgatggcgacgaacggcgcgacggcctccgtcaccggcacggcatcaccgacctgctgaacatcgacgccacgcattacccgctgacgctcgtggtcacgccgggcgaggaaacgcgggtcacgctgtcttaccgggacgacgtcatcgactccgccgaagcggcgtcggtgctcgaccggttcacgtcgctggtggcgcagatcgtcgacgacccggcgaaaccggtcgccgcgctggccacggtgtccgccgaggaccgggccgcgctggaagcgggctgggccgaagccgagcaccccgtcatcgaggacacgatcgcggatctgctcgccgtgcaggcggagcgcactcccggcgagacggcgctggtgttcggcgaagaacagctgacctacgccgaactcgacgcgcgcatcaaccggatggcccggctgctgctggccaggggagccgctccggaacaggtgatcgcgctcggcctgccgcgttcgatcgacatggtcgtcgcgctgttcgccgtgctgcggacgggtgcggcgtacctgccgctggaactcgactacccggtcgagcggctcgcggtgatgctggaagacgcccggccgctctgcctggtgtccacaaaggacgtttcggcgacgctgcccggtgacacgccccgggtgctcgtggacgaccacgaagacagggcgttcggcgacggcccgatctccgacgccgagcgcccgctgttcgcgcggagccgcgcggaccgcatggaacacccggcgtacgtcatctacacctcgggctcgaccgggaagccgaagggcgtcgtcacgccgtaccggggcctgacgaacatgcagctcaaccaccagggcgcgatcttcgccccggccatcgcgtcggcgggcggacggcggctgcggatcgcgcacaccgtgtcgttcgccttcgacatgtcctgggaagaactgctgtggctcgtggaggggcacgaggtccacgtctgcgacgaggaactgcgccgcgacgcccgggcgctcgtggcgtactgcgacgagcacgaggtcgacgtcgtcaacgtgacgcccacgtacgcccatctgctgatcgaggaagggctgctggaggggcaccggcccgcactcgtcctgctcggcggcgaggcggtgtcggagtcggtgtggaaccggctgcgcgacaccgacggcacctacggctacaacctttacggccccaccgaatacacgatcaacacgctcggcggcggcacgaccgacagcgacacccccaccgtgggcaagccgatctggaacacccgcgcgtacatcgtcgacgcctggctccggcccgtccgcgacggtgtcgcgggcgagctctacatcgcgggcgaaggcctggcccgcggttacctcggccgtcccgggctgacggccgaacggttcgtcgccgatcccttcgtgcccggtggcgggcgcatgtaccgcaccggtgacctggtgcgccgccgtcccgacgggaacctcgacttcctcggccgcaccgacgaccaggtgaagatccgcggctaccgggtggaactcggcgagatcgagaccgcgctcagccgctaccccgaggtcgcgcaggccgccgtcatcgcgcgcccggatccgtcggcgcccggtctgcagcggctgatcggttacgtcgtcccggcagagctgagcggggacgcgcgcgccgaagcggaagccgaccaggtcggcgaatggcagcagatctactcggacgagtacaccgagatcccgacggcgttgttcaccgaggacttcgcgggctgggactccagctacgacggcgacccgatcccgctggagcacatgcgggaatggcggcgcgcgaccgtcgaccggatcgccgagctgaaaccggaacgggtgctggagatcggcgtcggcaccggtctgctcatggggcagatcgcgcccctggtccaggagtactggggcaccgacctcgccgcgccggtgatcgcgaagctgaaccgcgagctggaacaggatccggcgctggcggccaaggtgaacctgcgggcccagccggcgcacgtgttcgacggcctgcccgccggccggttcgacacgatcgtgatcaactccgtcatccagtacttcccgagcgtcgactacctgaccaacgtcatcacgcgggcgatcggcctgctcgcccccggcggcgcgctgttcgtcggcgacgtccggaacctgcggctcgcccgcgccttccacaccgcgatccagctcacccgcgccgacgccacctccgacgtcgcccaggtgcgccgcgcgatcgaacgcggtgccgcgctggagaaggaactgctgatcgacccggactacttcaccgcgctcgcgcggcggctcccggacgtcgcggcgcaggtccggatcaaacgcgcccggctgcacaacgagctgagccggtaccgctacgacgcggtcctggtgaaagagcccgccgagaccttctcggtcgcgcaagcaccgcggttgtcctgggacgaagtgggctcgctcgccgctttggaggaccgcctcaccggtgcggagacgctgcgggtgagccggatcccggacgcccgccagacgcccgaggtcgaggcgacgcgggcgctggacgccggtgcgccgctcatcgacgtcctggacctcttccgtgctccggccggggtcgagcccgaggacgtgcacgaactcggcgaccggctcgggttccgggtgtactgcacctggtcggccgacggcgcgttcgaggccgtgttcgtccacaagggacagacggatgcgctggtcaccggggtgcatcagccggtctcggtgggtacgagcctggccaagtacgcgacgagcccgacggcggcacgtggtggcggcgaactcgtgcagcgcctgcgggaccggctcaagacggaactgccggactacatggtcccggccgcgttcgtcacgctgggcgccctgcccttgaccgacaacggcaagctcaacacccgcgcgctcccggacgccgagcagacggtgcggctcgccgaaagccgtccggccgagaccgccgaggaagagacgctgtgcgcgctcttcggcgaggtcctcggcgtggagggggtcggcgtcgaggacaacttcttcgacctcggtggccattccctgctcgccacccggctcatcagccgggcccgtaccgaactcggcgccgaactggccatccgtgacctgttcgaggcgccgacggtcgcggagctggccgctcgcgcgggcggcggcgaaccggcgcgggccgcggtggtccgcgccgagcgccccgaccggatcccgctttcggccgcccagcgcagtctttggctcgtggaccggatggaagccaacgcggtcgcctacaacttcccgctgaccttccgtctgcgcggagccctggatctcgacgcgctgcgggccgcgctcggcgacgtgctcggcaggcacgaagtcctgcgctctgtcttcgacgagcacgacggcgagccctaccagcgtgttctcgacgcgccggagatcccgttcgctgtcgaggactgcgacgaggccgagctggatgcccggatcgccgagctctcgcggacacccttcgacctggcgcgggagatccccgtccggctgcgggtgctgcggctcggtgaaggcgaccaggtgatctcgctggttctgcaccacagcgcgaccgacgaatggtccgaccggccgttcctgggcgacctcacgtcggcctatcaggcgcggctcgccggtgacagtcccgtgtggacggagctcccggtgcagtacgccgacttcgctctgtggcagaaggacttcctcgctcagcacggtgccgcgcaggtcgcctactgggccgaatcgctgcgcggggcaccggacgagctgaccctgccgctggatcggccgcgtccgctccagccgacgggcaagggcggcaaggtgaaatccccgctctccgaggaactgtcggccgcggtgcgtgacctggcggcgaaggcgggagcgagcccgttcatggtgctccaggcggcggtggccgcgctgttgcaccggaccggcgccggggaggacatcccgctgggcgcgccgatcgccgggcggaccgacgcggcgctcgacgacctcgtcgggttcttcgtgaacacgctggtgctgcgcaccgacctctcgggtgagccgacgttcgccgccctgctcgaaagggtgcgcgaggccgacttggccgcgttctcgcacggcgacctgccgttcgaaagtgtcgtcgagcggctgaacccgccccgggtgccggggcggaacccgctgttccaggtgatggtcggctaccaccggcagagcgacgcgcacgacgtgctcggactgcccgccgagtggttcgagatggacaccggcatggcgaagttcgatctgcacttcacgatggtcgacgacggcaccaccgccacattgatgctggagtacgccgaagacctcgccggcgaggcgacggccgcgcgtttgctggaccggctcacgtccgtgctggagcaggtcaccgccgagcccgaacggccggtcgccacgctcgacgtcctcgtcggtgacgaactggcgcgggtcaacgagtggaaccgcacggcccgggaagtcccggccgcgaccctgccggagttgttcgaggcgcaggtggcgcggacgccggacgcccccgctgtcgtgttcgagggcatcgagctgtcgtatgcggaattcgacacgcgggcgaatcggctcgcgtggtggctggccgagcagggtgtgggtgcggagtcggtggtggcggtgtcgctgccgcggtcgatcgacctggtcgtcgcgctgtacgccgtgcacaaggccggtggtgcttacctgccggtcgaccgcgactacccggccgatcgcatcgcgttcatgctcgaagacgccgcccccgcggtcgtgctcgaagaactgcccgttctcgacgggtactcctccgagagcctggggcggatcgtcgacccgtcgtctccggcgtatgtgatctacacgtcgggctcgacggggcgtccgaagggtgtcgtggtgccgcatgcgggcatcgtgaaccgcctgttgtggatgcaggacgagtacggtctgacggctgatgatcgggtgttgcagaagacgccgtcgagtttcgatgtgtcggtgtgggagttcttctggccgttgatcaccggagcgacgctggtggtcgccaaaccggaggggcacaaggacccggcgtatctcgcggagctgatccgcaccgccgggatcaccaccgtccacttcgtaccgtcgatgctcgcgctcttcctcgagaccgccgaatgcgccgggctgcggcgcgtgatctgcagtggcgaggcgctgccgtcggaactggcggcccgcgtggacgtcccgctgtacaacctctacggcccgaccgaagcgtccgtcgacgtgaccgcttggcgggccggtgccgagacgacgccgtccgtcccgatcgggcgtccggtgtggaacacgcggacgtacgtgctggacgcgcggctgaacccggtcccgccgggtgtccccggcgagctgtacctcgccggcgtgcagctcgctcgcgggtatctggcccggcccgggctgaccgccgaacggttcgtcgccgacccgttcggcggcccgggggagcgcatgtaccgcaccggggacctcgtgcgctggtcggacgacggcgtgctggagttcctgggccgcgtggacgatcaggtgaagatccgcggcttccgggtcgagctcggtgaggtcgaggcggcgctgaccgcgtccgggtccgtcacgcgcgcggtggtcgtcgcgcgtgaagaccggctggtggcgtacgtcgtcggctcggtcgacggactgcgggaggcggtggccgcgaagctgccggaccacatggtgccctcggcgttcgtcgtgctcgacgagatcccgctgaccccgaacggcaaactcgaccgcaaagccctgcccgcacccgagttcgcgaccatcgagggacgcgagccgaggaacgcgcgggaagaggccctgtgcgcgttgttcgccgacgtgctcggcgtcgaacgggtcgggatcgacgacggtttcttcgccctcggtggccattcgctcctggtgatgcgcctggtcagccggatctcggcgagtctgggcgccacggtgtcggtccgtgacgtcttcgacgcgccgacggtcgcggcactggccgaacgcctcgcggaaccgtcggcccagccggcactgagcgcggggaaccggccggagctgctgtcgctttcgcccgcccagcaacggttgtggttcctgtaccggctcgaaggggtcacgcccacctacaacatcccgctcgcgtggcggctgcacggcgagctggacgcggaggcgctgagcgcggcgatcggcgacctcgtcgagcggcacgagccgctccgcacggtgttcgtcgaggaaggcggcgtcgcggcacagcgcatcctgaccgacgctcgcccggaggtccgtttcgaggaagcgggcgacctttcggcgcagttggccgcggaggccggtcgcggattcgccctcgacgaggaactcccgatccgggccacggttttcgccgtgtcgccgcgggaacatgtcctgctgctggtgatccaccacgtggcgacggacgagtggtcgcagggaccgctcatggccgacctcgccaccgcctacgaggcgcgccttgctggaaccgctccacagtggacaaagctggcggtccagtacgcggattacgccctgtggcaacgcgaactgctcgattcgacggccgccgggcacctccggttctggcaggaagcgctggcggacctgccggacgaactgccgctgcccgcggaccggcggcgtccggccgaatcgagccatcgcggcggtctcgtcaccttcccgatcgacgccgcgctcaccgcacggctgcgtgcggtggcccagcggcacgacgtcagcatgttcatgctggcccaggccgcggtcgcggcactgctgcaccggctcggcgcgggggatgacatcccgctcggcgcaccgagttccggccgcctggacgagcggctggagccgctgatcggcttcttcgtgaactcgctcgtgctgcgcaccgacctgtccggcgagccggcgttcggcgaactgctctcccgggtacgcgcgaccgacctcgccgccttcgaacaccaggagctgccgttcgagcggctggtcgacgcggtcaacccggaacgttcgctggccaggcacccgctgttccaggtgatggtggtgtacctgcccgaggccgaggaccgactgacactgcccggacttcgtgcggagcaagaggacttcgcgcagggcgtcgcgaagttcgacctcgagttcggcttcctggagcacggcgacgggatctccggcgcgatcgagtacagcgccgacctgttcgaccacgccaccgccgagtcgttcgcgcggcgcctggtgaccgttctggagcaggtggccgccgaccccgcgatcccggtcggtgagctggacgtcttcggcgaggacgagcgcgcgctgctcaggcactggaacggcaccgcgcacgacgtcccggcgagtacgttgccggagttgttcgaggctcaggtggcgcggacgccggatgctcctgccgtggtgttcgagggcgtcgagctgtcgtatgcggagttcgacgcgcgggcgaatcggctcgcgtggtggctggtcgagcagggtgtgggtgcggagtcggtggtggcggtgtcgctgccgcggtcgatcgacctggtcgtcgcgctgtacgcggtgcacaaggcgggcgcggcgtacctgccgctcgacacggactatccggccgatcgcatcgcgttcatgctcgaagatgccgcccccacggtcgttctggacgagctgccggtcctggacggttattcgggagagagcctgcggcgtgccgtggaccccgcgtccccggcgtacgtgatctacacgtccggatcaaccggccgcccgaagggtgtggtggtgccgcatgagggcatcgtgaaccggttgctgtggatgcaggacgagtacggtctgacggctgatgatcgggtgttgcagaagacgccgtcgagtttcgatgtgtcggtgtgggagttcttctggccgctgatcaccggcgccacactggtggtcgccaaaccggacgggcaccgggatccgcggtatctcgcggagctgatccgcgaccggcggatcacgacgctgcacttcgtcccgtcgatgctggaggcgttcctcgccgaagagccggagtggcatggcgtccgccgcgtcctgtgcagtggcgaggcgctgccgccggaactggccgctcgcgtgaccgtgccgctgcacaacctctacgggccgaccgaggcctcggtggacgtgacctcgtgggagatcgtcgatgacaccgtccccatcggacgtccggtgtggaacacccaggtccacgtgctcgactcccggctgcggccggttccgccgggtgtggcgggcgagctgtacctggcgggcaagcagctcgcgcgcggctatctcggccgggccgggctcaccgcggaacggttcgtggcgaacccgttcgccgcgggggagcggatgtaccgcacgggtgacctcgcgcggtggcgggccgacggcgcgctcgactacctcggccgcgccgacgaccaggtcaaactgcgtggcttccggatcgagctgggcgagatcgagtccgtgctttccgcgcgggtggtcctgcgggagggcaggctcgtcgcgtacctgaccggggaggccgacgtcgagcaggcccggcggctcgccgaggccaccctgccggagtacatggtgccgtccgcgttcgtggtactggacgcgttcccgttgacacccaacgggaagctcgaccgcaaggcgctgcccgcgccggacttctccggcgaggtcaccgacgacgccccgcgcacacctcgcgaggaactgctcgcgtcgctcgtcgcgggggtgctcgggttggagaaggtcggtgtccacgacgacttcttccgcctcggcggcgacagcatcgtcgcgatgcagctggtcggccgcgtgcggaatgccgggctgctgctgagcccgcgtgacgtcttccggcaccggaccgtcgcggccctggccgaggtcagcgcggtgcggctcgacgcatccgcgcccgagagcgggaagccgttggtggagctcgacgcgagcgagcgcgcggagatcctcgacgtcgcgccggacacggccgaggtgtggccgctgacgccgttgcaggccgggttgctcttccacgcgacgatggacggcgacggaccggacgtctacaccgtccaggtgtccttcgatctcgaagggccgctcgaccgggctcggttgcgcgacgccgctcaggcgctcgtcgaccggaacgccgtcctgcggaccggctaccgctacctcgcgtccgggcgcccggtcgcgctggtggcgcgttcggtcacggttccttggcgcttcgcggatgcgccggacgccgtcgaggcggagatggcccacgacaggcgccgcttcgatccggcggccgcgccggtactgcgtttcctgctgctcgccctgccggacggagggcaccggctggtgttcacccaccagcacgtactgctcgacggctggtcggtgccgcagttgttcgccgagctgtcccagctctacgccggagccgacgtggcaccggcccgccagtaccgcgaccatctcggctggctcggaggccaggaccgggacgcggcggaggccgcctggcggcaggcgctcgacggcctgaccgagccgacacatctcgtcccgcacgacccgcagcggatccctgccctaccggagcggctgtcactcgagttgccggaacaggcgaccgaggcgctcaccgcactggcccgctcacgcggcctgaccctgaacacgctggtccagacggcgtggagcatcgtgctcggcaggctcaccggccgggcggacgtcgtcttcggcgcgacggtcgccggacgcccgcccgaactggccggggtcgagcggatgatcggcctgttcatcaacaccatcccggtgcggatcgtgctcgacccggccgagaagctgggcgacctgctcgaccgggtccaggacgagcagtccggattgctcgcgcatcagcacttcgggctcgccgacatccagcgcggtgccgggctgggggagctgttcgacaccctcatggtgttcgagagctacccgggcgcggacgaggacgagacgggcgccgggttgcgcggggagatcgtgcgccacgacgactccacgcactatccgctcacctgggcggtggagccgggagcacggctgaagctcaccgcggagtaccgcgccgacctgttcgacgccggcctgccggaacggcttctgcccgcgatggagcgggtgctcgcggcgatggtgtccgatgtggacgctcccgtcggccgggccgacatcctgggcgaggccgacctggacctgatcgtgcgcgagtggaacgggacggccctcgtcgtcgaaccgtccaccgtggccggtctgttcgaagctcaggtccggcgttcgcccgacgcgacggcggtggtgtccggtgtggtcaccctgaccttcaccgaactcaacgaacgcgccaaccggctggccagggcgctggtcgcccacggagtggggcctgaggacttcgtcgccttggcgctgccgcgttcggccgacgtgctgctggcgatcctcgcggtccacaaggccggggcggcatacctgccgctcgacccggactacccggccgaacggctggccgcgatgctcgccgacgcgcggcccaaggtgctcctgaccgagcccgagctgctggaggcgttgccccggaccgggatcgagacgctggcgctctgcgcgctcgtcaccgaagaccggcaggcacacgacctgaccgaccgggaccgcgtccgcccgctcgtcccggagcatccggcgtacgtgatctacacgtcgggctcgaccggcacgcccaagggtgtcgtcgtcccgcatcgcggcgtggtgaacctgttccacagccaccgcgccaccttgcaccggcccgcggtcgaggccaccggcaaaccgcatctgcgggtcgggcacgcgtggtcgttctccttcgacgcgtcctggcagccgcaactgtggctgctcgacgggcacgccgtgcacatcgtggacgaggagacgcggcgcgaccccgaactgctggccgcgatgatcgaacgggagggcttcgacttcatcgaggtcacgccgtcgttcttcgcgcagatggcggacgcgggcctgctcgacggcgaccggtgccccctcgcggtgatcggtgtcggcggcgaggcggtgcccgagagcctgtggcggcgtctggccactttggacggcaccgaggccttcaacctgtacggacctaccgagtccacagtggacgcattggtcgcgcggatcgccgacagcgagaagccgctggtgggccgtccggtcgcgaacacccacgcctacgtgctggacgcggcgctgcggccggtgccgcccgggatcaccggtgagctgtacctcgccggtgccgggctcgcccgtggttatctcgggcgggcggcgctgacggcggagcgattcgtcgcggatccgttcggccccgcgggtgcccggatgtaccgtaccggtgacctcgcccgctggaccgcggacgggcgtctggacttcggcgggcgtgccgacgaccaggcgaagatccgcgggttccgggtcgagcccggtgagatcgaggcggtgctcgaacggcacgacggcgtcgcgcaggctgtcgtcctggtccgtgaggatcgccctcgcgtcaagcaactcgtggcgtacgtcgtgtccactgtggagcgggacgagctgcggcggttcgcggccgagacgttgccggactacatggtcccggcggcgttcgtcacgctcgacgcgcttcccacgctgtcgaacgggaagctggaccgcaaggcgttgccggcgccggactactcggagctggccggtggccgcgcgcccgagacagcgctggaaaagacgttgtgcgcgcttttcgccgaggtgctcgatgtgccggagctgggtgccgatgacgacttcttcgccctcggcggggacagcatcgtcgcgatgcggctggtcggcagggcgcgcgcggccggactgaagatcacgccgcacgacgtgttcgcgggccggacggtcgcggcgctggcggaggtcgcggaacggatcgaggacgacggcgcgatcgcgctcacgccgatcatgcacgcgctccgggagctcggcggtccgatcgacggttaccaccaggccgctctggttcggacacccgccgggatgaccggcgagcagctgaagaccgtgctcggcgcgctcgtggcgaggcacgacatgctgcgcgcgcggctcgaccggtcgggcgacggctgggtgctccggggggccgaatcggccgaggtgcacgtcgagcgtgtcgacgccgaggacggcgatctgttcgctctgatcgagaagcacgcggtgccgactcgttcccggctgaacccggacgacggcgtgatgctgagcgcgacctggttcgatgcgggctcgtcggggccaggccggttgctgctgatgatccatcacctggccgtcgacggtgtctcgtggcggatcctgctgccggacctcgcttccgcgtgggaatcggtcgcccgcggtgaagcgccgacgttgcccactgtggagacctcgttccggcgatggtcgcgagcactgtccacacaggaccgttcacgtgagctggaccgatggaagtcggtgctgggcggtggggatccgatcccgctggaccggccgctcgacccggcccgggacgtgcaggcgaccgtgcggaaggtgtcgctgcggctgcccgccgagcggacggccccgctgctgtcccgggcgacgatcaacgacgtcctgctcaccggtctcgccctcgccgtcgcccgctggcggcgggacgggggacgggcggtgctggtcgacgtcgaggggcacggccgcgaagaggaactggcggacctttcgcaaaccgtcgggtggttcaccagcgtcgtcccgatgtgcctggacctgggcgacgtcgacgtggaggacgcgttcgcgggcggtcccgccgccgacgacgcgctgaagcgggtccaggctcacctcgatggggtgccggacggcggcatcgggcatggcgtgctccgctatctcgaccctcgtaccgggcctgaactcgcgaagttcgcgaagccgcagatcgagttcaactacctgggccggatcggcgtccccgaagcgaccgactggtcgtacgcggcggaggccgaggcggccgaactcggcgccgatgacggcatgccggtctcgcacgcgctgacggtcaacgtcgtggccgaggaccgccccggcggtcccgaactgagcgcacactggtcgtggccggaggccgtgctcaccgaagacgcggtgcgggaactcgcggaaggctggttcctcgcgctggacgcgctcactcgccgggctgattcaccggccgtacagaaggagaacaaggaatgacgaacccgttcgagaaccccgacggcacgtacctggtgctcgtcaacgacgaagggcagcacagcctgtggcccgagttcgtcgaggtcccggcgggctggaccacgaagttcggcccggcctcgcgtccggagtgcctcgaatacgtcgagaccaactggaccgaccttcggcccgcttcgctcgtcaaggccatggagtcctgacccgcttcggtccgtgaagg

SEQ ID NO: 2 - protein, 145 aa, Amycolatopsis orientalis
MPEKMTDDQRRAFLTEGTRTAVFATVRPDGRPHAVPVWFALDGDDILVNLGADTVKGKALKENPRVSVVADDPLPPYSFVSVEGVAEIVSDPDGVRLGSERIARRYLGEAGQETIDGWLAYATSPGKVIVRVRPEHFVAIAKVGG

SEQ ID NO: 3 - DNA, 438 bp, Amycolatopsis orientalis
atgcccgagaagatgaccgacgaccagcgccgcgccttcctcaccgagggcacccgcaccgccgtcttcgcgacggtccgcccggacggcaggccgcacgcggtgccggtctggttcgctctcgacggggacgacatcctggtcaacctcggtgcggacaccgtcaaaggcaaggcgctcaaggagaatccgcgggtctcggtggtcgccgacgatccgctcccgccgtactcgttcgtcagcgtcgagggtgtcgcggagatcgtttcggatccggacggcgtgcgtctcggctccgaacggatcgcccgccggtacctcggcgaggctgggcaggagaccatcgacggctggctggcgtacgcgacttcgccgggcaaggtgatcgtgcgggtgcggccggagcatttcgtggcgatcgcgaaggtcggcggctga

SEQ ID NO: 4 - protein, 325 aa, Amycolatopsis orientalis
MGGAMTDTIAAAKAGDETAFAALTGRHRRELHVHCYRMLGSFDEAEDVVQEALLNAWRGLDTFDGGNFRAWLYRIATNACLDAIRKKDRRVPSVRSFAEVPWIQPYPDRLLDEIAPPEAEPHALAVARETIELAFLAVLQMLPPRQRAVLILRDVLGWQAAETAELLDLSVPAANSALQRGRATLRERLPQQRGEWSVQAPSEEERALVRRLVEAHDNGDFGTLSSLVRDDVRVTMPPEPHCYIGLEHLARHGRIAEEMGEWRAVAVDANRMPAFASYLRRPGDTVFRAFKLDLVRVVGGKAAEFTTFDSHLFPAFGLGAVLEGK

SEQ ID NO: 5 - DNA, 978 bp, Amycolatopsis orientalis
atgggaggcgccatgaccgacacgatcgccgcggcgaaggcgggagacgaaaccgccttcgccgcgctcaccgggcggcaccgccgggaactccacgtccactgctaccggatgctcggctcgttcgacgaggcggaagacgtcgtccaggaggcgttgctcaacgcgtggcgcgggctggacaccttcgacggcggcaacttccgcgcgtggctgtaccggatcgccaccaacgcctgcctcgacgcgatccgcaagaaggaccggcgggtgccgtcggtccgctcgttcgccgaagtcccttggatccagccgtatccggaccgcctgctggacgagatcgcgccgcccgaggccgaaccgcacgcgctcgccgtcgccagggagaccatcgaactcgccttcctcgcggtcttgcagatgctgccgccccggcaacgcgcggtgctgatcctgcgcgacgtgctcggctggcaggccgccgaaaccgccgaactgctcgatctctcggtccccgccgcgaacagcgccctgcagcggggccgcgcgaccctgcgcgaacgcctcccgcagcagcgcggcgaatggtcggtccaggctccctccgaagaggaacgcgcactggtccggcggctcgtcgaagcccacgacaacggcgacttcggcacattgagcagcctggtccgcgacgacgtccgcgtgaccatgccgccggagccccactgctacatcggcctcgaacacctcgcccggcacggcaggatcgccgaagagatgggcgaatggcgtgccgtcgcggtcgacgccaaccggatgcccgccttcgcctcgtacctgcggcgtcccggcgacaccgtcttccgcgccttcaagctcgacctcgtccgcgtcgtcggcggcaaggccgcggagttcaccacgttcgacagccacctgttcccggccttcggcctgggcgccgtcctggagggaaagtaa

SEQ ID NO: 6 - protein, 315 aa, Amycolatopsis orientalis
MRVAMFGYQTWGHRTLRALIDAGHDVALVVTHPKSDHAYEKIWSDSVADLAEENGIRALLRQRPDDAEILAELKEADLDLIVANNWRTWLPPEIFEMPRYGTLNVHDSLLPAYAGFSPLIWALINGEKEVGLTAHMMDADLDAGDIVLQRSVPVGPKDTTTDLFHRTVDLIAPITTEAIELIAKGDFTPVKQDRSKASFFHKRALEDSLIDWSWPADEIERLVRAQSDPYPNAFTYHRGKQLRVIRSSVSVGQYGGTPGRIFIREGEGVVIVAGPESRRGRSHGLVIERVRTEDGTELAATDYFRTMGGYLTTRP

SEQ ID NO: 7 - DNA, 948 bp, Amycolatopsis orientalis
atgcgggtcgccatgttcggataccagacctggggacaccgcaccctgcgggcgttgatcgacgcgggccacgatgtcgccctcgtcgtcacccacccgaagagcgaccacgcctacgagaagatctggtccgactccgtcgcggacctcgccgaggagaacgggatccgcgcgctgctgcggcagcgtcccgacgacgcggaaatcctggcggagctgaaggaagccgatctcgacctgatcgtcgccaacaactggcgcacctggctgccgcccgagatcttcgagatgccgcggtacggcacgctgaacgtccacgactcgctgctgcccgcctacgccggcttctcgccgctgatctgggcgctcatcaacggcgagaaggaagtcggcctcaccgcccacatgatggacgccgacctcgacgcgggcgacatcgtgctgcagcgctccgtccccgtcggaccgaaggacacgacgaccgatctgttccaccggacggtcgacctcatcgcgccgatcaccaccgaagccatcgagctgatcgcgaagggggacttcaccccggtcaaacaggacaggtcgaaggccagtttcttccacaagcgcgccctcgaagacagcctcatcgactggtcgtggcccgcggacgagatcgagcggctggtgcgcgcccagtccgacccgtaccccaacgccttcacctaccaccgcggcaagcagctgcgcgtgatccgctcctcggtgtcggtgggccagtacggcggcacccccggccggatcttcatccgagagggtgaaggcgtcgtgatcgtcgccggccccgagtcgcgccgcggccgttcgcacgggctcgtcatcgagcgggtgcggaccgaggacggcacggaactggccgccaccgactacttccggaccatgggcggatacctcacgacccgcccctga

SEQ ID NO: 8 - protein, 447 aa, Amycolatopsis orientalis
MARAVVGERVPVYDVVGVGFGPSNLALAIAVTEHNAAPGVETVTAHFLERQARFGWHRGMLIDNATMQVSFLKDLATMRNPTSSFSFLSYLHSKDRLVDFINHKNLFPLRIEFHDYFEWAAEKVDDLVSYGTEVLSVTPVFDGDEIVFFDVHARTDGELVNLRARNLVMGTGLRPNLPEGVTPGTRIWHNSELLHRLEGMAAEEPRRFVVVGAGQSAAEVSALLHDRFPRAEVCAVFARYGYSPADDSAFANRIFDPEAVGRFYEAPEAVKDRLMRYHGATNYSAVDIELIDELYRRVYREKVQGIERLKLINVSRPTEVTDTGSEVRVTVEALETGARTRLDADFVVYATGYSPADPTPLLGELASTCARDDEGRLRVERDYRIVTEPPLDGGIYLQGGTEHTHGITSSLLSNTAVRVGEILQSIVDRRAAGASRPAYAVSGSRPA

SEQ ID NO: 9 - DNA, 1344 bp, Amycolatopsis orientalis
atggcacgagcagtggtcggtgaacgggttccggtctacgacgtggtcggggtcgggttcggtccctcgaacctcgcgctcgccatcgccgtcaccgagcacaacgccgcgcccggggtcgagaccgtcacggcccacttcctcgagcgtcaggctcgtttcggctggcaccgcgggatgttgatcgacaacgcgaccatgcaggtctccttcctcaaggacctggcgacgatgcggaacccgaccagttcgttcagcttcctgtcgtatttgcacagcaaggaccgattggtcgatttcatcaaccacaagaacctgttcccgttgcggatcgagttccacgactacttcgaatgggcggcagaaaaggtcgacgacctggtttcctatggcacggaagtgctttccgtcacgccggtgttcgacggcgacgagatcgtgttcttcgacgtgcacgcccgcaccgacggcgagctggtgaacctgcgcgcccgcaacctggtgatgggcaccggtctgcgcccgaatctccccgaaggtgtgacgccgggcacacgcatctggcacaacagcgaactcctgcaccggctcgagggcatggccgccgaggaaccgcgccggttcgtcgtcgtcggcgccggccagagcgcggcggaggtcagcgcgctgctgcacgaccgcttcccccgcgccgaggtgtgcgcggtgttcgcgcggtacggctacagcccggccgacgacagcgcgttcgccaaccggatcttcgaccccgaggcggtcggccggttctacgaggcgccggaagcggtcaaggaccgcctcatgcgctaccacggcgcgaccaactactcggccgtggacatcgagctcatcgacgagctgtaccggcgcgtctaccgcgagaaggtgcaggggatcgagcggctgaagctgatcaacgtctcccggcccaccgaggtcaccgacaccggttccgaagtgcgggtcaccgtcgaggcgctggagaccggggcgcggaccaggctcgacgccgatttcgtcgtgtacgccaccgggtacagcccggcggacccgacgccgttgctcggcgagctcgcctccacctgcgcgcgcgacgacgagggcaggctccgcgtcgagcgcgactaccggatcgtcaccgagccgccgctcgacggcgggatctacctgcagggcggcaccgagcacacgcacggcatcacgtcttcgctgctgtccaacaccgcggtccgggtcggcgagatcctccagtccattgtggacaggcgtgcggccggcgcgtcccggcccgcgtacgcggtgagcgggtcccggcccgcctga

SEQ ID NO: 10 - protein, 7403 aa, Amycolatopsis orientalis
MNRGIPLTPAQQGIWLAQQLDPADPVYTIGWVVELRGAVDLARLGDAVRRAVAEAECLHVTVGLDGDTPVQRPAAPGEVAVLDFSGEADPERAADEWARAELAVVADPARGPLTAHTLLILAGDRVRWFQRYHHLVMDAHGQAVLTRRAAALYSGTAEEVSWPLAGLAESGYDGTADREYWLGELAGRPNPVRLLGRASAGRVRLRRHSWELPAAEAGRLRKFALDAGTRLSRVAIAAVAAYAHRVTGAEDLVLGIPVTARTTRALREQPGMVSNVLPLRLTVRPETTPSRLVAQVAEKVSALLEHSRYRGEDLARELGASGGVHELVGLSVNYMAVDGELAFGDASATVHNLELGPISDVAIAVYDAGEGRGLRFDFDADEAVSSDDELADHRRRFAAVLDALLNGPDLPLAAIDLLSAEERSRVLELGTAVAESPEVSWPEAFARVVARKPDAEAVVCEDVRMTYAEVDAAANRLARLLRRCGVGHEDVVGVAMPRSADLVVALLGVMKAGAAYLPLDLDHPEDRLAYMLAHAGAGIVVSTVEESAQLPSGVDVERILLDDPAITAELAALDSSTVDVSGIALGQAAYVIYTSGSTGKPKGVVLSHDGIGSLISTATERIGIGENSRVVQFASTGFDVTVWDLVMSLCVGGTLIVVPSHRRVAGVELTGYIAANAATHMILPPSLVAALPADCELPKGAVLIVGTETVPPELIARWAEDLKVVAAYGLTEATVNSTLWAAEPGWSGPIPIGVPDPNTRCYVLDTALRPVPVGVEGELYVGGRGLARGYAGRPGLSAERFVADPFAGGGERMYRTGDRVRWRADGNLDFLGRTDHQVKIRGFRIEPGEVESALAAYAGVERIAVVPREVKPGDRRLVAYVVPQADGSPSLLKDLRAHSESALPHYMVPSAFVELDRLPIMPNGKLDRTALPLPDFGAAATGRAPATERERILCDVAARVLGLATIGADDDFFSLGGDSILSIRLVLGAAEHGLAITPRQVFQHRTPEALAACAEAVRAESTVDDRPLLELTDAERASLAEYADILPVTPLQEGFFFHAEFEGGAAADIYTVQEVLDLEGDVDADVLRRSTQALLDRYSSLRSGFRQLDGGRVVQTVARHAELPWREVAAEDAEAAFEADRARRFDFARPPLLRATFTRLGVDRAKLALTFHHIIADGWSVVVLLRELLAAYGGAPVLPEAGTRADHLRRLASRDHEVSREAWRTALSEVDEPTRLIDERAGVARRPSRLHLSLGERATAALSAMAREHGLTLGTVLHGAWGLLLGGLTGRDDVLFGSTVSGRDTGATGLESAVGLYINTVPVRLRWSGAETVAAVLRRLQDEQAALLDHQHLGLGELQRLAGAGQEELFDTLVVVENFPRDDERASGTLRLAGVEVTDAVHFPVAVIATPGDDLAFSLKFDAARIDAVAAGQLAERFARLLETLSANPGLPVAALDLLSPAERGRLAELNATSHPVPERTLAEAFAEQVAKTPSATAVMFKDTELSYADLDARAEQVARRLRSLGAGPEEIVAVAVPRSAELMVALLGVLKAGAAYLPIDLDYPADRLEYMLADSGARLVVSEPGTAARIPDVAGLTRVPVTGEPVDDVEGAAAGPDNTAYVIYTSGSTGRPKGVAVTHRAIVNRLAWMQHEYRLTASDRVLQKTPSSFDVSVWEFFWALCEGATVVLAEPDGHRDPAYLARVIRDRRITTLHFVPSMLAAFLGSDEATGDLGSLRQVFSSGEALPGETAARWRSLTGVPLHNLYGPTEAAVDVTFFASTGADGATVPIGRPVWNTRLHVLDGCLRPVPDGVAGELYLAGVQLARGYHGRPGLTAERFVADPFGEPGQRLYRTGDLVRRRADGEIEYLGRTDRQMKIRGNRIELGEIEAVLVAQPEVTAATVVAKDGALVGYVAGSADPENLRTALSEALPAPMVPQAFVVLDEFPLTPSGKLDVRALPAPEAGRAEAAPGNEQERALARIFADVLGLETVGVDGDFFLLGGDSISSIAVSSRARRAGFELSPKDVFEFRTPAALAASAAPAEPMADADGIGAVPLLPEMLRLRELGDAVQPESVLLTVPSGADLETLAEALQLVLDHHDGLRLTLSRVASVLWSLEVRPQGTVQAADLLRHASGALEDEYRDAEGRLTDNLLQAVWFDEPGRLLLIAHPVLLDARSLTTLSADLAIAWQAVTDGRPPAFTRARTSLRAIARRITGAAQDPALLGEVEHWVGTLAPVSFEVRGDVSERRITLSVEDTRTLVGALPSAVHGDVTDVLLTALKIAVDGDLLVDLHGRVDPSATIGALTSVRPVRITASGEPLAMLKAIKETVRKTPEGYGPLRYLDAQTGPLLAGLAKPRIALRYDGLVPAGPGEWQVAGYAATPPSGDQVVRIGVACEETEEGPRLTATFVGPGGDLAERWAAALVELAAVDTDAPVGLTPSDLELVSLTQEEIDRVERSSPVPVADVWPLSPLQEGLFFHSSYNSDRVDIYTIQEAIDFDHRLDAGRLRAAVSALLRRIPSLGAGFTSEGLRGPVQFVAAEVAPPVSEVDLSALPEDEQQAELDELMAKDRATRFDLGAPPLFRLKLVRLGNGRDRVVLNRHLLLWDGWSAWLFIEQLFALYDLGGDDRTLPAAGSYVDFLRWLDRQDIEAATNAWREALSGLAEPTVVGPEGRDLAPATPVNLDTVLPSGLSRALREQARRHGLTVNSVLNAAWGLVLSTMTGRHDVVFGAAVAGRPAAVPEIENTIGLFLNTVPTRVRLDAAESVLDLVRRLQSERMDLTPYEFMSLGVLQREAGHRVLFDTLFVLRNADGDERRDGLRHRHGITDLLNIDATHYPLTLVVTPGEETRVTLSYRDDVIDSAEAASVLDRFTSLVAQIVDDPAKPVAALATVSAEDRAALEAGWAEAEHPVIEDTIADLLAVQAERTPGETALVFGEEQLTYAELDARINRMARLLLARGAAPEQVIALGLPRSIDMVVALFAVLRTGAAYLPLELDYPVERLAVMLEDARPLCLVSTKDVSATLPGDTPRVLVDDHEDRAFGDGPISDAERPLFARSRADRMEHPAYVIYTSGSTGKPKGVVTPYRGLTNMQLNHQGAIFAPAIASAGGRRLRIAHTVSFAFDMSWEELLWLVEGHEVHVCDEELRRDARALVAYCDEHEVDVVNVTPTYAHLLIEEGLLEGHRPALVLLGGEAVSESVWNRLRDTDGTYGYNLYGPTEYTINTLGGGTTDSDTPTVGKPIWNTRAYIVDAWLRPVRDGVAGELYIAGEGLARGYLGRPGLTAERFVADPFVPGGGRMYRTGDLVRRRPDGNLDFLGRTDDQVKIRGYRVELGEIETALSRYPEVAQAAVIARPDPSAPGLQRLIGYVVPAELSGDARAEAEADQVGEWQQIYSDEYTEIPTALFTEDFAGWDSSYDGDPIPLEHMREWRRATVDRIAELKPERVLEIGVGTGLLMGQIAPLVQEYWGTDLAAPVIAKLNRELEQDPALAAKVNLRAQPAHVFDGLPAGRFDTIVINSVIQYFPSVDYLTNVITRAIGLLAPGGALFVGDVRNLRLARAFHTAIQLTRADATSDVAQVRRAIERGAALEKELLIDPDYFTALARRLPDVAAQVRIKRARLHNELSRYRYDAVLVKEPAETFSVAQAPRLSWDEVGSLAALEDRLTGAETLRVSRIPDARQTPEVEATRALDAGAPLIDVLDLFRAPAGVEPEDVHELGDRLGFRVYCTWSADGAFEAVFVHKGQTDALVTGVHQPVSVGTSLAKYATSPTAARGGGELVQRLRDRLKTELPDYMVPAAFVTLGALPLTDNGKLNTRALPDAEQTVRLAESRPAETAEEETLCALFGEVLGVEGVGVEDNFFDLGGHSLLATRLISRARTELGAELAIRDLFEAPTVAELAARAGGGEPARAAVVRAERPDRIPLSAAQRSLWLVDRMEANAVAYNFPLTFRLRGALDLDALRAALGDVLGRHEVLRSVFDEHDGEPYQRVLDAPEIPFAVEDCDEAELDARIAELSRTPFDLAREIPVRLRVLRLGEGDQVISLVLHHSATDEWSDRPFLGDLTSAYQARLAGDSPVWTELPVQYADFALWQKDFLAQHGAAQVAYWAESLRGAPDELTLPLDRPRPLQPTGKGGKVKSPLSEELSAAVRDLAAKAGASPFMVLQAAVAALLHRTGAGEDIPLGAPIAGRTDAALDDLVGFFVNTLVLRTDLSGEPTFAALLERVREADLAAFSHGDLPFESVVERLNPPRVPGRNPLFQVMVGYHRQSDAHDVLGLPAEWFEMDTGMAKFDLHFTMVDDGTTATLMLEYAEDLAGEATAARLLDRLTSVLEQVTAEPERPVATLDVLVGDELARVNEWNRTAREVPAATLPELFEAQVARTPDAPAVVFEGIELSYAEFDTRANRLAWWLAEQGVGAESVVAVSLPRSIDLVVALYAVHKAGGAYLPVDRDYPADRIAFMLEDAAPAVVLEELPVLDGYSSESLGRIVDPSSPAYVIYTSGSTGRPKGVVVPHAGIVNRLLWMQDEYGLTADDRVLQKTPSSFDVSVWEFFWPLITGATLVVAKPEGHKDPAYLAELIRTAGITTVHFVPSMLALFLETAECAGLRRVICSGEALPSELAARVDVPLYNLYGPTEASVDVTAWRAGAETTPSVPIGRPVWNTRTYVLDARLNPVPPGVPGELYLAGVQLARGYLARPGLTAERFVADPFGGPGERMYRTGDLVRWSDDGVLEFLGRVDDQVKIRGFRVELGEVEAALTASGSVTRAVVVAREDRLVAYVVGSVDGLREAVAAKLPDHMVPSAFVVLDEIPLTPNGKLDRKALPAPEFATIEGREPRNAREEALCALFADVLGVERVGIDDGFFALGGHSLLVMRLVSRISASLGATVSVRDVFDAPTVAALAERLAEPSAQPALSAGNRPELLSLSPAQQRLWFLYRLEGVTPTYNIPLAWRLHGELDAEALSAAIGDLVERHEPLRTVFVEEGGVAAQRILTDARPEVRFEEAGDLSAQLAAEAGRGFALDEELPIRATVFAVSPREHVLLLVIHHVATDEWSQGPLMADLATAYEARLAGTAPQWTKLAVQYADYALWQRELLDSTAAGHLRFWQEALADLPDELPLPADRRRPAESSHRGGLVTFPIDAALTARLRAVAQRHDVSMFMLAQAAVAALLHRLGAGDDIPLGAPSSGRLDERLEPLIGFFVNSLVLRTDLSGEPAFGELLSRVRATDLAAFEHQELPFERLVDAVNPERSLARHPLFQVMVVYLPEAEDRLTLPGLRAEQEDFAQGVAKFDLEFGFLEHGDGISGAIEYSADLFDHATAESFARRLVTVLEQVAADPAIPVGELDVFGEDERALLRHWNGTAHDVPASTLPELFEAQVARTPDAPAVVFEGVELSYAEFDARANRLAWWLVEQGVGAESVVAVSLPRSIDLVVALYAVHKAGAAYLPLDTDYPADRIAFMLEDAAPTVVLDELPVLDGYSGESLRRAVDPASPAYVIYTSGSTGRPKGVVVPHEGIVNRLLWMQDEYGLTADDRVLQKTPSSFDVSVWEFFWPLITGATLVVAKPDGHRDPRYLAELIRDRRITTLHFVPSMLEAFLAEEPEWHGVRRVLCSGEALPPELAARVTVPLHNLYGPTEASVDVTSWEIVDDTVPIGRPVWNTQVHVLDSRLRPVPPGVAGELYLAGKQLARGYLGRAGLTAERFVANPFAAGERMYRTGDLARWRADGALDYLGRADDQVKLRGFRIELGEIESVLSARVVLREGRLVAYLTGEADVEQARRLAEATLPEYMVPSAFVVLDAFPLTPNGKLDRKALPAPDFSGEVTDDAPRTPREELLASLVAGVLGLEKVGVHDDFFRLGGDSIVAMQLVGRVRNAGLLLSPRDVFRHRTVAALAEVSAVRLDASAPESGKPLVELDASERAEILDVAPDTAEVWPLTPLQAGLLFHATMDGDGPDVYTVQVSFDLEGPLDRARLRDAAQALVDRNAVLRTGYRYLASGRPVALVARSVTVPWRFADAPDAVEAEMAHDRRRFDPAAAPVLRFLLLALPDGGHRLVFTHQHVLLDGWSVPQLFAELSQLYAGADVAPARQYRDHLGWLGGQDRDAAEAAWRQALDGLTEPTHLVPHDPQRIPALPERLSLELPEQATEALTALARSRGLTLNTLVQTAWSIVLGRLTGRADVVFGATVAGRPPELAGVERMIGLFINTIPVRIVLDPAEKLGDLLDRVQDEQSGLLAHQHFGLADIQRGAGLGELFDTLMVFESYPGADEDETGAGLRGEIVRHDDSTHYPLTWAVEPGARLKLTAEYRADLFDAGLPERLLPAMERVLAAMVSDVDAPVGRADILGEADLDLIVREWNGTALVVEPSTVAGLFEAQVRRSPDATAVVSGVVTLTFTELNERANRLARALVAHGVGPEDFVALALPRSADVLLAILAVHKAGAAYLPLDPDYPAERLAAMLADARPKVLLTEPELLEALPRTGIETLALCALVTEDRQAHDLTDRDRVRPLVPEHPAYVIYTSGSTGTPKGVVVPHRGVVNLFHSHRATLHRPAVEATGKPHLRVGHAWSFSFDASWQPQLWLLDGHAVHIVDEETRRDPELLAAMIEREGFDFIEVTPSFFAQMADAGLLDGDRCPLAVIGVGGEAVPESLWRRLATLDGTEAFNLYGPTESTVDALVARIADSEKPLVGRPVANTHAYVLDAALRPVPPGITGELYLAGAGLARGYLGRAALTAERFVADPFGPAGARMYRTGDLARWTADGRLDFGGRADDQAKIRGFRVEPGEIEAVLERHDGVAQAVVLVREDRPRVKQLVAYVVSTVERDELRRFAAETLPDYMVPAAFVTLDALPTLSNGKLDRKALPAPDYSELAGGRAPETALEKTLCALFAEVLDVPELGADDDFFALGGDSIVAMRLVGRARAAGLKITPHDVFAGRTVAALAEVAERIEDDGAIALTPIMHALRELGGPIDGYHQAALVRTPAGMTGEQLKTVLGALVARHDMLRARLDRSGDGWVLRGAESAEVHVERVDAEDGDLFALIEKHAVPTRSRLNPDDGVMLSATWFDAGSSGPGRLLLMIHHLAVDGVSWRILLPDLASAWESVARGEAPTLPTVETSFRRWSRALSTQDRSRELDRWKSVLGGGDPIPLDRPLDPARDVQATVRKVSLRLPAERTAPLLSRATINDVLLTGLALAVARWRRDGGRAVLVDVEGHGREEELADLSQTVGWFTSVVPMCLDLGDVDVEDAFAGGPAADDALKRVQAHLDGVPDGGIGHGVLRYLDPRTGPELAKFAKPQIEFNYLGRIGVPEATDWSYAAEAEAAELGADDGMPVSHALTVNVVAEDRPGGPELSAHWSWPEAVLTEDAVRELAEGWFLALDALTRRADSPAVQKENKE

SEQ ID NO: 11 - DNA, 22212 bp, Amycolatopsis orientalis
atgaaccgcggcattcccctgaccccggcacagcaggggatctggctcgcgcagcagctcgacccggccgatccggtctacacgatcggctgggtcgtggagcttcgcggcgccgtcgacctcgcccggctgggtgacgccgtccggcgggcggtcgcggaggcggaatgcctgcacgtgacggtcgggctggacggtgacacgccggtccagcgtccggccgctcccggtgaggtcgccgtgctcgacttcagtggtgaggcggatccggagcgcgccgccgacgagtgggcgcgggccgaactcgccgtcgtggccgaccccgccagggggccgctcaccgcgcacacgctgctgatcctggccggggaccgggtccgctggttccagcgttaccaccatctcgtgatggacgcgcacgggcaggccgtcctgacccgccgcgcggcggcgctctattcgggtaccgccgaggaggtctcttggccgctggcggggttggccgagtccggctacgacgggacggccgaccgggagtactggctgggtgaactcgcgggcaggccgaacccggtgcggctgctcggccgcgcgtcggcgggccgggtccggctccggcggcattcgtgggaactgcccgcggccgaggcaggccgcctgcggaagttcgcgctggacgcggggacgaggttgtcccgggtcgccatcgccgccgtggcggcgtacgcccaccgcgtgacaggcgccgaagacctcgtcctaggcatccccgttaccgcgcgcaccacccgcgccctgcgtgaacagccgggcatggtctccaacgtcctgccgctgcggctgaccgtgcggccggagacgacgccgtcgcgactcgtcgcccaggtcgcggagaaggtgtccgcgctgctggaacacagccggtaccgcggggaggacctggcccgcgaactcggcgcgagcggcggggtgcacgaactcgtcgggctgagcgtgaactacatggccgtggacggcgaactcgcgttcggtgacgcgagcgcgacggtgcacaacctcgaactcgggccgatcagcgacgtcgccatcgccgtgtacgacgccggcgagggccggggtctccgcttcgacttcgacgccgacgaggccgtctcgtccgacgacgaactcgccgaccatcgccgccggttcgccgccgtactggacgccctgctgaatggcccggacctgccgctggccgccatcgacctgctctcggcggaggagcggtcccgggtcttggagctcgggaccgcggtcgcggagtcgcccgaggtgtcctggcccgaggcgttcgcccgggtggtggcgcggaagccggacgccgaggccgtcgtctgcgaggacgtccggatgacctacgccgaggtcgacgccgccgccaaccggctcgcgcggctgctgcgccggtgcggggtcggtcacgaggacgtcgtcggcgtggccatgccgcgttcggccgatctcgtggtcgccctgctcggcgtgatgaaggccggggccgcgtatctcccgctcgacctggaccaccccgaggaccgcctcgcgtacatgctcgcgcacgcgggcgcggggatcgtggtgtccactgtggaggaatcggcccagctcccgtccggggtggacgtcgagcggatcctgctcgacgacccggcgatcaccgccgagcttgcggcgctcgattcgtccactgtggacgtgagtgggatcgcgctgggccaggccgcgtacgtcatctacacctctggctcgacggggaagccgaagggcgtggtcctgtcccacgacgggatcggcagcctgatcagcacggcgacagagcggatcggcatcggcgaaaacagccgggtcgtgcagttcgcctccaccgggttcgacgtgacggtctgggatctggtcatgtcgctgtgcgtcggcgggacgctgatcgtcgtgccgtcgcaccgccgcgtcgcgggtgtggagctgaccgggtacatcgccgccaacgcggcgacgcatatgatcctgccgccgtcgctggtcgcggccctcccggcggactgcgagctgccgaagggcgcggtgctgatcgtcggcaccgagaccgtcccgccggaactgatcgcgcgctgggccgaagacctgaaggtcgtcgccgcgtacgggctgaccgaggcgacggtgaactcgacgctgtgggcggccgagcccggctggagcgggccgatcccgatcggtgtcccggatccgaacacccgctgctatgtcctcgacaccgccctgcgaccggttccggtcggtgtcgaaggggagctgtacgtcggcggacgcggtctcgcccggggctacgcgggacgtccggggctgtcggcggagcgtttcgtcgccgacccgttcgccggtggcggcgagaggatgtaccgcaccggcgaccgcgtccgctggcgcgcggacgggaacctcgacttcctcggccgcaccgatcaccaggtcaagatccgcggattccggatcgaacccggtgaagtcgaatcggcactcgccgcgtacgccggggtcgagcggatcgccgtcgtgccccgcgaggtcaaaccgggggaccggcgcctggtggcgtacgtcgtgccgcaggcggacggatcgccgtcactgctgaaagacctgcgcgcccactcggaatcggcgttgccgcactacatggtgccgtcggccttcgtggaactcgaccggctgccgatcatgccgaacggcaaactcgaccggaccgcgttgccgctgccggacttcggcgccgccgcgaccggccgcgcgccggccaccgaacgggaacggatcctctgcgacgtcgccgcacgagtcctcggcctggccacgatcggcgcggacgatgacttcttctcgctcggcggcgacagcatcctgtccatccggctggtgctgggcgcggcggaacacggtctcgcgatcaccccacgccaggttttccagcacaggactcccgaagcgctggccgcctgcgctgaagcggtgcgcgccgagtccactgtggacgaccgaccgctactggagctgaccgacgccgaacgtgcgtcgctcgccgagtacgccgatatcctgcctgtgacaccgttgcaggaagggttcttcttccacgcggaattcgaaggcggtgcggccgccgacatctacacggtccaggaggtgctggacctcgaaggcgatgtcgacgccgacgtgttgcgccggtcgacgcaggcgctgctggaccggtattcgtcgctgcggtccggtttccgccagctcgacggcggccgggtcgtccagacggtggcacggcacgccgaactgccttggcgcgaggtggccgccgaagacgccgaggcggccttcgaggccgatcgtgcgcgccggttcgacttcgcccggcctccgctgctgcgggcgaccttcacccggctcggcgtggatcgcgcgaagctggcgctcacgttccatcacatcatcgccgacggctggtcggtcgtggtgctgctgcgcgaactgctcgcggcgtacgggggagcacccgtactgccggaagcgggcacccgcgccgatcacctgcggcggctcgcgtcacgtgatcacgaggtctcacgcgaggcttggcggacggcgttgtccgaagtggacgaacccacccggctgatcgacgaacgagccggggtggcgagacggccgtcgcggctgcacctgagcctcggcgagcgggcgacggcggcgttgtccgcgatggcccgcgagcacgggctcaccctcggtactgtcctgcacggtgcctggggactcctgctcggcggcctcaccggacgggacgacgtcctgttcgggagcacggtctcgggccgtgacaccggcgccaccggtctcgaatcggcggtcggcctctacatcaacacggtgccggtgcggctgcgctggtcgggggccgagaccgtcgccgccgtgctgcgacggctgcaggacgagcaggcggccctgctggaccaccagcatctcggcctcggcgaactgcagcgcctcgcgggcgcggggcaggaagaactgttcgacacccttgtcgtcgtcgagaacttcccgcgcgacgacgagcgcgcctccggaaccctccgcctcgccggcgtcgaagtcaccgacgccgtgcacttcccggtcgcggtgatcgcgacaccgggggacgatctggcgttcagcctcaagttcgacgcggcgaggatcgacgcggtcgcggccgggcagctcgcggaacggttcgcgcggctgctggagacgctgagcgcgaaccccggtctgccggtcgccgcgctcgacctcctgtcgcccgccgaacgcggccgcctcgccgaactcaacgcgacctcgcaccccgtgccggagcgtacgctggccgaggccttcgccgagcaggtcgccaagaccccgtccgcgacggcggtgatgttcaaggacaccgaactgtcctatgcggacctcgacgcccgcgcggagcaggtggcacggaggctgcgttcgctgggagccggtcccgaagagatcgtggccgtcgccgtgccgaggtcggccgagctgatggtcgcattgctgggtgtcctcaaggccggggccgcctacctgccgatcgacctcgactatccggccgaccggctcgaatacatgctggccgattccggtgcgcgtctcgtggtctccgaaccggggacggccgcgcggatcccggatgtcgccgggctgactcgggtccccgtgaccggcgagcccgtcgacgatgtcgagggtgcggcagccggaccggacaacaccgcgtacgtcatctacacgtcgggttcgaccgggcggccgaagggcgtggccgtcacgcatcgcgcgatcgtcaaccggctcgcgtggatgcagcacgagtaccggctcacggcctcggaccgcgtgctgcagaagacgccgtcgagtttcgacgtgtccgtgtgggagttcttctgggcgctgtgcgaaggcgccacggtggtgctggccgaaccggacggccaccgcgatcccgcctatctagcgcgagtgatccgggaccggcggatcaccaccctgcacttcgtcccgtcgatgctcgcggccttcctcggcagcgacgaggccaccggcgatctcggatcgctgcgtcaggtgttctccagcggtgaggcactgcccggcgagaccgccgcacggtggcgctcgctcaccggtgtcccgctgcacaacctctacggccccaccgaggcggccgtggacgtgaccttcttcgccagcaccggtgccgacggcgccaccgtccccatcggacgtccggtgtggaacacgcggctgcacgtcctcgacggctgccttcgcccggtgcccgacggcgtggcgggcgagctgtacctcgccggtgtccagctggcccgcggctatcacggacggccggggctgaccgccgaacgtttcgtcgccgacccgttcggcgaaccggggcagcggttgtaccggaccggcgacctggtgcgccgccgcgccgacggcgagatcgagtacctcggccgcaccgatcggcagatgaagatccgcggcaaccggatcgagctcggcgagatcgaagcggttctcgtcgcacagccggaggtcacggccgcgacggtcgtggcgaaggacggcgcgctcgtcggctacgtcgccgggagcgcggatccggagaatctgcgaaccgccctcagtgaggcactgcccgcaccgatggtcccgcaggcgttcgtcgtcctcgacgaattccccttgacccccagcggaaagctcgacgtccgggccctgcccgcgcccgaggccggccgggccgaagccgcacccgggaacgagcaggagcgcgcgctcgcgcggatcttcgccgacgtcctcggcctggagaccgtcggcgtcgacggggacttcttcctgctgggcggcgacagcatctcgtcgatcgccgtgtcgagccgtgcccggcgggccggattcgagctgagccccaaggacgtgttcgaattccggaccccggcggcgctcgccgcgtccgccgcgccggcggagccgatggccgacgctgacggcatcggtgccgtccccctgctgccggaaatgctccggctgcgtgagctgggcgacgcggtccagccggagtccgtactgctgaccgtgccctccggcgccgacctcgagacgctcgccgaggccctgcagctcgtgctcgaccaccacgacggcctgcggctcacgctgtcgcgggtcgcctcggtgctctggtcgctcgaagtccgtccacaagggaccgtccaggccgcggacctgctccgtcacgcatccggagccctcgaagacgagtaccgggacgccgagggcaggctgaccgacaacctcctgcaggccgtctggttcgacgaaccgggcaggctgctgctgatcgcgcatccggtcctgctggacgcgcggagcctgacgacgctgtccgccgatctcgcgatcgcctggcaggccgtgaccgacggccgcccgccggcgttcacgcgagcgcggacgtcgctgcgggccatcgcccgccggatcaccggcgcggcacaggatcccgcgctgctcggcgaggtcgagcactgggtcgggacgctggcacccgtttccttcgaggtccgcggcgatgtcagcgaacgccggatcacgctgtccgtcgaggacacccggacgctcgtcggcgcgctgccgtccgccgtgcacggcgacgtgaccgacgtcctcctgaccgcgttgaagatcgccgtcgacggtgacctgctcgtcgatctgcacggccgggtcgacccgtcggccacgatcggcgcgctgacctcggtccgcccggtgcggatcaccgcgtccggcgagccgctcgcgatgctcaaggccatcaaggaaaccgtccggaagacccccgagggctacggcccgctgcggtacctcgacgcccagaccggaccgctgctcgccgggctagcgaagccgcggatcgccctgcgatacgacggcctcgtcccggccggaccgggggagtggcaggtcgccggatacgcggccacgccgccgtccggcgaccaggtcgtgcgtatcggcgtcgcctgcgaggagaccgaggaaggcccgaggctgaccgcgacgttcgtgggtccgggcggagacctggccgaacgctgggccgccgccctggtcgaactcgcggccgtggacaccgacgccccggtggggctgacgccgtccgacctcgaactggtgtcgctgacgcaggaggagatcgaccgcgtcgagcggtccagcccggtgccggtcgccgacgtctggccgttgtcgccgctgcaggaaggcctcttcttccactccagctacaactccgatcgggtggacatctacacgatccaggaggcgatcgacttcgatcaccgcctggacgccggcaggctccgggcggcggtgtccgcgctgctgcggcggatcccgagcctcggcgccggtttcaccagcgaagggctgcgcggtccggtccagttcgtggcggccgaggtcgcgccgccggtgtccgaagtggacctttcggcgctgcccgaagacgagcaacaggcggagctcgacgagctgatggccaaggatcgcgcgacgcggttcgacctcggtgccccgccgctgttccgcctgaaactggtgcggctcgggaacggccgcgaccgggtggtgctgaaccggcacctgctgctctgggacggctggtcggcctggctgttcatcgagcaactgttcgcactgtacgacctgggcggcgacgaccggacacttcccgcggccgggtcctatgtggacttcctgcgctggctggaccggcaggacatcgaggccgcgacgaacgcgtggcgcgaggcgctgtccggcctcgcggagccgaccgtcgtcgggcccgaaggccgcgacctggctccggcgaccccggtcaacctcgacaccgtgctgccctccgggctcagccgggccttgcgcgagcaggcccgcaggcacggcctcacggtgaactccgtgctcaacgccgcctggggcctggtcctgtcgaccatgaccgggcgccacgacgtcgtgttcggtgccgcggtcgcgggcaggcccgccgccgtgcccgagatcgagaacaccatcggcctgttcctcaacaccgtgcccacccgggtacggctcgacgcggccgaatccgtgctggacctggtgcggcgcctgcagtccgagcggatggacctgacgccgtacgagttcatgagcctcggggtgctgcaacgcgaggccgggcaccgtgtcctcttcgacacgctgttcgtgctgcgcaacgccgatggcgacgaacggcgcgacggcctccgtcaccggcacggcatcaccgacctgctgaacatcgacgccacgcattacccgctgacgctcgtggtcacgccgggcgaggaaacgcgggtcacgctgtcttaccgggacgacgtcatcgactccgccgaagcggcgtcggtgctcgaccggttcacgtcgctggtggcgcagatcgtcgacgacccggcgaaaccggtcgccgcgctggccacggtgtccgccgaggaccgggccgcgctggaagcgggctgggccgaagccgagcaccccgtcatcgaggacacgatcgcggatctgctcgccgtgcaggcggagcgcactcccggcgagacggcgctggtgttcggcgaagaacagctgacctacgccgaactcgacgcgcgcatcaaccggatggcccggctgctgctggccaggggagccgctccggaacaggtgatcgcgctcggcctgccgcgttcgatcgacatggtcgtcgcgctgttcgccgtgctgcggacgggtgcggcgtacctgccgctggaactcgactacccggtcgagcggctcgcggtgatgctggaagacgcccggccgctctgcctggtgtccacaaaggacgtttcggcgacgctgcccggtgacacgccccgggtgctcgtggacgaccacgaagacagggcgttcggcgacggcccgatctccgacgccgagcgcccgctgttcgcgcggagccgcgcggaccgcatggaacacccggcgtacgtcatctacacctcgggctcgaccgggaagccgaagggcgtcgtcacgccgtaccggggcctgacgaacatgcagctcaaccaccagggcgcgatcttcgccccggccatcgcgtcggcgggcggacggcggctgcggatcgcgcacaccgtgtcgttcgccttcgacatgtcctgggaagaactgctgtggctcgtggaggggcacgaggtccacgtctgcgacgaggaactgcgccgcgacgcccgggcgctcgtggcgtactgcgacgagcacgaggtcgacgtcgtcaacgtgacgcccacgtacgcccatctgctgatcgaggaagggctgctggaggggcaccggcccgcactcgtcctgctcggcggcgaggcggtgtcggagtcggtgtggaaccggctgcgcgacaccgacggcacctacggctacaacctttacggccccaccgaatacacgatcaacacgctcggcggcggcacgaccgacagcgacacccccaccgtgggcaagccgatctggaacacccgcgcgtacatcgtcgacgcctggctccggcccgtccgcgacggtgtcgcgggcgagctctacatcgcgggcgaaggcctggcccgcggttacctcggccgtcccgggctgacggccgaacggttcgtcgccgatcccttcgtgcccggtggcgggcgcatgtaccgcaccggtgacctggtgcgccgccgtcccgacgggaacctcgacttcctcggccgcaccgacgaccaggtgaagatccgcggctaccgggtggaactcggcgagatcgagaccgcgctcagccgctaccccgaggtcgcgcaggccgccgtcatcgcgcgcccggatccgtcggcgcccggtctgcagcggctgatcggttacgtcgtcccggcagagctgagcggggacgcgcgcgccgaagcggaagccgaccaggtcggcgaatggcagcagatctactcggacgagtacaccgagatcccgacggcgttgttcaccgaggacttcgcgggctgggactccagctacgacggcgacccgatcccgctggagcacatgcgggaatggcggcgcgcgaccgtcgaccggatcgccgagctgaaaccggaacgggtgctggagatcggcgtcggcaccggtctgctcatggggcagatcgcgcccctggtccaggagtactggggcaccgacctcgccgcgccggtgatcgcgaagctgaaccgcgagctggaacaggatccggcgctggcggccaaggtgaacctgcgggcccagccggcgcacgtgttcgacggcctgcccgccggccggttcgacacgatcgtgatcaactccgtcatccagtacttcccgagcgtcgactacctgaccaacgtcatcacgcgggcgatcggcctgctcgcccccggcggcgcgctgttcgtcggcgacgtccggaacctgcggctcgcccgcgccttccacaccgcgatccagctcacccgcgccgacgccacctccgacgtcgcccaggtgcgccgcgcgatcgaacgcggtgccgcgctggagaaggaactgctgatcgacccggactacttcaccgcgctcgcgcggcggctcccggacgtcgcggcgcaggtccggatcaaacgcgcccggctgcacaacgagctgagccggtaccgctacgacgcggtcctggtgaaagagcccgccgagaccttctcggtcgcgcaagcaccgcggttgtcctgggacgaagtgggctcgctcgccgctttggaggaccgcctcaccggtgcggagacgctgcgggtgagccggatcccggacgcccgccagacgcccgaggtcgaggcgacgcgggcgctggacgccggtgcgccgctcatcgacgtcctggacctcttccgtgctccggccggggtcgagcccgaggacgtgcacgaactcggcgaccggctcgggttccgggtgtactgcacctggtcggccgacggcgcgttcgaggccgtgttcgtccacaagggacagacggatgcgctggtcaccggggtgcatcagccggtctcggtgggtacgagcctggccaagtacgcgacgagcccgacggcggcacgtggtggcggcgaactcgtgcagcgcctgcgggaccggctcaagacggaactgccggactacatggtcccggccgcgttcgtcacgctgggcgccctgcccttgaccgacaacggcaagctcaacacccgcgcgctcccggacgccgagcagacggtgcggctcgccgaaagccgtccggccgagaccgccgaggaagagacgctgtgcgcgctcttcggcgaggtcctcggcgtggagggggtcggcgtcgaggacaacttcttcgacctcggtggccattccctgctcgccacccggctcatcagccgggcccgtaccgaactcggcgccgaactggccatccgtgacctgttcgaggcgccgacggtcgcggagctggccgctcgcgcgggcggcggcgaaccggcgcgggccgcggtggtccgcgccgagcgccccgaccggatcccgctttcggccgcccagcgcagtctttggctcgtggaccggatggaagccaacgcggtcgcctacaacttcccgctgaccttccgtctgcgcggagccctggatctcgacgcgctgcgggccgcgctcggcgacgtgctcggcaggcacgaagtcctgcgctctgtcttcgacgagcacgacggcgagccctaccagcgtgttctcgacgcgccggagatcccgttcgctgtcgaggactgcgacgaggccgagctggatgcccggatcgccgagctctcgcggacacccttcgacctggcgcgggagatccccgtccggctgcgggtgctgcggctcggtgaaggcgaccaggtgatctcgctggttctgcaccacagcgcgaccgacgaatggtccgaccggccgttcctgggcgacctcacgtcggcctatcaggcgcggctcgccggtgacagtcccgtgtggacggagctcccggtgcagtacgccgacttcgctctgtggcagaaggacttcctcgctcagcacggtgccgcgcaggtcgcctactgggccgaatcgctgcgcggggcaccggacgagctgaccctgccgctggatcggccgcgtccgctccagccgacgggcaagggcggcaaggtgaaatccccgctctccgaggaactgtcggccgcggtgcgtgacctggcggcgaaggcgggagcgagcccgttcatggtgctccaggcggcggtggccgcgctgttgcaccggaccggcgccggggaggacatcccgctgggcgcgccgatcgccgggcggaccgacgcggcgctcgacgacctcgtcgggttcttcgtgaacacgctggtgctgcgcaccgacctctcgggtgagccgacgttcgccgccctgctcgaaagggtgcgcgaggccgacttggccgcgttctcgcacggcgacctgccgttcgaaagtgtcgtcgagcggctgaacccgccccgggtgccggggcggaacccgctgttccaggtgatggtcggctaccaccggcagagcgacgcgcacgacgtgctcggactgcccgccgagtggttcgagatggacaccggcatggcgaagttcgatctgcacttcacgatggtcgacgacggcaccaccgccacattgatgctggagtacgccgaagacctcgccggcgaggcgacggccgcgcgtttgctggaccggctcacgtccgtgctggagcaggtcaccgccgagcccgaacggccggtcgccacgctcgacgtcctcgtcggtgacgaactggcgcgggtcaacgagtggaaccgcacggcccgggaagtcccggccgcgaccctgccggagttgttcgaggcgcaggtggcgcggacgccggacgcccccgctgtcgtgttcgagggcatcgagctgtcgtatgcggaattcgacacgcgggcgaatcggctcgcgtggtggctggccgagcagggtgtgggtgcggagtcggtggtggcggtgtcgctgccgcggtcgatcgacctggtcgtcgcgctgtacgccgtgcacaaggccggtggtgcttacctgccggtcgaccgcgactacccggccgatcgcatcgcgttcatgctcgaagacgccgcccccgcggtcgtgctcgaagaactgcccgttctcgacgggtactcctccgagagcctggggcggatcgtcgacccgtcgtctccggcgtatgtgatctacacgtcgggctcgacggggcgtccgaagggtgtcgtggtgccgcatgcgggcatcgtgaaccgcctgttgtggatgcaggacgagtacggtctgacggctgatgatcgggtgttgcagaagacgccgtcgagtttcgatgtgtcggtgtgggagttcttctggccgttgatcaccggagcgacgctggtggtcgccaaaccggaggggcacaaggacccggcgtatctcgcggagctgatccgcaccgccgggatcaccaccgtccacttcgtaccgtcgatgctcgcgctcttcctcgagaccgccgaatgcgccgggctgcggcgcgtgatctgcagtggcgaggcgctgccgtcggaactggcggcccgcgtggacgtcccgctgtacaacctctacggcccgaccgaagcgtccgtcgacgtgaccgcttggcgggccggtgccgagacgacgccgtccgtcccgatcgggcgtccggtgtggaacacgcggacgtacgtgctggacgcgcggctgaacccggtcccgccgggtgtccccggcgagctgtacctcgccggcgtgcagctcgctcgcgggtatctggcccggcccgggctgaccgccgaacggttcgtcgccgacccgttcggcggcccgggggagcgcatgtaccgcaccggggacctcgtgcgctggtcggacgacggcgtgctggagttcctgggccgcgtggacgatcaggtgaagatccgcggcttccgggtcgagctcggtgaggtcgaggcggcgctgaccgcgtccgggtccgtcacgcgcgcggtggtcgtcgcgcgtgaagaccggctggtggcgtacgtcgtcggctcggtcgacggactgcgggaggcggtggccgcgaagctgccggaccacatggtgccctcggcgttcgtcgtgctcgacgagatcccgctgaccccgaacggcaaactcgaccgcaaagccctgcccgcacccgagttcgcgaccatcgagggacgcgagccgaggaacgcgcgggaagaggccctgtgcgcgttgttcgccgacgtgctcggcgtcgaacgggtcgggatcgacgacggtttcttcgccctcggtggccattcgctcctggtgatgcgcctggtcagccggatctcggcgagtctgggcgccacggtgtcggtccgtgacgtcttcgacgcgccgacggtcgcggcactggccgaacgcctcgcggaaccgtcggcccagccggcactgagcgcggggaaccggccggagctgctgtcgctttcgcccgcccagcaacggttgtggttcctgtaccggctcgaaggggtcacgcccacctacaacatcccgctcgcgtggcggctgcacggcgagctggacgcggaggcgctgagcgcggcgatcggcgacctcgtcgagcggcacgagccgctccgcacggtgttcgtcgaggaaggcggcgtcgcggcacagcgcatcctgaccgacgctcgcccggaggtccgtttcgaggaagcgggcgacctttcggcgcagttggccgcggaggccggtcgcggattcgccctcgacgaggaactcccgatccgggccacggttttcgccgtgtcgccgcgggaacatgtcctgctgctggtgatccaccacgtggcgacggacgagtggtcgcagggaccgctcatggccgacctcgccaccgcctacgaggcgcgccttgctggaaccgctccacagtggacaaagctggcggtccagtacgcggattacgccctgtggcaacgcgaactgctcgattcgacggccgccgggcacctccggttctggcaggaagcgctggcggacctgccggacgaactgccgctgcccgcggaccggcggcgtccggccgaatcgagccatcgcggcggtctcgtcaccttcccgatcgacgccgcgctcaccgcacggctgcgtgcggtggcccagcggcacgacgtcagcatgttcatgctggcccaggccgcggtcgcggcactgctgcaccggctcggcgcgggggatgacatcccgctcggcgcaccgagttccggccgcctggacgagcggctggagccgctgatcggcttcttcgtgaactcgctcgtgctgcgcaccgacctgtccggcgagccggcgttcggcgaactgctctcccgggtacgcgcgaccgacctcgccgccttcgaacaccaggagctgccgttcgagcggctggtcgacgcggtcaacccggaacgttcgctggccaggcacccgctgttccaggtgatggtggtgtacctgcccgaggccgaggaccgactgacactgcccggacttcgtgcggagcaagaggacttcgcgcagggcgtcgcgaagttcgacctcgagttcggcttcctggagcacggcgacgggatctccggcgcgatcgagtacagcgccgacctgttcgaccacgccaccgccgagtcgttcgcgcggcgcctggtgaccgttctggagcaggtggccgccgaccccgcgatcccggtcggtgagctggacgtcttcggcgaggacgagcgcgcgctgctcaggcactggaacggcaccgcgcacgacgtcccggcgagtacgttgccggagttgttcgaggctcaggtggcgcggacgccggatgctcctgccgtggtgttcgagggcgtcgagctgtcgtatgcggagttcgacgcgcgggcgaatcggctcgcgtggtggctggtcgagcagggtgtgggtgcggagtcggtggtggcggtgtcgctgccgcggtcgatcgacctggtcgtcgcgctgtacgcggtgcacaaggcgggcgcggcgtacctgccgctcgacacggactatccggccgatcgcatcgcgttcatgctcgaagatgccgcccccacggtcgttctggacgagctgccggtcctggacggttattcgggagagagcctgcggcgtgccgtggaccccgcgtccccggcgtacgtgatctacacgtccggatcaaccggccgcccgaagggtgtggtggtgccgcatgagggcatcgtgaaccggttgctgtggatgcaggacgagtacggtctgacggctgatgatcgggtgttgcagaagacgccgtcgagtttcgatgtgtcggtgtgggagttcttctggccgctgatcaccggcgccacactggtggtcgccaaaccggacgggcaccgggatccgcggtatctcgcggagctgatccgcgaccggcggatcacgacgctgcacttcgtcccgtcgatgctggaggcgttcctcgccgaagagccggagtggcatggcgtccgccgcgtcctgtgcagtggcgaggcgctgccgccggaactggccgctcgcgtgaccgtgccgctgcacaacctctacgggccgaccgaggcctcggtggacgtgacctcgtgggagatcgtcgatgacaccgtccccatcggacgtccggtgtggaacacccaggtccacgtgctcgactcccggctgcggccggttccgccgggtgtggcgggcgagctgtacctggcgggcaagcagctcgcgcgcggctatctcggccgggccgggctcaccgcggaacggttcgtggcgaacccgttcgccgcgggggagcggatgtaccgcacgggtgacctcgcgcggtggcgggccgacggcgcgctcgactacctcggccgcgccgacgaccaggtcaaactgcgtggcttccggatcgagctgggcgagatcgagtccgtgctttccgcgcgggtggtcctgcgggagggcaggctcgtcgcgtacctgaccggggaggccgacgtcgagcaggcccggcggctcgccgaggccaccctgccggagtacatggtgccgtccgcgttcgtggtactggacgcgttcccgttgacacccaacgggaagctcgaccgcaaggcgctgcccgcgccggacttctccggcgaggtcaccgacgacgccccgcgcacacctcgcgaggaactgctcgcgtcgctcgtcgcgggggtgctcgggttggagaaggtcggtgtccacgacgacttcttccgcctcggcggcgacagcatcgtcgcgatgcagctggtcggccgcgtgcggaatgccgggctgctgctgagcccgcgtgacgtcttccggcaccggaccgtcgcggccctggccgaggtcagcgcggtgcggctcgacgcatccgcgcccgagagcgggaagccgttggtggagctcgacgcgagcgagcgcgcggagatcctcgacgtcgcgccggacacggccgaggtgtggccgctgacgccgttgcaggccgggttgctcttccacgcgacgatggacggcgacggaccggacgtctacaccgtccaggtgtccttcgatctcgaagggccgctcgaccgggctcggttgcgcgacgccgctcaggcgctcgtcgaccggaacgccgtcctgcggaccggctaccgctacctcgcgtccgggcgcccggtcgcgctggtggcgcgttcggtcacggttccttggcgcttcgcggatgcgccggacgccgtcgaggcggagatggcccacgacaggcgccgcttcgatccggcggccgcgccggtactgcgtttcctgctgctcgccctgccggacggagggcaccggctggtgttcacccaccagcacgtactgctcgacggctggtcggtgccgcagttgttcgccgagctgtcccagctctacgccggagccgacgtggcaccggcccgccagtaccgcgaccatctcggctggctcggaggccaggaccgggacgcggcggaggccgcctggcggcaggcgctcgacggcctgaccgagccgacacatctcgtcccgcacgacccgcagcggatccctgccctaccggagcggctgtcactcgagttgccggaacaggcgaccgaggcgctcaccgcactggcccgctcacgcggcctgaccctgaacacgctggtccagacggcgtggagcatcgtgctcggcaggctcaccggccgggcggacgtcgtcttcggcgcgacggtcgccggacgcccgcccgaactggccggggtcgagcggatgatcggcctgttcatcaacaccatcccggtgcggatcgtgctcgacccggccgagaagctgggcgacctgctcgaccgggtccaggacgagcagtccggattgctcgcgcatcagcacttcgggctcgccgacatccagcgcggtgccgggctgggggagctgttcgacaccctcatggtgttcgagagctacccgggcgcggacgaggacgagacgggcgccgggttgcgcggggagatcgtgcgccacgacgactccacgcactatccgctcacctgggcggtggagccgggagcacggctgaagctcaccgcggagtaccgcgccgacctgttcgacgccggcctgccggaacggcttctgcccgcgatggagcgggtgctcgcggcgatggtgtccgatgtggacgctcccgtcggccgggccgacatcctgggcgaggccgacctggacctgatcgtgcgcgagtggaacgggacggccctcgtcgtcgaaccgtccaccgtggccggtctgttcgaagctcaggtccggcgttcgcccgacgcgacggcggtggtgtccggtgtggtcaccctgaccttcaccgaactcaacgaacgcgccaaccggctggccagggcgctggtcgcccacggagtggggcctgaggacttcgtcgccttggcgctgccgcgttcggccgacgtgctgctggcgatcctcgcggtccacaaggccggggcggcatacctgccgctcgacccggactacccggccgaacggctggccgcgatgctcgccgacgcgcggcccaaggtgctcctgaccgagcccgagctgctggaggcgttgccccggaccgggatcgagacgctggcgctctgcgcgctcgtcaccgaagaccggcaggcacacgacctgaccgaccgggaccgcgtccgcccgctcgtcccggagcatccggcgtacgtgatctacacgtcgggctcgaccggcacgcccaagggtgtcgtcgtcccgcatcgcggcgtggtgaacctgttccacagccaccgcgccaccttgcaccggcccgcggtcgaggccaccggcaaaccgcatctgcgggtcgggcacgcgtggtcgttctccttcgacgcgtcctggcagccgcaactgtggctgctcgacgggcacgccgtgcacatcgtggacgaggagacgcggcgcgaccccgaactgctggccgcgatgatcgaacgggagggcttcgacttcatcgaggtcacgccgtcgttcttcgcgcagatggcggacgcgggcctgctcgacggcgaccggtgccccctcgcggtgatcggtgtcggcggcgaggcggtgcccgagagcctgtggcggcgtctggccactttggacggcaccgaggccttcaacctgtacggacctaccgagtccacagtggacgcattggtcgcgcggatcgccgacagcgagaagccgctggtgggccgtccggtcgcgaacacccacgcctacgtgctggacgcggcgctgcggccggtgccgcccgggatcaccggtgagctgtacctcgccggtgccgggctcgcccgtggttatctcgggcgggcggcgctgacggcggagcgattcgtcgcggatccgttcggccccgcgggtgcccggatgtaccgtaccggtgacctcgcccgctggaccgcggacgggcgtctggacttcggcgggcgtgccgacgaccaggcgaagatccgcgggttccgggtcgagcccggtgagatcgaggcggtgctcgaacggcacgacggcgtcgcgcaggctgtcgtcctggtccgtgaggatcgccctcgcgtcaagcaactcgtggcgtacgtcgtgtccactgtggagcgggacgagctgcggcggttcgcggccgagacgttgccggactacatggtcccggcggcgttcgtcacgctcgacgcgcttcccacgctgtcgaacgggaagctggaccgcaaggcgttgccggcgccggactactcggagctggccggtggccgcgcgcccgagacagcgctggaaaagacgttgtgcgcgcttttcgccgaggtgctcgatgtgccggagctgggtgccgatgacgacttcttcgccctcggcggggacagcatcgtcgcgatgcggctggtcggcagggcgcgcgcggccggactgaagatcacgccgcacgacgtgttcgcgggccggacggtcgcggcgctggcggaggtcgcggaacggatcgaggacgacggcgcgatcgcgctcacgccgatcatgcacgcgctccgggagctcggcggtccgatcgacggttaccaccaggccgctctggttcggacacccgccgggatgaccggcgagcagctgaagaccgtgctcggcgcgctcgtggcgaggcacgacatgctgcgcgcgcggctcgaccggtcgggcgacggctgggtgctccggggggccgaatcggccgaggtgcacgtcgagcgtgtcgacgccgaggacggcgatctgttcgctctgatcgagaagcacgcggtgccgactcgttcccggctgaacccggacgacggcgtgatgctgagcgcgacctggttcgatgcgggctcgtcggggccaggccggttgctgctgatgatccatcacctggccgtcgacggtgtctcgtggcggatcctgctgccggacctcgcttccgcgtgggaatcggtcgcccgcggtgaagcgccgacgttgcccactgtggagacctcgttccggcgatggtcgcgagcactgtccacacaggaccgttcacgtgagctggaccgatggaagtcggtgctgggcggtggggatccgatcccgctggaccggccgctcgacccggcccgggacgtgcaggcgaccgtgcggaaggtgtcgctgcggctgcccgccgagcggacggccccgctgctgtcccgggcgacgatcaacgacgtcctgctcaccggtctcgccctcgccgtcgcccgctggcggcgggacgggggacgggcggtgctggtcgacgtcgaggggcacggccgcgaagaggaactggcggacctttcgcaaaccgtcgggtggttcaccagcgtcgtcccgatgtgcctggacctgggcgacgtcgacgtggaggacgcgttcgcgggcggtcccgccgccgacgacgcgctgaagcgggtccaggctcacctcgatggggtgccggacggcggcatcgggcatggcgtgctccgctatctcgaccctcgtaccgggcctgaactcgcgaagttcgcgaagccgcagatcgagttcaactacctgggccggatcggcgtccccgaagcgaccgactggtcgtacgcggcggaggccgaggcggccgaactcggcgccgatgacggcatgccggtctcgcacgcgctgacggtcaacgtcgtggccgaggaccgccccggcggtcccgaactgagcgcacactggtcgtggccggaggccgtgctcaccgaagacgcggtgcgggaactcgcggaaggctggttcctcgcgctggacgcgctcactcgccgggctgattcaccggccgtacagaaggagaacaaggaatga

SEQ ID NO: 12 - protein, 69 aa, Amycolatopsis orientalis
MTNPFENPDGTYLVLVNDEGQHSLWPEFVEVPAGWTTKFGPASRPECLEYVETNWTDLRPASLVKAMES

SEQ ID NO: 13 - DNA, 210 bp, Amycolatopsis orientalis
atgacgaacccgttcgagaaccccgacggcacgtacctggtgctcgtcaacgacgaagggcagcacagcctgtggcccgagttcgtcgaggtcccggcgggctggaccacgaagttcggcccggcctcgcgtccggagtgcctcgaatacgtcgagaccaactggaccgaccttcggcccgcttcgctcgtcaaggccatggagtcctga

SEQ ID NO: 14 - protein, 7697 aa, Amycolatopsis orientalis
CCTTCACGGCGCAACGGCCGAAACCTCAGCTGGCCGGCTTGACCTCGGTCTTCGGGTCACCGTCGACCGCGGCGGCCAGCTGCGGGACGATCCGGTCGATCACGTACGGCAGGCTCAGTACGGTCACGAAGGAGACCGCGCTGCCGTAGTTGCCTTCGTTCTCGATGAAGACCTCGCGCTTTTCCTTCGCCACCTTGAGACCGGTGTAGAGCGCGTCCTTGCCGAGGATCTCCTGCGCCTTGGCCGGGGTGTCGATGAGCCACACGAGCGCGTCGCGGTCGAGCAGGTCGGTCCGTTCGGCGCTGAGACTCTTGCCGAACTTGTCACCGATGACGGCGTCGAGGTCGGCGGGCAGCTTGAAGCCGAGCGATTCGAGGATGTGCGAGCGGCCGTCCTGGCTGCCGTAGACGAAGTAGCCCTCGTAGGTCGACGCCATCAGCGCCGACTTGCCCGCGAACTCCGGGTGCTCCTTGCGAGCCTGCGCGAACTTGTCCTCGACGGCCTTGACCAGTTCGTTCGCCTTGGTGGCGCGCCCGACGGCCTGGCCGACCTTCTTGGTCGCCTCGTTCCACGGGATGCCGTAGTCGTTGTACTCCTTGGGCTGCGCGACGACCGGGACGCCGATCTGGCTCAGCTTGTCGTACTGCTCCTTGGTGAGCCCGGAGTACAGCGCGATGATCAGATCCGGCTTCAGCGCGGCGATCTTCTCGTACTGCGGCCCGTCCACGTCCTTGAGCACCTCGGGCAGCGCGGCACCGCCGAGCTTGTCGGTCGCCCACGGGCCGATCGTGCCGGGGAACTTGCCGAGCCACTCGGTGACCCCGACCGGGACCACGCCGAGGGCGAGCAGCGCGTCCTGTTCGGTCAGGCCGACCGTGACGACGCGCTTGGGCTCCTGGGTGATGGTGGTGCTGCCGTACTTGTGCTCGATGGTGGTCGGGAACGCGTTCGCGTCGACCGCGGAGCCGGCGGAGTTCTGCGCGGCCGGGCTGGCCGACTCGCCGCCTCCTCCGCACGCGGTGAGCGAGGCGGCCAGCACCAGGGTGGACGCCAGCAGGCCGGCGGTCTTCAGGCGCCCGCGCACGGCGGAGAGGGTGGGGATTCGGGCCATCATCCGTCCTTCGTGATCGGAGGTCCGGCAGGATGCCGGATTAATCGAGAATCAGGTTTGGCTAACCTAAGACGCCGAGGGGCCGATTGCAACCGGTGACGGTGCGTCCCGGGTCACGTCTGTGAACGGTTGTCCGACCAGGCCGCCCAAAGCGTTGCGTATTGACCGCCCGAGGCCACGAGTTCGTCGTGTGTCCCGCTTTCGACGATCGCGCCCGCGTCGAGCACGACGACGCGGTCCGACGCCGCCGCCTGCGTCAGGCGGTGCGCGACGACGAGACCGGTCCGTCCCTCCAAAGCGGCGGCCGCGGCGGCTTCGAGGACTTTCGCGCCCGCGCTCCCGGCTTCCGCGGTCGCCTCGTCGAGGATCGCGATCGGCGGGTCCGCGAGCACCAGCCGCGCCAGCGCCAGCTGCTGCGCTTGGGTGACCGTCAGCTGATGACCGCCCTCGCCGACGATCGTCGCGAGACCCAAGGCCTCCGCCCAGCCCCGTGCGCCGACCTTTTCCAGTGCTGCCAACAGATCCGAGTCGGACGCGGCCGGGCGCGCCAGACGAAGGTCGTCCGCCAGCGAACCGGCGAAGACGTGCACCTCCTGGCTGATCAGCGCGACCGCCCGCCGCGTGGCGGACGGCCCGAGCTCGTCGAGCGGGACCCCGCCGACGGAGATCGTTCCCGAAGCCGGGGCGTGGATCCCGGCGATCAGCTTGGCCAGCGTCGTCTTCCCGGCCCCGCTCGCCCCGACCAGCGCGACCCGCTCACCGGGGGAGAGGTCGAGGTGGACGTCCCGCAGCACCGGATGTCCGTCCACATAGGAATGAGAGATCCCGGCCGCCTTCACCGACGCGTCGACGGGCCTCGCGGGGCGCTCGGGTTCGCGTGGCGCGGGCAGGTCGGCGACCCCGATCAGCCGGGCGAGGCTCGCGGCGGCCGCTTGCGCGTCGTCCACCAGCGCGAGCGCGGTGTTGATCGGGCCGAACAGGCTGTGGAAGTACAACGCCGCCGCTGTCGCGGCACCGACGGTCACCGCGTCCGCGCCCACCAGCAGGAAACCGGCCGCGAGCACAGCCGAGAGACCGACGAACTCGGCGAGGTTGAGCCGGGCGAAGAACTTCGTCATCAGCCGGACACCGCGCAGCATCAGGTCCATCGCGCCCTCGGACCGCTGCCGGACGCGCTCCACGTGGGCGTCCGCGAGCCGGAACGCGCGGACGGTCTTCGCACCGCCGATCGTGTCGAGCAGTTGCTGCTGCTGCGAACCCGTCGCGATCCGCTGATTGGCGTACAACGGGACGGCGTTGCGGACGTACCAGCGCACGGTGTGCAGCTGGATCGGTGCCGCCAGCAGCGCGGCGAGCAGGAACCGCCAGTCGAGCACGGCGAGCGCGCCGAGGGTCAGCACGATGGTGAGCACCGAACGGCCCAGTTCGGGGAGGGCTTCGCGGACCGCCCGCGCGACCACCGTGACGTCGTTGGTGACGCGGGCGGTCAGATCGCCGGACCCCGCGCGTTCGATCTGCTCCAGCGGGAGCCCCAGCGCGCGGTCGACGAACCGTTCACGCAGTTCCGCCAGCAGGGTTTCGCCGAGCTTCGCGGTGAGCGAGACACCCAGCGCCGTCGCGATCGCCTGTGCCACGGCGACCAGGACCAGCGCGACCACCGGCGTCGCCAGTTCGGTCGCGGGCCGCTTCTCGATGACCAGATCGACGATGTGGCCGAGCAGGCGGGCGGTGAGCAGCCCGATCGCGGTGGCGCCGACGAGCATCGTGAGCGCGCCCGCGGCGCGGGCCTTCGATGTGCGGAGCAGCTCGCCGAGGACCGCCCGGATCCGTTTGCCGTCGGCGGTGGGCAGGAGTTCGCGGGTCATGACAGCACCGCCGCGCGGTAGTCGGCGCGATCACGGACGAGTTCGGCGTGGCCGCCCTCGCCGGTGATCCGGCCGCCGTCGAGTACGACGACGCGGTCCGCCACGGCGAGCAGGGCGGGGCTCGTGGTCACCAGGATCGTGGTCCGTCCTCGTCGTAGCCGGGCGAGCCCGGTGGCGATCTTCGCCTCGGTCACCGTGTCGACCGCCGTCGTCGGGTCGTGGACGACGAGCACCGGCGCGTCGGCGGCGAGCGCGCGGGCGAGCGCGACCCGTTGCCGCTGGCCGCCGGAGAGGGAACGGCCGCGTTCGGTGACCGCGGTCCCGGTGCCGTCCGGCAGCGCCGCGGCGACCTCGTCCGCCGCCGACGCGGCCAGCGCCTCGTCGAGCGGGCGGTCCCCGGCGTGGACGTTCTCGGCGAGCGTCCCTTCGAACAGGTCCGCGTCGTGCGCCGCGACCAAAACGACCTCCCGTACCCGTTCGGGGTCCACAGTGGACAGATCCACGCCGTCGACGCGCACCCGGCCCTCGGCGGGGTCGGCCGCGCGGCCGAGGCAGTCGAGCAGATCGGTCGCGGCGGCCGGATCCGCGGCGACGACGCCGATCAGCTGTCCGGCCGGGATGTCGAAGTCGAGATCGCGAAGGCTGCCGTATGAGACCGCGTCGAGCCGCACGTTCCCGCCGGCGGGTGACGGGAGTTCGCCGTCGCCCGGTTCCACCGCCGCGGGCGTGGTCAGCAGGTCGGCGACCCGGGACGCGGACGCGCGGCCCTGCGCCAGTTCGCCGTTGACCCACGAGAAGATCAGGAACGGCGTGAGCAGGAACTGGGCGAGCCCGACGGCCGCGACGAGGTCGCCGACGCTGATCGATCCCGACGACGCGAGATGTCCGCCGACGAGGGCGACGATCGCGATGAAGACGCCGGTCAGCGCGAGCACCGCGCCGTCGTGCCAGGCCTTGGCCCGCGCGGCACGCAGTGTCGCCTTGAGCGAGTCCTGGCTCGTGTCGCGGTAGCGGGCGACGGCGGCCGGTTCGGCGCCGATGCCCTTGAGCACCCGCAAGCCGGAGACCAGATCCGTGGCGACCCCCGAGGCGTGCGCCGACCGCTCCTGTTCCGCCTCGCTGCGGCGTTCGAGCGGTTTGCCGATGAGATGCGCGAGATACAGCAGCGGTGGCGTGCCCAGCAGCACGAGGAGCCCCAGCGGAAGCGAGATGCGCAGGAGGGCCACCGCGCTGACCAGTACCCCGGCCAGCGCCGCGATCCCGAACGGCAGCACCGCGTTCACCACCCCGACGCGTTGCGCGTCGCTGGTGCCGATGTTGACCAGTTCCCCGGCGAGCTTGCCGTTCTCGGCGCCGCCGCCGGGATGCAGGACGCGACGGCTGATCGCGATGCGGAGTTCGTGCGCGGCCCGCAGCGACGCGCCTTCGGCCGCGCGCGCGGCGAACCGGTAGCTGTAGGACAGTCCCGCGAAGACCACGGCGAGCACGGCGAGCCAGAACACGAGCGTGCCCACGGACCCGCCGTCGACCGCCTGGTCGATCACCACGCCGATGATCACCGGAACGAGTGCTTCGCCGCCTTGGTGCCCGGCCGCGAGCACGGATGCGAGCGTGACCTGCCGTCGCTGCCCGGAAATCGCCTGGCGCAGGACGCTTCGGCTGGTGGTGGCCGTTGCGGTCACCGGCGCCTCCTAGTTCGGGGTAGGTAAGGCTAGTCTAAGTAGGTAGCTCGTTGAGGTGAACTCCGGCGTTCGGTTAGCCTCGCCTCACTCGAAGGAGGTCCCGTGGTCGTCACCGAACGCCCGCCCGAAGCGGCGTCCCCGCCCAGCACGGCGGCACGGACGTTCCGCGCCGGCGGTCTGGTGGTCGGGCTCGCGCTGCTGGCGCTCGTCTTCCTGCTCAGCATCTGGGTGGGGTCGAAGAGCATCCCGTTCACCTCCACCTGGTCGGTGCTGTGGCACAACGACGGCTCGACCGACGCGGTGATCATCCACGACCTCCGTATCCCGCGGACCCTGCTGGGCCTGGTCGTCGGCGCGGCGCTCGGGCTCGCGGGCGCGGTGATGCAGGCGCTCACCAGGAACCCGCTCGCCGACCCCGGCCTGCTAGGTGTCAACACCGGTGCCGCGGCCGCCGTCGTCTCGGGGATCGCCTTCGCCGGTGTCACCAGCGTGCTCGGCTACATCTGGTTCGCTTTCGCCGGCGCCGCGATCGCCTCGGTCGTGGTCTACCTCCTCGGCTCGGCCGGCCGCAGCGCCACGCCGGACCGGCTCGTGCTCGCCGGTTCGGCCGTGATGGCGGTGCTCCAGGCGTACATCGTCGGTGTGCTGCTGCTGATGCCGGAGACGTTCAACCAGTTCCGGTTCTGGAACGTCGGCTCGCTCAGCGGCCGCAAATGGGACGTCTTCCTGCAGATCTCGCCGTTCGTCGTACTCGGCGTGATCATCGCGCTGCTGCTCGCGCGCCCGCTCAACGTCCTCGCGCTCGGTGATCAGACCGGCAAGGCGCTCGGCGCCCACGTGGGCCGCACCCGGGTGCTGGGCGCGGTCGTGGTGATGCTGCTCTGCGGCGCGGGCACCGCGGCGATCGGACCGGTCCTGTTCCTCGGGCTCGCCGTGCCCCACGCCGCGCGGATGCTGGTCGGCCCGGATCAGCGCTGGGTGCTGCCGTATTCGATGCTGCTCGCGCCGATCGCGCTGATCGGCGCCGACGTCGTCGGCCGCGTGCTGAACCCGCCGGAGGAACTGCAGGCGGGGATCGTCGTCGCGTTCCTCGGGGCGCCGGTGTTCATCGCGCTCTGCCGCCGCCGGAAGCTGGCCCGCCTGTGACCGCGATCAAGGAGCACCCACGGATGAGCACCCGCGTCCTGCGCACCCCGGGCGGCGGGCTTTCCCTGCGCGTCACCCCGCGGGCGGCGATCGTCGTCGCCCTGCTGGCCGTCGCCGTGTTCTTCGTCGGCGCGGTCAGCATGACCACCGGCGACTACCCGCTGTCCGTCGGCGAGGTGATCCAGACGCTGTTCGGCTTCGGCGACCGGGCCACCGAGTTCATCGTGACCACGCTGCGGCTGCCCCGGCTGCTGACCGCCCTGCTGGTCGGCGGCGCGCTCGCGGTCAGCGGCGCGATCCTGCAGAGCCTGTCCGGGAATCCCTTGGGCAGCCCCGATTTCATCGGCTTCACCGAAGGCGCGGCGGCCGGTGCGCTGCTGACGATCATCGTCGTGCACGGCGGCATGCTCCAGGTGTCGTTGGGAGCGCTGGTCGGCGGCGTCGCGACGGCGGCGTTGATCGCGCTGCTCGCGCTGAAAGGCGGCGTGCACACCTTCCGGCTGATCCTCGTCGGCATCGGCGTGAACGCGATGCTCATCGCGTTCAACTCCTACCTGATCACCAGGGCCTCCTGGCAGGACGGGCTCGCGGCGCAGGCCTGGCTGGTCGGCGGGCTCAACGGACGCGGCTGGGAGCACGTCGTCCCGGTCGCGATCGCCCTCGCGGTCCTGCTTCCGCCCGGGTTCGTCTACGGCAGAAGGCTTTCGCTGCTGGAGATGGGTGACGATTCGGCCATGGGGCTCGGCGTGCCGGTCCAGCGGAGCCGGATCGTGCTGCTCGCCGTCAGTGTCGGGCTGTGTTCGATCGCGACCGCGGCCGCCGGGCCGATCGCGTTCCTGGCCCTCGCCGCGCCGCAGCTTTCGAAGCGGCTCACCCGCTCTTCGGGGCCGGGGCTCGCCGCCGCCGCGTTGATGGGCATGCTCCTGCTGGTCACGAGCGATCTGGTCACGCAGCAGTTCTTCGCGGGACTGCCCGTGGGCCTGGTGACCGGGGCGATCGGCGGGCTGTACCTGGCCTGGCTGCTGGTGTCCCGGCGCGGGGTCAGCCCGGCTGCGCGCTGACCGGCTCGATCGGGGCGGGCTCGCCGAAGCGGGCCAGTGCGAGCGCGGCCGAAACGGCGAGCACGAAGCCGACCGCCGCCACGGCCGCGAAACCCGGTCGCGTGGTGTCGCCCAGCACGAGCACGCCGATCAGGGACGGGAAGACGGTCTCGCCGATCACCATCATCGCGGTCGAGGTGGTGACGCTGCCGCGCTGCAACGCGGTGGCGTAGAACAACATCGCCAGCGCGCCCGCCACACCCAGCGTGTACGTGGCCGGGTCGGCCAGCAGATCCAGCGGGGCGAGGCTGGTGAGCGCGCGGCCGGACAGCGCGACGACACCGAAGCACAGTCCGGCGATCAGGCCCAGCGACGGTGTCCGGATCCGCCGCGACGCGCGGCCCGCGAACATCCCGGCCGTGCCGAGGACGACCACCGCACCGATCAGCACGAGCCGGAAACCCAGCCCGGTCGGCTCCGAGCCCTCGCTCTCCGCCGACATCCCGAGCAACGCGAGCCCGGCGCAGACGACCGCGACCGCCGTCCACTCGCGACGGCCCAGCCGGACGCCGAGGGTCCGCGCGGCGACCGCGGTCACCGCGAGACTGCCGGCCAGCGCCGCCTGGACCACGAACAGCGGCAGCACGCGCAAGGCGGCCACCTGGGCGACGAACCCGATGACGTCCAGCGCCAGTCCCGCGACGAACTTCCATTGCCGCAGCACCCGCACGAGCAGCCGGGGATCGACGCGGTCTTCGGCGCCCGAAGTCGCCTTCGCGGCGATCGCCTGCATCACCGAAGCGACGCCGTAGGCACAGGCCGCGAAGAACGCGCAAATCAATCCCCACCACAT

SEQ ID NO: 15 - protein, 362 aa, Amycolatopsis orientalis
MMARIPTLSAVRGRLKTAGLLASTLVLAASLTACGGGGESASPAAQNSAGSAVDANAFPTTIEHKYGSTTITQEPKRVVTVGLTEQDALLALGVVPVGVTEWLGKFPGTIGPWATDKLGGAALPEVLKDVDGPQYEKIAALKPDLIIALYSGLTKEQYDKLSQIGVPVVAQPKEYNDYGIPWNEATKKVGQAVGRATKANELVKAVEDKFAQARKEHPEFAGKSALMASTYEGYFVYGSQDGRSHILESLGFKLPADLDAVIGDKFGKSLSAERTDLLDRDALVWLIDTPAKAQEILGKDALYTGLKVAKEKREVFIENEGNYGSAVSFVTVLSLPYVIDRIVPQLAAAVDGDPKTEVKPAS

SEQ ID NO: 16 - DNA, 1089 bp, Amycolatopsis orientalis
atgatggcccgaatccccaccctctccgccgtgcgcgggcgcctgaagaccgccggcctgctggcgtccaccctggtgctggccgcctcgctcaccgcgtgcggaggaggcggcgagtcggccagcccggccgcgcagaactccgccggctccgcggtcgacgcgaacgcgttcccgaccaccatcgagcacaagtacggcagcaccaccatcacccaggagcccaagcgcgtcgtcacggtcggcctgaccgaacaggacgcgctgctcgccctcggcgtggtcccggtcggggtcaccgagtggctcggcaagttccccggcacgatcggcccgtgggcgaccgacaagctcggcggtgccgcgctgcccgaggtgctcaaggacgtggacgggccgcagtacgagaagatcgccgcgctgaagccggatctgatcatcgcgctgtactccgggctcaccaaggagcagtacgacaagctgagccagatcggcgtcccggtcgtcgcgcagcccaaggagtacaacgactacggcatcccgtggaacgaggcgaccaagaaggtcggccaggccgtcgggcgcgccaccaaggcgaacgaactggtcaaggccgtcgaggacaagttcgcgcaggctcgcaaggagcacccggagttcgcgggcaagtcggcgctgatggcgtcgacctacgagggctacttcgtctacggcagccaggacggccgctcgcacatcctcgaatcgctcggcttcaagctgcccgccgacctcgacgccgtcatcggtgacaagttcggcaagagtctcagcgccgaacggaccgacctgctcgaccgcgacgcgctcgtgtggctcatcgacaccccggccaaggcgcaggagatcctcggcaaggacgcgctctacaccggtctcaaggtggcgaaggaaaagcgcgaggtcttcatcgagaacgaaggcaactacggcagcgcggtctccttcgtgaccgtactgagcctgccgtacgtgatcgaccggatcgtcccgcagctggccgccgcggtcgacggtgacccgaagaccgaggtcaagccggccagctga

SEQ ID NO: 17 - protein, 583 aa, Amycolatopsis orientalis
MTRELLPTADGKRIRAVLGELLRTSKARAAGALTMLVGATAIGLLTARLLGHIVDLVIEKRPATELATPVVALVLVAVAQAIATALGVSLTAKLGETLLAELRERFVDRALGLPLEQIERAGSGDLTARVTNDVTVVARAVREALPELGRSVLTIVLTLGALAVLDWRFLLAALLAAPIQLHTVRWYVRNAVPLYANQRIATGSQQQQLLDTIGGAKTVRAFRLADAHVERVRQRSEGAMDLMLRGVRLMTKFFARLNLAEFVGLSAVLAAGFLLVGADAVTVGAATAAALYFHSLFGPINTALALVDDAQAAAASLARLIGVADLPAPREPERPARPVDASVKAAGISHSYVDGHPVLRDVHLDLSPGERVALVGASGAGKTTLAKLIAGIHAPASGTISVGGVPLDELGPSATRRAVALISQEVHVFAGSLADDLRLARPAASDSDLLAALEKVGARGWAEALGLATIVGEGGHQLTVTQAQQLALARLVLADPPIAILDEATAEAGSAGAKVLEAAAAAALEGRTGLVVAHRLTQAAASDRVVVLDAGAIVESGTHDELVASGGQYATLWAAWSDNRSQT

SEQ ID NO: 18 - DNA, 1752 bp, Amycolatopsis orientalis
atgacccgcgaactcctgcccaccgccgacggcaaacggatccgggcggtcctcggcgagctgctccgcacatcgaaggcccgcgccgcgggcgcgctcacgatgctcgtcggcgccaccgcgatcgggctgctcaccgcccgcctgctcggccacatcgtcgatctggtcatcgagaagcggcccgcgaccgaactggcgacgccggtggtcgcgctggtcctggtcgccgtggcacaggcgatcgcgacggcgctgggtgtctcgctcaccgcgaagctcggcgaaaccctgctggcggaactgcgtgaacggttcgtcgaccgcgcgctggggctcccgctggagcagatcgaacgcgcggggtccggcgatctgaccgcccgcgtcaccaacgacgtcacggtggtcgcgcgggcggtccgcgaagccctccccgaactgggccgttcggtgctcaccatcgtgctgaccctcggcgcgctcgccgtgctcgactggcggttcctgctcgccgcgctgctggcggcaccgatccagctgcacaccgtgcgctggtacgtccgcaacgccgtcccgttgtacgccaatcagcggatcgcgacgggttcgcagcagcagcaactgctcgacacgatcggcggtgcgaagaccgtccgcgcgttccggctcgcggacgcccacgtggagcgcgtccggcagcggtccgagggcgcgatggacctgatgctgcgcggtgtccggctgatgacgaagttcttcgcccggctcaacctcgccgagttcgtcggtctctcggctgtgctcgcggccggtttcctgctggtgggcgcggacgcggtgaccgtcggtgccgcgacagcggcggcgttgtacttccacagcctgttcggcccgatcaacaccgcgctcgcgctggtggacgacgcgcaagcggccgccgcgagcctcgcccggctgatcggggtcgccgacctgcccgcgccacgcgaacccgagcgccccgcgaggcccgtcgacgcgtcggtgaaggcggccgggatctctcattcctatgtggacggacatccggtgctgcgggacgtccacctcgacctctcccccggtgagcgggtcgcgctggtcggggcgagcggggccgggaagacgacgctggccaagctgatcgccgggatccacgccccggcttcgggaacgatctccgtcggcggggtcccgctcgacgagctcgggccgtccgccacgcggcgggcggtcgcgctgatcagccaggaggtgcacgtcttcgccggttcgctggcggacgaccttcgtctggcgcgcccggccgcgtccgactcggatctgttggcagcactggaaaaggtcggcgcacggggctgggcggaggccttgggtctcgcgacgatcgtcggcgagggcggtcatcagctgacggtcacccaagcgcagcagctggcgctggcgcggctggtgctcgcggacccgccgatcgcgatcctcgacgaggcgaccgcggaagccgggagcgcgggcgcgaaagtcctcgaagccgccgcggccgccgctttggagggacggaccggtctcgtcgtcgcgcaccgcctgacgcaggcggcggcgtcggaccgcgtcgtcgtgctcgacgcgggcgcgatcgtcgaaagcgggacacacgacgaactcgtggcctcgggcggtcaatacgcaacgctttgggcggcctggtcggacaaccgttcacagacgtga

SEQ ID NO: 19 - protein, 561 aa, Amycolatopsis orientalis
VTATATTSRSVLRQAISGQRRQVTLASVLAAGHQGGEALVPVIIGVVIDQAVDGGSVGTLVFWLAVLAVVFAGLSYSYRFAARAAEGASLRAAHELRIAISRRVLHPGGGAENGKLAGELVNIGTSDAQRVGVVNAVLPFGIAALAGVLVSAVALLRISLPLGLLVLLGTPPLLYLAHLIGKPLERRSEAEQERSAHASGVATDLVSGLRVLKGIGAEPAAVARYRDTSQDSLKATLRAARAKAWHDGAVLALTGVFIAIVALVGGHLASSGSISVGDLVAAVGLAQFLLTPFLIFSWVNGELAQGRASASRVADLLTTPAAVEPGDGELPSPAGGNVRLDAVSYGSLRDLDFDIPAGQLIGVVAADPAAATDLLDCLGRAADPAEGRVRVDGVDLSTVDPERVREVVLVAAHDADLFEGTLAENVHAGDRPLDEALAASAADEVAAALPDGTGTAVTERGRSLSGGQRQRVALARALAADAPVLVVHDPTTAVDTVTEAKIATGLARLRRGRTTILVTTSPALLAVADRVVVLDGGRITGEGGHAELVRDRADYRAAVLS

SEQ ID NO: 20 - DNA, 1686 bp, Amycolatopsis orientalis
gtgaccgcaacggccaccaccagccgaagcgtcctgcgccaggcgatttccgggcagcgacggcaggtcacgctcgcatccgtgctcgcggccgggcaccaaggcggcgaagcactcgttccggtgatcatcggcgtggtgatcgaccaggcggtcgacggcgggtccgtgggcacgctcgtgttctggctcgccgtgctcgccgtggtcttcgcgggactgtcctacagctaccggttcgccgcgcgcgcggccgaaggcgcgtcgctgcgggccgcgcacgaactccgcatcgcgatcagccgtcgcgtcctgcatcccggcggcggcgccgagaacggcaagctcgccggggaactggtcaacatcggcaccagcgacgcgcaacgcgtcggggtggtgaacgcggtgctgccgttcgggatcgcggcgctggccggggtactggtcagcgcggtggccctcctgcgcatctcgcttccgctggggctcctcgtgctgctgggcacgccaccgctgctgtatctcgcgcatctcatcggcaaaccgctcgaacgccgcagcgaggcggaacaggagcggtcggcgcacgcctcgggggtcgccacggatctggtctccggcttgcgggtgctcaagggcatcggcgccgaaccggccgccgtcgcccgctaccgcgacacgagccaggactcgctcaaggcgacactgcgtgccgcgcgggccaaggcctggcacgacggcgcggtgctcgcgctgaccggcgtcttcatcgcgatcgtcgccctcgtcggcggacatctcgcgtcgtcgggatcgatcagcgtcggcgacctcgtcgcggccgtcgggctcgcccagttcctgctcacgccgttcctgatcttctcgtgggtcaacggcgaactggcgcagggccgcgcgtccgcgtcccgggtcgccgacctgctgaccacgcccgcggcggtggaaccgggcgacggcgaactcccgtcacccgccggcgggaacgtgcggctcgacgcggtctcatacggcagccttcgcgatctcgacttcgacatcccggccggacagctgatcggcgtcgtcgccgcggatccggccgccgcgaccgatctgctcgactgcctcggccgcgcggccgaccccgccgagggccgggtgcgcgtcgacggcgtggatctgtccactgtggaccccgaacgggtacgggaggtcgttttggtcgcggcgcacgacgcggacctgttcgaagggacgctcgccgagaacgtccacgccggggaccgcccgctcgacgaggcgctggccgcgtcggcggcggacgaggtcgccgcggcgctgccggacggcaccgggaccgcggtcaccgaacgcggccgttccctctccggcggccagcggcaacgggtcgcgctcgcccgcgcgctcgccgccgacgcgccggtgctcgtcgtccacgacccgacgacggcggtcgacacggtgaccgaggcgaagatcgccaccgggctcgcccggctacgacgaggacggaccacgatcctggtgaccacgagccccgccctgctcgccgtggcggaccgcgtcgtcgtactcgacggcggccggatcaccggcgagggcggccacgccgaactcgtccgtgatcgcgccgactaccgcgcggcggtgctgtcatga

SEQ ID NO: 21 - protein, 347 aa, Amycolatopsis orientalis
VVVTERPPEAASPPSTAARTFRAGGLVVGLALLALVFLLSIWVGSKSIPFTSTWSVLWHNDGSTDAVIIHDLRIPRTLLGLVVGAALGLAGAVMQALTRNPLADPGLLGVNTGAAAAVVSGIAFAGVTSVLGYIWFAFAGAAIASVVVYLLGSAGRSATPDRLVLAGSAVMAVLQAYIVGVLLLMPETFNQFRFWNVGSLSGRKWDVFLQISPFVVLGVIIALLLARPLNVLALGDQTGKALGAHVGRTRVLGAVVVMLLCGAGTAAIGPVLFLGLAVPHAARMLVGPDQRWVLPYSMLLAPIALIGADVVGRVLNPPEELQAGIVVAFLGAPVFIALCRRRKLARL

SEQ ID NO: 22 - DNA, 1044 bp, Amycolatopsis orientalis
gtggtcgtcaccgaacgcccgcccgaagcggcgtccccgcccagcacggcggcacggacgttccgcgccggcggtctggtggtcgggctcgcgctgctggcgctcgtcttcctgctcagcatctgggtggggtcgaagagcatcccgttcacctccacctggtcggtgctgtggcacaacgacggctcgaccgacgcggtgatcatccacgacctccgtatcccgcggaccctgctgggcctggtcgtcggcgcggcgctcgggctcgcgggcgcggtgatgcaggcgctcaccaggaacccgctcgccgaccccggcctgctaggtgtcaacaccggtgccgcggccgccgtcgtctcggggatcgccttcgccggtgtcaccagcgtgctcggctacatctggttcgctttcgccggcgccgcgatcgcctcggtcgtggtctacctcctcggctcggccggccgcagcgccacgccggaccggctcgtgctcgccggttcggccgtgatggcggtgctccaggcgtacatcgtcggtgtgctgctgctgatgccggagacgttcaaccagttccggttctggaacgtcggctcgctcagcggccgcaaatgggacgtcttcctgcagatctcgccgttcgtcgtactcggcgtgatcatcgcgctgctgctcgcgcgcccgctcaacgtcctcgcgctcggtgatcagaccggcaaggcgctcggcgcccacgtgggccgcacccgggtgctgggcgcggtcgtggtgatgctgctctgcggcgcgggcaccgcggcgatcggaccggtcctgttcctcgggctcgccgtgccccacgccgcgcggatgctggtcggcccggatcagcgctgggtgctgccgtattcgatgctgctcgcgccgatcgcgctgatcggcgccgacgtcgtcggccgcgtgctgaacccgccggaggaactgcaggcggggatcgtcgtcgcgttcctcggggcgccggtgttcatcgcgctctgccgccgccggaagctggcccgcctgtga

SEQ ID NO: 23 - protein, 345 aa, Amycolatopsis orientalis
MSTRVLRTPGGGLSLRVTPRAAIVVALLAVAVFFVGAVSMTTGDYPLSVGEVIQTLFGFGDRATEFIVTTLRLPRLLTALLVGGALAVSGAILQSLSGNPLGSPDFIGFTEGAAAGALLTIIVVHGGMLQVSLGALVGGVATAALIALLALKGGVHTFRLILVGIGVNAMLIAFNSYLITRASWQDGLAAQAWLVGGLNGRGWEHVVPVAIALAVLLPPGFVYGRRLSLLEMGDDSAMGLGVPVQRSRIVLLAVSVGLCSIATAAAGPIAFLALAAPQLSKRLTRSSGPGLAAAALMGMLLLVTSDLVTQQFFAGLPVGLVTGAIGGLYLAWLLVSRRGVSPAAR

SEQ ID NO: 24 - DNA, 1038 bp, Amycolatopsis orientalis
atgagcacccgcgtcctgcgcaccccgggcggcgggctttccctgcgcgtcaccccgcgggcggcgatcgtcgtcgccctgctggccgtcgccgtgttcttcgtcggcgcggtcagcatgaccaccggcgactacccgctgtccgtcggcgaggtgatccagacgctgttcggcttcggcgaccgggccaccgagttcatcgtgaccacgctgcggctgccccggctgctgaccgccctgctggtcggcggcgcgctcgcggtcagcggcgcgatcctgcagagcctgtccgggaatcccttgggcagccccgatttcatcggcttcaccgaaggcgcggcggccggtgcgctgctgacgatcatcgtcgtgcacggcggcatgctccaggtgtcgttgggagcgctggtcggcggcgtcgcgacggcggcgttgatcgcgctgctcgcgctgaaaggcggcgtgcacaccttccggctgatcctcgtcggcatcggcgtgaacgcgatgctcatcgcgttcaactcctacctgatcaccagggcctcctggcaggacgggctcgcggcgcaggcctggctggtcggcgggctcaacggacgcggctgggagcacgtcgtcccggtcgcgatcgccctcgcggtcctgcttccgcccgggttcgtctacggcagaaggctttcgctgctggagatgggtgacgattcggccatggggctcggcgtgccggtccagcggagccggatcgtgctgctcgccgtcagtgtcgggctgtgttcgatcgcgaccgcggccgccgggccgatcgcgttcctggccctcgccgcgccgcagctttcgaagcggctcacccgctcttcggggccggggctcgccgccgccgcgttgatgggcatgctcctgctggtcacgagcgatctggtcacgcagcagttcttcgcgggactgcccgtgggcctggtgaccggggcgatcggcgggctgtacctggcctggctgctggtgtcccggcgcggggtcagcccggctgcgcgctga

SEQ ID NO: 25 - protein, 283 aa, Amycolatopsis orientalis
MWWGLICAFFAACAYGVASVMQAIAAKATSGAEDRVDPRLLVRVLRQWKFVAGLALDVIGFVAQVAALRVLPLFVVQAALAGSLAVTAVAARTLGVRLGRREWTAVAVVCAGLALLGMSAESEGSEPTGLGFRLVLIGAVVVLGTAGMFAGRASRRIRTPSLGLIAGLCFGVVALSGRALTSLAPLDLLADPATYTLGVAGALAMLFYATALQRGSVTTSTAMMVIGETVFPSLIGVLVLGDTTRPGFAAVAAVGFVLAVSAALALARFGEPAPIEPVSAQPG

SEQ ID NO: 26 - DNA, 852 bp, Amycolatopsis orientalis
atgtggtggggattgatttgcgcgttcttcgcggcctgtgcctacggcgtcgcttcggtgatgcaggcgatcgccgcgaaggcgacttcgggcgccgaagaccgcgtcgatccccggctgctcgtgcgggtgctgcggcaatggaagttcgtcgcgggactggcgctggacgtcatcgggttcgtcgcccaggtggccgccttgcgcgtgctgccgctgttcgtggtccaggcggcgctggccggcagtctcgcggtgaccgcggtcgccgcgcggaccctcggcgtccggctgggccgtcgcgagtggacggcggtcgcggtcgtctgcgccgggctcgcgttgctcgggatgtcggcggagagcgagggctcggagccgaccgggctgggtttccggctcgtgctgatcggtgcggtggtcgtcctcggcacggccgggatgttcgcgggccgcgcgtcgcggcggatccggacaccgtcgctgggcctgatcgccggactgtgcttcggtgtcgtcgcgctgtccggccgcgcgctcaccagcctcgccccgctggatctgctggccgacccggccacgtacacgctgggtgtggcgggcgcgctggcgatgttgttctacgccaccgcgttgcagcgcggcagcgtcaccacctcgaccgcgatgatggtgatcggcgagaccgtcttcccgtccctgatcggcgtgctcgtgctgggcgacaccacgcgaccgggtttcgcggccgtggcggcggtcggcttcgtgctcgccgtttcggccgcgctcgcactggcccgcttcggcgagcccgccccgatcgagccggtcagcgcgcagccgggctga

SEQ ID NO: 27 - protein, 23 aa, Streptomyces aizunensis
  MISC_FEATURE (1)..(1): X is any amino acid
  MISC_FEATURE (3)..(3): X is any amino acid
  MISC_FEATURE (6)..(6): X is any amino acid
  MISC_FEATURE (9)..(12): X is any amino acid
  MISC_FEATURE (18)..(18): X is any amino acid
  MISC_FEATURE (22)..(22): X is any amino acid
XNXGNXFGXXXXNNNDDXNAGXA

SEQ ID NO: 28 - protein, 10 aa, Streptomyces aizunensis
  MISC_FEATURE (1)..(2): X is any amino acid
  MISC_FEATURE (4)..(9): X is any amino acid
XXVXXXXXXN

SEQ ID NO: 29 - protein, 5 aa, Streptomyces ghanaensis
  MISC_FEATURE (1)..(2): X is any amino acid
  MISC_FEATURE (4)..(5): X is any amino acid
XXTXX

SEQ ID NO: 30 - protein, 10 aa, Streptomyces ghanaensis
  MISC_FEATURE (3)..(3): X is any amino acid
  MISC_FEATURE (5)..(7): X is any amino acid
  MISC_FEATURE (10)..(10): X is any amino acid
TFXTXXXTTX

SEQ ID NO: 31 - protein, 9 aa, Streptomyces ghanaensis
  MISC_FEATURE (5)..(5): N-methyl Valine
  MISC_FEATURE (6)..(9): X is any amino acid
VFNTVXXXX